(19) United States
(12) Patent Application Publication
(10) Pub. No.: US 2015/0313238 A1
(43) Pub. Date: Nov. 5, 2015

(54) METHODS AND COMPOSITIONS FOR CONTROLLING PLANT VIRAL INFECTION

(71) Applicant: MONSANTO TECHNOLOGY LLC, St. Louis, MO (US)

(72) Inventors: Johannes C. Hemmes, Roseville, CA (US); Lijie Jia, Davis, CA (US)

(21) Appl. No.: 14/436,032

(22) PCT Filed: Oct. 16, 2013
(86) PCT No.: PCT/US13/65193

§ 371 Date: Apr. 15, 2015

Related U.S. Application Data
(60) Provisional application No. 61/786,032, filed on Mar. 14, 2013.
(60) Provisional application No. 61/714,733, filed on Oct. 16, 2012.

(51) Int. Cl. A01N 57/16; C12N 15/113; G01N 33/569; A01N 25/30

(52) U.S. Cl. CPC A01N 57/16; A01N 25/30; C12N 15/1131; G01N 33/56983; C12N 2310/11; G01N 2333/175; G01N 2333/08; G01N 2333/01

(57) ABSTRACT

The present invention provides methods for topical treatment and prevention of Tospovirus and/or Geminivirus disease in plants. The invention further provides compositions for treatment of Tospovirus and/or Geminivirus disease in plants, and methods for reducing expression of a Tospovirus and/or Geminivirus gene and for identifying polynucleotides useful in modulating gene expression in plant viruses.

### CROSS REFERENCE TO RELATED APPLICATIONS

This application claims the benefit of U.S. Provisional Patent Application No. 61/714,733, filed Oct. 16, 2012, and U.S. Provisional Patent Application No. 61/786,032, filed Mar. 14, 2013, which are incorporated herein by reference in their entirety.

### INCORPORATION OF SEQUENCE LISTING

The sequence listing that is contained in the file named “MONS317WOsequencelisting.txt”, which is 251 kilobytes as measured in Microsoft Windows operating system and was created on Oct. 11, 2013, is filed electronically herewith and incorporated herein by reference.

### FIELD OF THE INVENTION

The methods and compositions generally relate to the field of plant disease control. More specifically, the invention relates to methods and compositions for treating or preventing symptoms associated with plant Tospovirus or Geminivirus infection.

### BACKGROUND OF THE INVENTION

Plant viruses of the genus Tospovirus and Geminivirus are economically important, causing reduced vegetative output and death of infected plants. Growers seeking to protect their crops from tospoviruses have traditionally attempted to guard their crops from the insect vectors, either with insecticide application, or with reflective mulches or plastic covers. Because these strategies have had limited success, and are expensive and labor intensive, alternative strategies for controlling Tospovirus and Geminivirus infection are needed.

### SUMMARY OF THE INVENTION

The embodiments described herein relate to methods and compositions for the prevention or treatment of viral infection in a plant comprising the topical administration to a plant of a polynucleotide comprising at least 18 contiguous nucleotides that are essentially identical or essentially complementary to a viral gene. The polynucleotide may be single-stranded DNA (ssDNA), double-stranded DNA (dsDNA), single-stranded RNA (ssRNA), or double-stranded RNA (dsRNA).

In one aspect, the invention provides a method of treatment or prevention of a Tospovirus infection in a plant comprising: topically applying to said plant a composition comprising an antisense single-stranded DNA polynucleotide and a transfer agent, wherein said antisense single-stranded DNA polynucleotide is complementary to all or a portion of an essential Tospovirus gene sequence or an RNA transcript thereof, wherein the symptoms of viral infection or development of symptoms are reduced or eliminated in said plant relative to a plant not treated with said composition when grown under the same conditions. In some embodiments, the antisense single-stranded DNA polynucleotide comprises at least 18 contiguous nucleotides that are essentially complementary to a sequence selected from the group consisting of SEQ ID NOs:13-46. In one embodiment, the transfer agent is an organosilicone surfactant composition or compound contained therein. In another embodiment, the composition comprises more than one antisense single-stranded DNA polynucleotide complementary to all or a portion of an essential Tospovirus gene sequence, an RNA transcript of said essential Tospovirus gene sequence, or a fragment thereof. In another embodiment, the antisense single-stranded DNA polynucleotide is selected from the group consisting of SEQ NOs:1-12 or a fragment thereof. In another embodiment, the Tospovirus is selected from the group consisting of bean necrotic mosaic virus, Capsicum chlorosis virus, groundnut bud necrosis virus, groundnut ringspot virus, groundnut yellow spot virus, impatiens necrotic spot virus, iris yellow spot viris, melon yellow spot virus, peanut bud necrosis virus, peanut yellow spot virus, soybean vein necrosis-associated virus, tomato chlorotic spot virus, tomato necrotic ringspot virus, tomato spotted wilt virus, tomato zonate spot virus, watermelon bud necrosis virus, watermelon silver mottle virus, and zucchini lethal chlorosis virus. In another embodiment, the essential Tospovirus gene is selected from the group consisting of nucleocapsid gene (N), coat protein gene (CP), virulence factors NSm and NSs, and RNA-dependent RNA polymerase L segment (RdRp/L segment). In another embodiment, the essential gene sequence is selected from the group consisting of SEQ ID NOs:13-46. In another embodiment, composition is topically applied by spraying, dusting, or is applied to the plant surface as matrix-encapsulated DNA.

In another aspect, the invention provides a composition comprising an antisense single-stranded DNA polynucleotide and a transfer agent, wherein said antisense single-stranded DNA polynucleotide is complementary to all or a portion of an essential Tospovirus gene sequence or an RNA transcript thereof, wherein said composition is topically applied to a plant and wherein the symptoms of Tospovirus infection or development of symptoms are reduced or eliminated in said plant relative to a plant not treated with said composition when grown under the same conditions. In some embodiments, the essential gene sequence is selected from the group consisting of SEQ ID NOs:13-46, or the transfer agent is an organosilicone composition, or the antisense single-stranded DNA polynucleotide is selected from the group consisting of SEQ ID NOs:1-12.

In another aspect, the invention provides a method of reducing expression of an essential Tospovirus gene comprising contacting a Tospovirus particle with a composition comprising an antisense single-stranded DNA polynucleotide and a transfer agent, wherein said antisense single-stranded DNA polynucleotide is complementary to all or a portion of an essential gene sequence in said Tospovirus or an RNA transcript thereof, wherein the symptoms of Tospovirus infection or development of symptoms are reduced or eliminated in said plant relative to a plant not treated with said composition when grown under the same conditions. In one embodiment, the essential gene sequence is selected from the group consisting of SEQ ID NOs:13-46. In another embodiment, the transfer agent is an organosilicone compound. In another embodiment, the antisense single-stranded DNA polynucleotide is selected from the group consisting of SEQ ID NOs:1-12 or fragment thereof.

In another aspect, the invention provides a method of identifying antisense single-stranded DNA polynucleotides useful in modulating Tospovirus gene expression when topically treating a plant comprising: a) providing a plurality of antisense single-stranded DNA polynucleotides that comprise a region complementary to all or a part of an essential Tospovirus gene or RNA transcript thereof; b) topically treating said plant with one or more of said antisense single-stranded DNA polynucleotides and a transfer agent; c) analyzing said plant or extract for modulation of symptoms of Tospovirus infection; and d) selecting an antisense single-stranded DNA polynucleotide capable of modulating the symptoms or occurrence of Tospovirus infection. In an embodiment, the transfer agent is an organosilicone compound.

In another aspect, the invention provides an agricultural chemical composition comprising an admixture of an antisense single-stranded DNA polynucleotide and a pesticide, wherein said antisense single-stranded DNA polynucleotide is complementary to all or a portion of an essential Tospovirus gene sequence or RNA transcript thereof, wherein said composition is topically applied to a plant and wherein the symptoms of Tospovirus infection or development of symptoms are reduced or eliminated in said plant relative to a plant not treated with said composition when grown under the same conditions. In an embodiment, the pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.

In another aspect, the invention provides a method of treatment or prevention of a Tospovirus infection in a plant comprising: topically applying to said plant a composition comprising a double-stranded RNA polynucleotide and a transfer agent, wherein said double-stranded RNA comprises a polynucleotide that is essentially complementary to all or a portion of an essential Tospovirus gene sequence or an RNA transcript thereof, wherein the symptoms of viral infection or development of symptoms are reduced or eliminated in said plant relative to a plant not treated with said composition when grown under the same conditions. In some embodiments, the double-stranded RNA comprises a polynucleotide that is essentially identical or essentially complementary to at least 18 contiguous nucleotides of a sequence selected from the group consisting of SEQ ID NOs:13-46. In one embodiment, transfer agent is an organosilicone surfactant composition or compound contained therein. In another embodiment, the composition comprises more than one double-stranded RNA comprising a polynucleotide that is complementary to all or a portion of an essential Tospovirus gene sequence, an RNA transcript of said essential Tospovirus gene sequence, or a fragment thereof. In another embodiment, the double-stranded RNA polynucleotide comprises a polynucleotide that is essentially identical or essentially complementary to a nucleotide sequence as set forth in SEQ NOs:47-103, 448-483, or a fragment thereof. In some embodiments, the antisense polynucleotide of the dsRNA comprises a two (2) nucleotide overhang on the 3′ end that is complementary to the target gene. In another embodiment, the Tospovirus is selected from the group consisting of bean necrotic mosaic virus, Capsicum chlorosis virus, groundnut bud necrosis virus, groundnut ringspot virus, groundnut yellow spot virus, impatiens necrotic spot virus, iris yellow spot viris, melon yellow spot virus, peanut bud necrosis virus, peanut yellow spot virus, soybean vein necrosis-associated virus, tomato chlorotic spot virus, tomato necrotic ringspot virus, tomato spotted wilt virus, tomato zonate spot virus, watermelon bud necrosis virus, watermelon silver mottle virus, and zucchini lethal chlorosis virus. In another embodiment, the essential Tospovirus gene is selected from the group consisting of nucleocapsid gene (N), coat protein gene (CP), virulence factors NSm and NSs, and RNA-dependent RNA polymerase L segment (RdRp/L segment). In another embodiment, the essential Tospovirus gene is selected from the group consisting of SEQ ID NOs:13-46. In another embodiment, the composition is topically applied by spraying, dusting, or is applied to the plant surface as matrix-encapsulated RNA.

In another aspect, the invention provides a composition comprising a double-stranded RNA polynucleotide and a transfer agent, wherein said double-stranded RNA polynucleotide is complementary to all or a portion of an essential Tospovirus gene sequence or an RNA transcript thereof, wherein said composition is topically applied to a plant and wherein the symptoms of Tospovirus infection or development of symptoms are reduced or eliminated in said plant relative to a plant not treated with said composition when grown under the same conditions. In one embodiment, the essential gene sequence is selected from the group consisting of SEQ ID NOs:13-46. In another embodiment, the transfer agent is an organosilicone composition. In another embodiment, the double-stranded RNA comprises a polynucleotide that is essentially identical or essentially complementary to a nucleotide sequence selected from the group consisting of SEQ NOs:47-103 and 448-483. In some embodiments, the antisense polynucleotide of the dsRNA comprises a two (2) nucleotide overhang on the 3′ end that is complementary to the target gene.

In another aspect, the invention provides a method of reducing expression of an essential Tospovirus gene comprising contacting a Tospovirus particle with a composition comprising a double-stranded RNA polynucleotide and a transfer agent, wherein said double-stranded RNA comprises a polynucleotide that is complementary to all or a portion of an essential gene sequence in said Tospovirus or an RNA transcript thereof, wherein the symptoms of Tospovirus infection or development of symptoms are reduced or eliminated in said plant relative to a plant not treated with said composition when grown under the same conditions. In one embodiment, the essential gene sequence is selected from the group consisting of SEQ ID NOs:13-46. In another embodiment, the transfer agent is an organosilicone compound. In another embodiment, the double-stranded RNA comprises a polynucleotide that is essentially identical or essentially complementary to a nucleotide sequence selected from the group consisting of SEQ ID NOs:47-103, 448-483, or fragment thereof. In some embodiments, the antisense polynucleotide of the dsRNA comprises a two (2) nucleotide overhang on the 3′ end that is complementary to the target gene.

In another aspect, the invention provides a method of identifying a double-stranded RNA polynucleotide useful in modulating Tospovirus gene expression when topically treating a plant comprising: a) providing a plurality of double-stranded RNA polynucleotides that comprise a region complementary to all or a part of an essential Tospovirus gene or RNA transcript thereof; b) topically treating said plant with one or more of said double-stranded RNA polynucleotides and a transfer agent; c) analyzing said plant or extract for modulation of symptoms of Tospovirus infection; and d) selecting a double-stranded RNA polynucleotide capable of modulating the symptoms or occurrence of Tospovirus infection. In one embodiment, the transfer agent is an organosilicone compound. In some embodiments, the double-stranded RNA comprises a polynucleotide that is essentially identical or essentially complementary to at least 18 contiguous nucleotides of a sequence selected from the group consisting of SEQ ID NOs:13-46.

In another aspect, the invention provides an agricultural chemical composition comprising an admixture of a double-stranded RNA polynucleotide and a pesticide, wherein said double-stranded RNA comprises a polynucleotide that is essentially complementary to all or a portion of an essential Tospovirus gene sequence or RNA transcript thereof, wherein said composition is topically applied to a plant and wherein the symptoms of Tospovirus infection or development of symptoms are reduced or eliminated in said plant relative to a plant not treated with said composition when grown under the same conditions. In one embodiment, the pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.

In still another aspect, the invention provides a method of treatment or prevention of a Geminivirus infection in a plant comprising: topically applying to said plant a composition comprising a double-stranded RNA polynucleotide and a transfer agent, wherein said double-stranded RNA comprises a polynucleotide that is complementary to all or a portion of an essential Geminivirus gene sequence, or an RNA transcript thereof, wherein the symptoms of viral infection or development of symptoms are reduced or eliminated in said plant relative to a plant not treated with said composition when grown under the same conditions. In one embodiment, the transfer agent is an organosilicone surfactant composition or compound contained therein. In another embodiment, the composition comprises more than one double-stranded RNA comprising a polynucleotide that is essentially complementary to all or a portion of an essential Geminivirus gene sequence, an RNA transcript of said essential Geminivirus gene sequence, or a fragment thereof. In another embodiment, the double-stranded RNA comprises a polynucleotide that is essentially identical or esstentially complementary to at least 18 nucleotides of a sequence selected from the group consisting of SEQ NOs:104-268 or a fragment thereof. In another embodiment, the Geminivirus is selected from the group consisting of Barley yellow dwarf virus, Cucumber mosaic virus, Pepino mosaic virus, Cotton curl leaf virus, Tomato yellow leaf curl virus, Tomato golden mosaic virus, Potato yellow mosaic virus, Pepper leaf curl virus, Bean golden mosaic virus, Bean golden mosaic virus, Tomato mottle virus. In still another aspect, the essential Geminivirus gene is selected from the group consisting of nucleocapsid gene (N), a coat protein gene (CP), virulence factors NSm and NSs, and RNA-dependent RNA polymerase L segment (RdRp/L segment), a silencing suppressor gene, movement protein (MP), Nia, CP-N, a triple gene block, CP-P3, MP-P4, C2, and AC2. In another embodiment, the essential gene sequence is selected from the group consisting of SEQ ID NOs:269-447. In another embodiment, the composition is topically applied by spraying, dusting, or is applied to the plant surface as matrix-encapsulated RNA.

In another aspect, the invention provides a composition comprising a double-stranded RNA polynucleotide and a transfer agent, wherein said double-stranded RNA comprises a polynucleotide that is essentially complementary to all or a portion of an essential Geminivirus gene sequence, such as one set forth as SEQ ID NOs:104-268, 269-447, or an RNA transcript thereof, wherein said composition is topically applied to a plant and wherein the symptoms of Geminivirus infection or development of symptoms are reduced or eliminated in said plant relative to a plant not treated with said composition when grown under the same conditions. In one embodiment, the essential gene sequence is selected from the group consisting of SEQ ID NOs:269-447. In another embodiment, the transfer agent is an organosilicone composition. In another embodiment, the double-stranded RNA polynucleotide is selected from the group consisting of SEQ NOs:104-268.

In another aspect, a method of reducing expression of an essential Geminivirus gene comprising contacting a Geminivirus particle with a composition comprising a double-stranded RNA polynucleotide and a transfer agent, wherein said double-stranded RNA comprises a polynucleotide that is essentially complementary to all or a portion of an essential gene sequence in said Geminivirus or an RNA transcript thereof, wherein the symptoms of Geminivirus infection or development of symptoms are reduced or eliminated in said plant relative to a plant not treated with said composition when grown under the same conditions. In one embodiment, the essential gene sequence is selected from the group consisting of SEQ ID NOs:269-447. In another embodiment, the transfer agent is an organosilicone compound. In another embodiment, the double-stranded RNA comprises a polynucleotide that is essentially identical or essentially complementary to at least 18 nucleotides of a sequence selected from the group consisting of SEQ NOs:104-268 or fragment thereof.

In still another aspect, the invention provides a method of identifying a double-stranded RNA polynucleotide useful in modulating Geminivirus gene expression when topically treating a plant comprising: a) providing a plurality of double-stranded RNA polynucleotides that comprise a region complementary to all or a part of an essential Geminivirus gene or RNA transcript thereof; b) topically treating said plant with one or more of said double-stranded RNA polynucleotides and a transfer agent; c) analyzing said plant or extract for modulation of symptoms of Geminivirus infection; and d) selecting a double-stranded RNA polynucleotide capable of modulating the symptoms or occurrence of Geminivirus infection. In one embodiment, the transfer agent is an organosilicone compound. In some embodiments, the double-stranded RNA comprises a polynucleotide that is essentially identical or essentially complementary to at least 18 contiguous nucleotides of a sequence selected from the group consisting of SEQ ID NOs:269-447. In some embodiments, the Geminivirus is Cucumber Mosiac Virus and the double-stranded RNA comprises a polynucleotide that is essentially identical or essentially complementary to at least 18 contiguous nucleotides of a sequence selected from the to group consisting of SEQ ID NOs:269-316. In some embodiments, the Geminivirus is Pepino Mosaic Virus and the double-stranded RNA comprises a polynucleotide that is essentially identical or essentially complementary to at least 18 contiguous nucleotides of a sequence selected from the group consisting of SEQ ID NOs:317-349. In some embodiments, the Geminivirus is Tomato Yellow Curl Leaf Virus and the double-stranded RNA comprises a polynucleotide that is essentially identical or essentially complementary to at least 18 contiguous nucleotides of a sequence selected from the group consisting of SEQ ID NOs:386-421. In some embodiments, the Gemini virus is Cotton Leaf Curl Virus and the double-stranded RNA comprises a polynucleotide that is essentially identical or essentially complementary to at least 18 contiguous nucleotides of a sequence selected from the group consisting of SEQ ID NOs:422-441.

In another aspect, the invention provides an agricultural chemical composition comprising an admixture of a double-stranded RNA polynucleotide and a pesticide, wherein said double-stranded RNA polynucleotide is complementary to all or a portion of an essential Geminivirus gene sequence or RNA transcript thereof, wherein said composition is topically applied to a plant and wherein the symptoms of Geminivirus infection or development of symptoms are reduced or eliminated in said plant relative to a plant not treated with said composition when grown under the same conditions. In one embodiment, the pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.

In one aspect, the invention provides a method of treatment or prevention of a Geminivirus infection in a plant comprising: topically applying to said plant a composition comprising an antisense single-stranded DNA polynucleotide and a transfer agent, wherein said antisense single-stranded DNA polynucleotide is complementary to all or a portion of an essential Geminivirus gene sequence or an RNA transcript thereof, wherein the symptoms of viral infection or development of symptoms are reduced or eliminated in said plant relative to a plant not treated with said composition when grown under the same conditions. In some embodiments, the antisense single-stranded DNA polynucleotide comprises at least 18 contiguous nucleotides that are essentially complementary a sequence selected from the group consisting of SEQ ID NOs:104-268. In some embodiments, the antisense single-stranded DNA polynucleotide comprises at least 18 contiguous nucleotides that are essentially complementary a sequence selected from the group consisting of SEQ ID NOs:269-447. In one embodiment, the transfer agent is an organosilicone surfactant composition or compound contained therein. In another embodiment, the composition comprises more than one antisense single-stranded DNA polynucleotide complementary to all or a portion of an essential Geminivirus gene sequence, an RNA transcript of said essential Geminivirus gene sequence, or a fragment thereof. In another embodiment, the Geminivirus is selected from the group consisting of Barley yellow dwarf virus, Cucumber mosaic virus, Pepino mosaic virus, Cotton curl leaf virus, Tomato yellow leaf curl virus, Tomato golden mosaic virus, Potato yellow mosaic virus, Pepper leaf curl virus, Bean golden mosaic virus, Bean golden mosaic virus, and Tomato mottle virus. In still another aspect, the essential Geminivirus gene is selected from the group consisting of nucleocapsid gene (N), a coat protein gene (CP), virulence factors NSm and NSs, and RNA-dependent RNA polymerase L segment (RdRp/L segment), a silencing suppressor gene, movement protein (MP), Nia, CP-N, a triple gene block, CP-P3, MP-P4, C2, and AC2. In another embodiment, the essential gene sequence is selected from the group consisting of SEQ ID NOs:269-447. In another embodiment, the composition is topically applied by spraying, dusting, or is applied to the plant surface as matrix-encapsulated RNA.

In another aspect, the invention provides a composition comprising an antisense single-stranded DNA polynucleotide and a transfer agent, wherein said antisense single-stranded DNA polynucleotide is complementary to all or a portion of an essential Geminivirus gene sequence or an RNA transcript thereof, wherein said composition is topically applied to a plant and wherein the symptoms of Geminivirus infection or development of symptoms are reduced or eliminated in said plant relative to a plant not treated with said composition when grown under the same conditions. In some embodiments, the essential gene sequence is selected from the group consisting of SEQ ID NOs:104-447, or the transfer agent is an organosilicone composition.

In another aspect, the invention provides a method of reducing expression of an essential Geminivirus gene comprising contacting a Geminivirus particle with a composition comprising an antisense single-stranded DNA polynucleotide and a transfer agent, wherein said antisense single-stranded DNA polynucleotide is complementary to all or a portion of an essential gene sequence in said Geminivirus or an RNA transcript thereof, wherein the symptoms of Geminivirus infection or development of symptoms are reduced or eliminated in said plant relative to a plant not treated with said composition when grown under the same conditions. In one embodiment, the essential gene sequence is selected from the group consisting of SEQ ID NOs:104-447. In another embodiment, the transfer agent is an organosilicone compound.

In another aspect, the invention provides a method of identifying antisense single-stranded DNA polynucleotides useful in modulating Geminivirus gene expression when topically treating a plant comprising: a) providing a plurality of antisense single-stranded DNA polynucleotides that comprise a region complementary to all or a part of an essential Geminivirus gene or RNA transcript thereof; b) topically treating said plant with one or more of said antisense single-stranded DNA polynucleotides and a transfer agent; c) analyzing said plant or extract for modulation of symptoms of Geminivirus infection; and d) selecting an antisense single-stranded DNA polynucleotide capable of modulating the symptoms or occurrence of Geminivirus infection. In an embodiment, the transfer agent is an organosilicone compound. In some embodiments, the antisense single-stranded DNA is essentially complementary to at least 18 contiguous nucleotides of a sequence selected from the group consisting of SEQ ID NOs:269-447. In some embodiments, the Geminivirus is Cucumber mosaic virus and the antisense single-stranded DNA is essentially complementary to at least 18 contiguous nucleotides of a sequence selected from the group consisting of SEQ ID NOs:269-316. In some embodiments, the Geminivirus is Pepino mosaic virus and the antisense single-stranded DNA is essentially complementary to at least 18 contiguous nucleotides of a sequence selected from the group consisting of SEQ ID NOs:317-349. In some embodiments, the Geminivirus is Tomato yellow leaf curl virus and the antisense single-stranded DNA is essentially complementary to at least 18 contiguous nucleotides of a sequence selected from the group consisting of SEQ ID NOs:386-421. In some embodiments, the Geminivirus is Cotton leaf curl virus and the antisense single-stranded DNA is essentially complementary to at least 18 contiguous nucleotides of a sequence selected from the group consisting of SEQ ID NOs:422-441.

In another aspect, the invention provides an agricultural chemical composition comprising an admixture of an antisense single-stranded DNA polynucleotide and a pesticide, wherein said antisense single-stranded DNA polynucleotide is complementary to all or a portion of an essential Geminivirus gene sequence or RNA transcript thereof, wherein said composition is topically applied to a plant and wherein the symptoms of Geminivirus infection or development of symptoms are reduced or eliminated in said plant relative to a plant not treated with said composition when grown under the same conditions. In an embodiment, the pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.

### BRIEF DESCRIPTION OF THE DRAWINGS

The following drawings form part of the present specification and are included to further demonstrate certain aspects of the function of the compositions and methods. The function may be better understood by reference to one or more of these drawings in combination with the detailed description of specific embodiments presented herein. The function can be more fully understood from the following description of the figures:

FIG. 1: Shows a graph depicting the results of topical treatment of lettuce (SVR3606 L4) plants with antisense single stranded (ss) DNA oligonucleotides (oligos). Fresh weight aerial tissue (in grams) was plotted against treatments performed at −1 Day infection, 0 Day Infection and +1 Day Infection.

FIG. 2: Shows symptom development on lettuce (SVR3606 L4) plants 18 days after virus inoculation. (A) Plants on the right were sprayed with antisense ssDNA oligos at 20 psi using an airbrush several hours after virus inoculation. Left side shows control plants inoculated with impatiens necrotic spotted virus (INSV) only. Leaves were punctured with a hole puncture for ELISA analysis. (B) Graph depicting the results of visual scoring for INSV symptom development in null treated or antisense ssDNA treated plants.

FIG. 3: Shows a graph of the results of ELISA analysis of the effects of topical treatment with antisense ssDNA on reduction of virus accumulation in lettuce leaves. The unit of measure is protein absorbance at optical density (OD) of 450 nm. Circles represent data points collected from the control plants (virus only, no polynucleotide). Triangles represent data points collected from plants treated with a mixture of antisense ssDNA oligos (SEQ ID NO:1 and SEQ ID NO:2).

FIG. 4: Panels A, B, and D show graphs depicting the optical density (OD 450 nm) of extracts of lettuce plants at day 5 (A), day 8 (B), and day 14 (D) after treatment with antisense ssDNA oligos. (C) Shows a graph depicting the results of visual assessment of plants at day 13 after treatment with antisense ssDNA oligos.

FIG. 5: Shows results of the effects of topical treatment with antisense ssDNA oligos on lettuce plants. Panels A and B show the OD 450 nm ELISA data at 5 and 14 days after treatment, respectively. Panel C shows a graph of the mean effective yield of photosystem II (PSII) determined by a portable chlorophyll fluorometer at day 21 after treatment with antisense ssDNA oligos. Panel D shows a graph of the fresh weight aerial tissue (in grams) for null or antisense ssDNA treated plants at day 21 after treatment.

FIG. 6: Shows a field trial planting scheme and day 60 photo in which tomato and pepper plants were topically treated with antisense ssDNA oligos against tomato spotted wilt virus (TSWV).

FIG. 7: Shows tomato plants both untreated (circled) and topically treated with antisense ssDNA oligos against TSWV.

FIG. 8: Shows graphs of the results of the effects of treatment of tomato plants with antisense ssDNA oligos. Panels A, B, and D show graphs depicting the OD 450 nm ELISA data for plants treated with buffer only or sprayed once or twice with antisense ssDNA oligonucleotides at 15 (A), 60 (B), and 78 (D) days post-treatment. Panel C shows a graph depicting the results of visual scoring of the tomato plants for symptoms at day 78 post-treatment.

FIG. 9: Shows graphs of the results of the effects of treatment of pepper plants with antisense ssDNA oligos. Panels A, B, and D show graphs depicting the OD 450 nm ELISA data for pepper plants treated with buffer only or sprayed once or to twice with antisense ssDNA oligonucleotides at 15 (A), 60 (B), and 78 (D) days post-treatment. Panel C shows a graph depicting the results of visual scoring of the pepper plants for symptoms at day 78 post-treatment.

FIG. 10: Shows a graph of the effects of oligo treatment on reduction of virus accumulation in pepper leaves. The OD 450 nm was measured to assess the amount of virus present. The dots represent data points collected from the control plants (virus only, no oligo treatment). Diamonds (SEQ ID NOs:5-8) and triangles (SEQ ID NOs:9-12) represent data points collected from samples topically treated with the antisense ssDNA oligonucleotide solution. The left side shows data from inoculated leaves, and the right side shows data from systemic non-infected, non-oligo-treated leaves.

FIG. 11: Shows graphs of the results of the effects of oligo treatment on onion plants. Panel A shows a graph depicting the bulb diameter prior to treatment with topical oligonucleotides. Panel B shows a graph depicting the different bulb diameters in 4 different sections of the field. Panel C shows a graph depicting the bulb diameter after treatment with buffer or topical antisense ssDNA oligonucleotides. Panel D shows a graph depicting the OD 450 nm measurement for buffer and antisense ssDNA treated plants.

FIG. 12: Panel A shows a graph of the plant height for the different treatments. T25748, T25753, T25755, T25763, T25769, T25770, T25773, T25776, and T25778 are dsRNA triggers. Panel B shows a graph of the plant height for Healthy (uninfected), Virus infected but untreated, Virus infected buffer treated (Buffer), Virus infected T25748 dsRNA trigger treated (T25748), and Virus infected T25773 dsRNA trigger treated (T25773) plants.

FIG. 13: Shows a graph of the plant height for the different treatments. T25748, T25755, T25763, T25769, T25770, T25772, T25775, and T25776 are dsRNA triggers.

### DETAILED DESCRIPTION OF THE INVENTION

Provided are compositions and methods useful for treating or preventing viral infection in plants. Aspects of the methods and compositions disclosed herein can be applied to treat or prevent viral infection in plants in agronomic and other cultivated environments.

Several embodiments relate to methods and compositions for the prevention or treatment of Tospovirus infection in a plant comprising the topical administration of a polynucleotide comprising at least 18 contiguous nucleotides that are essentially identical or essentially complementary to a Tospoviral gene. In some embodiments, the Tospoviral gene is selected from the group consisting of a nucleocapsid (N) gene, a suppressor (NSs) gene, a movement (NSm) gene, and a RNA dependent RNA polymerase (RdRp) gene. In some embodiments, methods and compositions for the prevention or treatment of Tospovirus infection in a plant comprising the topical administration of single-stranded (ss) DNA in antisense (as) orientation as set forth in SEQ ID NOs:1-12 (Tables 1-3) are provided. Also provided are methods and compositions for the prevention or treatment of Tospovirus infection in a plant comprising the topical administration of double-stranded (ds) RNA comprising a polynucleotide that is essentially identical or essentially complementary to a nucleotide sequence as set forth in SEQ ID NOs:47-103 (Table 5) or SEQ ID NOs:448-483 (Table 12). In some embodiments, the antisense polynucleotide of the dsRNA comprises a two (2) nucleotide overhang on the 3′ end that is complementary to the target gene. In certain embodiments, the methods and compositions of the invention provide regulation, repression, or delay and/or modulation of symptoms or disease caused by Tospovirus.

Several embodiments relate to methods and compositions for the prevention or treatment of Geminivirus infection in a plant comprising the topical administration of a polynucleotide comprising at least 18 contiguous nucleotides that are essentially identical or essentially complementary to a Geminiviral gene. In some embodiments, the Geminiviral gene is selected from the group consisting of a coat protien (CP) gene, a silencing suppressor gene, and a movement gene. Also provided are methods and compositions for the prevention or treatment of Geminivirus infection in a plant comprising the topical administration of dsRNA comprising a polynucleotide that is essentially identical or essentially complementary to a nucleotide sequence as set forth in SEQ ID NOs:104-268 (Table 6). Aspects of the methods and compositions can be applied to manage plant viral diseases in agronomic and other cultivated environments.

Compositions of the present invention may include ssDNA, dsDNA, ssRNA, or dsRNA polynucleotides and/or ssDNA, dsDNA, ssRNA, or dsRNA oligonucleotides designed to target single or multiple viral genes, or multiple segments of one or more viral genes, such as genes from a Tospovirus or other plant disease, including, but not limited to the viral gene sequences set forth in SEQ ID NOs:1-46 (Tables 1-4). In another embodiment, such polynucleotides and oligonucleotides may be designed to target single or multiple viral genes, or multiple segments of one or more viral genes, such as genes from a Geminivirus, including, but not limited to the viral gene sequences set forth in SEQ ID NOs:269-447 (Tables 7-11). In an embodiment, any viral gene from any plant virus may be targeted by compositions of the present invention. The target gene may include multiple consecutive segments of a target gene, multiple non-consecutive segments of a target gene, multiple alleles of a target gene, or multiple target genes from one or more Tospovirus species. In some embodiments, the polynucleotides or oligonucleotides are essentially identical or essentially complementary to a consensus nucleotide sequence.

Polynucleotides of the invention may be complementary to all or a portion of a viral gene sequence, including a promoter, intron, coding sequence, exon, 5′ untranslated region, and 3′ untranslated region. Compositions of the present invention further comprise a transfer agent that facilitates delivery of the polynucleotide of the invention to a plant, and may include solvents, diluents, a pesticide that complements the action of the polynucleotide, a herbicide or additional pesticides or that provides an additional mode of action different from the polynucleotide, various salts or stabilizing agents that enhance the utility of the composition as an admixture of the components of the composition.

In certain aspects, methods of the invention may include one or more applications of a polynucleotide composition and one or more applications of a transfer agent for conditioning of a plant or plant virus to permeation by polynucleotides or activity or stability of the polynucleotides. When the agent for conditioning to permeation is an organosilicone composition or compound contained therein, the polynucleotide molecules may be ssDNA, dsDNA, ssRNA, or dsRNA oligonucleotides; or ssDNA, dsDNA, ssRNA, or dsRNA polynucleotides, chemically modified DNA oligonucleotides or polynucleotides, or mixtures thereof.

In one embodiment, the present invention provides a method for controlling Tospovirus or Geminivirus infection of a plant including treatment of the plant with at least a first antisense ssDNA complementary to all or a portion of a target viral gene, wherein the polynucleotide molecules are capable of modulation of the target gene and controlling Tospovirus or Geminivirus infection. In another embodiment, the present invention provides a method for controlling Tospovirus or Geminivirus infection of a plant including treatment of the plant with at least a first antisense dsDNA complementary to all or a portion of a target viral gene, wherein the polynucleotide molecules are capable of modulation of the target gene and controlling Tospovirus or Geminivirus infection. In another embodiment, the invention provides a method for controlling Tospovirus or Geminivirus infection of a plant including treatment of the plant with at least a first dsRNA complementary to all or a portion of a target viral gene, wherein the polynucleotide molecules are capable of modulation of the target gene and controlling Tospovirus or Geminivirus infection.

In certain embodiments, a conditioning step to increase permeability of a plant to the polynucleotide may be included. The conditioning and polynucleotide application can be performed separately or in a single step. When the conditioning and polynucleotide application are performed in separate steps, the conditioning can precede or can follow the polynucleotide application within minutes, hours, or days. In some embodiments, more than one conditioning step or more than one polynucleotide molecule application can be performed on the same plant.

In specific embodiments of the method, a polynucleotide of the invention can be cloned or identified from (a) coding (protein-encoding), (b) non-coding (promoter and other gene related molecules), or (c) both coding and non-coding parts of the target viral gene. Non-coding parts may include DNA, such as promoter regions or an RNA transcribed by the DNA that provides RNA regulatory molecules, including but not limited to: introns, cis-acting regulatory RNA elements, 5′ or 3′ untranslated regions, and microRNAs (miRNA), trans-acting siRNAs, natural antisense siRNAs, and other small RNAs with regulatory function or RNAs having structural or enzymatic function including but not limited to: ribozymes, ribosomal RNAs, t-RNAs, aptamers, and riboswitches.

As used herein, “Tospovirus” refers to a virus from the genus Tospovirus, which may include bean necrotic mosaic virus, Capsicum chlorosis virus, groundnut bud necrosis virus, groundnut ringspot virus, groundnut yellow spot virus, impatiens necrotic spot virus, iris yellow spot viris, melon yellow spot virus, peanut bud necrosis virus, peanut yellow spot virus, soybean vein necrosis-associated virus, tomato chlorotic spot virus, tomato necrotic ringspot virus, tomato spotted wilt virus, tomato zonate spot virus, watermelon bud necrosis virus, watermelon silver mottle virus, or zucchini lethal chlorosis virus.

As used herein, a “Geminivirus” refers to a virus from the Geminiviridae Family of plant viruses. A Geminivirus may include, but is not limited to, Barley yellow dwarf virus (BYDW), Cucumber mosaic virus (CMV), Pepino mosaic virus (PepMV), Cotton curl leaf virus (CuCLV), Tomato yellow leaf curl virus (TYLCV), Tomato golden mosaic virus, Potato yellow mosaic virus, Pepper leaf curl virus (PepLCV), Bean golden mosaic virus (BGMV-PR), Bean golden mosaic virus (BGMV-DR), Tomato mottle virus (TMV), and the like.

The DNA or RNA polynucleotide compositions of the present invention are useful in compositions, such as liquids that comprise DNA or RNA polynucleotide molecules, alone or in combination with other components either in the same liquid or in separately applied liquids that provide a transfer agent. As used herein, a transfer agent is an agent that, when combined with a polynucleotide in a composition that is topically applied to a target plant surface facilitates the use of the polynucleotide in controlling a Tospovirus or Geminivirus. In one embodiment, the transfer agent enhances the ability of the polynucleotide to enter a plant cell. In certain embodiments, a transfer agent is therefore an agent that conditions the surface of plant tissue, e. g., leaves, stems, roots, flowers, or fruits, to permeation by the polynucleotide molecules into plant cells. The transfer of polynucleotides into plant cells can be facilitated by the prior or contemporaneous application of a polynucleotide-transferring agent to the plant tissue. In some embodiments the transferring agent is applied subsequent to the application of the polynucleotide composition. The polynucleotide transfer agent enables a pathway for polynucleotides through cuticle wax barriers, stomata and/or cell wall or membrane barriers into plant cells. Suitable transfer agents to facilitate transfer of the to polynucleotide into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof.

Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e. g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g., plant-sourced oils, crop oils (such as those listed in the 9^{th} Compendium of Herbicide Adjuvants, publicly available on the worldwide web (internet) at herbicide.adjuvants.com can be used, e.g., paraffinic oils, polyol fatty acid esters, or oils with short-chain molecules modified with amides or polyamines such as polyethyleneimine or N-pyrrolidine. Transfer agents include, but are not limited to, organosilicone preparations.

In certain embodiments, an organosilicone preparation that comprises an organosilicone compound comprising a trisiloxane head group is used in the methods and compositions provided herein. In certain embodiments, an organosilicone preparation that comprises an organosilicone compound comprising a heptamethyltrisiloxane head group is used in the methods and compositions provided herein. In certain embodiments of the methods and compositions provided herein, a to composition that comprises a polynucleotide molecule and one or more effective organosilicone compound in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Organosilicone preparations used in the methods and compositions provided herein can comprise one or more effective organosilicone compounds. As used herein, the phrase “effective organosilicone compound” is used to describe any organosilicone compound that is found in an organosilicone preparation that enables a polynucleotide to enter a plant cell. In certain embodiments, an effective organosilicone compound can enable a polynucleotide to enter a plant cell in a manner permitting a polynucleotide mediated suppression of a target gene expression in the plant cell. In general, effective organosilicone compounds include, but are not limited to, compounds that can comprise: i) a trisiloxane head group that is covalently linked to, ii) an alkyl linker including, but not limited to, an n-propyl linker, that is covalently linked to, iii) a poly glycol chain, that is covalently linked to, iv) a terminal group. Trisiloxane head groups of such effective organosilicone compounds include, but are not limited to, heptamethyltrisiloxane. Alkyl linkers can include, but are not limited to, an n-propyl linker. Poly glycol chains include, but are not limited to, polyethylene glycol or polypropylene glycol. Poly glycol chains can comprise a mixture that provides an average chain length “n” of about “7.5.” In certain embodiments, the average chain length “n” can vary from about 5 to about 14. Terminal groups can include, but are not limited to, alkyl groups such as a methyl group. Effective organosilicone compounds are believed to include, but are not limited to, trisiloxane ethoxylate surfactants or polyalkylene oxide modified heptamethyl trisiloxane.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, N.Y. can be used to prepare a polynucleotide composition. In certain embodiments where a Silwet L-77 organosilicone preparation is used as a pre-spray treatment of plant leaves or other plant surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation comprising Silwet L-77 in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

In certain embodiments, any of the commercially available organosilicone preparations provided such as the following Breakthru S 321, Breakthru S 200 Cat#67674-67-3, Breakthru OE 441 Cat#68937-55-3, Breakthru S 278 Cat #27306-78-1, Breakthru S 243, Breakthru S 233 Cat#134180-76-0, available from manufacturer Evonik Goldschmidt (Germany), Silwet® HS 429, Silwet® HS 312, Silwet® HS 508, Silwet® HS 604 (Momentive Performance Materials, Albany, N.Y.) can be used as transfer agents in a polynucleotide composition. In certain embodiments where an organosilicone preparation is used as a pre-spray treatment of plant leaves or other surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Delivery of a polynucleotide according to the invention can be accomplished by a variety of methods including, without limitation, (1) loading liposomes with a ssDNA, dsDNA, ssRNA, or dsRNA molecule provided herein and (2) complexing a ssDNA, dsDNA, ssRNA, or dsRNA molecule with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged, nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidyl glycerol, dioleoyl phosphatidylethanolamine or liposomes comprising dihydrosphingomyelin (DHSM). Numerous lipophilic agents are commercially available, including Lipofectin® (Invitrogen/Life Technologies, Carlsbad, Calif.) and Effectene™ (Qiagen, Valencia, Calif.). In addition, systemic delivery methods can be optimized using commercially available cationic lipids such as DDAB or DOTAP, each of which can be mixed with a neutral lipid such as DOPE or cholesterol. In some eases, liposomes such as those described by Templeton et al. (Nature Biotechnology, 15:647-652, 1997) can be used. In other embodiments, polycations such as polyethyleneimine can be used to achieve delivery in vivo and ex vivo (Boletta et al., J. Am Soc. Nephrol. 7:1728, 1996). Additional information regarding the use of liposomes to deliver nucleic acids can be found in U.S. Pat. No. 6,271,359, PCT Publication WO 96/40964 and Morrissey et al. (Nat Biotechnol. 23(8):1002-7, 2005).

The following definitions and methods are provided to guide those of ordinary skill in the art. Unless otherwise noted, terms are to be understood according to conventional usage by those of ordinary skill in the relevant art. Where a term is provided in the singular, the inventors also contemplate aspects described by the plural of that term.

By “non-transcribable” polynucleotides is meant that the polynucleotides do not comprise a complete polymerase II transcription unit.

As used herein “solution” refers to homogeneous mixtures and non-homogeneous mixtures such as suspensions, colloids, micelles, and emulsions.

A “trigger” or “trigger polynucleotide” is a DNA polynucleotide molecule that is homologous or complementary to a target gene polynucleotide. The trigger polynucleotide molecules modulate expression of the target gene when topically applied to a plant surface with a transfer agent, whereby a virus-infected plant that is treated with said composition is able to sustain its growth or development or reproductive ability, or said plant is less sensitive to a virus as a result of said polynucleotide-containing composition relative to a plant not treated with a composition containing the trigger molecule. A plant treated with such a composition may be resistant to viral expression as a result of said polynucleotide-containing composition relative to a plant not treated with a composition containing the trigger molecule. Trigger polynucleotides disclosed herein may be generally described in relation to the target gene sequence in an antisense (complementary) or sense orientation as ssDNA, dsDNA, ssRNA, or dsRNA molecules or nucleotide variants and modified nucleotides thereof depending on the various regions of a gene being targeted.

It is contemplated that the composition may contain multiple DNA or RNA polynucleotides and/or pesticides that include, but are not limited to, anti-viral compounds, insecticides, fungicides, nematocides, bactericides, acaricides, growth regulators, chemosterilants, semiochemicals, repellents, attractants, pheromones, feeding stimulants, and biopesticides. Essential genes are genes in a plant that provide key enzymes or other proteins, for example, a biosynthetic enzyme, metabolizing enzyme, receptor, signal transduction protein, structural gene product, transcription factor, or transport protein; or regulating RNAs, such as, microRNAs, that are essential to the growth or survival of the organism or cell or involved in the normal growth and development of the plant (Meinke et al., Trends Plant Sci. 2008:13(9):483-91). Essential genes in a virus may include genes responsible for capsid production, virus assembly, infectivity, budding, and the like. The suppression of an essential gene in a virus affects the function of a gene product that enables viral infection in a plant. The compositions may include various trigger DNA or RNA polynucleotides that modulate the expression of an essential gene in a Tospovirus.

As used herein, the term “DNA,” “DNA molecule,” or “DNA polynucleotide molecule” refers to a ssDNA or dsDNA molecule of genomic or synthetic origin, such as a polymer of deoxyribonucleotide bases or a DNA polynucleotide molecule. As used herein, the term “DNA sequence,” “DNA nucleotide sequence,” or “DNA polynucleotide sequence” refers to the nucleotide sequence of a DNA molecule. Unless otherwise stated, nucleotide sequences in the text of this specification are given, when read from left to right, in the 5′ to 3′ direction. The nomenclature used herein is that required by Title 37 of the United States Code of Federal Regulations §1.822 and set forth in the tables in WIPO Standard ST.25 (1998), Appendix 2, Tables 1 and 3.

As used herein, the term “RNA,” “RNA molecule,” or “RNA polynucleotide molecule” refers to a ssRNA or dsRNA molecule of genomic or synthetic origin, such as a polymer of ribonucleotide bases or an RNA polynucleotide molecule. As used herein, the term “RNA sequence,” “RNA nucleotide sequence,” or “RNA polynucleotide sequence” refers to the nucleotide sequence of an RNA molecule. Unless otherwise stated, nucleotide sequences in the text of this specification are given, when read from left to right, in the 5′ to 3′ direction. The nomenclature used herein is that required by Title 37 of the United States Code of Federal Regulations §1.822 and set forth in the tables in WIPO Standard ST.25 (1998), Appendix 2, Tables 1 and 3.

As used herein, “polynucleotide” refers to a DNA or RNA molecule containing multiple nucleotides and generally also refers to “oligonucleotides” (a polynucleotide molecule of typically 50 or fewer nucleotides in length). Embodiments include compositions including oligonucleotides having a length of 18-25 nucleotides (18-mers, 19-mers, 20-mers, 21-mers, 22-mers, 23-mers, 24-mers, or 25-mers), for example, oligonucleotides as set forth by SEQ ID NOs:1-12, 47-268, and 448-483 or fragments thereof. A target gene comprises any polynucleotide molecule in a plant cell or fragment thereof for which the modulation of the expression of the target gene is provided by the methods and compositions. A gene has noncoding genetic elements (components) that provide for the function of the gene, these elements are polynucleotides that provide gene expression regulation, such as, a promoter, an enhancer, a 5′ untranslated region, intron regions, and a 3′ untranslated region. Oligonucleotides and polynucleotides can be made to any of the genetic elements of a gene and to polynucleotides spanning the junction region of a genetic element, such as, an intron and exon, the junction region of a promoter and a to transcribed region, the junction region of a 5′ leader and a coding sequence, the junction of a 3′ untranslated region and a coding sequence.

Polynucleotide compositions used in the various embodiments include compositions including oligonucleotides or polynucleotides, or a mixture of both, of DNA or RNA, or chemically modified oligonucleotides or polynucleotides or a mixture thereof. In some embodiments, the polynucleotide includes chemically modified nucleotides. Examples of chemically modified oligonucleotides or polynucleotides are well known in the art; see, for example, US Patent Publication 20110171287, US Patent Publication 20110171176, and US Patent Publication 20110152353, US Patent Publication, 20110152346, US Patent Publication 20110160082, herein incorporated in its entirety by reference hereto. For example, including, but not limited to, the naturally occurring phosphodiester backbone of an oligonucleotide or polynucleotide can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (for example, fluorescein or rhodamine) or other label (for example, biotin).

The term “gene” refers to components that comprise chromosomal DNA, RNA, plasmid DNA, cDNA, intron and exon DNA, artificial DNA polynucleotide, or other DNA that encodes a peptide, polypeptide, protein, or RNA transcript molecule, and the genetic elements flanking the coding sequence that are involved in the regulation of expression, such as, promoter regions, 5′ leader regions, 3′ untranslated region that may exist as native genes or transgenes in a plant genome. The gene or a fragment thereof is isolated and subjected to polynucleotide sequencing methods that determines the order of the nucleotides that comprise the gene. Any of the components of the gene are potential targets for a trigger oligonucleotide and polynucleotides.

The trigger polynucleotide molecules are designed to modulate expression by inducing regulation or suppression of a viral gene and are designed to have a nucleotide sequence essentially identical or essentially complementary to the nucleotide sequence of a viral gene or to the sequence of RNA transcribed from a viral gene of a plant, the sequence thereof determined by isolating the gene or a to fragment of the gene from the plant, which can be coding sequence or non-coding sequence. Effective molecules that modulate expression are referred to as “a trigger molecule, or trigger polynucleotide”. By “essentially identical” or “essentially complementary” is meant that the trigger polynucleotides (or at least a portion of a polynucleotide) are designed to hybridize to the endogenous gene noncoding sequence or to RNA transcribed (known as messenger RNA or an RNA transcript) from the endogenous gene to effect regulation or suppression of expression of the endogenous gene. Trigger molecules are identified by “tiling” the gene targets with partially overlapping probes or non-overlapping probes of antisense polynucleotides that are essentially identical or essentially complementary to the nucleotide sequence of an endogenous gene. Multiple target sequences can be aligned and sequence regions with homology in common, according to the methods, are identified as potential trigger molecules for the multiple targets. Multiple trigger molecules of various lengths, for example 18-25 nucleotides, 26-50 nucleotides, 51-100 nucleotides, 101-200 nucleotides, 201-300 nucleotides or more can be pooled into a few treatments in order to investigate polynucleotide molecules that cover a portion of a gene sequence (for example, a portion of a coding versus a portion of a noncoding region, or a 5′ versus a 3′ portion of a gene) or an entire gene sequence including coding and noncoding regions of a target gene. Polynucleotide molecules of the pooled trigger molecules can be divided into smaller pools or single molecules in order to identify trigger molecules that provide the desired effect.

The target gene ssDNA polynucleotide molecules, including SEQ ID NOs:1-12, or dsRNA molecules, including SEQ ID NOs:47-268 and 448-483 may be sequenced by any number of available methods and equipment known in the art. Some of the sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.). In addition to the single molecule sequencing performed using sequencing-by-synthesis of Helicos Biosciences, other single molecule sequencing technologies are encompassed and include the SMRT™ technology of Pacific Biosciences, the Ion Torrent™ technology, and nanopore sequencing being developed for example, by to Oxford Nanopore Technologies. A viral target gene comprising DNA or RNA can be isolated using primers or probes essentially complementary or essentially homologous to the target gene or a fragment thereof. A polymerase chain reaction (PCR) gene fragment can be produced using primers essentially complementary or essentially homologous to a viral gene or a fragment thereof that is useful to isolate a viral gene from a plant genome. Various sequence capture technologies can be used to isolate additional target gene sequences, for example, including but not limited to Roche NimbleGen® (Madison, Wis.) and Streptavdin-coupled Dynabeads® (Life Technologies, Grand Island, N.Y.) and US20110015084, herein incorporated by reference in its entirety.

Embodiments of functional single-stranded or double-stranded polynucleotides have sequence complementarity that need not be 100 percent, but is at least sufficient to permit hybridization to RNA transcribed from the target gene or DNA of the target gene to form a duplex to permit a gene silencing mechanism. Thus, in embodiments, a polynucleotide fragment is designed to be complementary to all or a portion of an essential target Tospovirus or Geminivirus gene sequence. For instance, the fragment may be essentially identical or essentially complementary to a sequence of 18 or more contiguous nucleotides in either the target viral gene sequence or messenger RNA transcribed from the target gene. By “essentially identical” is meant having 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the target gene or RNA transcribed from the target gene; by “essentially complementary” is meant having 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene. In some embodiments, polynucleotide molecules are designed to have 100 percent sequence identity with or complementarity to one allele or one family member of a given target gene (coding or non-coding sequence of a gene); in other embodiments the polynucleotide molecules are designed to have 100 percent sequence identity with or complementarity to multiple alleles or family members of a given target gene.

“Identity” refers to the degree of similarity between two polynucleic acid or protein sequences. An alignment of the two sequences is performed by a suitable computer program. A widely used and accepted computer program for performing sequence alignments is CLUSTALW v1.6 (Thompson, et al. Nucl. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there are 100 matched amino acids between a 200 and a 400 amino acid protein, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Trigger molecules for specific viral gene family members can be identified from coding and/or non-coding sequences of gene families of a plant virus or multiple plant viruses, by aligning and selecting 200-300 polynucleotide fragments from the least homologous regions among the aligned sequences and evaluated using topically applied polynucleotides (antisense ssDNA or dsRNA) to determine their relative effectiveness in providing the anti-viral phenotype. In some embodiments, the viral gene family is Tospovirus and the sequences are selected from SEQ ID NOs:13-46. In some embodiments, the viral gene family is Cucumber mosaic virus and the sequences are selected from SEQ ID NOs:269-316. In some embodiments, the viral gene family is Pepino mosaic virus and the sequences are selected from SEQ ID NOs:317-349. In some embodiments, the viral gene family is Barley yellow dwarf virus and the sequences are selected from SEQ ID NOs:350-385. In some embodiments, the viral gene family is Tomato yellow leaf curl virus and the sequences are selected from SEQ ID NOs:386-421. In some embodiments, the viral gene family is Cotton leaf curl virus and the sequences are selected from SEQ ID NOs:422-441. The effective segments are further subdivided into 50-60 polynucleotide fragments, prioritized by least homology, and reevaluated using topically applied polynucleotides. The effective 50-60 polynucleotide fragments are subdivided into 19-30 polynucleotide fragments, prioritized by least homology, and again evaluated for induction of the anti-viral phenotype. Once relative effectiveness is determined, the fragments are utilized singly, or again evaluated in combination with one or more other fragments to determine the trigger composition or mixture of trigger polynucleotides for providing the anti-viral phenotype.

Trigger molecules for broad anti-viral activity can be identified from coding and/or non-coding sequences of gene families of a plant virus or multiple plants viruses, by aligning and selecting 200-300 polynucleotide fragments from the most homologous regions amongst the aligned sequences and evaluated using topically applied polynucleotides (antisense ssDNA or dsRNA) to determine their relative effectiveness in inducing the anti-viral phenotype. In some embodiments, the viral gene family is Tospovirus and the sequences are selected from SEQ ID NOs:13-46. In some embodiments, the viral gene family is Cucumber mosaic virus and the sequences are selected from SEQ ID NOs:269-316. In some embodiments, the viral gene family is Pepino mosaic virus and the sequences are selected from SEQ ID NOs:317-349. In some embodiments, the viral gene family is Barley yellow dwarf virus and the sequences are selected from SEQ ID NOs:350-385. In some embodiments, the viral gene family is Tomato yellow leaf curl virus and the sequences are selected from SEQ ID NOs:386-421. In some embodiments, the viral gene family is Cotton leaf curl virus and the sequences are selected from SEQ ID NOs:422-441. The effective segments are subdivided into 50-60 polynucleotide fragments, prioritized by most homology, and reevaluated using topically applied polynucleotides. The effective 50-60 polynucleotide fragments are subdivided into 19-30 polynucleotide fragments, prioritized by most homology, and again evaluated for induction of the anti-viral phenotype. Once relative effectiveness is determined, the fragments may be utilized singly, or in combination with one or more other fragments to determine the trigger composition or mixture of trigger polynucleotides for providing the anti-viral phenotype.

Methods of making polynucleotides are well known in the art. Chemical synthesis, in vivo synthesis and in vitro synthesis methods and compositions are known in the art and include various viral elements, microbial cells, modified polymerases, and modified nucleotides. Commercial preparation of oligonucleotides often provides two deoxyribonucleotides on the 3′ end of the sense strand. Long polynucleotide molecules can be synthesized from commercially available kits. Long polynucleotide molecules can also be assembled from multiple DNA fragments. In to some embodiments design parameters such as Reynolds score (Reynolds et al. Nature Biotechnology 22, 326-330 (2004), Tuschl rules (Pei and Tuschl, Nature Methods 3(9):670-676, 2006), i-score (Nucleic Acids Res 35:e123, 2007), i-Score Designer tool and associated algorithms (Nucleic Acids Res 32:936-948, 2004. Biochem Biophys Res Commun 316:1050-1058, 2004, Nucleic Acids Res 32:893-901, 2004, Cell Cycle 3:790-5, 2004, Nat Biotechnol 23:995-1001, 2005, Nucleic Acids Res 35:e27, 2007, BMC Bioinformatics 7:520, 2006, Nucleic Acids Res 35:e123, 2007, Nat Biotechnol 22:326-330, 2004) are known in the art and may be used in selecting polynucleotide sequences effective in gene silencing. In some embodiments the sequence of a polynucleotide is screened against the genomic DNA of the intended plant to minimize unintentional silencing of other genes.

Ligands can be tethered to a ssDNA or dsRNA polynucleotide. Ligands in general can include modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids (e.g., cholesterol, a bile acid, or a fatty acid (e.g., lithocholic-oleyl, lauroyl, docosnyl, stearoyl, palmitoyl, myristoyl oleoyl, linoleoyl), steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. The ligand may also be a recombinant or synthetic molecule, such as a synthetic polymer, e.g., polyethylene glycol (PEG), PEG-40K, PEG-20K and PEG-5K. Other examples of ligands include lipophilic molecules, e.g., cholesterol, cholic acid, adamantane acetic acid, 1-pyrene butyric acid, dihydrotestosterone, glycerol (e.g., esters and ethers thereof, e.g., C₁₀, C₁₁, C₁₂, C₁₃, C₁₄, C₁₅, C₁₆, C₁₇, C₁₈, C₁₉, or C₂₀ alkyl; e.g., lauroyl, docosnyl, stearoyl, oleoyl, linoleoyl 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dodecanoyl, lithocholyl, 5β-cholanyl, N,N-distearyl-lithocholamide, 1,2-di-O-stearoylglyceride, dimethoxytrityl, or phenoxazine) and PEG (e.g., PEG-5K, PEG-20K, PEG-40K). Preferred lipophilic moieties include lipid, cholesterols, oleyl, retinyl, or cholesteryl residues.

The method of the invention may be applied to plants that are or are not transgenic. Non-limiting examples of transgenic plants include those that comprise one or more transgene conferring a trait selected from the group consisting of insect resistance, pesticide resistance, enhanced shelf life, fruit coloring, fruit ripening, fruit sweetness, nutritional value, and the like.

In specific embodiments of the invention, a plant disease control composition as provided herein may further be provided in a composition formulated for application to a plant that comprises at least one other active ingredient. Examples of such active ingredients may include, but are not limited to, an insecticidal protein such as a patatin, a Bacillus thuringiensis insecticidal protein, a Xenorhabdus insecticidal protein, a Photorhabdus insecticidal protein, a Bacillus laterosporous insecticidal protein, and a Bacillus sphearicus insecticidal protein. In another non-limiting example, such an active ingredient is a herbicide, such as one or more of acetochlor, acifluorfen, acifluorfen-sodium, aclonifen, acrolein, alachlor, alloxydim, allyl alcohol, ametryn, amicarbazone, amidosulfuron, aminopyralid, amitrole, ammonium sulfamate, anilofos, asulam, atraton, atrazine, azimsulfuron, BCPC, beflubutamid, benazolin, benfluralin, benfuresate, bensulfuron, bensulfuron-methyl, bensulide, bentazone, benzfendizone, benzobicyclon, benzofenap, bifenox, bilanafos, bispyribac, bispyribac-sodium, borax, bromacil, bromobutide, bromoxynil, butachlor, butafenacil, butamifos, butralin, butroxydim, butylate, cacodylic acid, calcium chlorate, cafenstrole, carbetamide, carfentrazone, carfentrazone-ethyl, CDEA, CEPC, chlorflurenol, chlorflurenol-methyl, chloridazon, chlorimuron, chlorimuron-ethyl, chloroacetic acid, chlorotoluron, chlorpropham, chlorsulfuron, chlorthal, chlorthal-dimethyl, cinidon-ethyl, cinmethylin, cinosulfuron, cisanilide, clethodim, clodinafop, clodinafop-propargyl, clomazone, clomeprop, clopyralid, cloransulam, cloransulam-methyl, CMA, 4-CPB, CPMF, 4-CPP, CPPC, cresol, cumyluron, cyanamide, cyanazine, cycloate, cyclosulfamuron, cycloxydim, cyhalofop, cyhalofop-butyl, 2,4-D, 3,4-DA, daimuron, dalapon, dazomet, 2,4-DB, 3,4-DB, 2,4-DEB, desmedipham, dicamba, dichlobenil, ortho-dichlorobenzene, para-dichlorobenzene, dichlorprop, dichlorprop-P, diclofop, diclofop-methyl, diclosulam, difenzoquat, difenzoquat metilsulfate, diflufenican, diflufenzopyr, dimefuron, dimepiperate, dimethachlor, dimethametryn, dimethenamid, dimethenamid-P, dimethipin, dimethylarsinic acid, to dinitramine, dinoterb, diphenamid, diquat, diquat dibromide, dithiopyr, diuron, DNOC, 3,4-DP, DSMA, EBEP, endothal, EPTC, esprocarb, ethalfluralin, ethametsulfuron, ethametsulfuron-methyl, ethofumesate, ethoxyfen, ethoxysulfuron, etobenzanid, fenoxaprop-P, fenoxaprop-P-ethyl, fentrazamide, ferrous sulfate, flamprop-M, flazasulfuron, florasulam, fluazifop, fluazifop-butyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, fluoroglycofen, fluoroglycofen-ethyl, flupropanate, flupyrsulfuron, flupyrsulfuron-methyl-sodium, flurenol, fluridone, fluorochloridone, fluoroxypyr, flurtamone, fluthiacet, fluthiacet-methyl, fomesafen, foramsulfuron, fosamine, glufosinate, glufosinate-ammonium, glyphosate, halosulfuron, halosulfuron-methyl, haloxyfop, haloxyfop-P, HC-252, hexazinone, imazamethabenz, imazamethabenz-methyl, imazamox, imazapic, imazapyr, imazaquin, imazethapyr, imazosulfuron, indanofan, iodomethane, iodosulfuron, iodosulfuron-methyl-sodium, ioxynil, isoproturon, isouron, isoxaben, isoxachlortole, isoxaflutole, karbutilate, lactofen, lenacil, linuron, MAA, MAMA, MCPA, MCPA-thioethyl, MCPB, mecoprop, mecoprop-P, mefenacet, mefluidide, mesosulfuron, mesosulfuron-methyl, mesotrione, metam, metamifop, metamitron, metazachlor, methabenzthiazuron, methylarsonic acid, methyldymron, methyl isothiocyanate, metobenzuron, metolachlor, S-metolachlor, metosulam, metoxuron, metribuzin, metsulfuron, metsulfuron-methyl, MK-66, molinate, monolinuron, MSMA, naproanilide, napropamide, naptalam, neburon, nicosulfuron, nonanoic acid, norflurazon, oleic acid (fatty acids), orbencarb, orthosulfamuron, oryzalin, oxadiargyl, oxadiazon, oxasulfuron, oxaziclomefone, oxyfluorfen, paraquat, paraquat dichloride, pebulate, pendimethalin, penoxsulam, pentachlorophenol, pentanochlor, pentoxazone, pethoxamid, petrolium oils, phenmedipham, phenmedipham-ethyl, picloram, picolinafen, pinoxaden, piperophos, potassium arsenite, potassium azide, pretilachlor, primisulfuron, primisulfuron-methyl, prodiamine, profluazol, profoxydim, prometon, prometryn, propachlor, propanil, propaquizafop, propazine, propham, propisochlor, propoxycarbazone, propoxycarbazone-sodium, propyzamide, prosulfocarb, prosulfuron, pyraclonil, pyraflufen, pyraflufen-ethyl, pyrazolynate, pyrazosulfuron, pyrazosulfuron-ethyl, pyrazoxyfen, pyribenzoxim, pyributicarb, pyridafol, pyridate, pyriftalid, pyriminobac, pyriminobac-methyl, pyrimisulfan, pyrithiobac, pyrithiobac-sodium, quinclorac, quinmerac, quinoclamine, quizalofop, quizalofop-P, rimsulfuron, sethoxydim, siduron, simazine, simetryn, SMA, sodium arsenite, sodium azide, sodium chlorate, sulcotrione, sulfentrazone, sulfometuron, sulfometuron-methyl, sulfosate, sulfosulfuron, sulfuric acid, tar oils, 2,3,6-TBA, TCA, TCA-sodium, tebuthiuron, tepraloxydim, terbacil, terbumeton, terbuthylazine, terbutryn, thenylchlor, thiazopyr, thifensulfuron, thifensulfuron-methyl, thiobencarb, tiocarbazil, topramezone, tralkoxydim, tri-allate, triasulfuron, triaziflam, tribenuron, tribenuron-methyl, tricamba, triclopyr, trietazine, trifloxysulfuron, trifloxysulfuron-sodium, trifluralin, triflusulfuron, triflusulfuron-methyl, trihydroxytriazine, tritosulfuron, [3-[2-chloro-4-fluoro-5-(-methyl-6-trifluoromethyl-2,4-dioxo-,2,3,4-t-etrahydropyrimidin-3-yl)phenoxy]-2-pyridyloxy]acetic acid ethyl ester (CAS RN 353292-3-6), 4-[(4,5-dihydro-3-methoxy-4-methyl-5-oxo)-H-,2,4-triazolylcarbonyl-sulfamoyl]-5-methyl-thiophene-3-carboxylic acid (BAY636), BAY747 (CAS RN 33504-84-2), topramezone (CAS RN 2063-68-8), 4-hydroxy-3-[[2-[(2-methoxyethoxy)methyl]-6-(trifluoro-methyl)-3-pyridi-nyl]carbonyl]-bicyclo[3.2.]oct-3-en-2-one (CAS RN 35200-68-5), and 4-hydroxy-3-[[2-(3-methoxypropyl)-6-(difluoromethyl)-3-pyridinyl]carbon-yl]-bicyclo[3.2]oct-3-en-2-one.

The trigger DNA or RNA polynucleotide and/or oligonucleotide molecule compositions are useful in compositions, such as liquids that comprise the polynucleotide molecules at low concentrations, alone or in combination with other components, for example one or more herbicide molecules, either in the same solution or in separately applied liquids that also provide a transfer agent. While there is no upper limit on the concentrations and dosages of polynucleotide molecules that can useful in the methods, lower effective concentrations and dosages will generally be sought for efficiency. The concentrations can be adjusted in consideration of the volume of spray or treatment applied to plant leaves or other plant part surfaces, such as flower petals, stems, tubers, fruit, anthers, pollen, or seed. In one embodiment, a useful treatment for herbaceous plants using 25-mer oligonucleotide molecules is about 1 nanomole (nmol) of oligonucleotide molecules per plant, for example, from about 0.05 to 1 nmol per plant. Other embodiments for herbaceous plants include useful ranges of about 0.05 to about 100 nmol, or about 0.1 to about 20 nmol, or about 1 nmol to about 10 nmol of polynucleotides per plant. Very large plants, trees, or to vines may require correspondingly larger amounts of polynucleotides. To illustrate embodiments, the factor 1×, when applied to oligonucleotide molecules is arbitrarily used to denote a treatment of 0.8 nmol of polynucleotide molecule per plant; 10×, 8 nmol of polynucleotide molecule per plant; and 100×, 80 nmol of polynucleotide molecule per plant.

An agronomic field in need of virus control may be treated by application of an agricultural chemical composition directly to the surface of the growing plants, such as by a spray. For example, the method is applied to control virus infection in a field of crop plants by spraying the field with the composition. The composition can be provided as a tank mix with one or more pesticidal or herbicidal chemicals to control pests and diseases of the crop plants in need of pest and disease control, a sequential treatment of components (generally the polynucleotide containing composition followed by the pesticide), or a simultaneous treatment or mixing of one or more of the components of the composition from separate containers. Treatment of the field can occur as often as needed to provide virus control and the components of the composition can be adjusted to target specific Tospoviruses or Geminiviruses through utilization of specific polynucleotides or polynucleotide compositions capable of selectively targeting the specific virus to be controlled. The composition can be applied at effective use rates according to the time of application to the field, for example, preplant, at planting, post planting, or post harvest. The polynucleotides of the composition can be applied at rates of 1 to 30 grams per acre depending on the number of trigger molecules needed for the scope of virus infection in the field.

Crop plants in which virus control may be needed include but are not limited to corn, soybean, cotton, canola, sugar beet, alfalfa, sugarcane, rice, barley, and wheat; vegetable plants including, but not limited to, tomato, sweet pepper, hot pepper, melon, watermelon, cucumber, zucchini, eggplant, cauliflower, broccoli, lettuce, spinach, onion, peas, carrots, sweet corn, Chinese cabbage, leek, fennel, pumpkin, squash or gourd, radish, potato, Brussels sprouts, tomatillo, peanut, garden beans, dry beans, or okra; culinary plants including, but not limited to, basil, parsley, coffee, or tea; or fruit plants including but not limited to apple, pear, cherry, peach, plum, apricot, banana, plantain, table grape, wine grape, citrus, avocado, mango, or berry; a tree grown for ornamental or commercial use, including, but not limited to, a fruit or nut tree; ornamental plant (e.g., an ornamental flowering plant or shrub or turf to grass), such as iris and impatiens. The methods and compositions provided herein can also be applied to plants produced by a cutting, cloning, or grafting process (i.e., a plant not grown from a seed) including fruit trees and plants that include, but are not limited to, avocados, tomatoes, eggplant, cucumber, melons, watermelons, and grapes, as well as various ornamental plants.

The trigger polynucleotide compositions may also be used as mixtures with various agricultural chemicals and/or insecticides, miticides and fungicides, pesticidal and biopesticidal agents. Examples include, but are not limited to, azinphos-methyl, acephate, isoxathion, isofenphos, ethion, etrimfos, oxydemeton-methyl, oxydeprofos, quinalphos, chlorpyrifos, chlorpyrifos-methyl, chlorfenvinphos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafenthion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofencarb, carbaryl, carbosulfan, xylylcarb, thiodicarb, pirimicarb, fenobucarb, furathiocarb, propoxur, bendiocarb, benfuracarb, methomyl, metolcarb, XMC, carbofuran, aldoxycarb, oxamyl, acrinathrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cypermethrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, bensultap, acetamiprid, imidacloprid, clothianidin, dinotefuran, thiacloprid, thiamethoxam, nitenpyram, chlorfluazuron, diflubenzuron, teflubenzuron, triflumuron, novaluron, noviflumuron, bistrifluoron, fluazuron, flucycloxuron, flufenoxuron, hexaflumuron, lufenuron, chromafenozide, tebufenozide, halofenozide, methoxyfenozide, diofenolan, cyromazine, pyriproxyfen, buprofezin, methoprene, hydroprene, kinoprene, triazamate, endosulfan, chlorfenson, chlorobenzilate, dicofol, bromopropylate, acetoprole, fipronil, ethiprole, pyrethrin, rotenone, nicotine sulphate, BT (Bacillus Thuringiensis) agent, spinosad, abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, fluazinam, flufenzin, hexythiazox, propargite, benzomate, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, triadimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triticonazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuarimol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D, carbam, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, to machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tiadinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine-acetate, quintozene, tolylfluanid, anilazine, nitrothalisopropyl, fenitropan, dimethirimol, benthiazole, harpin protein, flumetover, mandipropamide and penthiopyrad.

All publications, patents, and patent applications are herein incorporated by reference to the same extent as if each individual publication or patent application was specifically and individually indicated to be incorporated by reference.

The following Examples are presented for the purposes of illustration and should not be construed as limitations. Those of skill in the art should, in light of the present disclosure, appreciate that many changes can be made in the specific embodiments that are disclosed herein and still obtain a like or similar result without departing from the spirit and scope.

### Example 1

### Topical Application of Antisense ssDNA Oligonucleotides to Lettuce Plants for Control of Impatiens Necrotic Spotted Virus (INSV)

Single-stranded DNA (ssDNA) fragments in antisense (as) orientation were identified and mixed with a transfer agent and other components. This composition was topically applied to lettuce plants to effect repression of the target INSV nucleocapsid (N) gene to reduce or eliminate symptoms of viral infection in the plants. The procedure was as follows.

Growing lettuce plants (Lactuca sativa, c.v. SVR3606-L4) were topically treated with a composition for inducing suppression of a target gene in a plant. The composition included: (a) an agent to enable permeation of the polynucleotides into the plant, and (b) at least one polynucleotide strand including at least one segment of 17-25 contiguous nucleotides of the target gene in antisense orientation. Lettuce plants were topically treated with an adjuvant solution comprising antisense ssDNA, essentially homologous or essentially complementary to the INSV N protein coding sequence. Plants were grown and treated in growth chambers [22° C., 8 hour light (˜50 μmol), 16 hour dark cycles].

Lettuce plants were germinated for approximately 16-21 days prior to assay. Single leaves of lettuce plants (40 plants total) were infected with approximately 200 nanograms (100 ng/μL in phosphate buffer) of INSV virus. Approximately 3 hours after virus infection, 20 plants were sprayed with a mixture of oligonucleotides in solution (SEQ ID NO:1 and SEQ ID NO:2, mixed together) using an airbrush at 20 psi. The sequences of the antisense ssDNA oligonucleotides are listed in Table 1. The remaining 20 plants were not treated with oligonucleotides and served as the control.

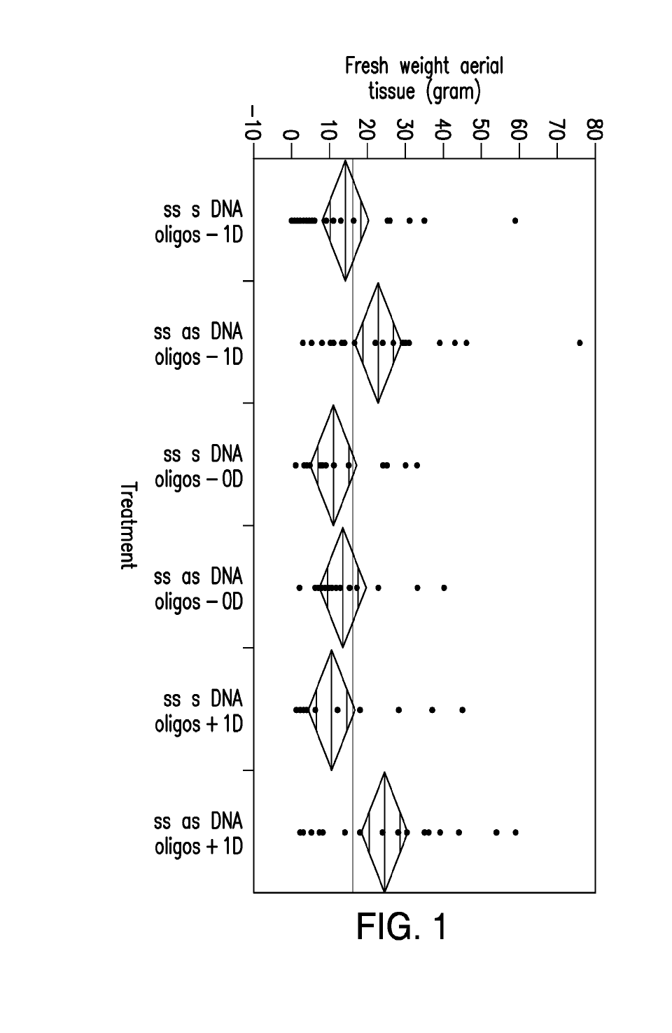

The final concentration of each oligonucleotide or polynucleotide was 20 nMoles for ssDNA (in 0.1% Silwet L-77, 2% ammonium sulfate, 5 mM sodium phosphate buffer, pH 6.8) unless otherwise stated. The spray solution was applied to the plant to provide a total of 200-300 μL volume. The fresh weight of aerial tissue was measured (see FIG. 1).

TABLE 1
The sequence of antisense ssDNA oligonucleotides
directed to INSV nucleocapsid gene N.

| SEQ | | | | |
|---|---|---|---|---|
| ID NO | Sequence (5′-3′) | Length | Virus | Target |
| 1 | GCTATAAACAGC | 23 | INSV | Nucleocapsid |
| | CTTCCAAGTCA | | | Gene (N) |
| 2 | GTCATTAAGAGT | 23 | INSV | Nucleocapsid |
| | GCTGACTTCAC | | | Gene (N) |

### Example 2

### Quantification of Virus Using ELISA

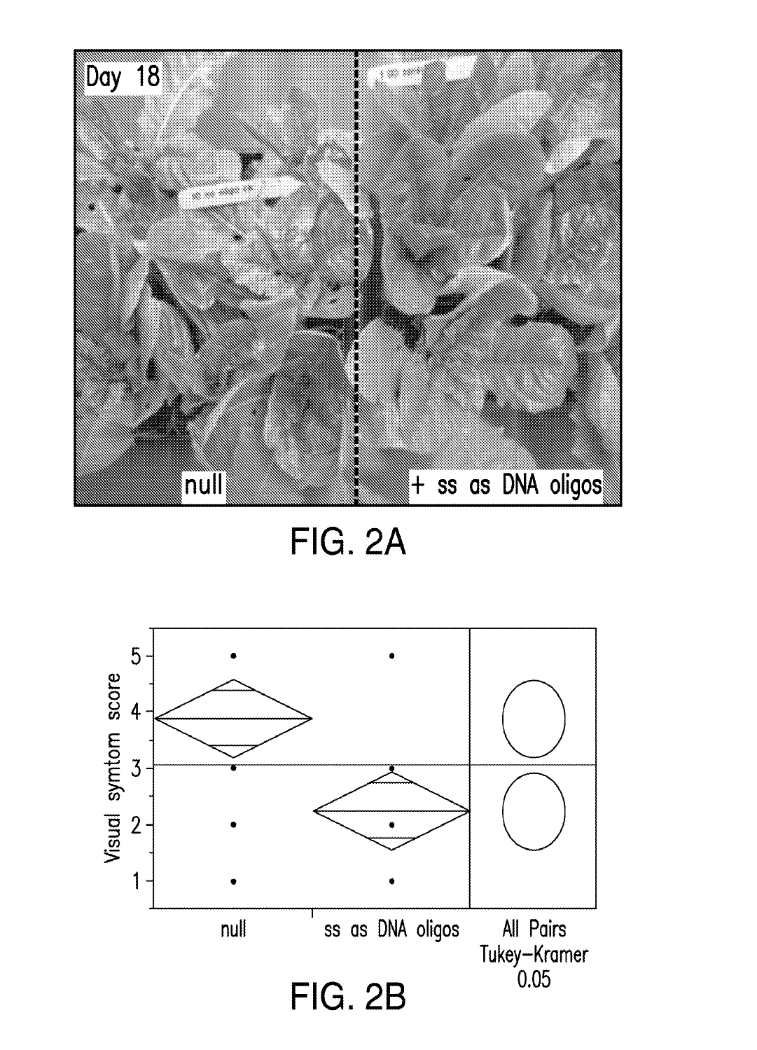

Leaf punctures harvested from untreated or treated plants lettuce plants (FIG. 2) as described in Example 1 were crushed in antigen buffer using a mortar and pestle. The homogenate was centrifuged at 10,000 rpm for 5 minutes at 4° C. The supernatant was extracted and subjected to indirect-ELISA against the anti-INSV N protein.

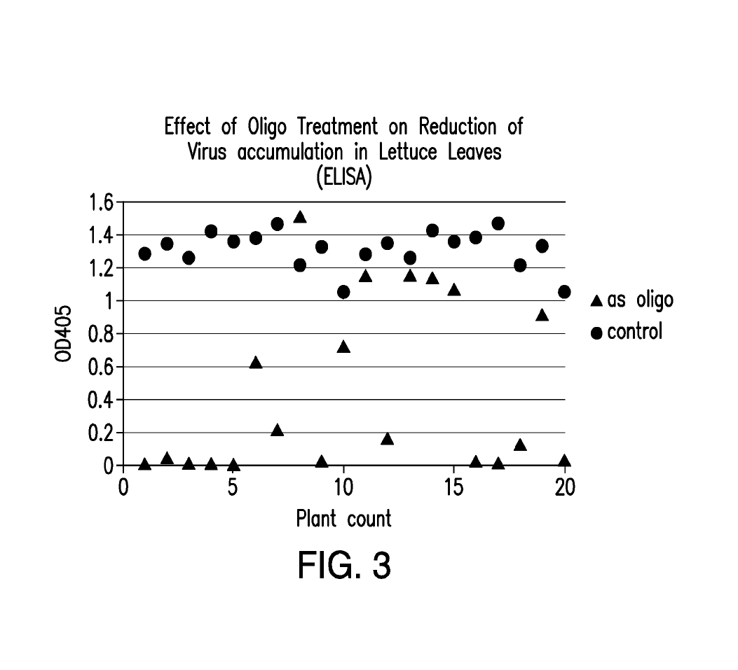

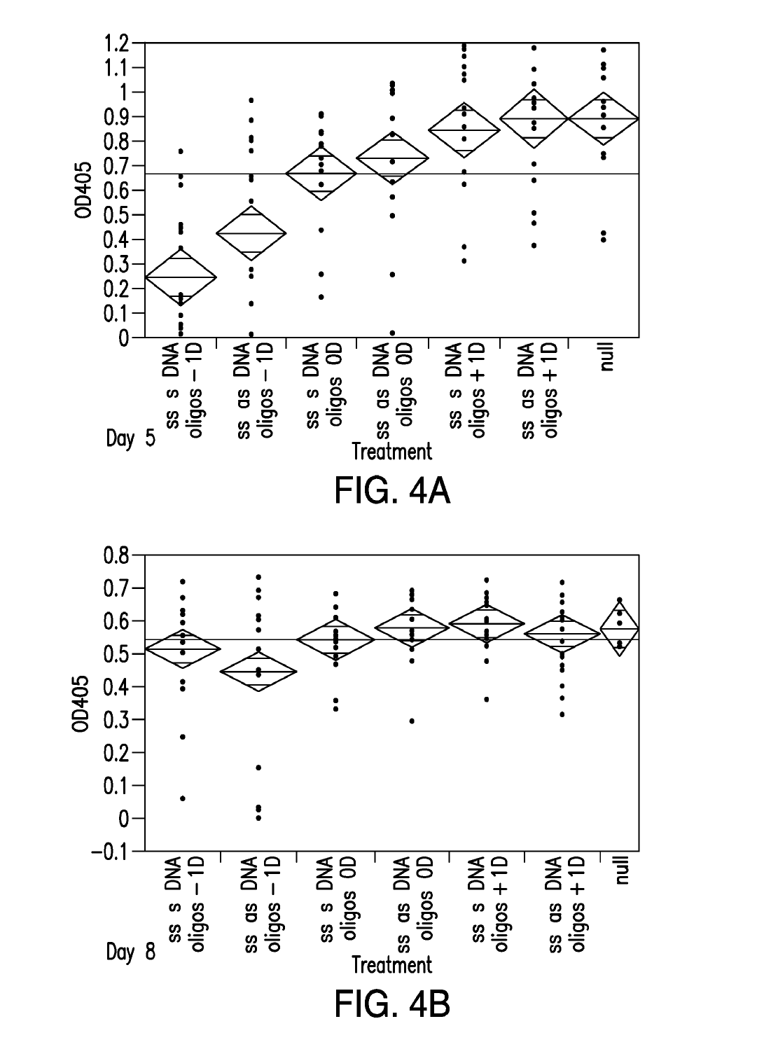

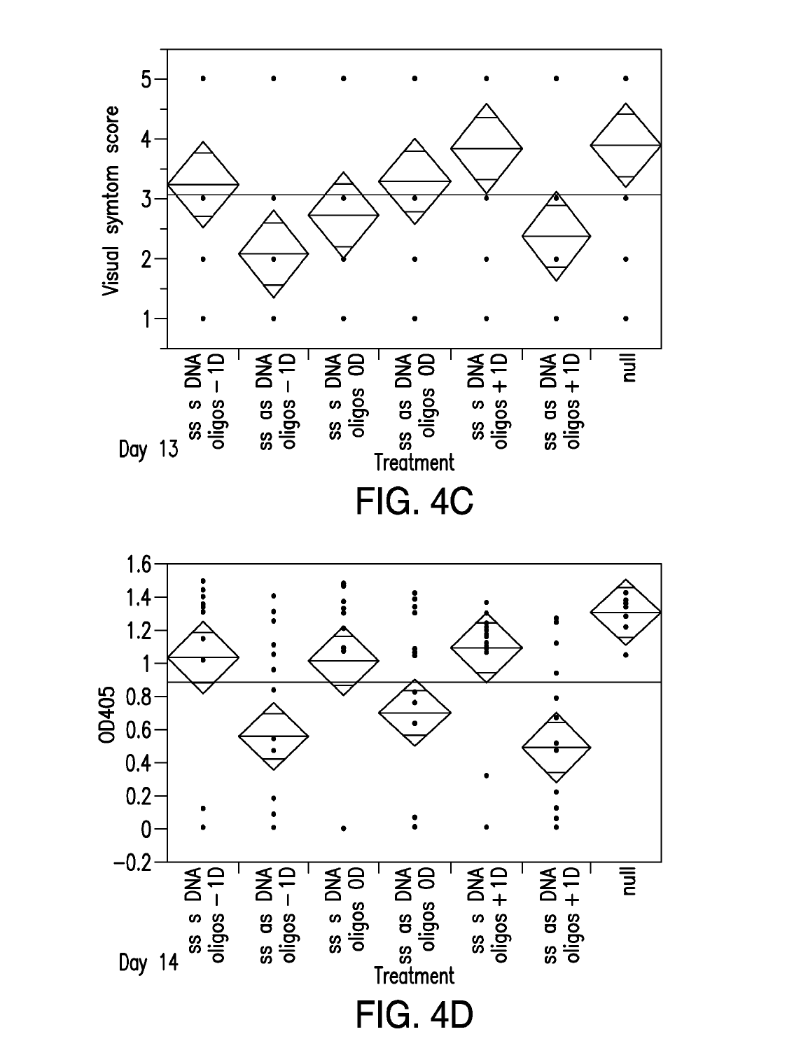

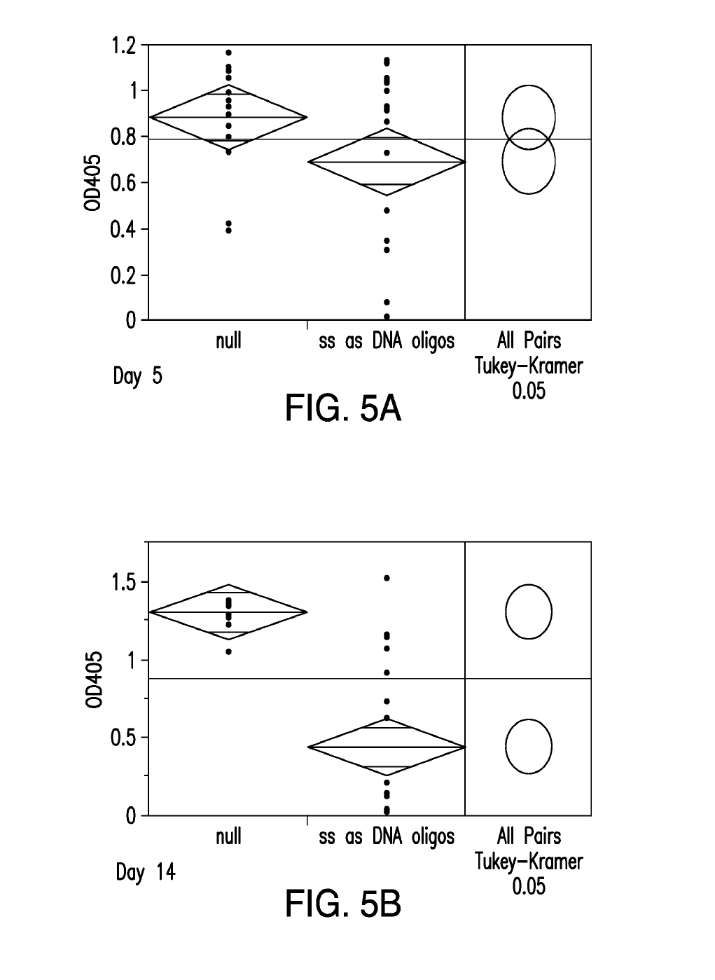

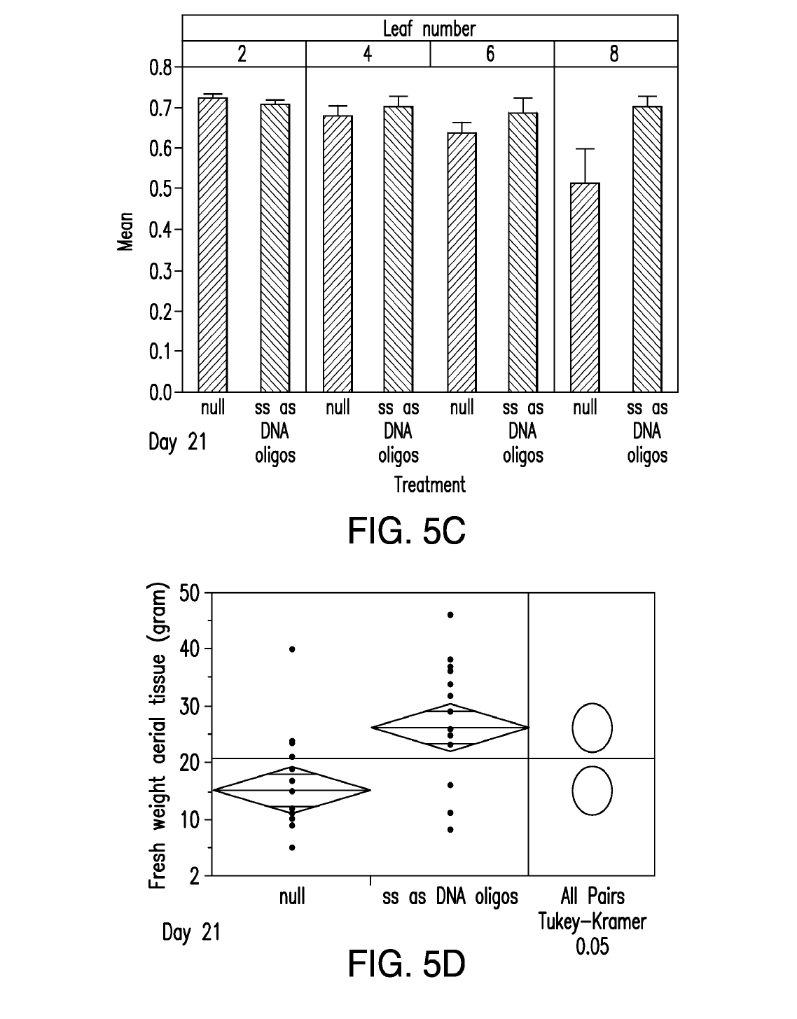

As shown in FIG. 3, circles represent a readout of INSV N protein in individual leaf punches collected from the control plants (virus only, no polynucleotide). Triangles represent a readout of INSV N protein in individual leaf punches collected from plants treated with a mixture of antisense ssDNA oligonucleotides (SEQ ID NO:1 and SEQ ID NO:2). Approximately 65% of the oligo-treated plants exhibited OD₄₀₅ values of 0.2 or lower, and 100% of the control plants exhibited an OD₄₀₅ value of 1 or higher. FIG. 4 and FIG. 5 show optical density (OD) and visual assessment of extracts of lettuce plants after treatment with antisense ssDNA oligos.

### Example 3

### Topical Application of Antisense ssDNA Oligonucleotides to Lettuce Plants after Virus Treatment Improves Photosystem II Function

In this example, lettuce plants that were untreated (null) or that had been infected with INSV virus and treated with ss antisense oligonucleotides were measured using a portable chlorophyll fluorometer (PAM-2500). This measurement gives an effective yield of photosystem II (PSII) function, a measure of overall yield. A group of six randomly picked non-treated and six randomly picked treated plants were measured at leaf number 2, 4, 6 and 8. The leaf number is indicative of the age of the lettuce head with the youngest leaf (leaf 2) being inside the forming lettuce head and the oldest leaf (leaf 8) located on the outside of the forming lettuce head. Plants treated with ss antisense DNA oligos exhibited the most protection on the outer leaves compared to untreated (null) plants.

### Example 4

### Topical Application of Antisense ssDNA Oligonucleotides to Tomato and Pepper Plants for Control of Tomato Spotted Wilt Virus (TSWV)

Single-stranded or double-stranded DNA or RNA fragments in sense or antisense orientation, or both, were identified and mixed with a transfer agent and other components. This composition was topically applied to tomato plants to effect expression of the target TSWV nucleocapsid or capsid genes to reduce or eliminate symptoms of viral infection in the plants. The procedure was as follows.

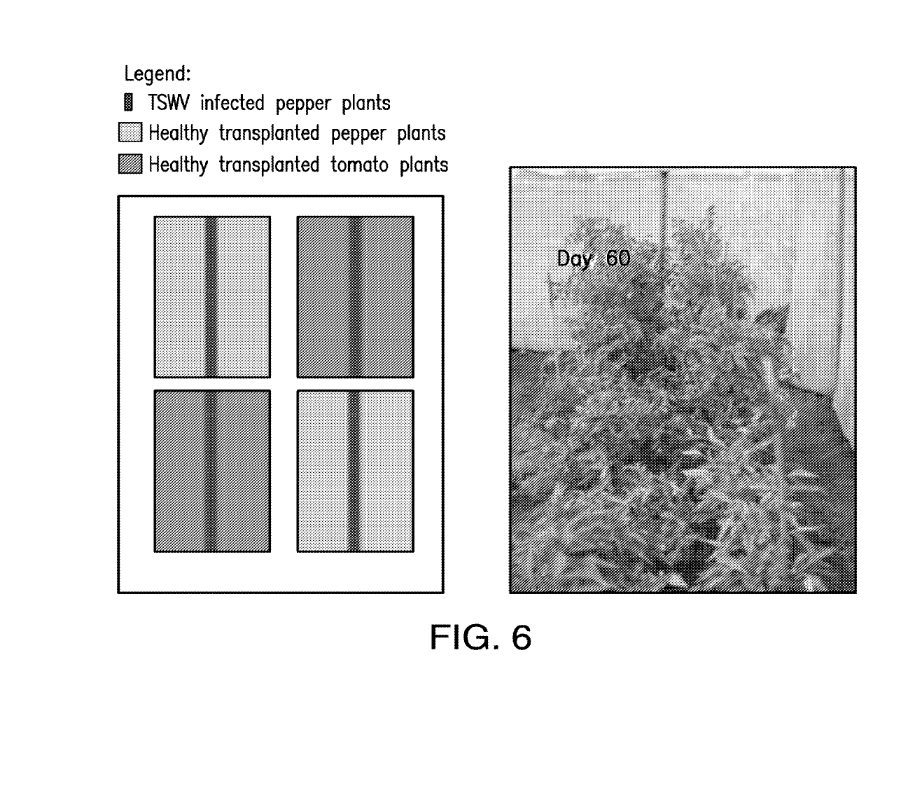

Tomato plants (Solanum lycopersicum HP375) and pepper plants (c.v. Yolo Wonder B) were grown in a cage outdoors. Pepper plants infected with TSWV, a negative-sense RNA virus, were transplanted from a breeder's infected pepper field in the center of the rows containing either tomato or pepper plants. Any subsequent infection was due to thrips transmitting TSWV from the infected center plants, thus mimicking a natural TSWV infection (see FIG. 6). Topical treatment with a mixture of at least one polynucleotide strand including at least one segment of 17-25 contiguous nucleotides of the target gene in either antisense or sense orientation was performed. Plants were treated with a topically applied adjuvant solution of trigger molecules comprising ssDNA oligonucleotides essentially homologous or essentially complementary to the TSWV nucleocapsid coding sequence. The sequence of the trigger molecule used in each treatment is shown in Table 2.

TABLE 2
The sequence of antisense ssDNA oligonucleotides
directed to TSWV nucleocapsid gene N.

| SEQ | | | | |
|---|---|---|---|---|
| ID NO | Sequence (5′-3′) | Length | Virus | Target |
| 3 | CATCTCAAAGCT | 22 | TSWV | Nucleocapsid |
| | ATCAACTGAA | | | gene (N) |
| 4 | TGATCTTCATTC | 22 | TSWV | Nucleocapsid |
| | ATTTCAAATG | | | gene (N) |

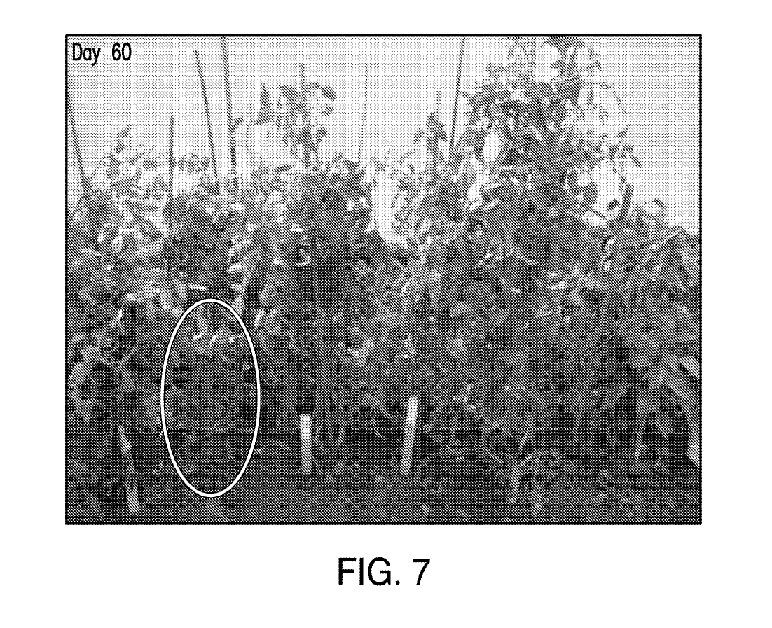

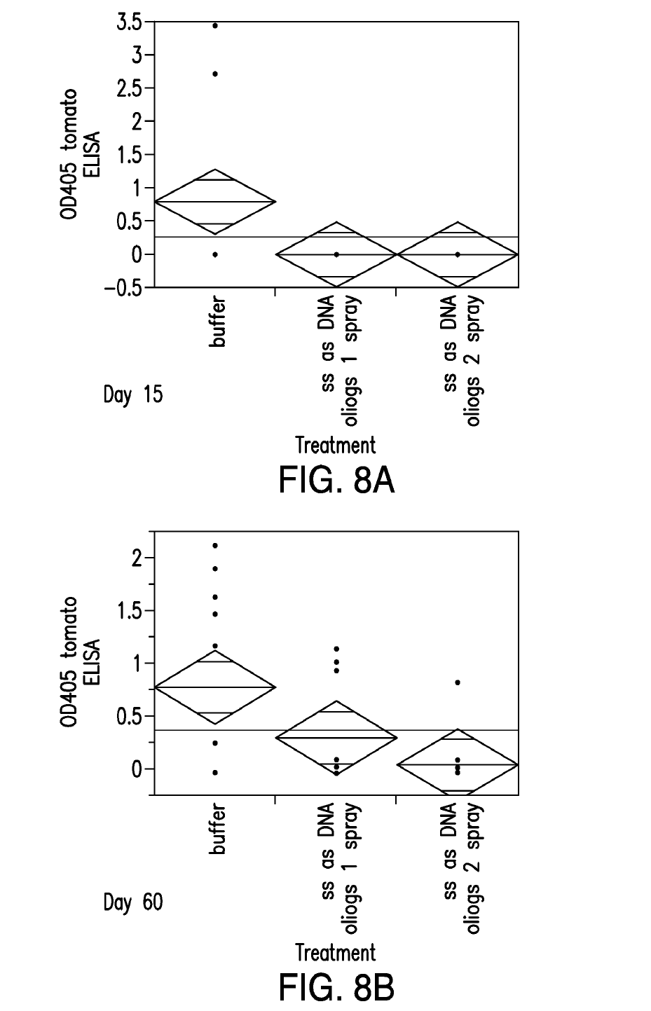

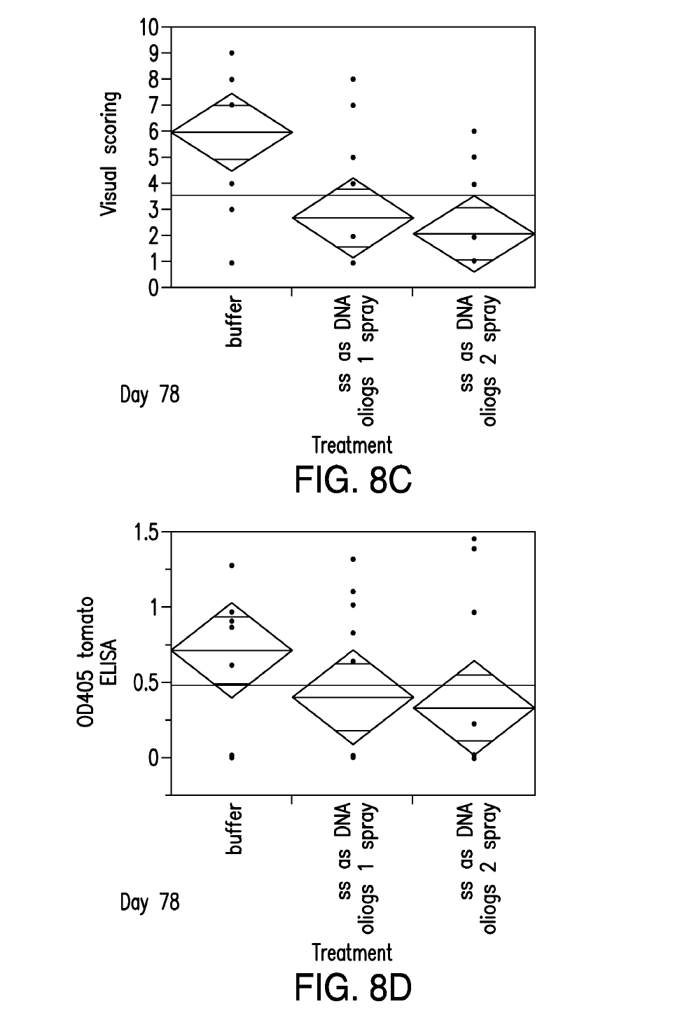

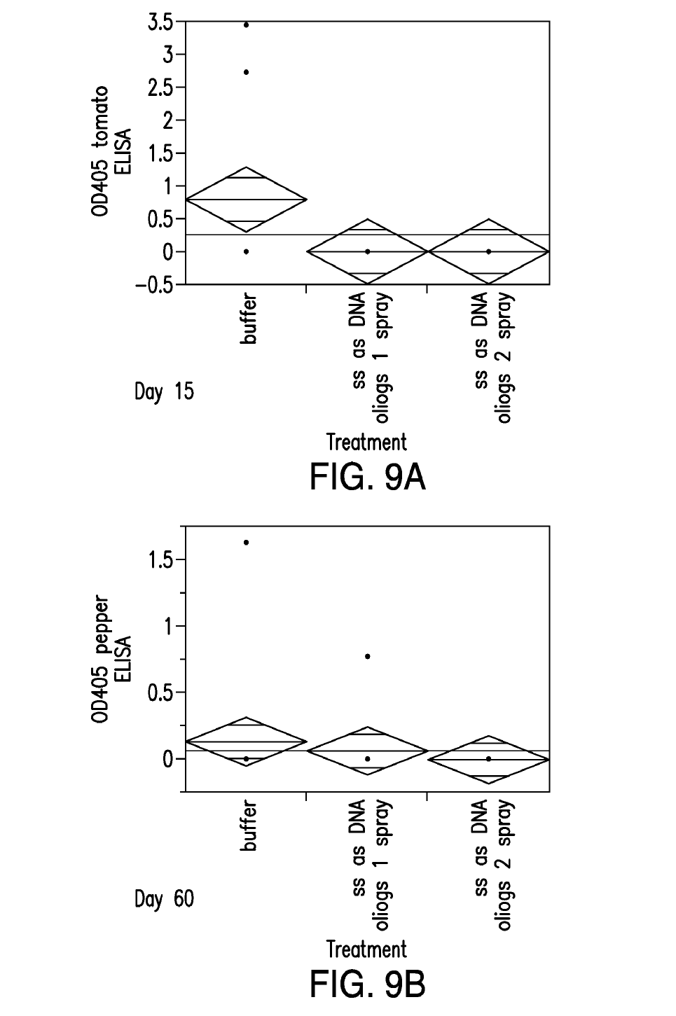

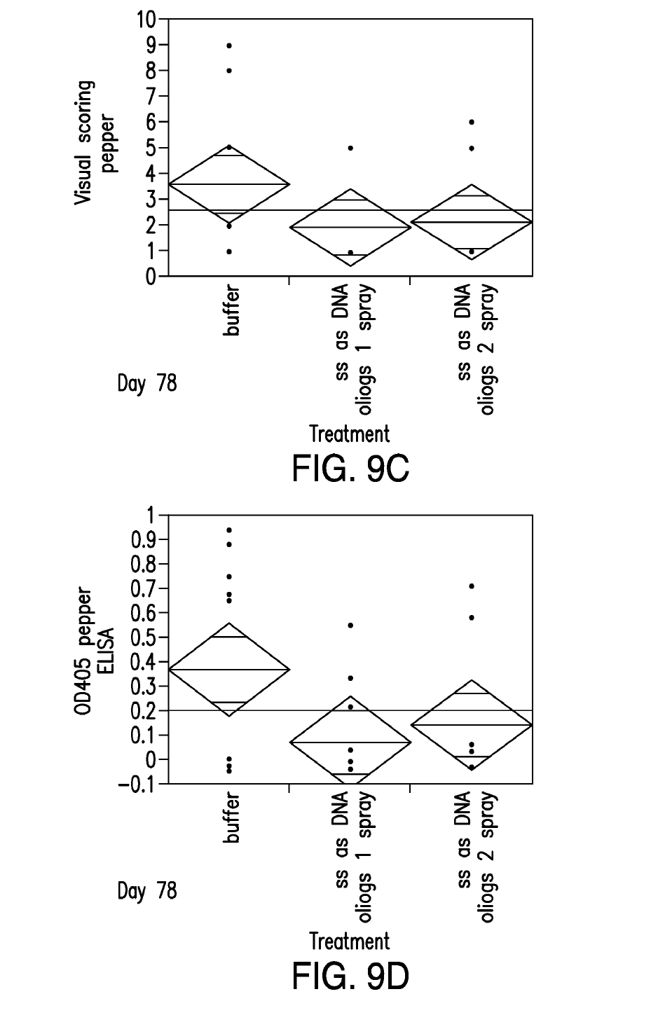

Plants at the 2-5 fully expanded leaf stage were used in these assays. Seven or 8 plants were treated as control (virus infection only) and 7 or 8 plants were treated with polynucleotides. Two fully expanded leaves per plant were treated with the polynucleotide/Silwet L-77 solution. The final concentration for each oligonucleotide or polynucleotide was 10 nmoles for ssDNA (in 0.1% Silwet L-77, 2% ammonium sulfate, 5 mM sodium phosphate buffer, pH 6.8) unless otherwise stated. Twenty microliters of the solution was applied to the top surface of each of the two leaves to to provide a total of 40 μL for each plant. FIG. 7 shows tomato plants both untreated (circled) and topically treated with antisense ssDNA oligos against TSWV, while FIGS. 8 and 9 show the results of the topical treatment of tomato and pepper plants, respectively.

### Example 5

### Topical Application of Antisense ssDNA Oligonucleotides to Pepper Plants for Control of Cucumber Mosaic Virus (CMV)

In this example, growing pepper plants (c.v. Yolo Wonder B) were inoculated with cucumber mosaic virus (CMV), a positive strand RNA virus, and the plants were separated into two groups. The experimental group was then topically treated with a mixture of at least one polynucleotide strand including at least one segment of 17-25 contiguous nucleotides of the target gene in either antisense or sense orientation. The trigger molecules in the topical adjuvant solution comprised dsRNA and ssDNA essentially homologous or essentially complementary to the CMV capsid coding sequence. The sequences of the trigger molecules used in each treatment are shown in Table 3.

TABLE 3
The sequence of antisense ssDNA oligonucleotides
directed to CMV coat protein (CP).

| SEQ | | | | |
|---|---|---|---|---|
| ID NO | Sequence (5′-3′) | Length | Virus | Target |
| 5 | AGACGTGGGAAG | 21 | CMV | Coat Protein |
| | TGCGTTGGT | | | (CP) |
| 6 | CTCGACGTCAAC | 21 | CMV | Coat Protein |
| | ATGAAGTAC | | | (CP) |
| 7 | GCTTGGACTCCA | 21 | CMV | Coat Protein |
| | GATGCAGCA | | | (CP) |
| 8 | TACTGATAAACC | 21 | CMV | Coat Protein |
| | AGTACCGGT | | | (CP) |
| 9 | CGAATTTGAATG | 21 | CMV | Coat Protein |
| | CGCGAAACA | | | (CP) |
| 10 | AGTTTCTTGTCA | 21 | CMV | Coat Protein |
| | TATTCTGTG | | | (CP) |
| 11 | GACGACCAGCTG | 21 | CMV | Coat Protein |
| | CCAACGTCT | | | (CP) |
| 12 | TATTAAGTCGCG | 21 | CMV | Coat Protein |
| | AAAGCTGCT | | | (CP) |

Pepper plants at the 2-5 fully expanded leaf stage were used in the assays. Seven or 8 plants were used as the control (virus infection only) and 7 or 8 plants were treated with virus followed by a polynucleotide trigger solution. Two fully expanded leaves per plant were treated with the polynucleotide/Silwet L-77 solution. One set of plants was treated with a mixture of polynucleotides comprising SEQ ID NOs:5-8 and another set of plants was treated with a mixture of polynucleotides comprising SEQ ID NOs:9-12. The final concentration for each oligonucleotide or polynucleotide was 5 nmol for ssDNA (in 0.1% Silwet L-77, 2% ammonium sulfate, 5 mM sodium phosphate buffer, pH 6.8) unless otherwise stated. Twenty microliters of the solution was applied to the top surface of each of the two leaves to provide a total of 40 μL for each plant.

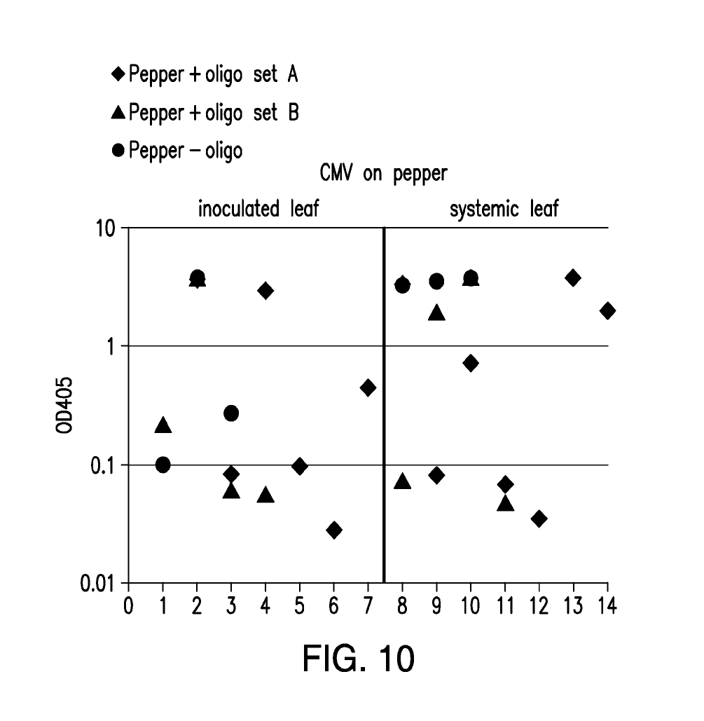

As shown in FIG. 10, circles represent data points collected from the control plants (virus only, no oligo treatment). Diamonds (SEQ ID NOs:5-8) and triangles (SEQ ID NOs:9-12) represent data points collected from samples topically treated with the antisense ssDNA oligonucleotide solution. The left part shows data from inoculated leaves, and the right part shows data from systemic non-infected, non-oligo-treated leaves.

### Example 6

### Topical Application of Antisense ssDNA Oligonucleotides to Onion Plants for Control of Iris Yellow Spot Virus (IYSV)

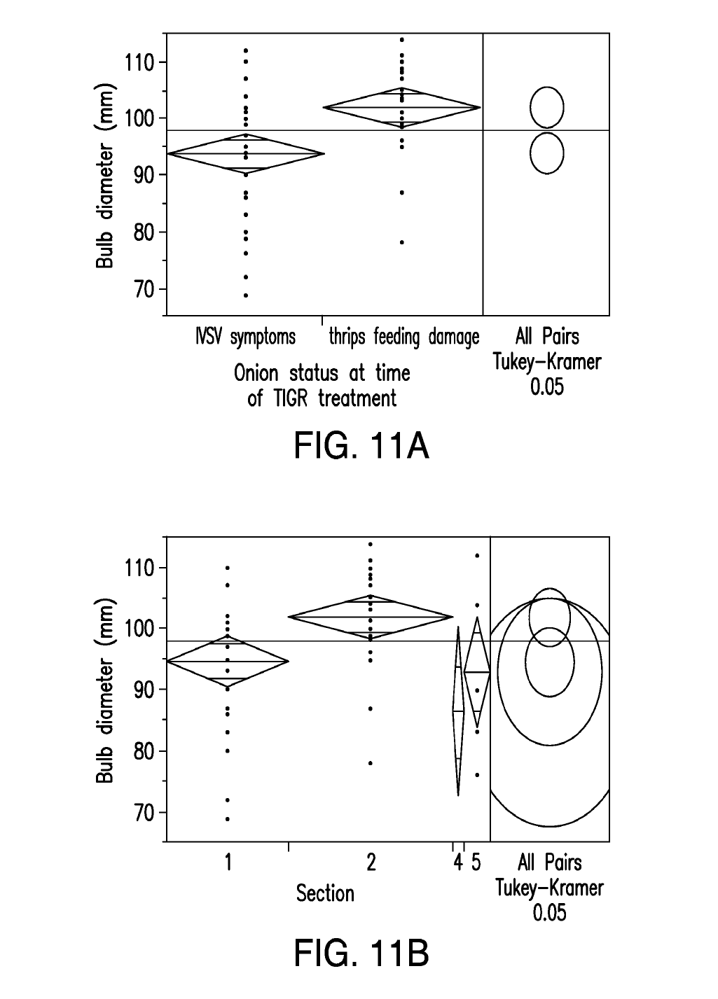

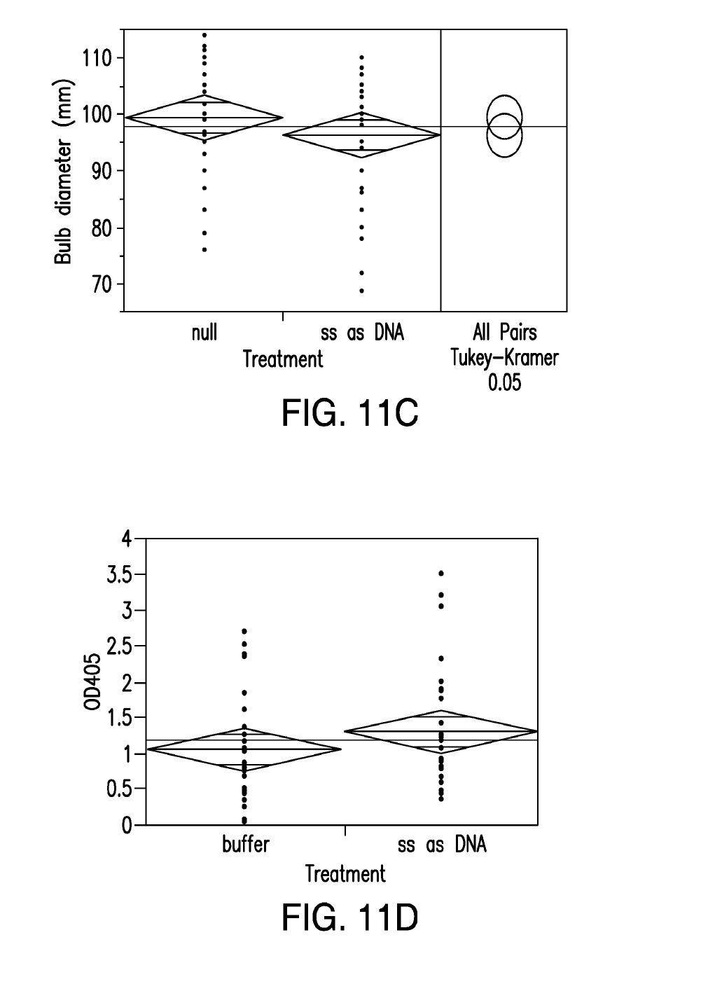

In this example, growing onion plants were inoculated with iris yellow spot virus (IYSV), and the plants were separated into two groups (31 plants per group). The experimental group was then topically treated with a mixture of at least one polynucleotide strand including at least one segment of 17-25 contiguous nucleotides of the target gene in antisense orientation. The trigger molecules in the topical adjuvant solution comprised ssDNA essentially homologous or essentially complementary to an IYSV coding sequence. The results of treatment of onion plants with antisense ssDNA are shown in FIG. 11.

### Example 7

### Topical Application of Polynucleotide Triggers for Control of Commercially Relevant Tospovirus Isolates

In Table 4 of this example, the sequences of genes of Tospovirus isolates considered to be commercially relevant because of yield losses in tomato, pepper, potato, or soybean were identified and constitute SEQ ID NOs:13-46.

A computer alignment was used to identify highly conserved areas within the Nucleocapsid (N), Silencing Suppressor (NSs), Movement (NSm), and RNA-dependent RNA polymerase genes (SEQ ID NOs:47-103 in Table 5) to serve as candidates for antisense ssDNA or dsRNA polynucleotides homologous to the gene sequence for topical application treatment to control Tospovirus infection (Table 5). These polynucleotides can be tested on Tospovirus-infected tomato plants to control viral infection.

TABLE 4
RNA Sequences of Tospoviruses.

| SEQ | | | | | |
|---|---|---|---|---|---|
| ID NO: | Species | Gene | Host | Isolate | Accession No. |
| 13 | Groundnut ringspot | N | Florida tomato | FL, USA | HQ634665.1 |
| | virus isolate | | | | |
| 14 | Groundnut ringspot and | N | Solanum lycopersicum | FL, USA | gi\|332290587 |
| | Tomato chlorotic spot | | | | |
| | virus reassortant | | | | |
| 15 | Tomato spotted wilt | N | Eustoma grandiflorum | USA | HQ655877.1 |
| | virus | | | | |
| 16 | Tomato spotted wilt | N | Pepper | Brazil | DQ915948.1 |
| | virus | | | | |
| 17 | Tomato spotted wilt | N | Potato | NC, USA | AY856344 |
| | virus | | | | |
| 18 | Tomato chlorotic spot | N | Florida tomato | FL, USA | HQ634664.1 |
| | virus | | | | |
| 19 | Tomato chlorotic spot | N | | FL, USA | JX244198.1 |
| | virus | | | | |
| 20 | Tomato chlorotic spot | N | | FL, USA | JX244196 |
| | virus | | | | |
| 21 | Tomato spotted wilt | N | Solanum lycopersicum | FL, USA | HQ634670 |
| | virus | | | | |
| 22 | Tomato spotted wilt | N | Solanum lycopersicum | FL, USA | HQ634668.1 |
| | virus | | | | |
| 23 | Tomato spotted wilt | N | Solanum lycopersicum | FL, USA | HQ634669.1 |
| | virus | | | | |
| 24 | Tomato spotted wilt | N | Solanum lycopersicum | FL, USA | HQ634667.1 |
| | virus | | | | |
| 25 | Groundnut ringspot | NSm | Florida tomato | FL, USA | HQ634675.1 |
| | virus isolate | | | | |
| 26 | Groundnut ringspot | NSm | Glycine max | S.A | HQ634674 |
| | virus isolate | | | | |
| 27 | Tomato spotted wilt | NSm | | USA | NC_002050 |
| | virus | | | | |
| 28 | Tomato chlorotic spot | NSm | Florida tomato | FL, USA | HQ634671.1 |
| | virus | | | | |
| 29 | Tomato chlorotic spot | NSm | Solanum lycopersicum | FL, USA | JX244201.1 |
| | virus | | | | |
| 30 | Tomato spotted wilt | NSm | Solanum lycopersicum | FL, USA | HQ634676.1 |
| | virus | | | | |
| 31 | Tomato spotted wilt | NSm | Solanum lycopersicum | FL, USA | AY956380 |
| | virus | | | | |
| 32 | Groundnut ringspot and | NSs | Solanum lycopersicum | FL, USA | gi\|332290587 |
| | Tomato chlorotic spot | | | | |
| | virus reassortant | | | | |
| 33 | Groundnut ringspot | NSs | Groundnut | S.A | JN571117 |
| | virus isolate | | | | |
| 34 | Tomato spotted wilt | NSs | Solanum lycopersicum | USA | FR693044 |
| | virus | | | | |
| 35 | Tomato spotted wilt | NSs | Pepper | Brazil | D00645.1 |
| | virus | | | | |
| 36 | Tomato spotted wilt | NSs | | USA | AF020659.1 |
| | virus | | | | |
| 37 | Tomato spotted wilt | NSs | | USA | AF020659 |
| | virus | | | | |
| 38 | Groundnut ringspot | RdRp/L | Florida tomato | FL, USA | HQ634677.1 |
| | virus isolate | segment | | | |
| 39 | Groundnut ringspot | RdRp/L | Florida tomato | FL 34945, | HQ634679.1 |
| | virus isolate | segment | | USA 95/0188 | |
| 40 | Groundnut ringspot | RdRp/L | Florida tomato | FL, USA | HQ634678.1 |
| | vims isolate | segment | | 95/0137 | |
| 41 | Tomato spotted wilt | RdRp/L | strain = “BR-01 (CNPH1 | Brazil | NC_002052 |
| | virus | segment | | | |
| 42 | Tomato chlorotic spot | RdRp/L | Florida tomato | FL, USA | HQ634680.1 |
| | virus | segment | | | |
| 43 | Tomato chlorotic spot | RdRp/L | Solanum lycopersicum | Brazil | HQ700667.1 |
| | virus | segment | | | |
| 44 | Tomato chlorotic spot | RdRp/L | Solanum lycopersicum | FL, USA | JX244205.1 |
| | virus | segment | | | |
| 45 | Tomato chlorotic spot | RdRp/L | Solanum lycopersicum | FL, USA | JX244203 |
| | virus | segment | | | |
| 46 | Tomato chlorotic spot | RdRp/L | Solanum lycopersicum | USA | FR692596 |
| | virus | segment | | | |

TABLE 5
The sequence of dsRNA oligonucleotides directed to Tospoviruses.

| SEQ | | | |
|---|---|---|---|
| ID NO: | Type | Length | Gene, Virus, Description |
| 47 | dsRNA | 101 | N gene, Groundnut ringspot virus |
| 48 | dsRNA | 47 | N gene, Groundnut ringspot virus, 2NT overhangs at 3′ |
| 49 | dsRNA | 47 | N gene, Groundnut ringspot virus, 2NT overhangs at 3′ |
| 50 | dsRNA | 47 | N gene, Groundnut ringspot virus, 2NT overhangs at 3′ |
| 51 | dsRNA | 47 | N gene, Groundnut ringspot virus, 2NT overhangs at 3′ |
| 52 | dsRNA | 100 | N gene, Tomato spotted wilt virus |
| 53 | dsRNA | 47 | N gene, Tomato spotted wilt virus, 2NT overhangs at 3′ |
| 54 | dsRNA | 51 | N gene, Tomato spotted wilt virus, 2NT overhangs at 3′ |
| 55 | dsRNA | 51 | N gene, Tomato spotted wilt virus, 2NT overhangs at 3′ |
| 56 | dsRNA | 100 | N gene, Tomato chlorotic spot virus |
| 57 | dsRNA | 47 | N gene, Tomato chlorotic spot virus, 2NT overhangs at 3′ |
| 58 | dsRNA | 47 | N gene, Tomato chlorotic spot virus, 2NT overhangs at 3′ |
| 59 | dsRNA | 47 | N gene, Tomato chlorotic spot virus, 2NT overhangs at 3′ |
| 60 | dsRNA | 47 | N gene, Tomato chlorotic spot virus, 2NT overhangs at 3′ |
| 61 | dsRNA | 47 | N gene, Tomato chlorotic spot virus, 2NT overhangs at 3′ |
| 62 | dsRNA | 100 | NSm, Groundnut ringspot virus + TCSV |
| 63 | dsRNA | 47 | NSm, Groundnut ringspot virus + TCSV, 2NT overhangs at 3′ |
| 64 | dsRNA | 47 | NSm, Groundnut ringspot virus; long stretches of A/T's, 2NT overhangs at 3′ |
| 65 | dsRNA | 47 | NSm, Groundnut ringspot virus + TCSV, 2NT overhangs at 3′ |
| 66 | dsRNA | 201 | NSm, Tomato chlorotic spot virus + GRV |
| 67 | dsRNA | 47 | NSm, Tomato chlorotic spot virus + GRV, 2NT overhangs at 3′ |
| 68 | dsRNA | 23 | NSm, Tomato chlorotic spot virus + GRV |
| 69 | dsRNA | 51 | NSm, Tomato chlorotic spot virus + GRV, 2NT overhangs at 3′ |
| 70 | dsRNA | 150 | NSm, Tomato spotted wilt virus |
| 71 | dsRNA | 47 | NSm, Tomato spotted wilt virus, 2NT overhangs at 3′ |
| 72 | dsRNA | 47 | NSm, Tomato spotted wilt virus, 2NT overhangs at 3′ |
| 73 | dsRNA | 47 | NSm, Tomato spotted wilt virus, 2NT overhangs at 3′ |
| 74 | dsRNA | 100 | NSs, Tomato spotted wilt virus |
| 75 | dsRNA | 47 | NSs, Tomato spotted wilt virus, 2NT overhangs at 3′ |
| 76 | dsRNA | 47 | NSs, Tomato spotted wilt virus, 2NT overhangs at 3′ |
| 77 | dsRNA | 47 | NSs, Tomato spotted wilt virus, 2NT overhangs at 3′ |
| 78 | dsRNA | 47 | NSs, Tomato spotted wilt virus, 2NT overhangs at 3′ |
| 79 | dsRNA | 201 | RdRp, Groundnut ringspot virus isolate |
| 80 | dsRNA | 47 | RdRp, Groundnut ringspot virus isolate, 2NT overhangs at 3′ |
| 81 | dsRNA | 47 | RdRp, Groundnut ringspot virus isolate, 2NT overhangs at 3′ |
| 82 | dsRNA | 47 | RdRp, Groundnut ringspot virus isolate, 2NT overhangs at 3′ |
| 83 | dsRNA | 201 | RdRp, Tomato spotted wilt virus |
| 84 | dsRNA | 47 | RdRp, Tomato spotted wilt virus, 2NT overhangs at 3′ |
| 85 | dsRNA | 47 | RdRp, Tomato spotted wilt virus, 2NT overhangs at 3′ |
| 86 | dsRNA | 47 | RdRp, Tomato spotted wilt virus, 2NT overhangs at 3′ |
| 87 | dsRNA | 201 | RdRp, Tomato chlorotic spot virus |
| 88 | dsRNA | 47 | RdRp, Tomato chlorotic spot virus, 2NT overhangs at 3′ |
| 89 | dsRNA | 47 | RdRp, Tomato chlorotic spot virus, 2NT overhangs at 3′ |
| 90 | dsRNA | 47 | RdRp, Tomato chlorotic spot virus, 2NT overhangs at 3′ |
| 91 | dsRNA | 100 | Nsm, Tomato chlorotic spot virus |
| 92 | dsRNA | 47 | Nsm, Tomato chlorotic spot virus, 2NT overhangs at 3′ |
| 93 | dsRNA | 47 | Nsm, Tomato chlorotic spot virus, long stretches of T's, 2NT overhangs at 3′ |
| 94 | dsRNA | 47 | Nsm, Tomato chlorotic spot virus, 2NT overhangs at 3′ |
| 95 | dsRNA | 47 | Nsm, Tomato chlorotic spot virus, 2NT overhangs at 3′ |
| 96 | dsRNA | 47 | Nsm, Tomato chlorotic spot virus, 2NT overhangs at 3′ |
| 97 | dsRNA | 47 | Nsm, Tomato chlorotic spot virus, 2NT overhangs at 3′ |
| 98 | dsRNA | 47 | Nsm, Tomato chlorotic spot virus, 2NT overhangs at 3′ |
| 99 | dsRNA | 201 | NSs, Groundnut ringspot and Tomato chlorotic spot virus reassortant |
| 100 | dsRNA | 47 | NSs, Groundnut ringspot and Tomato chlorotic spot virus reassortant, 2NT |
| | | | overhangs at 3′ |
| 101 | dsRNA | 47 | NSs, Groundnut ringspot and Tomato chlorotic spot virus reassortant, 2NT |
| | | | overhangs at 3′ |
| 102 | dsRNA | 47 | NSs, Groundnut ringspot and Tomato chlorotic spot virus reassortant, 2NT |
| | | | overhangs at 3′ |
| 103 | dsRNA | 47 | NSs, Groundnut ringspot and Tomato chlorotic spot virus reassortant, 2NT |
| | | | overhangs at 3′ |

### Example 8

### Topical Application of Polynucleotide Triggers for Control of Other Commercially Relevant Plant Viruses in Agriculture

In Table 6 of this example, a commonly used computer algorithm was used to identify highly conserved regions in the coat protein (CP), Movement Protein (MP), and Silencing Suppressor protein, of plant virus isolates that are commercially relevant in agriculture. These viruses may be of different families, such as Geminiviruses (i.e., Cotton leaf curl virus, Barley yellow dwarf virus), or Bromoviruses (i.e., CMV), or Potexviruses (i.e., PepMV). The triggers identified in to Table 6 constitue SEQ ID NOs:104-268 and can be topically applied with a transfer agent to tomato, or pepper plants to test the efficacy against infection by the respective viruses.

TABLE 6
The sequence of dsRNA oligonucleotides directed to viruses of commercial relevance.

| SEQ | | | |
|---|---|---|---|
| ID NO: | Type | Length | Alias |
| 104 | dsRNA | 150 | BYD_CP |
| 105 | dsRNA | 150 | BYD_CP |
| 106 | dsRNA | 25 | BYD_CP_Conserved_across_strains_Overhangs |
| 107 | dsRNA | 140 | BYD_CP_Conserved_across_Strains |
| 108 | dsRNA | 25 | BYD_CP_overhangs |
| 109 | dsRNA | 21 | BYD_CP_overhangs |
| 110 | dsRNA | 150 | BYD_MP_Conserved_Across_Strains_Blunt |
| 111 | dsRNA | 22 | BYD_MP |
| 112 | dsRNA | 25 | BYD_MP |
| 113 | dsRNA | 150 | BYD_MP |
| 114 | dsRNA | 25 | BYD_MP |
| 115 | dsRNA | 25 | BYD_MP |
| 116 | dsRNA | 150 | BYD_Silencing_Suppressor |
| 117 | dsRNA | 25 | BYD_Silencing_Suppressor |
| 118 | dsRNA | 21 | BYD_Silencing_Suppressor_Blunt |
| 119 | dsRNA | 25 | BYD_Silencing_Suppressor_Overhang |
| 120 | dsRNA | 150 | CMV_CP |
| 121 | dsRNA | 25 | CMV_CP_Overhang_Conserved_Across_Strains |
| 122 | dsRNA | 25 | CMV_CP_Overhang_Conserved_Across_Strains |
| 123 | dsRNA | 25 | CMV_CP_Conserved_Across_Strains |
| 124 | dsRNA | 150 | CMV_CP |
| 125 | dsRNA | 150 | CMV_Silencing_Suppressor_Overhangs_Semi-Conserved_Across_Strains |
| 126 | dsRNA | 25 | CMV_Silencing_Suppressor |
| 127 | dsRNA | 25 | CMV_Silencing_Suppressor_Overhangs_Conserved_Across_Strains |
| 128 | dsRNA | 25 | CMV_Silencing_Suppressor_Overhangs_Conserved_Across_Strains |
| 129 | dsRNA | 21 | CMV_Silencing_Suppressor_Overhangs |
| 130 | dsRNA | 25 | CMV_MP_Overhangs_Semi-Conserved_Across_Strains |
| 131 | dsRNA | 21 | CMV_MP_Overhangs |
| 132 | dsRNA | 21 | CMV_MP_Overhangs |
| 133 | dsRNA | 21 | CMV_MP_Overhangs |
| 134 | dsRNA | 21 | CMV_MP_Overhangs_Semi-Conserved_Across_Strains |
| 135 | dsRNA | 21 | CMV_MP_Overhangs_Conserved_Across_Strains |
| 136 | dsRNA | 21 | CMV_MP_Overhangs_Conserved_Across_Strains |
| 137 | dsRNA | 21 | CMV_MP_Overhangs_Conserved_Across_Strains |
| 138 | dsRNA | 21 | CMV_MP_Overhangs |
| 139 | dsRNA | 150 | CMV_MP_Overhangs |
| 140 | dsRNA | 150 | CMV_MP_Overhangs |
| 141 | dsRNA | 25 | CMV_MP_Overhangs |
| 142 | dsRNA | 25 | CMV_MP_Overhangs |
| 143 | dsRNA | 25 | CMV_MP_Overhangs |
| 144 | dsRNA | 25 | CMV_MP_Overhangs |
| 145 | dsRNA | 21 | CMV_MP_Overhangs |
| 146 | dsRNA | 150 | PepMV_CP |
| 147 | dsRNA | 25 | PepMV_CP_Overhangs_Semi_Conserved_Across_Strains |
| 148 | dsRNA | 25 | PepMV_CP_Overhangs_Semi_Conserved_Across_Strains |
| 149 | dsRNA | 25 | PepMV_CP_Overhangs_Semi_Conserved_Across_Strains |
| 150 | dsRNA | 21 | PepMV_CP |
| 151 | dsRNA | 21 | PepMV_CP |
| 152 | dsRNA | 21 | PepMV_CP |
| 153 | dsRNA | 150 | PepMV_CP |
| 154 | dsRNA | 150 | PepMV_MP |
| 155 | dsRNA | 150 | PepMV_MP_Triple Gene Block1_{—} |
| 156 | dsRNA | 25 | PepMV_MP_Triple Gene Block1_Overhangs_Conserved_Across_Strains |
| 157 | dsRNA | 21 | PepMV_MP_Triple Gene Block1_Overhangs_Conserved_Across_Strains |
| 158 | dsRNA | 21 | PepMV_MP_Triple Gene Block1_Overhangs_Conserved_Across_Strains |
| 159 | dsRNA | 21 | PepMV_MP_Triple Gene Block1_Overhangs_Conserved_Across_Strains |
| 160 | dsRNA | 21 | PepMV_MP_Triple Gene Block1_Overhangs_Conserved_Across_Strains |
| 161 | dsRNA | 21 | PepMV_MP_Triple Gene Block1_Overhangs_Conserved_Across_Strains |
| 162 | dsRNA | 21 | PepMV_MP_Triple Gene Block1_Overhangs_Conserved_Across_Strains |
| 163 | dsRNA | 150 | PepMV_MP_Triple Gene Block2 |
| 164 | dsRNA | 21 | PepMV_MP_Triple Gene Block2_Overhangs_Conserved_Across_Strains |
| 165 | dsRNA | 21 | PepMV_MP_Triple Gene Block2_Overhangs_Conserved_Across_Strains |
| 166 | dsRNA | 21 | PepMV_MP_Triple Gene Block2_Overhangs_Conserved_Across_Strains |
| 167 | dsRNA | 21 | PepMV_MP_Triple Gene Block2_Overhangs_Conserved_Across_Strains |
| 168 | dsRNA | 21 | PepMV_MP_Triple Gene Block2_Overhangs_Conserved_Across_Strains |
| 169 | dsRNA | 150 | PepMV_MP_Triple Gene Block2 |
| 170 | dsRNA | 150 | PepMV_MP_Triple Gene Block3 |
| 171 | dsRNA | 21 | PepMV_MP_Triple Gene Block3_Overhangs |
| 172 | dsRNA | 21 | PepMV_MP_Triple Gene Block3_Overhangs |
| 173 | dsRNA | 21 | PepMV_MP_Triple Gene Block3_Overhangs |
| 174 | dsRNA | 21 | PepMV_MP_Triple Gene Block3_Overhangs |
| 175 | dsRNA | 150 | PepMV_MP_Triple Gene Block3_Overhangs |
| 176 | dsRNA | 21 | PepMV_MP_Triple Gene Block3_Overhangs |
| 177 | dsRNA | 150 | PepMV_MP_Triple Gene Block3 |
| 178 | dsRNA | 150 | CuCLV_CP_Overhangs_Conserved_across_Strains |
| 179 | dsRNA | 21 | CuCLV_CP_Overhangs_Conserved_across_Strains |
| 180 | dsRNA | 21 | CuCLV_CP_Overhangs_Conserved_across_Strains |
| 181 | dsRNA | 21 | CuCLV_CP_Overhangs_Conserved_across_Strains |
| 182 | dsRNA | 21 | CuCLV_CP_Overhangs_Conserved_across_Strains |
| 183 | dsRNA | 25 | CuCLV_CP_Overhangs_Conserved_across_Strains |
| 184 | dsRNA | 21 | CuCLV_CP_Overhangs_Conserved_across_Strains |
| 185 | dsRNA | 21 | CuCLV_CP_Overhangs_Conserved_across_Strains |
| 186 | dsRNA | 25 | CuCLV_CP_Overhangs_Conserved_across_Strains |
| 187 | dsRNA | 21 | CuCLV_CP_Overhangs_Conserved_across_Strains |
| 188 | dsRNA | 150 | CuCLV_Silencing Suppressor |
| 189 | dsRNA | 21 | CuCLV_Silencing Suppressor_Overhangs |
| 190 | dsRNA | 21 | CuCLV_Silencing Suppressor_Overhangs |
| 191 | dsRNA | 21 | CuCLV_Silencing Suppressor_Overhangs |
| 192 | dsRNA | 21 | CuCLV_Silencing Suppressor_Overhangs |
| 193 | dsRNA | 21 | CuCLV_Silencing Suppressor_Overhangs |
| 194 | dsRNA | 21 | CuCLV_Silencing Suppressor_Overhangs |
| 195 | dsRNA | 21 | CuCLV_Silencing Suppressor_Overhangs |
| 196 | dsRNA | 150 | CuCLV_MP_Overhang_Conserved_Across_Strains |
| 197 | dsRNA | 21 | CuCLV_MP_Overhang |
| 198 | dsRNA | 21 | CuCLV_MP_Overhang |
| 199 | dsRNA | 21 | CuCLV_MP_Overhang_Conserved_Across_Strains |
| 200 | dsRNA | 21 | CuCLV_MP_Overhang_Conserved_Across_Strains |
| 201 | dsRNA | 21 | CuCLV_MP_Overhang_Conserved_Across_Strains |
| 202 | dsRNA | 21 | CuCLV_MP_Overhang_Conserved_Across_Strains |
| 203 | dsRNA | 21 | CuCLV_MP_Overhang_Conserved_Across_Strains |
| 204 | dsRNA | 25 | CuCLV_MP_Overhang_Conserved_Across_Strains |
| 205 | dsRNA | 150 | TYLCV_CP |
| 206 | dsRNA | 21 | TYLCV_CP_Overhangs |
| 207 | dsRNA | 21 | TYLCV_CP_Overhangs |
| 208 | dsRNA | 21 | TYLCV_CP_Overhangs |
| 209 | dsRNA | 21 | TYLCV_CP_Overhangs |
| 210 | dsRNA | 21 | TYLCV_CP_Overhangs |
| 211 | dsRNA | 21 | TYLCV_CP_Overhangs |
| 212 | dsRNA | 21 | TYLCV_CP_Overhangs |
| 213 | dsRNA | 150 | TYLCV_CP |
| 214 | dsRNA | 150 | TYLCV_CP |
| 215 | dsRNA | 21 | TYLCV_CP_Overhangs |
| 216 | dsRNA | 150 | TYLCV_MP |
| 217 | dsRNA | 21 | TYLCV_MP_Overhangs_Conserved |
| 218 | dsRNA | 21 | TYLCV_MP_Overhangs_Conserved |
| 219 | dsRNA | 21 | TYLCV_MP_Overhangs_Conserved |
| 220 | dsRNA | 21 | TYLCV_MP_Overhangs_Conserved |
| 221 | dsRNA | 21 | TYLCV_MP_Overhangs_Conserved |
| 222 | dsRNA | 21 | TYLCV_MP_Overhangs_Conserved |
| 223 | dsRNA | 21 | TYLCV_MP_Overhangs_Conserved |
| 224 | dsRNA | 150 | TYLCV_Silencing Suppressor_C2 |
| 225 | dsRNA | 21 | TYLCV_Silencing Suppressor_C2_Overhangs |
| 226 | dsRNA | 21 | TYLCV_Silencing Suppressor_C2_Overhangs |
| 227 | dsRNA | 21 | TYLCV_Silencing Suppressor_C2_Overhangs |
| 228 | dsRNA | 21 | TYLCV_Silencing Suppressor_C2_Overhangs |
| 229 | dsRNA | 21 | TYLCV_Silencing Suppressor_C2_Overhangs |
| 230 | dsRNA | 21 | TYLCV_Silencing Suppressor_C2_Overhangs |
| 231 | dsRNA | 21 | TYLCV_Silencing Suppressor_C2_Overhangs |
| 232 | dsRNA | 150 | TYLCV_Silencing Suppressor_C2 |
| 233 | dsRNA | 150 | WSMV_CP |
| 234 | dsRNA | 21 | WSMV_CP_Overhangs |
| 235 | dsRNA | 21 | WSMV_CP_Overhangs |
| 236 | dsRNA | 21 | WSMV_CP_Overhangs |
| 237 | dsRNA | 21 | WSMV_CP_Overhangs |
| 238 | dsRNA | 21 | WSMV_CP_Overhangs |
| 239 | dsRNA | 21 | WSMV_CP_Overhangs |
| 240 | dsRNA | 21 | WSMV_CP_Overhangs |
| 241 | dsRNA | 150 | WSMV_CP |
| 242 | dsRNA | 150 | WSMV_CP |
| 243 | dsRNA | 21 | WSMV_CP_Overhangs |
| 244 | dsRNA | 21 | WSMV_CP_Overhangs |
| 245 | dsRNA | 21 | WSMV_CP_Overhangs |
| 246 | dsRNA | 21 | WSMV_CP_Overhangs |
| 247 | dsRNA | 21 | WSMV_CP_Overhangs |
| 248 | dsRNA | 21 | WSMV_CP_Overhangs |
| 249 | dsRNA | 21 | WSMV_CP_Overhangs |
| 250 | dsRNA | 25 | WSMV_CP_Blunt |
| 251 | dsRNA | 150 | WSMV_Nia_Vpg |
| 252 | dsRNA | 21 | WSMV_Nia_Vpg_Overhang |
| 253 | dsRNA | 21 | WSMV_Nia_Vpg_Overhang |
| 254 | dsRNA | 21 | WSMV_Nia_Vpg_Overhang |
| 255 | dsRNA | 21 | WSMV_Nia_Vpg_Overhang |
| 256 | dsRNA | 150 | WSMV_Nia_Vpg |
| 257 | dsRNA | 25 | WSMV_Nia_Vpg_Overhang |
| 258 | dsRNA | 21 | WSMV_Nia_Vpg_Overhang |
| 259 | dsRNA | 150 | WSMV_Nia_Pro_Overhang |
| 260 | dsRNA | 21 | WSMV_Nia_Pro_Overhang |
| 261 | dsRNA | 21 | WSMV_Nia_Pro_Overhang |
| 262 | dsRNA | 150 | WSMV_Nia_Pro_Overhang |
| 263 | dsRNA | 21 | WSMV_Nia_Pro_Overhang |
| 264 | dsRNA | 150 | WSMV_Nia_Pro |
| 265 | dsRNA | 21 | WSMV_Nia_Pro_Overhang |
| 266 | dsRNA | 25 | WSMV_Nia_Pro_Overhang |
| 267 | dsRNA | 21 | WSMV_Nia_Pro_Overhang |
| 268 | dsRNA | 21 | WSMV_Nia_Pro_Overhang |

### Example 9

### Topical Application of Polynucleotide Triggers for Control of Cucumber Mosaic Virus

In this example, the sequences of the Coat Protein (CM) Movement Protein (MP) or Silencing Suppressor (S) for different Cucumber Mosaic Viruses were identified and can be seen in Table 7. Topical application of ss antisense DNA or dsRNA sequences derived from the listed sequences (SEQ ID NOs:269-316) will be performed in pepper plants infected by Cucumber Mosaic Virus (CMV) using a transfer reagent and the plants will be scored by ELISA analysis and visual to assessment for reduction of symptoms.

TABLE 7
Sequences of target genes in Cucumber Mosaic Virus (CMV).

| SEQ | Sequence | | | | |
|---|---|---|---|---|---|
| ID NO: | ID | Host | Strain | Isolate | Gene |
| 269 | CMV | | | | CP -N Gene |
| 270 | AB004780 | | KM | Japan | CP -N Gene |
| 271 | D10538 | | Fny | USA (NY) | CP -N Gene |
| 272 | D00462 | | C | USA (NY) | CP -N Gene |
| 273 | L36251 | | Kor | Korea | CP -N Gene |
| 274 | U66094 | | Sny | Israel | CP -N Gene |
| 275 | U22821 | | Ny | Australia | CP -N Gene |
| 276 | D28487 | | FT | Japan | CP -N Gene |
| 277 | D10544 | | FC | USA | CP -N Gene |
| 278 | AJ890464 | Oriental Lily | OL | India | CP -N Gene |
| | | (Expression) | | | |
| 279 | AJ831578 | | Ll | India | CP -N Gene |
| 280 | AJ890465 | | Lt | India | CP -N Gene |
| 281 | D42079 | | C7-2 | Japan | CP -N Gene |
| 282 | AJ271416 | | 2A1-A | USA | CP -N Gene |
| 283 | AF013291 | | As | Korea | CP -N Gene |
| 284 | Y16926 | | Tfn | Italy | CP -N Gene |
| 285 | AB042294 | | IA-3a | Japan | CP -N Gene |
| 286 | D28780 | | NT9 | Taiwan | CP -N Gene |
| 287 | U31220 | Banana in Hawaii | Oahu | USA | CP -N Gene |
| 288 | D49496 | | Tai | Taiwan | CP -N Gene |
| 289 | X89652 | | Phym | India | CP -N Gene |
| 290 | AF281864 | | D | India | CP -N Gene |
| 291 | AF350450 | | H | India | CP -N Gene |
| 292 | L15336 | | Trk7 | Hungary | CP -N Gene |
| 293 | M21464 | | Q | Australia | CP -N Gene |
| 294 | AF063610 | | S | USA | CP -N Gene |
| 295 | AF127976 | | LS | USA | CP -N Gene |
| 296 | U10923 | Spinacia oleracea | SP103 | USA | CP -N Gene |
| 297 | AB006813 | | m2 | Japan | CP -N Gene |
| 298 | U22822 | | Sn | Australia | CP -N Gene |
| 299 | L40953 | | Wem | Unknown | CP -N Gene |
| 300 | AJ585086 | | AL | India | CP -N Gene |
| 301 | FN555197 | Capsicum sp | AN | India | Supressor Gene - |
| | | | | | 2b |
| 302 | FN555198 | Capsicum sp | CN04 | China | Supressor Gene - |
| | | | | | 2b |
| 303 | FN555199.1 | Capsicum sp | KS44 | Thailand | Supressor Gene - |
| | | | | | 2b |
| 304 | FN555200 | Capsicum sp | P522 | China | Supressor Gene - |
| | | | | | 2b |
| 305 | | | P3613 | China | Supressor Gene - |
| | | | | | 2b |
| 306 | HQ916353 | Oilseed pumpkin | | | Supressor Gene - |
| | | | | | 2b |
| 307 | aj517801 | Raphanus sativus | | | Supressor Gene - |
| | | | | | 2b |
| 308 | ay827561 | Paprika | | | Supressor Gene - |
| | | | | | 2b |
| 309 | jq074218 | Solanum lycopersicum | | | Supressor Gene - |
| | | | | | 2b |
| 310 | EU432184.1 | | CMV-NEP | | MP |
| 311 | EU432178.1 | | CMV-ANC | | MP |
| 312 | JF918963.1 | | | | MP |
| 313 | JN593375.1 | | | Italy | MP |
| 314 | EU414791.1 | tobacco | CMV-RZ | China | MP |
| 315 | JF918961.1 | | N1-03 | USA: Ohio | MP |
| 316 | JN593378 | | PhA_Italy | Italy | MP |

### Example 10

### Topical Application of Polynucleotide Triggers for Control of Pepino Mosaic Virus Infection

In this example the sequences of the Coat Protein (CM) and Movement Protein (MP) for different Pepino Mosaic Virus isolates were identified and can be seen in Table 8. Topical application of ss antisense DNA or dsRNA sequences derived from the listed sequences (SEQ ID NOs:317-349) will be performed in tomato plants infected by Pepino Mosaic Virus (PepMV) using a transfer reagent and the plants will be scored by ELISA analysis and visual assessment for reduction of symptoms.

TABLE 8
Sequences of target genes in Pepino Mosaic Virus (PepMV).

| SEQ | | | | | | |
|---|---|---|---|---|---|---|
| ID NO: | Sequence ID | Host | Strain | Isolate | Gene | Length |
| 317 | Orignal_file | | | | CP | 714 |
| 318 | FJ820177.1 | Solanum lycopersicum | | | CP | 714 |
| 319 | FJ820182.1 | Solanum lycopersicum | | | CP | 597 |
| 320 | FJ384784.1 | Lycopersicon esculentum | | | CP | 702 |
| 321 | FN429033 | Solanum lycopersicum | PV-0554 | | CP | 693 |
| 322 | AM040187 | Lycopersicon esculentum | Mu 04.12 | | CP | 488 |
| 323 | FJ263316.1 | Solanum lycopersicum | PMU05/5 | Spain | MP; Triple Gene Block1 | 708 |
| 324 | FJ263326.1 | Solanum lycopersicum | PMU08/47 | Spain | MP; Triple Gene Block1 | 705 |
| 325 | GQ438737.1 | Solanum lycopersicum | Al 2-01 | Spain | MP; Triple Gene Block1 | 705 |
| 326 | FJ263325.1 | Solanum lycopersicum | PMU08/42 | Spain | MP; Triple Gene Block1 | 705 |
| 327 | FJ384784.1 | Lycopersicon esculentum | isolate 4988 | Spain | MP; Triple Gene Block1 | 705 |
| 328 | AM041982.1 | Lycopersicon esculentum | isolate 1 | Spain: Murcia | MP; Triple Gene Block1 | 705 |
| 329 | AM041968 | Lycopersicon esculentum | isolate 1 | Spain: Murcia | MP; Triple Gene Block1 | 705 |
| 330 | AM041967.1 | Lycopersicon esculentum | isolate 1 | Spain: Murcia | MP; Triple Gene Block1 | 705 |
| 331 | AM041956.1 | Lycopersicon esculentum | Mu 03.2 | Spain: Murcia | MP; Triple Gene Block1 | 705 |
| 332 | AM041955.1 | Lycopersicon esculentum | Mu 03.1 | Spain: Murcia | MP; Triple Gene Block1 | 705 |
| 333 | AM041952.1 | Lycopersicon esculentum | Al 01.1 | Spain: Alicante | MP; Triple Gene Block1 | 706 |
| 334 | FJ263323.1 | Solanum lycopersicum | PMU08/38 | Spain | MP; Triple gene block protein 2 | 372 |
| | | | | | (TGBp2) | |
| 335 | FJ263322.1 | Solanum lycopersicum | PMU07/36 | Spain | MP; Triple gene block protein 2 | 372 |
| | | | | | (TGBp2) | |
| 336 | FJ820184.1 | Solanum lycopersicum | virus isolate | Spain | MP; Triple gene block protein 2 | 373 |
| | | | 4911 | | (TGBp2) | |
| 337 | FJ820181 | Solanum lycopersicum | isolate 7156 | Spain | MP; Triple gene block protein 2 | 373 |
| | | | | | (TGBp2) | |
| 338 | FJ820176 | Solanum lycopersicum | isolate 5577 | Spain | MP; Triple gene block protein 2 | 373 |
| | | | | | (TGBp2) | |
| 339 | FJ820174.1 | Solanum lycopersicum | isolate 4983 | Spain | MP; Triple gene block protein 2 | 372 |
| | | | | | (TGBp2) | |
| 340 | GU130080.1 | Solanum lycopersicum | isolate CI-05 | Spain | MP; Triple gene block protein 2 | 372 |
| | | | | | (TGBp2) | |
| 341 | GQ438737.1 | Solanum lycopersicum | Al 2-01 | Spain | MP; Triple gene block protein 2 | 372 |
| | | | | | (TGBp2) | |
| 342 | FJ263320.1 | Solanum lycopersicum | PMU07/27 | Spain | MP; Triple gene block protein 2 | 372 |
| | | | | | (TGBp2) | |
| 343 | FJ263317.1 | Solanum lycopersicum | PMU06/17a | Spain | MP; Triple gene block protein 2 | 372 |
| | | | | | (TGBp2) | |
| 344 | AM041992.1 | Lycopersicon esculentum | isolate 1 | Spain | MP; Triple gene block protein 2 | 372 |
| | | | | | (TGBp2) | |
| 345 | FJ820184.1 | Solanum lycopersicum | isolate 4911 | Spain | MP; Triple gene block protein 3 | 255 |
| 346 | FJ263325 | Solanum lycopersicum | PMU08/42 | Spain | MP; Triple gene block protein 3 | 255 |
| 347 | FJ820174 | Solanum lycopersicum | isolate 4983 | Spain | MP; Triple gene block protein 3 | 255 |
| 348 | FJ820173.1 | Solanum lycopersicum | isolate 4910-10 | Spain | MP; Triple gene block protein 3 | 255 |
| 349 | GQ438737.1 | Solanum lycopersicum | Al 2-01 | Spain | MP; Triple gene block protein 3 | 715 |

### Example 11

### Topical Application of Polynucleotide Triggers for Control of Infection by Barley Yellow Dwarf Virus (BYDV)

In this example, the sequences of the Coat Protein (CM), Movement Protein (MP), and Silencing Suppressor (SS) for different Barley yellow dwarf virus isolates were identified and are set forth in Table 9. Topical application of antisense ssDNA or dsRNA sequences derived from the listed sequences (SEQ ID NOs:350-385) can be performed in barley plants infected by BYDV using a transfer reagent and the plants can be scored by ELISA analysis and visual assessment for reduction of symptoms.

TABLE 9
Sequences of target genes in Barley Yellow Dwarf Virus (BYDV).

| SEQ | | | | | |
|---|---|---|---|---|---|
| ID NO: | Sequence ID | Strain | Isolate | Gene | Length |
| 350 | Orignal_file | | | CP-P3 and MP P4 | 603 |
| | | | | (overlap) | |
| 351 | BYDPCT | | | CP | 605 |
| 352 | JX402456.1 | B-Keb | Tunisia: Kebili | CP - P3, Partial CDS | 531 |
| 353 | JX402454.1 | B-Bej2 | Tunisia: Beja | CP - P3, Partial CDS | 532 |
| 354 | HM488005 | | Jordan | CP - P3, Partial CDS | 139 |
| 355 | EF408184.1 | MAV LMB2a | | CP - P3, Partial CDS | 593 |
| 356 | EU332334.1 | PAV isolate 06WH1 | | CP - P3, Partial CDS | 600 |
| 357 | EU332332.1 | PAV isolate 06KM14 | | CP - P3, Partial CDS | 603 |
| 358 | EU332330.1 | PAV isolate 05ZZ12 | | CP - P3, Partial CDS | 600 |
| 359 | EU332328.1 | PAV isolate 05ZZ9 | | CP - P3, Partial CDS | 600 |
| 360 | EU332326.1 | PAV isolate 05ZZ6 | | CP - P3, Partial CDS | 600 |
| 361 | EU332320.1 | PAV isolate 05ZZ1 | | CP - P3, Partial CDS | 600 |
| 362 | HM488005.1 | SGV | | CP - P3, Partial CDS | 139 |
| 363 | GU002361 | BYDV-MAV-OA1 | New Zealand: Lincoln | CP - P3, Partial CDS | 501 |
| 364 | GU002328 | BYDV-PAV-OA4 | New Zealand: Lincoln | CP - P3, Partial CDS | 502 |
| 365 | GU002324.1 | BYDV-PAS-DC2 | New Zealand: Lincoln | CP - P3, Partial CDS | 412 |
| 366 | GU002322.1 | BYDV-MAV-WC5 | New Zealand: Lincoln | CP - P3, Partial CDS | 412 |
| 367 | GU002360.1 | BYDV-MAV-O1LU | New Zealand: Lincoln | CP - P3, Partial CDS | 502 |
| 368 | GU002329.1 | BYDV-PAV-PC3 | New Zealand: Lincoln | CP - P3, Partial CDS | 490 |
| 369 | GU002325.1 | BYDV-PAV-327 | | CP - P3, Partial CDS | 502 |
| 370 | EF408184.1 | | | CP - P3, Partial CDS | 593 |
| 371 | EF408180.1 | isolate MAV SI-o4 | | CP - P3, Partial CDS | 593 |
| 372 | AF235167.1 | | | CP - P3, Partial CDS | 603 |
| 373 | ABR26505 | | | CP - P3, Partial CDS | 596 |
| 374 | AAZ93695. | UCD2-PAV | USA: California | MP-P4 | 462 |
| 375 | EF408167.1 | PAV sim10-2 | New | MP-P4 | 462 |
| | | | Zealand: Coromandel | | |
| 376 | EF408166.1 | PAV sim10-1 | New | MP-P4 | 462 |
| | | | Zealand: Coromandel | | |
| 377 | AY855920.1 | PAV-CN | China | MP-P4 | 462 |
| 378 | GU002330.1 | BYDV-PAV-WC2 | New Zealand: Lincoln | MP-P4 | 400 |
| 379 | X07653.1 | | | Silencing suppressor, P6 | 192 |
| 380 | EF521828.1 | | | Silencing suppressor, P6 | 126 |
| 381 | AJ007492.1 | | | Silencing suppressor, P6 | 129 |
| 382 | EU332332.1 | 05GG2 | China: Gangu | Silencing suppressor, P6 | 129 |
| 383 | EF521850.1 | PAV isolate 064 | USA: Alaska | Silencing suppressor, P6 | 120 |
| 384 | EU332335.1 | | China: Zhengzhou | Silencing suppressor, P6 | 123 |
| 385 | EF521849.1\| | PAV 0102 | USA: California | Silencing suppressor, P6 | 87 |

### Example 12

### Topical Application of Polynucleotide Triggers for Control of Infection by Tomato Yellow Leaf Curl Virus (TYLCV)

In this example, the sequences of the Coat Protein (CM), Movement Protein (MP), and Complement (C2) protein for different Tomato yellow leaf curl virus isolates were identified and are set forth in Table 10. Topical application of antisense ssDNA or dsRNA sequences derived from the listed sequences (SEQ ID NOs:386-421) can be performed in tomato plants infected by TYLCV using a transfer reagent and the plants scored by ELISA analysis and visual assessment for reduction of to symptoms.

TABLE 10
Sequences of target genes in Tomato Yellow Curl Leaf Virus (TYCLV).

| SEQ | | | | | | |
|---|---|---|---|---|---|---|
| ID NO: | Sequence ID | Host | Strain | Isolate | Gene | Note |
| 386 | AJ519441.1 | | | | CP | |
| 387 | JX075187.1 | | | South Korea | CP | |
| 388 | HM856915.1 | | | | CP | |
| 389 | HM856913.1\| | | | | CP | |
| 390 | EF210554.1 | | | Arizona | CP | |
| 391 | AB116631.1 | Stellaria aquatica | TYLCV-IL[JR: Mis: Ste] | Japan | CP | |
| 392 | L27708.1 | | Almeria | Spain | CP | |
| 393 | X15656.1 | | | | CP | |
| 394 | X61153.1 | | | | CP | |
| 395 | X76319.1 | | | | CP | |
| 396 | GU723744.1 | | | Thailand | CP | |
| 397 | EF110890.1 | Lycopersicon esculentum | | USA: Texas | CP | |
| 398 | HE603246.1 | Solanum lycopersicum | New | Israel | MP | |
| | | | Caledonia: Ouvea: 2010 | | | |
| 399 | HM448447.1 | Solanum lycopersicum | | Mauritius | MP | |
| 400 | EU143754.1 | Squash | | Jordan | MP | |
| 401 | AJ842308.1 | | | Saint Gilles | MP | |
| 402 | AJ842307.1 | | | Saint Gilles | MP | |
| 403 | EU143745.1 | Cucumber | | Jordan | MP | |
| 404 | AM409201.1 | Solanum lycopersicum | | Reunion: Saint-Gilles les | MP | |
| | | | | Hauts | | |
| 405 | JX456639.1 | | KYCTo18 | China | MP | |
| 406 | JN183880.1 | | Andong 2 | South Korea: Andong | MP | |
| 407 | FR851297.1 | | | Israel | MP | |
| 408 | HM856914.1 | | | Gwangyang 6 | MP | |
| 409 | HM856912.1 | | | South Korea: Gunwi | MP | |
| 410 | GU348995.1 | Solanum lycopersicum | | China: Hebei | MP | |
| 411 | EF490995.1 | Solanum lycopersicum | | Martinique | MP | |
| 412 | EF110890.1 | Lycopersicon esculentum | | USA: Texas | | |
| 413 | DQ144621.1 | Lycopersicon esculentum | | Italy: Sicily | C2 | Complement |
| 414 | AB116632 | Lycopersicon esculentum | | Japan | C2 | Complement |
| 415 | AB110218.1 | | | Israel | C2 | Complement |
| 416 | GU325634.1 | Lycopersicon esculentum | | South Korea: Boseong | C2 | Complement |
| 417 | EU143745.1 | Cucumber | | Jordan: Homrat Al-Sahen | C2 | Complement |
| 418 | GU178814 | Solanum lycopersicum | | Australia: Brisbane2: 2006 | C2 | Complement |
| 419 | EF523478.1 | | | Mexico | C2 | Complement |
| 420 | EF433426.1 | cucumber | | Jordan | C2 | Complement |
| 421 | EF110890 | Lycopersicon esculentum | | USA: Texas | C2 | Complement |

### Example 13

### Topical Application of Polynucleotide Triggers for Control of Infection by Cotton Leaf Curl Virus (CLCuV)

In this example the sequences of the Coat Protein (CM), Movement Protein (MP) and AC2 protein for different Cotton Leaf Curl Virus isolates were identified and can be seen in Table 11. Topical application of ss antisense DNA or dsRNA sequences derived from the listed sequences (SEQ ID NOs:422-447) will be performed in cotton plants infected by CLCuV using a transfer reagent and the plants will be scored by ELISA analysis and visual assessment for reduction of symptoms.

TABLE 11
Sequences of target genes in Cotton Leaf Curl Virus (CLCuV).

| SEQ | | | | | | |
|---|---|---|---|---|---|---|
| ID NO: | Sequence ID | Host | Species | Isolate | Gene | length |
| 422 | EF057791.1 | | Cotton leaf curl virus | | CP | 771 |
| 423 | JN558352.1 | papaya | Cotton leaf curl virus | | CP | 771 |
| 424 | FJ218487.1 | Gossypium hirsutum | Cotton leaf curl virus | | CP | 771 |
| 425 | AF521594.1 | | Cotton leaf curl virus | India: Hisar | CP | 771 |
| 426 | AY765254 | | Cotton leaf curl virus | India: Sirsa, Haryana | CP | 771 |
| 427 | JX914662.1 | | | | CP | 771 |
| 428 | EF465535.1 | | Hibiscus rosa-sinensis | | CP | 771 |
| 429 | FJ159268.1 | | Hibiscus cannabinus | Amadalavalasa: South | CP | 771 |
| | | | | India | | |
| 430 | JX286658.1 | | Hibiscus rosa-sinensis | China | CP | 772 |
| 431 | JN968573.1 | | Hibiscus rosa-sinensis | China: Guangdong | CP | 771 |
| 432 | GU574208.1 | | Okra | China | CP | 771 |
| 433 | GU112008.1 | | Abelmoschus esculentus (okra) | India: Karnal, Haryana | CP | 771 |
| 434 | AJ002455.1 | | | | CP | 771 |
| 435 | AJ002455.1 | | | Pakistan | CP | 771 |
| 436 | JX286660 | | Hibiscus rosa-sinensis | China | CP | 771 |
| 437 | HQ455367.1 | | Hibiscus rosa-sinensis (Rose Mallow) | China | CP | 771 |
| 438 | EU384573 | | Gossypium hirsutum subsp. Latifolium | Pakistan: Multan | CP | 772 |
| 439 | AJ002458.1 | | Cotton leaf curl Multan virus-[26] | Pakistan | CP | 772 |
| 440 | AY028808.1 | | | India | MP | 359 |
| 441 | AF363011.1 | | | | MP | 358 |
| 442 | HM235774.1 | | Gossypium hirsutum | India | MP | 358 |
| 443 | AY028808.1 | | | India | MP | 357 |
| 444 | AY146959.1 | | | India | MP | 358 |
| 445 | AY146960.1 | | | | MP | 357 |
| 446 | AY146957.1 | | | India: Sirsa | MP | 367 |
| 447 | HM037923.1 | | Gossypium hirsutum | Sirsa-Haryana-En(P) | AC2 | 454 |

### Example 14

### Topical Application of dsRNA Oligonucleotides to Pepper Plants for Control of Tomato Spotted Wilt Virus (TSWV)

In this example, growing pepper plants (c.v. Yolo Wonder B) were inoculated with tomato spotted wilt virus (TSWV), a negative strand ssRNA virus, and the plants were separated into different groups. The experimental group was topically treated with a liquid composition containing at least one dsRNA polynucleotide comprising an approximately 100 bp sequence that is homologous to a transcript of the nucleocapsid (N), suppressor (NSs) or movement (NSm) gene of TSWV and its complement. The sequences of the sense strand of the trigger molecules used in the experiments outlined in this Example are shown in Table 12.

TABLE 12
dsRNA polynucleotides directed to TSWV nucleocapsid (N),
suppressor (NSs) or movement (NSm) gene transcripts.

| SEQ | | | | |
|---|---|---|---|---|
| ID NO | Trigger ID | Length | Virus | Target |
| 448 | T25748 | 99 | TSWV | Nucleocapsid (N) |
| 449 | T25749 | 101 | TSWV | Nucleocapsid (N) |
| 450 | T25750 | 101 | TSWV | Nucleocapsid (N) |
| 451 | T25751 | 101 | TSWV | Nucleocapsid (N) |
| 452 | T25752 | 101 | TSWV | Nucleocapsid (N) |
| 453 | T25753 | 101 | TSWV | Nucleocapsid (N) |
| 454 | T25754 | 108 | TSWV | Nucleocapsid (N) |
| 455 | T25755 | 101 | TSWV | Nucleocapsid (N) |
| 456 | T25756 | 97 | TSWV | Nucleocapsid (N) |
| 457 | T25757 | 103 | TSWV | Movement (NSm) |
| 458 | T25758 | 100 | TSWV | Movement (NSm) |
| 459 | T25759 | 99 | TSWV | Movement (NSm) |
| 460 | T25760 | 101 | TSWV | Movement (NSm) |
| 461 | T25761 | 101 | TSWV | Movement (NSm) |
| 462 | T25762 | 96 | TSWV | Movement (NSm) |
| 463 | T25763 | 101 | TSWV | Movement (NSm) |
| 464 | T25764 | 97 | TSWV | Movement (NSm) |
| 465 | T25765 | 98 | TSWV | Movement (NSm) |
| 466 | T25766 | 109 | TSWV | Movement (NSm) |
| 467 | T25767 | 100 | TSWV | Suppressor (NSs) |
| 468 | T25768 | 100 | TSWV | Suppressor (NSs) |
| 469 | T25769 | 97 | TSWV | Suppressor (NSs) |
| 470 | T25770 | 101 | TSWV | Suppressor (NSs) |
| 471 | T25771 | 95 | TSWV | Suppressor (NSs) |
| 472 | T25772 | 100 | TSWV | Suppressor (NSs) |
| 473 | T25773 | 102 | TSWV | Suppressor (NSs) |
| 474 | T25774 | 103 | TSWV | Suppressor (NSs) |
| 475 | T25775 | 97 | TSWV | Suppressor (NSs) |
| 476 | T25776 | 96 | TSWV | Suppressor (NSs) |
| 477 | T25777 | 102 | TSWV | Suppressor (NSs) |
| 478 | T25778 | 101 | TSWV | Suppressor (NSs) |
| 479 | T25779 | 98 | TSWV | Suppressor (NSs) |
| 480 | T25780 | 103 | TSWV | Suppressor (NSs) |
| 481 | T25781 | 101 | TSWV | Suppressor (NSs) |
| 482 | T25782 | 102 | TSWV | Suppressor (NSs) |
| 483 | T34084 | 100 | CMV | Coat Protein (CP) |

Plants were sown in a growth chamber [22° C., 8 hour light (˜50 μmol), 16 hour dark cycles] and transferred to a green house a couple of days before treatment. Pepper plants at the 2-5 fully expanded leaf stage were used in this assay. The experimental setup consisted of between 20-24 plants per treatment. Treatments consisted of: (a) healthy controls (no viral infection), (b) virus only control (no polynucleotide solution), (c) formulation only (no polynucleotides), or (d) experimental application with polynucleotide/Silwet L-77 trigger solution comprising a trigger molecule selected from the list of SEQ ID NOs:448-483 following virus to infection. Virus infection was carried out using standard mechanical inoculation technique and using Tomato spotted wilt virus (TSWV) or Cucumber mosaic virus (CMV), a positive strand RNA virus unrelated to TSWV. The final concentration used for each dsRNA polynucleotide was between 14.2-15.15 pmol/plant (in 0.1% Silwet L-77, 2% ammonium sulfate, 5 mM sodium phosphate buffer, pH 6.8). One thousand micro-liters of the polynucleotide/Silwet L-77 solution was applied using an airbrush (Badger 200G) at 10 psi to each plant group. Plants were arranged in the greenhouse following a randomized complete block design and monitored visually for symptom development. Plant height and ELISA analysis were both carried out at 32 days post-infection (32 DPI). ELISA analysis was performed on supernatant extracts from control and systemic leaf tissue punctures using an antibody to TSWV nucleocapsid (N) protein. The experiment was repeated twice (see Tables 13-17).

TABLE 13
Experiment 1: Plant height measurements at 32
DPI after treatment with dsRNA polynucleotides.

| Treatment | Mean | Group | | | | N | Std Dev |
|---|---|---|---|---|---|---|---|
| Healthy | 39.9 | A | | | | 24 | 5.4 |
| T25748 | 33.4 | | B | | | 24 | 10.0 |
| T25773 | 32.9 | | | BC | | 24 | 7.9 |
| T25763 | 32.7 | | | BC | | 24 | 8.7 |
| T25769 | 32.5 | | | BC | | 24 | 9.5 |
| T25755 | 32.3 | | | BC | | 24 | 7.8 |
| T25776 | 32.3 | | | BC | | 24 | 7.8 |
| T25770 | 31.9 | | | BC | | 24 | 8.8 |
| T25778 | 31.7 | | | BC | | 24 | 7.1 |
| T25753 | 31.6 | | | BC | | 24 | 9.9 |
| Virus (TSWV) | 31.3 | | | BC | | 24 | 8.7 |
| CMV | 29.9 | | | BC | | 24 | 7.5 |
| Buffer | 29.2 | | | | C | 24 | 7.1 |
| (Formulation) | | | | | | | |

*Levels not connected by the same letter are significantly different.

TABLE 14
Experiment 1: Statistical analysis of best performing
trigger sequences compared to controls.

| Treatment | Mean | Std Deviation | Std Err |
|---|---|---|---|
| Healthy | 39.9 | 5.4 | 1.10486 |
| Virus (TSWV) | 31.3 | 8.7 | 1.77702 |
| Buffer (Formulation) | 29.2 | 7.1 | 1.44554 |
| T25748 | 33.4 | 10.0 | 2.05127 |
| T25773 | 32.9 | 7.9 | 1.61158 |

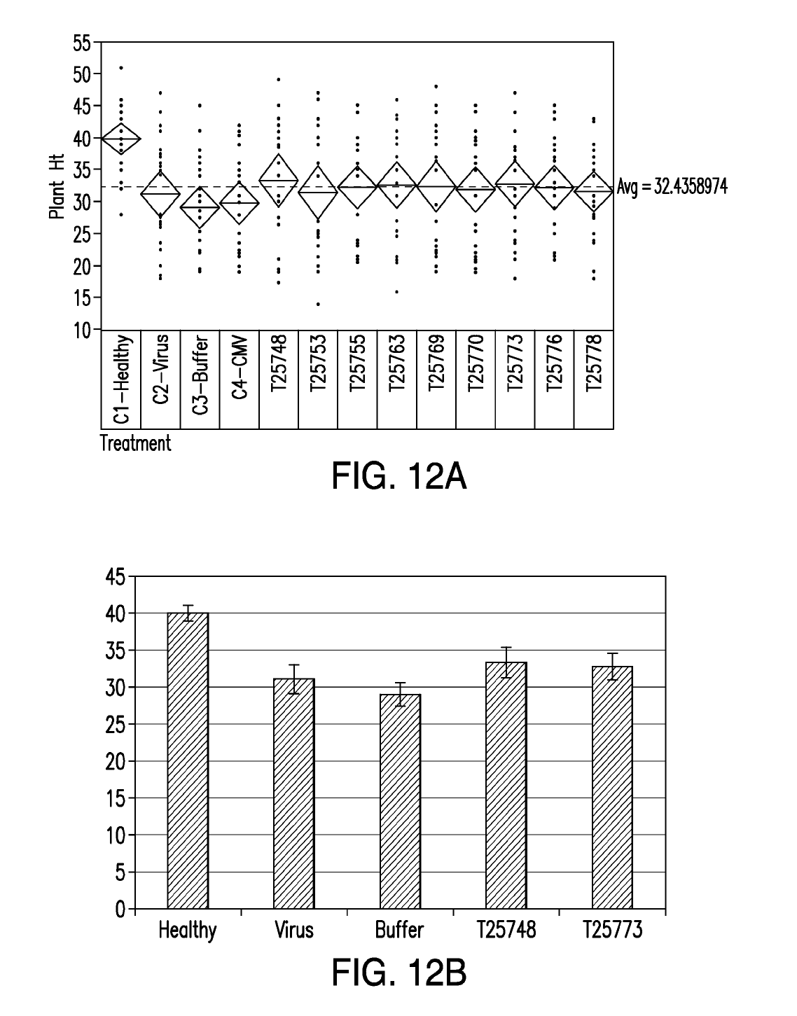

Plants treated with polynucleotide trigger sequence T25748 corresponding to SEQ ID NO:448 in the TSWV Nucleocapsid (N) gene were significantly taller than plants treated with other polynucleotides. This is also shown in FIGS. 12A and B which shows a graphical representation of these results.

TABLE 15
Experiment 1: ELISA analysis at 32 DPI after
treatment with dsRNA polynucleotides.

| Treatment | Mean | Std Err |
|---|---|---|
| Healthy | 0.06 | 0.02 |
| T25773 | 0.15 | 0.06 |
| Virus (TSWV) | 0.23 | 0.09 |
| T25763 | 0.24 | 0.09 |
| T25778 | 0.25 | 0.12 |
| Buffer (Form.) | 0.27 | 0.13 |
| T25755 | 0.28 | 0.13 |
| T25776 | 0.28 | 0.14 |
| CMV | 0.29 | 0.16 |
| T25769 | 0.30 | 0.13 |
| T25748 | 0.40 | 0.17 |
| T25753 | 0.47 | 0.20 |
| T25770 | 0.61 | 0.23 |

TABLE 16
Experiment 2: Plant height measurements at 32
DPI after treatment with dsRNA polynucleotides.

| Treatment | Mean | Group | | | | N | Std Dev |
|---|---|---|---|---|---|---|---|
| Healthy | 30.1 | A | | | | 24 | 7.2 |
| T25772 | 25.6 | | B | | | 24 | 7.1 |
| T25748 | 25.1 | | | BC | | 24 | 7.0 |
| T25769 | 24.8 | | | BC | | 24 | 5.7 |
| T25755 | 24.3 | | | BC | | 24 | 8.0 |
| T25775 | 24.2 | | | BC | | 24 | 6.3 |
| T25776A | 23.9 | | | BC | | 24 | 6.6 |
| Virus | 23.6 | | | BC | | 24 | 6.2 |
| T25763 | 23.3 | | | BC | | 24 | 5.4 |
| CMV | 23.2 | | | BC | | 24 | 7.1 |
| T25770 | 23.1 | | | BC | | 24 | 6.1 |
| Buffer | 22.6 | | | BC | | 24 | 6.6 |
| T25776B | 22.0 | | | | C | 24 | 6.6 |

*Levels not connected by the same letter are significantly different.

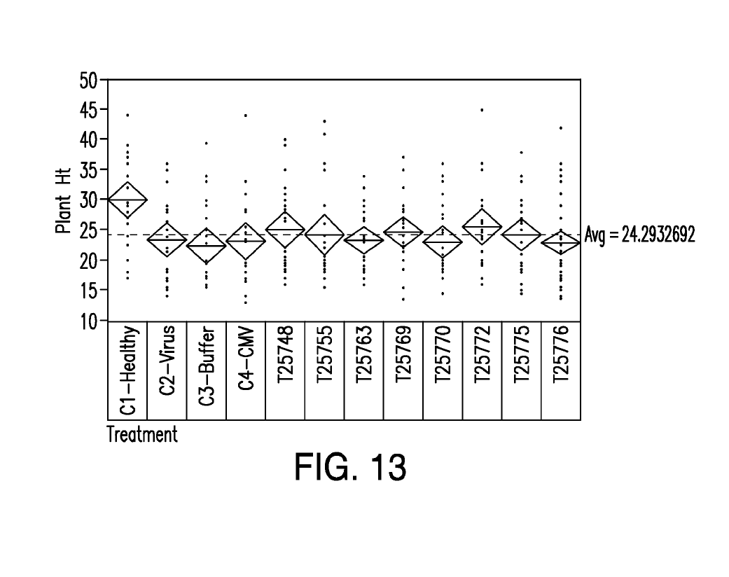

In this experiment treatment with trigger sequence T25748 (SEQ ID NO:448) was the best performer of the “BC” group. FIG. 13 shows a graphical display of the results of this experiment.

TABLE 17
Experiment 2: ELISA analysis at 32 DPI
after treatment with dsRNA polynucleotides.
Experiment 2

| Treatment | Mean | StdErr |
|---|---|---|
| T25776A | 0.05 | 0.01 |
| Healthy | 0.06 | 0.01 |
| T25776B | 0.06 | 0.02 |
| T25772 | 0.44 | 0.17 |
| Virus (TSWV) | 0.45 | 0.16 |
| T25769 | 0.53 | 0.20 |
| T25755 | 0.55 | 0.20 |
| T25775 | 0.58 | 0.21 |
| T25770 | 0.61 | 0.18 |
| T25763 | 0.79 | 0.19 |
| T25748 | 0.83 | 0.24 |
| Buffer (Form.) | 1.05 | 0.24 |
| CMV | 1.11 | 0.24 |
| T25776 | 1.98 | 0.20 |

1. A method of treatment or prevention of a Tospovirus infection in a plant comprising: topically applying to said plant a composition comprising an antisense single-stranded DNA polynucleotide and a transfer agent, wherein said antisense single-stranded DNA polynucleotide is complementary to all or a portion of an essential Tospovirus gene sequence or an RNA transcript thereof, wherein the symptoms of viral infection or development of symptoms are reduced or eliminated in said plant relative to a plant not treated with said composition when grown under the same conditions.

2. The method of claim 1, wherein said transfer agent is an organosilicone surfactant composition or compound contained therein.

3. The method of claim 1, wherein said composition comprises more than one antisense single-stranded DNA polynucleotide complementary to all or a portion of an essential Tospovirus gene sequence, an RNA transcript of said essential Tospovirus gene sequence, or a fragment thereof.

4. The method of claim 1, wherein said antisense single-stranded DNA polynucleotide is selected from the group consisting of SEQ NO:1-12 or a fragment thereof.

5. The method of claim 1, wherein said Tospovirus is selected from the group consisting of bean necrotic mosaic virus, Capsicum chlorosis virus, groundnut bud necrosis virus, groundnut ringspot virus, groundnut yellow spot virus, impatiens necrotic spot virus, iris yellow spot viris, melon yellow spot virus, peanut bud necrosis virus, peanut yellow spot virus, soybean vein necrosis-associated virus, tomato chlorotic spot virus, tomato necrotic ringspot virus, tomato spotted wilt virus, tomato zonate spot virus, watermelon bud necrosis virus, watermelon silver mottle virus, and zucchini lethal chlorosis virus.

6. The method of claim 1, wherein said essential Tospovirus gene is selected from the group consisting of nucleocapsid gene (N), coat protein gene (CP), virulence factors NSm and NSs, and RNA-dependent RNA polymerase L segment (RdRp/L segment).

7. The method of claim 6, wherein said essential gene sequence is selected from the group consisting of SEQ ID NOs:13-46.

8. The method of claim 1, wherein said composition is topically applied by spraying, dusting, or is applied to the plant surface as matrix-encapsulated DNA.

9. A composition comprising an antisense single-stranded DNA polynucleotide and a transfer agent, wherein said antisense single-stranded DNA polynucleotide is complementary to all or a portion of an essential Tospovirus gene sequence or an RNA transcript thereof, wherein said composition is topically applied to a plant and wherein the symptoms of Tospovirus infection or development of symptoms are reduced or eliminated in said plant relative to a plant not treated with said composition when grown under the same conditions.

10. The composition of claim 9, wherein said essential gene sequence is selected from the group consisting of SEQ ID NOs:13-46.

11. The composition of claim 9, wherein said transfer agent is an organosilicone composition.

12. The composition of claim 9, wherein said antisense single-stranded DNA polynucleotide is selected from the group consisting of SEQ ID NOs:1-12.

13. A method of reducing expression of an essential Tospovirus gene comprising contacting a Tospovirus particle with a composition comprising an antisense single-stranded DNA polynucleotide and a transfer agent, wherein said antisense single-stranded DNA polynucleotide is complementary to all or a portion of an essential gene sequence in said Tospovirus or an RNA transcript thereof, wherein the symptoms of Tospovirus infection or development of symptoms are reduced or eliminated in said plant relative to a plant not treated with said composition when grown under the same conditions.

14. The method of claim 13, wherein said essential gene sequence is selected from the group consisting of SEQ ID NOs:13-46.

15. The method of claim 13, wherein said transfer agent is an organosilicone compound.

16. The method of claim 13, wherein said antisense single-stranded DNA polynucleotide is selected from the group consisting of SEQ ID NOs:1-12 or fragment thereof.

17. A method of identifying antisense single-stranded DNA polynucleotides useful in modulating Tospovirus gene expression when topically treating a plant comprising: a) providing a plurality of antisense single-stranded DNA polynucleotides that comprise a region complementary to all or a part of an essential Tospovirus gene or RNA transcript thereof; b) topically treating said plant with one or more of said antisense single-stranded DNA polynucleotides and a transfer agent; c) analyzing said plant or extract for modulation of symptoms of Tospovirus infection; and d) selecting an antisense single-stranded DNA polynucleotide capable of modulating the symptoms or occurrence of Tospovirus infection.

18. The method of claim 17, wherein said transfer agent is an organosilicone compound.

19. An agricultural chemical composition comprising an admixture of an antisense single-stranded DNA polynucleotide and a pesticide, wherein said antisense single-stranded DNA polynucleotide is complementary to all or a portion of an essential Tospovirus gene sequence or RNA transcript thereof, wherein said composition is topically applied to a plant and wherein the symptoms of Tospovirus infection or development of symptoms are reduced or eliminated in said plant relative to a plant not treated with said composition when grown under the same conditions.

20. The agricultural chemical composition of claim 19, wherein said pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.

21. A method of treatment or prevention of a Tospovirus infection in a plant comprising: topically applying to said plant a composition comprising a double-stranded RNA polynucleotide and a transfer agent, wherein said double-stranded RNA polynucleotide is complementary to all or a portion of an essential Tospovirus gene sequence or an RNA transcript thereof, wherein the symptoms of viral infection or development of symptoms are reduced or eliminated in said plant relative to a plant not treated with said composition when grown under the same conditions.

22. The method of claim 21, wherein said transfer agent is an organosilicone surfactant composition or compound contained therein.

23. The method of claim 21, wherein said composition comprises more than one double-stranded RNA polynucleotide complementary to all or a portion of an essential Tospovirus gene sequence, an RNA transcript of said essential Tospovirus gene sequence, or a fragment thereof.

24. The method of claim 21, wherein said double-stranded RNA polynucleotide is selected from the group consisting of SEQ NO:47-103 or a fragment thereof.

25. The method of claim 21, wherein said Tospovirus is selected from the group consisting of bean necrotic mosaic virus, Capsicum chlorosis virus, groundnut bud necrosis virus, groundnut ringspot virus, groundnut yellow spot virus, impatiens necrotic spot virus, iris yellow spot viris, melon yellow spot virus, peanut bud necrosis virus, peanut yellow spot virus, soybean vein necrosis-associated virus, tomato chlorotic spot virus, tomato necrotic ringspot virus, tomato spotted wilt virus, tomato zonate spot virus, watermelon bud necrosis virus, watermelon silver mottle virus, and zucchini lethal chlorosis virus.

26. The method of claim 21, wherein said essential Tospovirus gene is selected from the group consisting of nucleocapsid gene (N), coat protein gene (CP), virulence factors NSm and NSs, and RNA-dependent RNA polymerase L segment (RdRp/L segment).

27. The method of claim 26, wherein said essential Tospovirus gene is selected from the group consisting of SEQ ID NOs:13-46.

28. The method of claim 21, wherein said composition is topically applied by spraying, dusting, or is applied to the plant surface as matrix-encapsulated RNA.

29. A composition comprising a double-stranded RNA polynucleotide and a transfer agent, wherein said double-stranded RNA polynucleotide is complementary to all or a portion of an essential Tospovirus gene sequence or an RNA transcript thereof, wherein said composition is topically applied to a plant and wherein the symptoms of Tospovirus infection or development of symptoms are reduced or eliminated in said plant relative to a plant not treated with said composition when grown under the same conditions.

30. The composition of claim 29, wherein said essential gene sequence is selected from the group consisting of SEQ ID NOs:13-46.

31. The composition of claim 29, wherein said transfer agent is an organosilicone composition.

32. The composition of claim 29, wherein said double-stranded RNA polynucleotide is selected from the group consisting of SEQ NO:47-103.

33. A method of reducing expression of an essential Tospovirus gene comprising contacting a Tospovirus particle with a composition comprising a double-stranded RNA polynucleotide and a transfer agent, wherein said double-stranded RNA polynucleotide is complementary to all or a portion of an essential gene sequence in said Tospovirus or an RNA transcript thereof, wherein the symptoms of Tospovirus infection or development of symptoms are reduced or eliminated in said plant relative to a plant not treated with said composition when grown under the same conditions.

34. The method of claim 33, wherein said essential gene sequence is selected from the group consisting of SEQ ID NOs:13-46.

35. The method of claim 33, wherein said transfer agent is an organosilicone compound.

36. The method of claim 33, wherein said double-stranded RNA polynucleotide is selected from the group consisting of SEQ ID NOs:47-103 or fragment thereof.

37. A method of identifying double-stranded RNA polynucleotide useful in modulating Tospovirus gene expression when topically treating a plant comprising: a) providing a plurality of double-stranded RNA polynucleotides that comprise a region complementary to all or a part of an essential Tospovirus gene or RNA transcript thereof; b) topically treating said plant with one or more of said double-stranded RNA polynucleotides and a transfer agent; c) analyzing said plant or extract for modulation of symptoms of Tospovirus infection; and d) selecting a double-stranded RNA polynucleotide capable of modulating the symptoms or occurrence of Tospovirus infection.

38. The method of claim 37, wherein said transfer agent is an organosilicone compound.

39. An agricultural chemical composition comprising an admixture of a double-stranded RNA polynucleotide and a pesticide, wherein said double-stranded RNA polynucleotide is complementary to all or a portion of an essential Tospovirus gene sequence or RNA transcript thereof, wherein said composition is topically applied to a plant and wherein the symptoms of Tospovirus infection or development of symptoms are reduced or eliminated in said plant relative to a plant not treated with said composition when grown under the same conditions.

40. The agricultural chemical composition of claim 39, wherein said pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.

41. A method of treatment or prevention of a Geminivirus infection in a plant comprising: topically applying to said plant a composition comprising a double-stranded RNA polynucleotide and a transfer agent, wherein said double-stranded RNA polynucleotide is complementary to all or a portion of an essential Geminivirus gene sequence, or an RNA transcript thereof, wherein the symptoms of viral infection or development of symptoms are reduced or eliminated in said plant relative to a plant not treated with said composition when grown under the same conditions.

42. The method of claim 41, wherein said transfer agent is an organosilicone surfactant composition or compound contained therein.

43. The method of claim 41, wherein said composition comprises more than one double-stranded RNA polynucleotide complementary to all or a portion of an essential Geminivirus gene sequence, an RNA transcript of said essential Geminivirus gene sequence, or a fragment thereof.

44. The method of claim 41, wherein said double-stranded RNA polynucleotide is selected from the group consisting of SEQ NO:104-268 or a fragment thereof.

45. The method of claim 41, wherein said Geminivirus is selected from the group consisting of Barley yellow dwarf virus, Cucumber mosaic virus, Pepino mosaic virus, Cotton curl leaf virus, Tomato yellow leaf curl virus, Tomato golden mosaic virus, Potato yellow mosaic virus, Pepper leaf curl virus, Bean golden mosaic virus, Bean golden mosaic virus, Tomato mottle virus.

46. The method of claim 41, wherein said essential Geminivirus gene is selected from the group consisting of nucleocapsid gene (N), a coat protein gene (CP), virulence factors NSm and NSs, and RNA-dependent RNA polymerase L segment (RdRp/L segment), a silencing suppressor gene, movement protein (MP), Nia, CP-N, a triple gene block, CP-P3, MP-P4, C2, and AC2.

47. The method of claim 46, wherein said essential gene sequence is selected from the group consisting of SEQ ID NOs:269-447.

48. The method of claim 41, wherein said composition is topically applied by spraying, dusting, or is applied to the plant surface as matrix-encapsulated RNA.

49. A composition comprising a double-stranded RNA polynucleotide and a transfer agent, wherein said double-stranded RNA polynucleotide is complementary to all or a portion of an essential Geminivirus gene sequence, such as one set forth as SEQ ID NOs:269-447, or an RNA transcript thereof, wherein said composition is topically applied to a plant and wherein the symptoms of Geminivirus infection or development of symptoms are reduced or eliminated in said plant relative to a plant not treated with said composition when grown under the same conditions.

50. The composition of claim 49, wherein said essential gene sequence is selected from the group consisting of SEQ ID NOs:269-447.

51. The composition of claim 49, wherein said transfer agent is an organosilicone composition.

52. The composition of claim 49, wherein said double-stranded RNA polynucleotide is selected from the group consisting of SEQ NO:104-268.

53. A method of reducing expression of an essential Geminivirus gene comprising contacting a Geminivirus particle with a composition comprising a double-stranded RNA polynucleotide and a transfer agent, wherein said double-stranded RNA polynucleotide is complementary to all or a portion of an essential gene sequence in said Geminivirus or an RNA transcript thereof, wherein the symptoms of Geminivirus infection or development of symptoms are reduced or eliminated in said plant relative to a plant not treated with said composition when grown under the same conditions.

54. The method of claim 53, wherein said essential gene sequence is selected from the group consisting of SEQ ID NOs:269-447.

55. The method of claim 53, wherein said transfer agent is an organosilicone compound.

56. The method of claim 53, wherein said double-stranded RNA polynucleotide is selected from the group consisting of SEQ NO:104-268 or fragment thereof.

57. A method of identifying double-stranded RNA polynucleotide useful in modulating Geminivirus gene expression when topically treating a plant comprising: a) providing a plurality of double-stranded RNA polynucleotides that comprise a region complementary to all or a part of an essential Geminivirus gene or RNA transcript thereof; b) topically treating said plant with one or more of said double-stranded RNA polynucleotides and a transfer agent; c) analyzing said plant or extract for modulation of symptoms of Geminivirus infection; and d) selecting a double-stranded RNA polynucleotide capable of modulating the symptoms or occurrence of Geminivirus infection.

58. The method of claim 57, wherein said transfer agent is an organosilicone compound.

59. An agricultural chemical composition comprising an admixture of a double-stranded RNA polynucleotide and a pesticide, wherein said double-stranded RNA polynucleotide is complementary to all or a portion of an essential Geminivirus gene sequence or RNA transcript thereof, wherein said composition is topically applied to a plant and wherein the symptoms of Geminivirus infection or development of symptoms are reduced or eliminated in said plant relative to a plant not treated with said composition when grown under the same conditions.

60. The agricultural chemical composition of claim 59, wherein said pesticide is selected from the group consisting of anti-viral compounds, insecticides, fungicides, nematocides, bactericides, acaricides, growth regulators, chemosterilants, semiochemicals, repellents, attractants, pheromones, feeding stimulants, and biopesticides.


SEQUENCE LISTING (483 sequences)

SEQ ID NO: 1 - DNA, 23 bp, Impatiens Necrotic Spot Virus
gctataaacagccttccaagtca

SEQ ID NO: 2 - DNA, 23 bp, Impatiens Necrotic Spot Virus
gtcattaagagtgctgacttcac

SEQ ID NO: 3 - DNA, 22 bp, Tomato spotted wilt virus
catctcaaagctatcaactgaa

SEQ ID NO: 4 - DNA, 22 bp, Tomato spotted wilt virus
tgatcttcattcatttcaaatg

SEQ ID NO: 5 - DNA, 21 bp, Cucumber Mosaic Virus
agacgtgggaatgcgttggtg

SEQ ID NO: 6 - DNA, 21 bp, Cucumber Mosaic Virus
ctcgacgtcaacatgaagtac

SEQ ID NO: 7 - DNA, 21 bp, Cucumber Mosaic Virus
gcttggactccagatgcagca

SEQ ID NO: 8 - DNA, 21 bp, Cucumber Mosaic Virus
tactgataaaccagtaccggt

SEQ ID NO: 9 - DNA, 21 bp, Cucumber Mosaic Virus
cgaatttgaatgcgcgaaaca

SEQ ID NO: 10 - DNA, 21 bp, Cucumber Mosaic Virus
agtttcttgtcatattctgtg

SEQ ID NO: 11 - DNA, 21 bp, Cucumber Mosaic Virus
gacgaccagctgccaacgtct

SEQ ID NO: 12 - DNA, 21 bp, Cucumber Mosaic Virus
tattaagtcgcgaaagctgct

SEQ ID NO: 13 - DNA, 569 bp, Groundnut ringspot virus isolate
atgtctaaggtcaagctcacaaaagaaaacattgtctctcttttgactcaatctgaggatgttgagtttgaagaagaccagaaccaggttgcattcaactttaagactttttgtcaggaaaatcttgacctgattaagaaaatgagtatcacttcatgtttgactttcttgaagaatcgccaaagcatcatgaaagttgtgaaacaaagtgattttacttttggcaaggtcacgataaagaaaaattctgagaaggttggagctaaagacatgactttcagaaggcttgatagcatgataagagtgaaactcatagaagagactgcaaacaatgagaatcttgctatcatcaaggcaaaaattgcctcccatcctttggtccaagcttacgggctgcctttggcagatgcaaaatctgtgagacttgccataatgcttggaggtagtatccctctcattgcttctgttgacagcttcgaaatgatcagtgttgttcttgccatatatcaagatgcaaagtacaaggagttagggattgaaccaactaagtacaacactaaggaagctct

SEQ ID NO: 14 - DNA, 777 bp, Groundnut ringspot and Tomato chlorotic spot virus reassortant
ttatgcaacaccagcaattttggcctctttcttgactccaaacatttcatagaatttgtcaagattgtcactgtaatagtccatagcaatgcttcccttagcattgggattgcaagaactaagtatcttggcatattctttccctttgtttatctgtgcatcatccattgtaaatcctttgcttttaagcactgtgcaaaccttccccagagcttccttagtgttgtacttagttggttcaatccctaactccttgtactttgcatcttgatatatggcaagaacaacactgatcatttcgaagctgtcaacagaagcaatgagagggatactacctccaagcattatagcaagtcttacagattttgcatctgccagaggcagcccgtaagcttggaccaaagggtgggaggcaatttttgccttgataatagcaagattctcattgtttgcagtctcttctatgagcttcactcttatcatgctatcaagcctcctgaaagtcatatctttagctccaactctctcagaatttttctttatcgtgaccttaccaaaagtaaaatcactctggttcacaactttcataatgctttgacgattcttcaagaaagtcaaacatgaagtgatactcattttcttaatcaggtcaagattttcctgacagaaagtcttgaagttgaatgcgacctggttttggtcttcttcaaactcaacatcttcagattgagttaaaagagagacaatgttttcttttgtgagcttgaccttagacat

SEQ ID NO: 15 - DNA, 757 bp, Tomato spotted wilt virus
aagctcactaaggaaagcattgttgctttgttgacacaaggcaaagaccttgagtttgaggaagatcagaatctggtagcatttaacttcaagactttttgtctggaaaaccttgaccagatcaaaaaaatgagcattatttcatgtctgacattcctgaagaatcgtcagagcataatgaaggttatcaagcaaagtgattttacttttggtaaaattaccataaagaaaacttcagacaggattggagccactgacatgaccttcagaaggcttgatagcttgattagggtcaggcttgttgaggaaactgggaattctgagaatctcaatactattaaatctaagattgcttcccaccctttgattcaagcctatggattacctcttgatgatgcaaagtctgtgaggcttgccataatgctaggaggtagcttacctcttattgcttcagttgatagctttgagatgatcagtgttgtcttggctatatatcaggatgcaaaatacaaggacctcgggattgacccaaagaagtatgacaccagggaagccttagggaaagtttgcactgtgctgaaaagcaaagcatttgaaatgactgaagatcaggtgaagaaggggaaagagtatgctgctatacttagttcctgcaatcctaatgctaaaggaagtgttgctatggaacattacagtgaaactcttaacaagttctatgaaatgtttggggttaaaaaacaggcaaaactcacagaac

SEQ ID NO: 16 - DNA, 777 bp, Tomato spotted wilt virus
tcaagcaagttctgcgagttttgcctgctttttaaccccgaacatttcatagaacttgttaagagtttcactgtaatgttccatagcaacactccctttagcattaggattgctggagctaagtatagcagcatactctttccccttcttcacctgatcttcattcatttcaaatgctttgcttttcagcacagtgcaaacttttcctaaggcttccttggtgtcatacttctttgggtcgatcccgaggtccttgtattttgcatcctgatatatagccaagacaacactgatcatctcaaagctatcaactgaagcaataagaggtaagctacctcccagcattatggcaagtctcacagactttgcatcatcgagaggtaatccataggcttgaatcaaagggtgggaagcaatcttagatttgatagtattgagattctcagaattcccagtttcttcaacaagcctgaccctgatcaagctatcaagccttctgaaggtcatgtcagtgcctccaatcctgtctgaagttttctttatggtaattttaccaaaagtaaaatcgctttgcttaataaccttcattatgctctgacgattctttaggaatgtcagacatgaaataacgctcatcttcttgatctggtcgatgttttccagacaaaaagtcttgaagttgaatgctaccagattctgatcttcctcaaactcaaggtctttgccttgtgtcaacaaagcaacaatgctttccttagtgagcttaaccttagacat

SEQ ID NO: 17 - DNA, 777 bp, Tomato spotted wilt virus
atgtctaaggttaagctcactaaggaaagcattgttgctttgttgacacaaggcaaagaccttgagtttgaggaagatcagaatctggtagcattcaacttcaagactttttgtctgggaaaccttgaccagatcaaaaagatgagcattatttcatgtctgacattcctgaagaatcgtcagagcataatgaaggttattaagcaaagtgattttacttttggtaaaattaccataaagaaaacttcagacaggattggagccactgacatgaccttcagaaggcttgatagcttgatcagggtcaggcttgttgaggaaactgggaattctgagaatctcaatactatcaaatctaagattgcttcccaccctttgattcaagcctatggattacctcttgatgatgcaaagtctgtgaggcttgccataatgctgggaggtagcttacctcttattgcttcagttgatagctttgagatgatcagtgttgtcttggctatatatcaggatgcaaaatacaaggacctcgggatcgacccaaagaagtatgacaccagggaagccttagggaaagtttgcactgtgctgaaaagcaaagcatttgaaatgactgaagatcaggtgaagaaggggaaagagtatgctgctatacttagttccagcaatcctaatgctaaaggaagtgttgctatggaacattacagtgaaactcttaacaagttctatgaaatgtttggggttaaaaaacaggcaaaactcacagaacttgcttaa

SEQ ID NO: 18 - DNA, 704 bp, Tomato chlorotic spot virus
atgtctaaggtcaagctcaccagagagaacattatctctcttctaactcaggctggagaaatcgagtttgaagaagatcaaaatcaagccacattcaacttcaagactttttgcggagaaaatcttgattcaatcaagaaaatgagcattacctcatgtttgactttcctgaaaaatcgccagagcatcatgaaagttgtgaaccaaagtgattttacctttgggaaaatcacaatcaaaaagaattctggaagggttggagctaatgacatgactttcagaaggcttgatagtatgataagagttaagctgattgaagaaactggaaaagcagaaaacctcgctattatcaagtctaagattgcctctcatcctcttgttcaagcttatggtctgcctttgacagatgcaaagtctgtgaggcttgccataatgctaggaggtagtatccctctgattgcttctggggacagctttgaaatgatcagcatcatccttgccatataccaagatgctaaatataaagatcttggaattgaaccttcgaagtataacactaaagaagctttaggaaaggtctgcactgtgttaaaaagtaaaggatttacaatggatgaagcacaagtgcagaaagggaaagaatatgctacaatactcagctcttgcaatcctaatgctaaaggaagcattgctctggatccggg

SEQ ID NO: 19 - DNA, 777 bp, Tomato chlorotic spot virus
ttatgcaacacctgaaattttggcttctttccttactccgaacattgcatagaatttgtcaagatgctcactgtaatgttccatagcaatgcttcctttagcattgggattgcaagagctgagtattgtagcatattctttccctttctgtatttgtgcttcatccattgtaaatcctttactttttaacacagtgcagacctttcctaaagcttctttagtgttatacttcgaaggctcaattccaagatctttatatttagcatcttggtatatggcaaggatgatgctgatcatttcaaagctgtccacagaagcaatcagagggatactacctcctagcattatggcaagcctcacagactttgcatctgtcagaggcagaccataagcttgaacaagaggatgagaggcaatcttagacttgataatagcaaggttttctgctttcccagtttcttcaatcagcttaactcttatcatactatcaagcctcctgaaagtcatatcattagctccaacccttccagaattctttttgattgtgattttcccaaaggtaaaatcactttggttcacaactttcatgatgctctggcggtttttcaggaaagtcaaacatgaggtaatgctcattttcttgattgaatcaagattttctccgcaaaaagtcttgaagttgaatgcagcttgattttgatcttcttcaaactcgatttctccagcctgagttagaagagagataatgttatctttggtgagcttgaccttagacat

SEQ ID NO: 20 - DNA, 777 bp, Tomato chlorotic spot virus
ttatgcaacacctgaaattttggcttctttccttactccgaacattgcatagaatttgtcaagatgctcactgtaatgttccatagcaatgcttcctttagcattgggattgcaagagctgagtattgtagcatattctttccctttctgtatctgtgcttcatccattgtaaatcctttactttttaacacagtgcagacctttcctaaagcttctttagtgttatactttgaaggctcaattccaagatctttatatttagcatcttggtatatggcaaggatgatgctgatcatttcaaagctgtccacagaagcaatcagagggatactacctcctagcattatggcaagcctcacagactttgcatctgtcagaggcagaccataagcttgaacaagaggatgagaggcaatcttagacttgataatagcaaggttttctgctttcccagtttcttcaatcagcttaactcttatcatactatcaagccttctgaaagtcatatcattagctccaacccttccagaattctttttgattgtgattttcccaaaggtaaaatcactttggttcacaactttcatgatgctctggcgatttttcaggaaagtcaaacatgaggtaatgctcattttcttgattgaatcaagattttctccgcaaaaagtcttgaagttgaatgcagcttgattttgatcttcttcaaactcgatttctccagcctgagttagaagagagataatgttctctttggtgagcttgaccttagacat

SEQ ID NO: 21 - DNA, 477 bp, Tomato spotted wilt virus
gtttgaggaagatcagaatctggtagcattcaacttcaagactttttgtctgggaaaccttgaccagatcaaaaagatgagcattatttcatgtctgacattcctgaagaatcgtcagagcataatgaaggtcatcaagcaaagtgattttacttttggtaaaattaccataaagaaaacttcagacaggattggagccactgacatgaccttcagaaggcttgatagcttgattagggtcaggcttgttgaggaaactgggaattctgagaatctcaatactatcaaatctaagattgcttcccaccctttgattcaagcctatggattacctcttgatgatgcaaagtctgtgaggcttgccataatgctaggaggtagcttacctcttattgcttcagttgatagctttgagatgatcagtgttgtcttggctatatatcaggatgcaaaatacaaggacctcgggatcgaccc

SEQ ID NO: 22 - DNA, 610 bp, Tomato spotted wilt virus
cacaaggcaaagaccttgagtttgaggaagatcagaatctggtagcattcaacttcaagactttttgtctgggaaaccttgaccagatcaaaaagatgagcattatttcatgtctgacattcctgaagaatcgtcagagcataatgaaggtcatcaagcaaagtgattttacttttggtaaaattaccataaagaaaacttcagacaggattggagccactgacatgaccttcagaaggcttgatagcttgattagggtcaagcttgttgaggaaactgggaattctgagaatctcaatactatcaaatctaagattgcttcccaccctttgattcaagcctatggattacctcttgatgatgcaaagtctgtgaggcttgccataatgctaggaggtagcttacctcttattgcttcagttgatagctttgagatgatcagtgttgtcttggctatatatcaggatgcaaaatacaaggacctcgggatcgacccaaagaagtatgacaccagggaagccttagggaaagtttgcactgtgctgaaaagcaaagcatttgaaatgactgaagatcaggtgaaaggggaaagagtatgctgctatactta

SEQ ID NO: 23 - DNA, 620 bp, Tomato spotted wilt virus
acaaggcaaagaccttgagtttgaggaagatcagaatctggtagcattcaacttcaagactttttgtctgggaaaccttgaccagatcaaaaagatgagcattatttcatgtctgacattcctgaagaatcgtcagagcataatgaaggtcatcaagcaaagtgattttacttttggtaaaattaccataaagaaaacttcagacaggattggagccactgacatgaccttcagaaggcttgatagcttgattagggtcaagcttgttgaggaaactgggaattctgagaatctcaatactatcaaatctaagattgcttcccaccctttgattcaagcctatggattacctcttgatgatgcaaagtctgtgaggcttgccataatgctaggaggtagcttacctcttattgcttcagttgatagctttgagatgatcagtgttgtcttggctatatatcaggatgcaaaatacaaggacctcgggatcgacccaaagaagtatgacaccagggaagccttagggaaagtttgcactgtgctgaaaagcaaagcatttgaaatgactgaagatcaggtgaagaaggggaaagagtatgctgctatacttagctccagca

SEQ ID NO: 24 - DNA, 467 bp, Tomato spotted wilt virus
gatcagtatctggtagcattcaacttcaagactttttgtctgggaaaccttgaccagatcaaaaagatgagcattatttcatgtctgacattcctgaagaatcgtcagagcataatgaaggtcatcaagcaaagtgattttacttttggtaaaattaccataaagaaaacttcagacaggattggagccactgacatgaccttcagaaggcttgatagcttgattagggtcaagcttgttgaggaaactgggaattctgagaatctcaatactatcaaatctaagattgcttcccaccctttgattcaagcctatggattacctcttgatgatgcaaagtctgtgaggcttgccataatgctaggaggtagcttacctcttattgcttcagttgatagctttgagatgatcagtgttgtcttggctatatatcaggatgcaaaatacaaggacctcgggatcgaccc

SEQ ID NO: 25 - DNA, 556 bp, Groundnut ringspot virus isolate
ctacatgggatagcagaaagcaatacatgatggtttcaaggattgtgatatgggtttgtcctaccataccagatcctacaggtaagctcttaatagcattaattgaccctaacatgccttcagacaagcaggtgattttaaagggtcagggaacaattacagatccaatatgttttgttttctatttaaactggtctatcccaaaagcaaacaacacgcctgaaaactgctgtcaactgcatctaatgtgcaatcaagagtataaaaaaggagtgttatttgcaagtgtcatgtactcttggacaaaagaattttgtgattctccaagagctgacaaagacaagtgttgcactgtcatacctttgaatagagctatccgggctagatctcaagcctttattgaagcctgtaagctaataattcctaaaggcaatagtgcaaaacaaatcaagaaacagcttaaagatcttagcaccaatttggaaaaatctgttgaggaggaagaagaaggtgtttgtgatgacatagccaagctttcgtttatggacgaaatata

SEQ ID NO: 26 - DNA, 556 bp, Groundnut ringspot virus isolate
ctacatgggatagcagaaagcaatacatgatggtttcaaggattgtgatatgggtttgtcctaccataccagatcctacaggcaagctcttaacagcattaattgaccctaacatgccctctgacaagcaggtgattttaaagggtcaaggaacgatcacagatccaatatgttttgttttctatttaaactggtccatcccaaaagcaaacaacacatctgaaaactgctgtcaactgcatctaatgtgcaatcaagagtataaaaaaggagtttcatttgcaagtgtcatgtactcttggacaaaggaattttgtgattctccaagagctgacaaagataagtgttgcactgtcatacctttgaatagagctatccgggctagatctcaagcctttattgaagcctgcaagctaataattcctaaaggcaacagtgcaaaacaaatcaagaagcagcttaaggatcttagtaccaatttggaaaaatctgttgaagaagaagaagaaggtgtttgtgacgacatagcgaaactttcatttgtggatgaaatata

SEQ ID NO: 27 - DNA, 909 bp, Tomato spotted wilt virus
atgttgactcttttcggtaacaagaggccttctaagtctgtaatggcagtgttgaagtctcaaaaccatggtcttcttctgatgaaaagcttgctttaacccggaaaggatgaaggtcctttagtttcacttgctaaacacaaagccatggatgcatccaaaggaaagatactgttaaacattgagggaacatcttcctttggaacctatgaatctgattccatcacagagtcagaaggttatgatctttctgctagaatgatagtagatacaaaccatcatatctcaaactggaaaaatgatctttttgttggcaacggaaagcaaaatgctaataaggttatcaagatctgtccgacttgggacagcagaaaacaatacatgatgatttccagaattgtgatatgggtctgccccactataccaaaccctacaggaaaacttgtggttgctttaattgatcccaacatgccatctggaaagcaagtcatcctgaagggtcaggggacaataactgatcctatctgctttgttttttatctgaactggtctattccgaagatgaacaacaccccagaaaactgttgtcagctgcatttgatgtgtagccaagaatacaagaaaggggtttcttttggtagtgtcatgtattcttggacaaaagagtttggtgattcacccagagctgataaagacaaaagttgtatggttatacctctaaacagggccattagagctagatctcaggcattcattgaagcctgcaagctgataattcctaaaggaaacagtgagaagcagataaaaaaacagcttaaagaactgagctcaaatcttgagagatcagttgaagaggaagaggaaggaatttctgacagtgttgctcagttatcctttgatgaaatatag

SEQ ID NO: 28 - DNA, 556 bp, Tomato chlorotic spot virus
ctacatgggatagcagaaagcaatacatgatggtttcaaggattgtgatatggatttgtccaaccataccagaccctacaggcaagctcttaatagcattgattgatcctaacatgccttctgataagcaggtgattctaaagggtcagggaacaataacagatccaatatgctttgttttttatctaaactggtctatcccaaaagcgaacaacacacctgaaaactgctgtcagctgcatttaatgtgcagtcaagagtataaaaaaggagtctcatttgcaagtgtcatgtactcatggacaaaagaattttgtgattctccaagagctgataaagataagtgttgcactgtcatacccttgaatagagctatccgggctagatcccaagcctttattgaagcctgcaagctaataataccaaaaggcaacagtgcaaagcaaattaagaagcagcttaaagatcttagtaccaacttggagaaatctgttgaagaagaagaagagggtgtttgtgataacattgctaaactctcttttgtggatgagatata

SEQ ID NO: 29 - DNA, 765 bp, Tomato chlorotic spot virus
atgttgacatttttcggcagcaagaaaccctctgagtctgaaaagacagatgaaggtcccttggtttcattgaccaaacgcaatgggaatgttgaagtctctagatcatggtctcaatctaatgagaaagaagctctggcaaaaaatatggatgtatctaaaggtaagatattattaaacactgaaggaacttcctctctaggaacctatgagtcagactctatcacagaatcagagggttatgatctagctgcaagaatgatagtggatacaaaccatcatatttccaactggaaaaatgatttgtttgttggcaacgggaagcagaatgcaactaagatcattaagatatgccctacatgggatagcagaaagcaatacatgatggtttcaaggattgtgatatggatttgtccaaccataccagaccctacaggcaagctcttaatagcattaattgaccctaacatgccttctgacaagcaggtgattctaaagggtcagggaacaataacagatccaatatgctttgttttttatctaaactggtctatcccaaaagcgaacaacacacctgaaaactgctgtcagctgcatttaatgtgcagccaagagtataaaaaaggagtctcatttgcaagtgtcatgtactcatggacaaaggaattttgtgattctccaagagctgataaagataagtgttgcactgttatacccttaaatagagctatccgggctaggtcccaagcattcattgaagcctgc

SEQ ID NO: 30 - DNA, 553 bp, Tomato spotted wilt virus
ctacatgggatagcagaaagcaatacatgatgatttccaggagtgtgatatgggtctgccctactataccaaaccctacaggaaaacttgtggttgccctggttgatcccaacatgccacctgaaaagcaagtcattctgaagggtcaggggacaataattgatcctatatgttttgtcttttatctgaactggtctattccgaaaatgaataacactccagagaactgctgtcagctgcatttgatgtgcagccaagaatacaagaagggggtttcttttggtagtatcatgtattcttggacaaaggagttttgtgattcacccagagctgataaagataaaagttgcatggtcatacctctaaacagagctattagagctagatctcaagcattcattgaggcttgtaagctgataatccctaaaggaaacagtgaaaagcagattaaaaaacagcttaaagaattgagcttaaatcttgagagatcagttgaagaagaagaggaagagatttctgatagtgttgctcagttatcttttgatgaaatata

SEQ ID NO: 31 - DNA, 909 bp, Tomato spotted wilt virus
atgttgactcttttcggtaataaggggtcttctaagtctgccagaaaggacgaaggtcctttagtttcacttgctaaacataatggtaatgttgaagtctctaagccatggtcttcttctgatgaaaagcttgctttgactaaagctatggatacatccaaaggaaagatactgttgaacacagagggaacatcttcttttggaacctatgaatctgattctatcacagaatcagagggttacgatctttctgcaagaatgatagtagatacaaaccatcatatctcaaactggaaaaatgatctttttgttggcaacgggaagcaaaacgcaaataaggtcatcaagatctgtccgacttgggacagcagaaaacaatacatgatgatttccaggattgtgatatgggtctgccccactataccaaaccctacaggaaaacttgtggttgccttggttgatcccaacatgccatctgaaaagcaagtcattctgaaaggtcaggggacaataattgatcctatatgttttgtcttttatttgaactggtctattccgaaaatgaataacactccagaaaactgctgtcagctgcatttgatgtgcagccaagaatacaagaagggggtttcttttggtagtatcatgtattcttggacaaaggagttttgtgattcacccagagctgataaagataaaagttgcatggtcatacctctaaacagagctattagagctagatctcaagcattcattgaggcttgcaagctgataattcctaaaggaaacagtgaaaagcagattaaaaaacagcttaaagaattgagcttaaatcttgagagatcagttgaagaagaagaggaagggatttctgacagtgttgctcagttgtcttttgatgaaatataa

SEQ ID NO: 32 - DNA, 1404 bp, Groundnut ringspot and Tomato chlorotic spot virus reassortant
atgtcatcaggtgtttatgaatcgatcattcagacaaaggcttcagtttggggatcgacagcatctggtaagtccatcgtggattcttactggatttatgagtttccaactggttctccattggttcaaactcagttgtactctgattcgaggagcaaaagtagcttcggctacacttcaaaaattggtgatattcctgctgtagaggaggaaattttatctcagaacgttcatatcccagtgtttgatgatattgatttcagcatcaatatcaatgattcttttttggcaatttctgtttgttccaacacagttaacactaatggagtgaagcatcagggtcatcttaaggtcctttctcttgcccaattgcatccctttgaacctgtgatgagcaggtcagagattgctagcagattccggctccaagaagaagatataattcctgatgacaaatatatatctgctgctaacaagggatctctctcctgtgtcaaggaacatacttacaaagtcgaaatgagccacaatcaggctttaggcaaagtgaatgttctttctcctaacagaaatgttcatgagtggctgtatagtttcaaaccaaatttcaaccagatcgaaagtaacaacaggactgtaaattctcttgcagtcaaatctttgctcatggctacagaaaacaacattatgcctaactctcaagcttttgttaaagcttctactgattctcattttaagttgagcctttggctgagaattccaaaagttttgaagcaaatagccatacagaagctcttcaagtttgcaggagatgaaaccggtaaaagtttctatttgtctattgcatgcatcccaaatcataacagtgtagaaacagctttaaatgttactgttatatgtaaacatcagcttccaatccctaagtccaaagctccttttgaattatcaatgattttctccgatctgaaagagccttacaacactgtgcatgatccttcataccctcaaaggattgttcatgctttgcttgaaactcacacttcctttgcacaagttctttgcaacaagctgcaagaagatgtgatcatatatactataaacagccctgaactaaccccagctaagctggatctaggtgaaagaaccttgaactacagtgaagatgcttcgaagaagaagtattttctttcaaaaacactcgaatgcttgccagtaaatgtgcagactatgtcttatttggatagcatccagattccttcatggaagatagattttgccagaggagagatcagaatctcccctcaatctactcctattgcaagatctttgctcaagctggatttgagcaagatcaaggaaaagaagtccttgacttgggaaacatccagctatgatctagaataa

SEQ ID NO: 33 - DNA, 1404 bp, Groundnut ringspot virus isolate
atgtcatcaggtgtttatgaatcgatcattcagacaaaggcttcagtttggggatcgacagcatctggtaagtccatcgtggattcttactggatttatgagttcccgactggttctccactggttcaaactcagttgtactctgattcgaggagcaaaagtagcttcggctacacttcaaaagttggtgatattcctgctgtagaagaggaaattttatctcagaacgttcatatcccagtgtttgatgatattgatttcagcatcagtatcaatgattctttcttggcaatttctgcttgttccaacacagttaacaccaatggagtgaagcatcagggtcatcttaaagttctttctcttgctcaattgcatccttttgaacctgtgatgagcaggtcagagattgctagcagattccggctccaagaagaagacataattcctgatgacaaatatatatctgctgctaacaagggttctctctcctgtgtcaaagaacatacttacaaagttgaaatgagctacaatcaggctttaggcaaagtgaatgttctttctcctaacagaaatgttcatgagtggctgtatagtttcaaaccaaatttcaaccagatcgaaagtaataacagaactgtgaattctcttgcagtcaaatctttgctcatggctacagaaaacaacattatgcctaactctcaggcttttgtggaagcttctactgattctcattttaggttgagcctttggctgagaattccaaaagttttgaagcaaatagctatacagaaactcttcaggtttgcaggagatgaaaccggtaaaagtttctatttgtccattgcatgcattccaaatcataacagtgtggaaacagctttgaatgttactgttatatgtaagcatcagcttccaattcctaagtccaaagctccttttgaattatcaatgatcttttctgatctgaaagagccttacaacactgtgcatgatccttcatatcctcaaaggattgttcatgctttgcttgagactcacacttcctttgcacaagttctctgcaacaagctgcaaggagatgtaatcatatatactataaacagccctgaactaaccccagctaagctagatctaggtgaaagaaccctgaactacagtgaagatgcttcgaagaagaggtattttctttcaaaaacactcgaatgcttgccagtgaatgtgcagactatgtcttatctagatagcatccaaattccttcatggaagatagactttgccagaggagagatcagaatctcccctcaatctactcctattgcaagatctttgctcaagctggatttgagcaagatcaaggaaaagaagtccttgatttgggaaacatccagctatgatctagaataa

SEQ ID NO: 34 - DNA, 1404 bp, Tomato spotted wilt virus
atgtcttcaagtgtttatgagtcaatcattcagacaagagcttcagtctggggatcaactgcatctggtaaagctgttgtagattcttactggattcatgaacttggtactggttctcaactagttcagacccagctgtattctgactcaagaagcaaaagtagctttggctatactgcaaaagtaggggatcttccttgtgaagaagaagagattctttctcagcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccattctattggatctatcatgagcagatctgatattacagaccgattccagctccaagaaaaagacataattcccaatgacagatacattgaagctgcaaacaaaggctctttgtcttgtgtcaaagagcatacctataagatcgagatgtgctataatcaagctttaggcaaagtgaatgttctatctcctaacagaaatgtccatgaatggctgtacagtttcaagccaaatttcaatcaagttgaaagcaacaacagaactgtgaattctcttgcagtgaaatctctgctcatgtcagcagaaaacaacatcatgcctaactctcaagcttttgtcaaagcttccactgattctcatttcaagctgagcctctggctaagggttccaaaggttttgaagcagatttccattcagaaattgttcaaggttgcaggagatgagacaaataaaacattttatttatctattgcctgcattccaaaccataacagtgttgagacagctttaaacattactgttatttgcaagcatcagctcccaattcgtaaatgcaaagctcctttcgaattatcaatgatgttttctgatttaaaggagccttacaacattgttcatgatccttcatatccccaaaggattgttcatgctctgcttgaaactcacacatcttttgcacaagttcttagcaacaatttgcaagaagatgtgatcatctacaccttgaacaaccatgagctaacccctggaaagttagatttaggtgaaagaaccttgaattacagtgaagatgcctacaaaaggaaatatttcctttcaaaaacacttgaatgtcttcctactaacacacaaactatgtcttatttagacagcatccaaatcccttcctggaagatagactttgccaggggagaaattaaaatttctccgcagtctatttcagttgcaaaatctttgttaaagcttgatttaagcgggatcaaaaagaaagaatctaaggttaaggaagcatatgcttcaggatcaaaatga

SEQ ID NO: 35 - DNA, 494 bp, Tomato spotted wilt virus
tcccaattcgcaaatgcaaagctccttttgaattatcaatgatgttttctgatttaaaggagccttacaacattgttcatgacccttcatacccccaaaggatcgttcatgctctgctcgaaactcacacatcttttgcacaagttctttgcaacaacttgcaagaagatgtaatcatctacactttgaacaaccttgagctaactcctggaaagttagatttaggtgaaagaaccttgaattacagtgaagatgcctacaagaggaaatatttcctttcaaaaacacttgaatgtcttccatctaacacacaaactatgtcttacttaaacagcatccaaatcccttcatggaagatagactttgccagaggagaaattaaaatttctccacaatctatttcagttgcaaaatctttgttaaagcttgatttaagcgggatcaaaaagaaagaatctaaggttaaggaagcgtatgcttcaggatcaaaataa

SEQ ID NO: 36 - DNA, 1404 bp, Tomato spotted wilt virus
atgtcttcaagtgtttatgagtcaatcattcagacaagagcttcagtctggggatcaactgcatctggtaaagctgttgtagattcttactggattcatgaacttggtactggttctcaactagttcagacccagctgtattctgattcaagaagcaaaagtagctttggctatactgcaaaagtaggggatcttccttgtgaagaagaagagattctttctcagcatgtgtatatccctatttttgatgatattgattttagcatcaatattgatgactctgttctggcactatctgtttgctcaaatacagtcaatgctaacggagtgaaacatcaaggtcatttgaaggttttgtctcctgctcagctccattctattggatctatcatgagcagatctgatattacagaccgattccagctccaagaaaaagacataattcccaatgtcagatacattgaagctgcaaacaaaggctctttgtcttgtgtcaaagagcatacctataagatcgaaatgtgctataatcaagctttaggcaaagtgaatgttctatctcctaacagaaatgtccatgaatggctgtacagtttcaagccaaatttcaatcaagttgaaagcaacaacagaactgtgaattctcttgcagtgaaatctctgctcatgtcagcagaaaacaacatcatgcctaactctcaagcttttgtcaaagcttccactgattctcatttcaagctgagcctctggctaagggttccaaaggtcttgaagcagatttccattcagaaattgttcaaggttgcaggagatgagacaaataaaacattttatttatctatagcctgcattacaaaccataacagtgttgagacagctttaaacattactgttatttgcaagcatcagctcccaattcgtaaatgcaaagctcctttcgaattatcaatgatgttttctgatttaaaggagccttacaacattgttcatgatccttcatatccccaaaggattgttcatgctctgcttgaaactcacacatcttttgcacaagttcttagcaacaatttgcaagaagatgtgatcatctacaccttgaacaaccatgagctaactcctggaaagttagatataggtgaaagaaccttgaattacagtgaagatgcctacaaaaggaaatatttcctttcaaaaacacttgaatgtcttccaactaacacacaaactatgtcttatttagacagcatccaaatcccttcctggaagatagactttgccaggggagaaattaaaatttctccgcagtccatttcagttgaaaaatctttgttaaagcttgatttaagcgggatcaaaaagaaagaatctaaggttaaggaagcatatgcttcaggatcaaaatga

SEQ ID NO: 37 - DNA, 768 bp, Tomato spotted wilt virus
aaagccatggatgcatccaaaggaaagatactgttaaacattgagggaacatcttcctttggaacctatgaatctgattccatcacagagtcagaaggttatgatctttctgctagaatgatagtagatacaaaccatcatatctcaaactggaaaaatgatctttttgttggcaacggaaagcaaaatgctaataaggttatcaagatctgtccgacttgggacagcagaaaacaatacatgatgatttccagaattgtgatatgggtctgccccactataccaaaccctacaggaaaacttgtggttgctttaattgatcccaacatgccatctggaaagcaagtcatcctgaagggtcaggggacaataactgatcctatctgctttgttttttatctgaactggtctattccgaagatgaacaacaccccagaaaactgttgtcagctgcatttgatgtgtagccaagaatacaagaaaggggtttcttttggtagtgtcatgtattcttggacaaaagagtttggtgattcacccagagctgataaagacaaaagttgtatggttatacctctaaacagggccattagagctagatctcaggcattcattgaagcctgcaagctgataattcctaaaggaaacagtgagaagcagataaaaaaacagcttaaagaactgagctcaaatcttgagagatcagttgaagaggaagaggaaggaatttctgacagtgttgctcagttatcctttgatgaaatatag

SEQ ID NO: 38 - DNA, 599 bp, Groundnut ringspot virus isolate
atgaatattcagaaaataagaaagttaatagaaaacggaaccactctattactatccattgaggactgtgtaggttctaaccatgatctagcattagacctgcacaaaaggaatagtgatgagattcctgaagatgttataataaataacaatgcaaaaaattatgagactatgagagagcttattgtaaagatctctactgatggggaaggtttgaacacaggaatagcaacagtggatgttaaaaaacttaatgaactagtgtctctgtttgaacagaagtaccttgaaacagaactctcaagacatgatttgtttggagaactagtatctagacatttgaggatcaagccgaagcaaaagaatgaggtagaaatagagttagcattgagagactatctggaggaattaaacaagaaacaatgtgtaaatagtctcagcagtgatgagtttgaaaggatcaacagagagtatgtggcaactaatgctacacctgacaattatgtcatatataaggaatcgaagaacagcgaactatgtttgatgatctacgactggaagatttctgtagatgcaaaaaacaaaaacaaaaggatcca

SEQ ID NO: 39 - DNA, 599 bp, Groundnut ringspot virus isolate
atgaatattcagaaaataagaaagttaatagaaaacggaaccactctattactatccattgaggactgtgtaggttctaaccatgatctagcattagacctgcacaaaaggaatagtgatgagattcctgaagatgttataataaataacaatgcaaaaaattatgagactatgagagagcttattgtaaagatctctactgacggggaaggtttgaacacaggaatagcaacagtggatgttaaaaaacttaatgaactagtgtctctgtttgaacaaaagtaccttgaaacagaactctcaaggcatgatttatttggagaactagtgtctagacatttgaggatcaagccaaagcaaaggaatgaggtagaaatagagttagcattgagagactatctggaggaactgaacaagaaacaatgtgtaaatagtctcagcaatgatgagtttgaaaggatcaacagagagtatgtggcaactaatgctacacctgataattatgtcatatataaggaatcgaagaacagcgaactatgtttgatgatctacgactggaagatttctgtagatgcaaaaactgaaacaaaaaccatgga

SEQ ID NO: 40 - DNA, 633 bp, Groundnut ringspot virus isolate
agagcaatcaggtaacaacgattttaagcaaaaaatgaatattcagaaaataagaaagttaatagaaaacggaaccactctattactatccattgaggactgtgtaggttctaaccatgatctagcattagacctgcacaaaaggaatagtgatgagattcctgaagatgttataataaataacaatgcaaaaaattatgagactatgagagagcttattgtaaagatctctactgacggggaaggtttgaacacaggaatagcaacagtggatgttaaaaaacttaatgaactagtgtctctatttgaacagaagtaccttgaaacagaactctcaaggcatgatttatttggagaactagtgtctagacatttgaggatcaagccgaagcaaaggaatgaggtagaaatagagttagcattgagagactatctggaggaactgaacaaaaaacaatgtacaaataggatcagcagtgatgagtttgaaaggatcaacagagagtatgtggcaactaatgctacacctgacaattatgtcatatataaggaatcaaagaacagcgaactatgtttgatgatctacgactggaagatttctgtagatgcaaaaaacaaaaacaaaaggatcca

SEQ ID NO: 41 - DNA, 8628 bp, Tomato spotted wilt virus
atgaacatccagaaaatacaaaaattaatagaaaatggaaccactttactgttgtctattgaggattgtgtaggttctaaccacgatctagctttggatttacataagagaaatagtgatgagatcccagaagatgtgattataaataataatgcaaaaaattatgagacaatgagagagttaattgtcaaaatcactgctgatggtgaaggactaaacaaagggatggcaactgtggatgtcaaaaagctaagtgagatggtctctctgtttgagcaaaaatacctagaaacagagttagcaaggcatgacatttttggagagctgatctccaggcacctgagaataaagcccaaacaaagaaatgaagtggagatagagcatgcactaagagaatatctggatgaactcaacaaaaagtcctgcattaacaagctctctgatgatgagtttgagagaataaataaagaatatgtagcaactaatgccacccctgataactatgtgatatataaagaatcaaaaaacagtgagctttgtttaatcatttatgattggaaaatatctgtcgatgccaggactgaaaccaaacaatggagaaatacctacaagaatatttggaaatctttcaaagatataaaagtgaatggaaagccattcctggaagagcatcctgttttcgtttctatagttatattgaaacctattgctgggatgccaatcactgttactagtagcagggttttggagaaattcgaagattctccatcagcattgcacggagaaagaataaagcatgctaaaaatgccaaattgctaaatatttcttatgttgggcaaatagttggaaccacacccacagtggtgagaaactattatgcaaacactcaaagaatcaaatctgaagtcagaggaatcttaggtgatgattttggatctaaagatgtgtttttcagtcactggaccagcaaatacaaagaaagaaatcctactgagatagcctattccgaagatattgaaagaataattgattcacttgttacagatgaaatccctagagaggaaataatacattttttgtttggaaatttctgtttccacattgaaacaatgaatgaccagcatatagctgacaaatttaaagggtaccaaaactcttgtatcaatttaaaaatagagccaaaagctgatttagctgatttgaaagaccacttaatccaaaagcagcaaatatgggaatctctgtatggaaaacaccttgagaagatcatgcttagaattagagaaaaaaagagaaaagaaaaagagatacctgacataaccacagcttttaaccagaatgctgctgaatatgaagaaaggtatcctaactgtttcaatgatctctctgaactaaaactaactttccatgacttggtccccagtttgaagatagaattgagctcagaggtagattacaacaacgcaattattaacaagtttcgggagagcttcaaaagttcttcaagggttatttataatagcccatatagtagcataaataaccaaacaaataaagcaagagatataacaaacttagttagactgtgtttagcagagctaagttgtgatacaacgaaaatggaaaagcaggaacttgaagatgaaatagatataaacaccgggagtatcaaagttgagagaacaaaaaagtctaaagaatggaataagcaaggttcgtgtttaaccagaaacaaaaatgaattttgcatgaaagatacaggcagggagaacaaaactacctattttaaaggcttagcagtaatgaatataggaatgagttctaagaaaagaattctaaaaaaagaagaaataaaagaaaggatctctaaaggcttggaatatgatacctctgaaaggcaggctgacccaaatgatgattactcaagtatagacatgtcttctctgactcatatgaaaaaactgataaggcatgacaatgatgatagcttaagtggtaaaagatttaagggctctttttttctacttcataattttaatataatagaggatggtaagatcacatctgttttcaataattatgctaaaaatcctgaatgcttgtacattcaagattcagtactgaagactgaattagagacttgcaaaaagataaacaaattatgcaatgatctagccatttaccattactctgaagacatgatgcaattctccaaaggtttaatggtggctgacaggtacatgactaaagaaagtttcaagatattaaccacagcaaatactagcatgatgctattagcattcaaaggggatggaatgaacaccggaggatcgggagttccttacatagcattgcatatagtggatgaagacatgtcagatcattttaacatatgttatactaaagaaatttatagctatttccgaagtggtagtaattacatttatataatgaggccgcagagactaaaccaggtgaggctgctgaggcttttcaaaacgcctagtaaagttcctgtatgttttccacaattttcaaagaaagctaatgaaatcggaaaatcgctgaaaaataaagatatagaaaaagtaaatctcttttctatgacaatgactgtaaaacagatattaataaatattgtgttttcatctgtcatgataggaactgtgacaaagctcagtagaatgggaatttttgatttcatgcggtatgcaggttttttgcgactatccgattattctaacataaaagaatacattagagacaaatctgatcctgatataactaactgtggcagatatctatttcgtaatggaatcaaaaaactattgttcagaatggaagatctcaatttaagcacaaatgccaagcctgttgttgtggaccacgaaaatgatattataggagggataacaaacttgaatataaaatgtcctataacaggatcaactctactgacacttgaggacctgtacaataatgtttatttggctatttatatgatgcctaaatcactgcacaatcatgttcacaatctaacaagcttattaaatctccctgctgagtgggagctaaagttcagaaaagaattaggtttcaacatatttgaagacatataccctaagaaagcaatgtttgatgacaaagacctattctccataaatggagctttgaacgtgaaagcattatctgattactatctaggaaatatagaaaatgttggtttaatgagatcagaaatagaaaataaagaagatttcctaagcccttgttataaaatatctactttaaaatcttcaaaaaaatgctcacaatcaaacattataagtactgatgagataatagagtgtcttcaggatgcaaagattcaagatatagaaaattggaaaggaaataacctagctattataaaagggcttataagaacctacaatgaggagaaaaatagattggtggaattttttgaagataattgtgtcaattcattatatcttatagaaaagcttaaagagataattagtagtggatcaataactgtagggaaatctgtaacatctaaattcataagaaacaatcatcctttaacagtagaaacatatctcaaaacaaaactatattatagaaataatgtaacagttttaaagtctaaaaaagtgtcagaggagctctatgaccttgtaaaacagttccatgacatgatggaaatagacctagattctgttatgaacctcgggaaaggtacagaaggaaaaaaactcacattcttgcagatgcttgaatttgtcatgtccaaggctaaaaatgtcaccgggtctgtagattttctagtttctgtttttgaaaaaatgcagagaaccaaaacagacagagaaatatacttgatgagcatgaaagtgaaaatgatgctttattttatagagcatacattcaaacatgtagcgcagagtgatccatcggaagccatatctataagtggagacaataaaataagagcactttctacgttatctttggacacaatcacgtcttacaatgatattttaaacaaaaattcaaagaagtcaagattggctttcctatctgcagatcagtcgaaatggtcggcatcaggccttaccacctataaatatgttttagctatcatattaaatccaattttaactactggtgaagctagcttaatgatagaatgcatcttaatgtatgttaaattgaagaaggtttgtataccaacagatatttttttgaatctaagaaaagctcaacaaacttttggggaaaatgaaactgccataggacttttgaccaaaggcttgacgacaaacacataccctgttagcatgaattggttgcaaggcaatttaaattatctgtcttctgtttatcactcttgtgcaatgaaagcttatcacaacactttggaatgttacaaaaactgtgatttccaaactagatggattgtgcactctgatgataatgcaacatcattaatagccagtggagaggttgataaaatgctgacagacttttcaagctcatctctgccagaaatgttgtttagaagcattgaagctcatttcaaaagtttttgcataactttgaacccaaaaaagagttatgcttcttcatcagaagtagagtttatatctgaaagaattagtaaatggagcgattattcctctctattgcaggcatttagcaaactgttgcacagaatcttcgcatataagttatttgatgatctaatgtcactcagtatacatgttacaatgcttctgagaaaaggctgtcctaatgaagttataccttttgcttatggggctgtgcaggtacaagcgttaagcatctattcaatgcttcctggtgaagtgaatgatagtattagaatttttaacaagcttggagtaagtttaaagtcaaacgagattcccacaaacatggggggctggttgacctctcctatagagccgttgtctatattaggtccatcatcaaatgatcaaatcatctattacaatgtgataagagattttttgaacaaaaaaagtttagaagaagtaaaagatagtgtctcttcttccagttatctacagatgagattcagagagctaaaagaaaagtatgaaagaggaactctggaagaaaaagataaaaagatgatatttcttatcaatctgtttgagaaagcatcagtgtctgaagattcagacgttctaacaattgggatgaaatttcaaactatgttaactcagattataaaattacctaattttataaatgagaatgctttaaacaagatgtcaagttataaagatttttcaaaactttatcctaatttaaaaaagaatgaagatttatataaaagcactaagaacttaaagatagacgaggatgctgttttagaggaagatgagttatataagaagattgcatctagcttagaaatggaatctgtccatgacataatgataaaaaatcctgaaacaattctgatagcaccattgaatgatagagattttttacttagtcagctgttcatgtacacaagcccttctaaaagaaaccagttatcgaaccaatctacagagaaacttgctttagatagagtgttaaggtcaaaagctagaacatttgtaaacatttcttccactgtgaagatgacttatgaagaaaacatggaaaagaaaatcttagaaatgctaaaatttgatttagattcatattgttcatttaaaacatgtgtaaatctagttatcaaggatgttaatttcagcatgctgattccaatattagattctgcatacccttgtgaatctaggaaaagagataactacaatttcaggtggtttcagactgagagatggatacctgttgttgaaggctctccgggactagtagtaatgcatgctgtctatggatcaaattatatagagaacttaggtttaaaaaacatccctctaacagacgatagtattaatgttttaacaagcacgtttggaacaggtttaatcatggaagatgtaaaatccctagttaatggcaaagacagcttcgaaacagaggcttttagcaattctaatgaatgtcaaagattggtgaaagcatgcaattatatgatagcagcacaaaacaggcttttagcaattaacacatgctttactaggaaaagctttcccttctattctaagttcaatctagggagagggtttatctcaaacacattagctctcctatccaccatctacagtaaagaagaatcctatcattttgtttctacagctagttataaattagacaaaactatcagaactgtggtaagtgctcagcaagatatgaacttagagaaaatactggacactgctgtatacatatcagataaattgcagtcacttttcccaacaattacaagagaggatatagttttaatattgcaaaatgtttgccttgacagtaaacctatatggcagagtctagaagacaaaatgaaaaagattaacaattcaacagcaagtggcttcacagtgtcaaatgtgattctatcacataacagtgaattgaacacaatccagaaacaaattgtctggatgtggaacatgggtttgtgttctcacagaacattagattttgttatcaggtatattagaagaagggatgtaagatatgtaaaaactgaagaacaagatgaatcaggaaattatgtctctggaactatgtacaaaatagggatcatgacaagaagctgctatgtggaattgatagcatctgatcaagatgtagcagtttctttgagaacaccatttgagatattgaatgaaagagagtatctttttgacacatacagagaaagtatagagaaattattggcagaaattatgtttgataaagtgaacataataaatcaaacaaccacagattgttttcttagaaccaggagatcttgcatcagaatgaccacagacaacaaaatgattgtaaaggttaatgctacatcaagacaaataagactagagaatgtaaaattagttgtaaagataaaatatgaaaatgtgaattccgatgtatgggatattatagaaagccaaaaatctctagtcttaaggctccctgaagtaggggaatttttctctgatatgtataaaactgcagactctgaaactgaaacaatcaaaaccataaaaaacaggcttatgacttctttaactttcatagaagcctttggaaacttatcacagcagatcaaagagattgtagatgatgatatcagagaaacgatggatgaattcttaatgaacatccgggatacctgcttagaaggtttggaaaactgcaaaagtgtggaagaatatgatagctatcttgatgaaaatggatttaatgacacagtagaactattcgaaaacttgctaagaacacatgacaactttgaaaatgagtatagtcctcttttttcagagattgttgacaaagcaaaacagtatactagagatttagaaggtttcaaagaaatactgctcatgcttaaatattctctaataaatgatgcatcaggatttaaaagctatagagccactggaatgcatgctgttgagctaatggcaaaaaagcacatagagataggggaattcaacttgttaggaatgatccaattgattaaagcttgtgaaacatgccacaacaatgactctatattaaacttagcaagtttaaggaatgttcttagcaggacatatgccacatttgggaggagaataagattggatcatgatctggacttgcaaaacaacttaatggaaaaaagttatgatttcaagacgctggttttaccagaaataaaattatcagaactatctagggaaatactgaaagaaaatgggtttgttatatctggagagaatctaaaaatggataggtctgatgaagaatttgtgggtcttgccagttttaatgtgttgaggctagatgaggaagaaatgtatgaaggtttgatcaaagaaatgaaaattaaaaggaaaaagaaagggtttttatttccagcaaacacacttctactaagtgagttgataaagttcttgattggaggaataaagggaaccagctttgatatagagacattgttacggaacagttttagaccagacatattttcaactgacagattgggaagattaagttccagtgtacctgcactcaaagtttatgcaactgtttatatggaatataagaatgtcaattgtcctttaaatgagatagctgacagcttagaaggttatctaaaactgacaaaaagcaggtccaaggaacatttcttgtctggaagagttaaaaaagctttgatacaattaagagatgaacaatcgcgaactaaaaaactagaggtctataaggatatcgcaaatttccttgctaggcacccactatgtttatcagaaaaaacattgtatggaagatatacctactctgatatcaatgattatatcatgcaaacaagagagattattttgagtaaaataagtgagttggacgaggttgttgaaacagatgaagacaatttcttgcttagttatctaagaggggaagaagatgcctttgatgaagatgagcttgatgaagaagaagacacagattaa

SEQ ID NO: 42 - DNA, 599 bp, Tomato chlorotic spot virus
atgaacattcagaagattaaaaagttaatagagaacggaaccacactactgttatctattgaagattgcgttggttctaaccacgaccttgctttagacctgcacaaaaagaatagcgatgagatccctgaggatgtaataataaataataatgctaagaattatgagactatgagagagcttattgtaaaaattacttctgacggagaaggcttgaacacaggaatagcaacagtggatattaaaaaactcaatgaattagtttcactgttcgaacaaaaatatctggaaactgaactctctagacatgatatgttcggtgagctggtgtcaaggcacttaaggatcaaacccaaacaaagaaatgaagtagaaatcgagctagcattgagagagtatctggaggaattgaacaaaaagcaatgcataaatagcatcactaatgatgagtttgaaaggatcaataaagaatatgtggccaccaatgccacgccagacaattatgtaatctacaaagagtcaaaaaacagtgagctgtgtttaatgatatacgattggaaaatttctgtggatgcaaaaaacaaaaacaaaaggatcca

SEQ ID NO: 43 - DNA, 8622 bp, Tomato chlorotic spot virus
atgaacattcagaaaattaaaaagttaatagagaacggaaccacactactgttatctattgaagattgcgttggttctaaccacgaccttgctttagacctgcacaaaaagaatagcgacgagatccctgaggatgtaataataaataataatgctaagaattatgagactatgagagagcttattgtaaaaattacttctgacggagaaggcttgaacacaggaattgcaacagtggatattaaaaaactcaatgaattagtttcactgttcgaacaaaaatatctagaaactgaactctctagacatgatatgttcggtgggctggtgtcaaggcatttgaggatcaaacccaaacaaagaagtgaagtagaaatcgaactagcattgagagagtatctggaggaattgaacaaaaggcaatgcataaatagcatcactaatgatgagtttgaaaggatcaataaagaatatgtggccaccaatgccacgccagacaattatgtaatctacaaagagtcaaaaaacagtgagctgtgtttaatgatatatgattggaaaatttctgtggatgcaaaaactgaaacaaaaactatggaaaaatactacaaaaacatctggaaatcttttaaagacattcatgttaatggaaaacctttcctagaagaccaccctgttttcataacaatagtaatccttaaacctatgggaggaatgccaataacagtcaccagcagcagggttttaggaaaatttgaagattcaccttctgcactccatggagagagatcaagacatgctaaaaatgccaagctactaaacatatatcatgtgggtcagatagttggaacaacaccgaccatagttcgaaataattatgcaaacactcaaaaactcaaatctgaagttagaggaatattaggtgatgatttcggttccaaagatgtgtttttcagccactgggcaaacaaatataaagatagaaatcccactgaaatcgcttactctgaagatatagaaaagattatagagtctttggccacagatgagattcccagagatgaaatcatccatttcttatttgggaatttctgctaccacatagagactatgaacgatcagcatattgcagacaggtttaaaggataccaagaagcatgcatgaatctcaaaataatcccaaaaaatgatttatctgatttgaaggatcatttgatccagaagcaacaagtctgggaatctttgtatgggaagcatctagggaaaattatgcatagaataaaaaagaaaaaagagaaagagagagaaattcctgacataacaactgctttcaatcagaatgccatagaatatgaagaaaaatacccgaactgtttcaccaatgacctttctgagacaaaaacgaatttttctatgacttggtctcctagttttgaaaaagttgatctgaactcagaaatagattacaacaatgcaattataaagaaatttagagacagcttcctcgcagcatctagaatttcttataatagtccatacagcactatacacaatcaaacaaacaaggcaagagatgttacaaaactggttgaactctgcttgacagaacttagctgtgatacatcaaaatttagcaagcaagagctagaagatgaaatagatgttaacactggaagcataaaagtagaaaggacaaaaaaatctcaagaatggtataagcatggctcctgtctgactagaaataaaaacgagttctgcatgaaagaaacaagcaaagataacaaggctatctatttcaaaggtttagctgttatgaatataggaatgagctctaaaaaaagaatacttaaaaaagaagaaataaaagaaagaatctctcgaggattagagtatgacacttcttctagacaatctgatccaaatgatgattacaccagtgtggacatgtcatctttaacccacatgaaaaagttaatacgccacgacaacgaagaaagtttaagctggtgtgataagataaaggattctttgtttgtccttcataatggtgacataagagatgaaggcaaggtgacagctgtttacaataattatgcaagaaatccagaatgcttgtacactcaagattcagttttgaaaacggaaatggagacttgtaaaaagataaacaagttatgcaatgatctagctatatatcactattcagaagacatgatgcaatttgccaaaggtttaatggtagcagatagatacatgactaaggaaagcttcaagatattgaccactgcaaatactagcatgatcttgctggcttttaaaggcgatgggatgaacacaggaggttcaggagtcccttacatagcattacatatggtggatgaagatatgtcagaacagttcaatatatgttacaccaaagaaatttacagctacttccgtaatggtagcaactatgtttatatcatgaggccgcaacggttgaatcaggttaggttattaagcctttttaaatctcctagcaaagttccagtctgttttgctcaattttcaaaaaaagctagtgaattggaaaaatggttgaagaataaagatacagaaaaagtaaatgtctatgccatgactgtgacagtaaaacaaatcttaataaatatagtgttttcatctgtgctgataggaactgttactaaactcagcagaatgggaatttttgattttatgggatatgctggatttttaccattgtctgactattctaatataaaagaatatattagagataaatttgatcctgatattactaatgttgcagatatttactttgttaatggtataaaaaagcttttattcagaatggaggaccttaatctaagtaccagtgcaaaacctgttgttgtagatcatgaaaatgatgtcattggaggtataactaatctaaacataaagtgtccaataactgggtccactttactcactttggaagacttatataataatgtctatttagctatttacatgatgccgaaatcacttcacaatcatgtacacaatctaactagtttgcttaatgtgccagctgaatgggaactaaagtttagaaaagaactcgggttcactatatatgaagatatctatcctaaaaaagaaatgtttgatgataaagatttattctctataaatggacttttgaatttgaaagctctatcagattattatattgaaaatgttgataatgttggtctaatgagatcagagatagagaataaagaagattttctaagtccatgctacaaaataacaactctaaaatcttctaaaaagtgttcacaatcaaatataataaacacagatgatataataaattgtcttcaagaagtaaaggttaaagatgttgagaattggaagggtaatacattagccataataaaaggattaattagaacttacaatgaagaaaaaaacaggttagttgagtttcttgatgacaactgtgtaaattcgttgtatcttctagagaaattgagagaaattattgctagtggtgctgttactgtagggaagtctatgacttctaaattcatcagaaacaatcatccattgacagtggaaacgtatctgaagactaaattatattacagaaacaacgtaactgttctgaaatcaaaaaaagtttctgaagaactctatgatctagtaaaacaatttcacaacatgatggagatagatatagattctgtcatgaatctagggaaaggacttgaagggaaaagacacacttttttgcagatgctagaatttgtgatttctaaagcaaaaaatgtcacaggatctgtagatttcttggtctctgtatttgaaaaaatgcaaaggacaaagacagacagagaaatatatttgatgagcatgaaagttaaaatgatgctatatttcatagaacacacattcaagcacattgctcaaagtgatccttcagaggctatatctatcagtggagataataagataagagcattatcaacattatctcttgacacaataacttcttacaatgacatcttaaacaaaagttcaaagaaatcaagattggcatttctgtctgcagaccaatctaaatggtcagcatctgatcttacttacaagtacattctagcaattcttctgaaccctgtcttgactacaggtgaagcaagcttaatggtggagtgtttgttaatgtacatcaaacttaaaaaggtttgcataccaacagatatttttctgaatcttaaaaaatcccagagcacttttgggcagaatgagactgctattggattacttacaaagggtttgactaccaatacataccctgtcagcatgaactggttacaagggaatttgaattatttatcatcagtttatcactcgtgcgcaatgaaagcttaccataagactttggaatgttacaaaagctgtgagttccaaactagatggattgttcattctgatgataatgccacatcattgatagctaatgaagaagtggataaaatgttgcacgacttctcaagcgtttctcttccagagatgttgttcagaagcatagaagcacattttaagagcttttgcataacattaaatccaaagaaaagttatgcttcttcctcagaagtagaattcatttctgaaaggatagtgaatggggctattatacctctttactgtaggcatttagcaaattgttgcacagaatcttctcacattagctactttgatgatctgatgtcacttagcatacatgtgaccatgttattgaggaaagggtgcccaaatgaagttataccctttgcttatggtgccgtccaagttcaagcattaagcatctactccatgcttccgggtgaagtcaatgatagcataagaatttttcaaaaactgggtgtaagtttaaaacctaatgaaataccaacaaacatgggaggttggctaactgctcctatagagcctttgtctatacttggaccttcttcaaatgaccagatcatctactacaatgtaataagagacttcttgaacaaaaaaagtctggaagaagtcaaaagtagtgtatctaccttaagctatcttcaaatgaggtttaaagagctagaagaaaaacatgaaaaagggagactagaagaaaaagataaaaagatgattttcttgataaatctttttgagaaagcttcagtatctgaagactcagatgttttaacaatagggatgaaattccaaactatgctgacacaaatcatcaaactacctcaatttataaacgaaaccgcattaaataaaatgtcaagttataaggatttttcgaagctttatcctaacttaaaaaagaacgaggacctttataaaagcacaaaaaatataaggttcaacgaagactcagtcttagaagaagatgatctatatgaaaagctggctcctagtgctgaaatggagtcagtccacaatataatgatacaaaatccagagacaatcttaatcgcccctttaaatgaccgagattttttgctcagccagctgtttatgtacactagtccgtcaaaaagaaatcaattgtccaaccaatcaacagaaaaattagctttggatagagttttgagatctaaagccaaaacatttatagacattgattctcatgtgaaaatgacttacgaagaaaatatggaaaggaaaatcttagaaatgctgaaatttgaccctggttcttactgttctttcaaaacatgtataaacttagtcattaaagatgtgaattttagcatgctaacccctatactggattctgcttacccttgtgaatcaaggaaaagggacaattacaacttcaggtggtttcaaacagaaaaatggataccagttgttgaaggttctcctggcttggtggtaatgcatgctgtgtatggatctaattatattgaaaatttaggcttaaaaaacatacctttgacagatgatagcataaatgttcttacaagcacttttgggacaaacttattaatggatgatgtgaaatcctttgtagctggaagaagcagttttgaaactgaggctttctcagaagctaataattgccagagattagtaagagcatgcaactacatgataacagctcaaaatagactgcttgctataaatacttgtttttcaaggaagagtttccctttttactccaagtttaacctaggtagaggatttatctccaacactttggcattactttccactatttatagtaaagaagaatctttccattttgtctccacagctaattacaaactggacaaaactataagaaccgtgctgagcgcgcagcaagacatgaatttagaaaaaatacttgatacagcagtgtacatctcagacaagatgcagtccctcttcccaaccataaccagagaagatatatctttaatactccaaaatatttgtttggatagcaagcctatatggcaaagccttgaagaaaaaatgaggaagattaacaactctacagcgagtggattcacagtatcaaatgttatattgtcacataacagtgaactgaacactatacaaaaacaaatagtttggttatggaatatgggtttatgctctaacaggacattagattttgttatcagatacatacgacggagcgatgttagatatgtcaagactgaagaacaagacgagttaggaaattatgtttctggcacaatttacaaaataggaataatgactaggagttgttatgtgcaattaattgcatctgatcaagatgtggcagtgacattaaggactccttttgaaatattgaatgagagagattttttatatgacacatacagagagagtatcgagaaacttttgcagaaattcatgttcgacaaaataaacataataaagtccaaacagcctcaaatagtttttttagagccaggagatgcttgtttaagaatgaccacagataacaaaatgattgtcaaagtaaatgccacaccgaagcagattaggctagaaaatgtcaaattggtgattagaataaagtatgaaaatgtgaactcagatgtatgggatattatagaaagccaaaaagcacttgttttaaggatccctgaagtaggagaatgtttttctgacatgtataaaactattgattctgaaaccgaagcaattaaagtaattaaggctggattaatgacctcactgacattcatagaaacctttggtaatttatctaagcaaatagatgaaatagaagacgagactgcaagagagacaatgcatgacttcttgatgaacattaaagacacttgtttggaagggttagaaagttgtaaaagcgttgaggaatatgatgagtttttggaagaaaatggattcaatgacactgtagaattatttgaagaacttctaaagacacaagataattttgaaaacgagtatagcccattgttttctgaaattgtggacaaagcaaaaaaatatacaaaagatctagaaggattcaaagaaatcttattaatgctcaagtattctttaataaatgatgcatcaggatttaagagctacagagcaacaggatcgcatgctgtagagctgatgactaagaaacatatagaaataggtgactttaacctcctgggaatgatccaattgataaaagcttgtgaaacatgccacaacaacgattcaatactaaatttagctagcctgaggaatgtgttgagcagaacatacaccacgtccgggagacgtataaatttaaatcatgaactagatctacagaataatttaatggaaaaaagttatgattttaaaacattggttctacctgacataaagctatctgacatgtctagagagattttaaaagataatgggttcgttatttcaggagaaaacttgaaaatcgacaaatcaggagaagagtttgaaggtttggcaacttttgatatgatgagattggatgaagaagggatgtatgaaggtcttataaaggaaatgaaaattaagaggaaaaagaaagggttccttttccctgcaaatactttgttactgagtgagttaataaaatttttgatcggagggatcaagggcactagctttgatattgaaacactattgagaaacagttttagacctgacatgttttcaacagacagactaggagctaagctctatgttccagcactgcgagtttacgccactgtttatatggaatataaaaatacaaactgtccattaaatgaaattgcagatagtctagaagggtttttaaaattgactaaaagcaaagctaaagaacagctcttggatgggagagtcaaaagagctctaatacagctcagagatgagcattctagatcaaaaaagttagaagtgtataaagatatagctaacttcttgtcaagacatccgctatgtttatcagagaaaacattatatggcagatacacttattatgagataaatgattatatcatgcagactagagaaattatactaagcaaaattaatgaactggacgattatatcgaaacaaatgaggacgaattcttgttaagttacctaaaaggggaagaagaagcttttgatgaagatgaagaaagtgattga

SEQ ID NO: 44 - DNA, 1532 bp, Tomato chlorotic spot virus
tcatcagagtgtaccatccatctagtttgaaactcacagcttttgtaacattccagagtcttatggtaagctttcattgcgcacgaatgataaactgatgataaataattcaaattcccttgtaaccagttcatactgacagggtatgtattagtagtcaaaccttttgtaagtaatccaatagcagtctcattctgcccaaaagtgctctgggattttttaagattcagaaaaatatctgttggtatgcaaacctttttaagtttgatgtacattaacaaacactccaccattaagcttgcttcacctgtagtcaagacagggttcagaagaattgctagaatgtacttgtaagtaagatcagatgctgaccatttagattggtctgcagatagaaatgccaatcttgatttctttgaacttttgtttaagatgtcattgtaagaagttattgtgtcgagagataatgttgataatgctcttatcttattatctccactgatagatatagcctctgaaggatcactttgagcaatgtgcttgaatgtgtgttctatgaaatatagcatcattttaactttcatgctcatcaaatatatttctctatctgtctttgtcctttgcatcttttcaaatacagagaccaagaaatctacagatcctgtgacattttttgctttagaaatcacaaattctagcatctgcaaaaaagtgtgtcttttcccttcaagtcctttccctagattcatgacagaatctatatctatctccatcatgttgtgaaattgttttactagatcatagagttcttcagaaacttttttttgattcagaacagttacgttgtttctgtagtataatttagtcttcagatacgtttccactgtcaatggatgattgtttctgatgaatttagaagtcatagacttccctacagtaacagcaccactagcaataatttctctcaatttctctagaaggtacaatgaatttacacagttgtcatcaagaaactcaactaacctgtttttttcttcattgtaagttctaattaatccttttattatggctaatgtattacctttccaattctcaacatctttaacctttacttcttgaagacaatttattatatcatctgtgtttattatatttgattgtgaacactttttagaagattttagagttgttattttgtagcatggacttagaaagtcttctttattctctatctctgatctcattagaccaacattatcaacattttcaatataataatctgatagagctttcaaattcaaaagtccatttatagagaataaatctttatcatcaaacatttcttttttaggatagatatcttcatatatagtgaacccgagttcttttctaaactttagttcccattcagctggcacattaagcaaactagttaggttgtgtacatgattgtgaagtgatttcggcatcatgtaaatagctaaatagacattattatataagtcttctaaagtgagtaaagtggacccagt

SEQ ID NO: 45 - DNA, 1532 bp, Tomato chlorotic spot virus
tcatcggagtgcaccatccatctagtttgaaactcacagcttttgtaacattccagagtcttatggtaagctttcattgcgcacgaatgataaactgatgataaataattcaaattcccttgtaaccagttcatactgacagggtatgtattagtagtcaaaccttttgtaagtaatccaatagcagtctcattctgcccaaaagtgctttgggattttttaagattcagaaaaatatctgttggtatgcaaacctttttaagtttgatgtacattaacaaacactccaccattaagcttgcttcacctgtagtcaagacagggttcagaagaattgctagaatgtacttgtaagtaagatcagatgctgaccatttagattggtctgcagatagaaatgccaatcttgatttctttgaacttttgtttaagatgtcattgtaagaagttattgtgtcgagagataatgttgataatgctcttatcttattatctccactgatagatatagcctctgaaggatcactttgagcaatgtgcttgaatgtgtgttctatgaaatatagcatcattttaactttcatgctcatcaaatatatttctctatctgtctttgtcctttgcatcttttcaaatacgaagaccaagaaatctacagatcctgtgacattttttgctttagaaatcacaaattctagcatctgcaaaaaagtgtgtcttttcccttcaagtcctttccctagattcatgacagaatctatatctatctccatcatgttgtgaaattgttttactagatcatagagttcttcagaaactttttttgatttcagaacagttacgttgtttctgtagtataatttagtcttcagatacgtttccactgtcaatggatgattgtttctgatgaatttagaagtcatagacttccctacagtaacagcaccactagcaataatttctctcaatttctctagaaggtacaatgaatttacacagttgtcatcaagaaactcaactaacctgtttttttcttcattgtaagttctaattaatccttttattatggctaatgtattacccttccaattctcaacatctttaacctttacttcttgaagacaatttattatatcatctgtgtttattatatttgattgtgaacactttttagaagattttagagttgttattttgtagcatggacttagaaagtcttctttattctctatctctgatctcattagaccaacattatcaacattttcaatataataatctgatagagctttcaaattcaaaagtccatttatagagaataaatctttatcatcaaacatttcttttttaggatagatatcttcatatatagtgaacccgagttcttttctaaactttagttcccattcagctggcacattaagcaaactagttaggttgtgtacatgattgtgaagtgatttcggcatcatgtaaatagctaaatagacattattatataagtcttctaaagtgagtaaagtggacccagt

SEQ ID NO: 46 - DNA, 747 bp, Tomato chlorotic spot virus
atgtcaagctataaagatttttcaaaactttaccctaatttgaaaaagaatgaagatttatataaaagtactaagaacttgaagatagacgaggatgctattttagaggaagatgaattatatgagaagattgcatctagtttagaaatggaatctgttcatgacataatgataaaaaatcccgaaactattttgatagcaccattgaatgatagagattttttacttagtcagctgttcatgtacacaagcccttccaagaggaaccagttatcgaaccaatccacagagaaacttgctttagatagagtgctaaggtcaaaagctaaaacatttgtagacatttcttctgctgtgaagatgacttatgaagaaaacatggaaaagaaaatcttagaaatgctaaaatttgatttagattcatattgttcatttaaaacatgtgtaaatctggtgatcaaggatgttaatttcagcatgctaattccaatattggattctgcatacccttgtgaatctaggaaaagagataactacaatttcaggtggttccagactgaaaaatggatacctgttgtcgaaggctctccgggactagtagtgatgcatgctgtgtatggatcaaattatatagaaaatttaggtctaaaaaacatccctctaacagatgatagcattaatgttttaacaagcacgtttggaacaggtttaatcatggaagatgtaaaatccctagttaatggcaaa

SEQ ID NO: 47 - RNA, 101 nt, Groundnut ringspot virus
augucuaaggucaagcucacaaaagaaaacauugucucucuuuugacucaaucugaggauguugaguuugaagaagaccagaaccagguugcauucaacuu

SEQ ID NO: 48 - RNA, 23 nt, Groundnut ringspot virus
uugacuuucuugaagaaucgcca

SEQ ID NO: 49 - RNA, 23 nt, Groundnut ringspot virus
gaaaaauucugagaagguuggag

SEQ ID NO: 50 - RNA, 23 nt, Groundnut ringspot virus
gacuuucagaaggcuugauagca

SEQ ID NO: 51 - RNA, 23 nt, Groundnut ringspot virus
ugagacuugccauaaugcuugga

SEQ ID NO: 52 - RNA, 100 nt, Tomato spotted wilt virus
aagcucacuaaggaaagcauuguugcuuuguugacacaaggcaaagaccuugaguuugaggaagaucagaaucugguagcauuuaacuucaagacuuuuu

SEQ ID NO: 53 - RNA, 23 nt, Tomato spotted wilt virus
aaaaccuugaccagaucaaaaaa

SEQ ID NO: 54 - RNA, 25 nt, Tomato spotted wilt virus
agcauuauuucaugucugacauucc

SEQ ID NO: 55 - RNA, 25 nt, Tomato spotted wilt virus
guuaaaaaacaggcaaaacucacag

SEQ ID NO: 56 - RNA, 100 nt, Tomato chlorotic spot virus
uuaugcaacaccugaaauuuuggcuucuuuccuuacuccgaacauugcauagaauuugucaagaugcucacuguaauguuccauagcaaugcuuccuuua

SEQ ID NO: 57 - RNA, 23 nt, Tomato chlorotic spot virus
uuugaucuucuucaaacucgauu

SEQ ID NO: 58 - RNA, 23 nt, Tomato chlorotic spot virus
gguaaaaucacuuugguucacaa

SEQ ID NO: 59 - RNA, 23 nt, Tomato chlorotic spot virus
aucuuuggugagcuugaccuuag

SEQ ID NO: 60 - RNA, 23 nt, Tomato chlorotic spot virus
gaugaugcugaucauuucaaagc

SEQ ID NO: 61 - RNA, 23 nt, Tomato chlorotic spot virus
uagacuugauaauagcaagguuu

SEQ ID NO: 62 - RNA, 100 nt, Groundnut ringspot virus
cuacaugggauagcagaaagcaauacaugaugguuucaaggauugugauauggguuuguccuaccauaccagauccuacagguaagcucuuaauagcauu

SEQ ID NO: 63 - RNA, 23 nt, Groundnut ringspot virus
aaagggucagggaacaauuacag

SEQ ID NO: 64 - RNA, 23 nt, Groundnut ringspot virus
ugcaaucaagaguauaaaaaagg

SEQ ID NO: 65 - RNA, 23 nt, Groundnut ringspot virus
ucuaucccaaaagcaaacaacac

SEQ ID NO: 66 - RNA, 100 nt, Tomato chlorotic spot virus
cuacaugggauagcagaaagcaauacaugaugguuucaaggauugugauauggauuuguccaaccauaccagacccuacaggcaagcucuuaauagcauu

SEQ ID NO: 67 - RNA, 23 nt, Tomato chlorotic spot virus
uugcaagugucauguacucaugg

SEQ ID NO: 68 - RNA, 23 nt, Tomato chlorotic spot virus
aacaauaacagauccaauaugcu

SEQ ID NO: 69 - RNA, 25 nt, Tomato chlorotic spot virus
uaauaauaccaaaaggcaacagugc

SEQ ID NO: 70 - RNA, 150 nt, Tomato spotted wilt virus
gcuugcaagcugauaauuccuaaaggaaacagugaaaagcagauuaaaaaacagcuuaaagaauugagcuuaaaucuugagagaucaguugaagaagaagaggaagggauuucugacaguguugcucaguugucuuuugaugaaauauaa

SEQ ID NO: 71 - RNA, 23 nt, Tomato spotted wilt virus
uucgguaauaaggggucuucuaa

SEQ ID NO: 72 - RNA, 23 nt, Tomato spotted wilt virus
uuacgaucuuucugcaagaauga

SEQ ID NO: 73 - RNA, 23 nt, Tomato spotted wilt virus
uugggacagcagaaaacaauaca

SEQ ID NO: 74 - RNA, 100 nt, Tomato spotted wilt virus
augucuucaaguguuuaugagucaaucauucagacaagagcuucagucuggggaucaacugcaucugguaaagcuguuguagauucuuacuggauucaug

SEQ ID NO: 75 - RNA, 23 nt, Tomato spotted wilt virus
auucugacucaagaagcaaaagu

SEQ ID NO: 76 - RNA, 23 nt, Tomato spotted wilt virus
gaagaagagauucuuucucagca

SEQ ID NO: 77 - RNA, 23 nt, Tomato spotted wilt virus
gcucuuugucuugugucaaagag

SEQ ID NO: 78 - RNA, 23 nt, Tomato spotted wilt virus
ucugcucaugucagcagaaaaca

SEQ ID NO: 79 - RNA, 100 nt, Groundnut ringspot virus isolate
augaauauucagaaaauaagaaaguuaauagaaaacggaaccacucuauuacuauccauugaggacuguguagguucuaaccaugaucuagcauuagacc

SEQ ID NO: 80 - RNA, 23 nt, Groundnut ringspot virus isolate
ucaacagagaguauguggcaacu

SEQ ID NO: 81 - RNA, 23 nt, Groundnut ringspot virus isolate
ugaggaucaagccgaagcaaaag

SEQ ID NO: 82 - RNA, 23 nt, Groundnut ringspot virus isolate
aaguaccuugaaacagaacucuc

SEQ ID NO: 83 - RNA, 100 nt, Tomato spotted wilt virus
augaacauccagaaaauacaaaaauuaauagaaaauggaaccacuuuacuguugucuauugaggauuguguagguucuaaccacgaucuagcuuuggauu

SEQ ID NO: 84 - RNA, 23 nt, Tomato spotted wilt virus
ggacuaaacaaagggauggcaac

SEQ ID NO: 85 - RNA, 23 nt, Tomato spotted wilt virus
ugagcaaaaauaccuagaaacag

SEQ ID NO: 86 - RNA, 23 nt, Tomato spotted wilt virus
ucuggaugaacucaacaaaaagu

SEQ ID NO: 87 - RNA, 100 nt, Tomato chlorotic spot virus
ucaucagaguguaccauccaucuaguuugaaacucacagcuuuuguaacauuccagagucuuaugguaagcuuucauugcgcacgaaugauaaacugaug

SEQ ID NO: 88 - RNA, 23 nt, Tomato chlorotic spot virus
gauuuuuuaagauucagaaaaau

SEQ ID NO: 89 - RNA, 23 nt, Tomato chlorotic spot virus
aacacuccaccauuaagcuugcu

SEQ ID NO: 90 - RNA, 23 nt, Tomato chlorotic spot virus
ccaaucuugauuucuuugaacuu

SEQ ID NO: 91 - RNA, 100 nt, Tomato chlorotic spot virus
auguugacauuuuucggcagcaagaaacccucugagucugaaaagacagaugaaggucccuugguuucauugaccaaacgcaaugggaauguugaagucu

SEQ ID NO: 92 - RNA, 23 nt, Tomato chlorotic spot virus
uguugcacuguuauacccuuaaa

SEQ ID NO: 93 - RNA, 23 nt, Tomato chlorotic spot virus
gauccaauaugcuuuguuuuuua

SEQ ID NO: 94 - RNA, 23 nt, Tomato chlorotic spot virus
gguuucaaggauugugauaugga

SEQ ID NO: 95 - RNA, 23 nt, Tomato chlorotic spot virus
accuaugagucagacucuaucac

SEQ ID NO: 96 - RNA, 23 nt, Tomato chlorotic spot virus
uacaaaccaucauauuuccaacu

SEQ ID NO: 97 - RNA, 23 nt, Tomato chlorotic spot virus
gcaacuaagaucauuaagauaug

SEQ ID NO: 98 - RNA, 23 nt, Tomato chlorotic spot virus
cucauuugcaagugucauguacu

SEQ ID NO: 99 - RNA, 100 nt, Groundnut ringspot and Tomato chlorotic spot virus reassortant
guucauaucccaguguuugaugauauugauuucagcaucaauaucaaugauucuuuuuuggcaauuucuguuuguuccaacacaguuaacacuaauggag

SEQ ID NO: 100 - RNA, 23 nt, Groundnut ringspot and Tomato chlorotic spot virus reassortant
guauaguuucaaaccaaauuuca

SEQ ID NO: 101 - RNA, 23 nt, Groundnut ringspot and Tomato chlorotic spot virus reassortant
gaaacagcuuuaaauguuacugu

SEQ ID NO: 102 - RNA, 23 nt, Groundnut ringspot and Tomato chlorotic spot virus reassortant
aacagcccugaacuaaccccagc

SEQ ID NO: 103 - RNA, 23 nt, Groundnut ringspot and Tomato chlorotic spot virus reassortant
aaugugcagacuaugucuuauuu

SEQ ID NO: 104 - RNA, 150 nt, Barley yellow dwarf virus
augaauucaguaggucguagaggaccuagacgcgcaaaucaaaauggcacaagaaggaggcgccguagaacaguucggccagugguugugguccaacccaaucgagcaggacccagacgacgaaauggucgacgcaagggaagaggaggg

SEQ ID NO: 105 - RNA, 150 nt, Barley yellow dwarf virus
accuuccggucagaggcaauuaaugggaaggaauuccaggaaucaacgauagaccaauuuuggaugcucuacaaggccaauggaacuaccacugacacggcaggacaauuuaucauuacgaugagugucaguuugaugacggccaaauag

SEQ ID NO: 106 - RNA, 25 nt, Barley yellow dwarf virus
gcaaauuuuguauuuagaccaacag

SEQ ID NO: 107 - RNA, 140 nt, Barley yellow dwarf virus
agggaagaggaggggcaaauuuuguauuuagaccaacaggcgggacugagguauucguauucucaguugacaaccuuaaagccaacuccuccggggcaaucaaauucggccccagucuaucgcaaugcccagcgcuuuca

SEQ ID NO: 108 - RNA, 25 nt, Barley yellow dwarf virus
cguccgccaauacggcaggcgcuau

SEQ ID NO: 109 - RNA, 21 nt, Barley yellow dwarf virus
aucguuacaagaucacaagua

SEQ ID NO: 110 - RNA, 150 nt, Barley yellow dwarf virus
auggcacaagaaggaggcgccguagaacaguucggccagugguugugguccaacccaaucgagcaggacccagacgacgaaauggucgacgcaagggaagaggaggggcaaauccuguauuuagaccaacaggcgggacugagguauucg

SEQ ID NO: 111 - RNA, 22 nt, Barley yellow dwarf virus
cugagguauucguauucucagu

SEQ ID NO: 112 - RNA, 25 nt, Barley yellow dwarf virus
cagcggagauuggagggacccaguu

SEQ ID NO: 113 - RNA, 150 nt, Barley yellow dwarf virus
gacccagacgacgaaaugcuagacgcgcaagaggaagaaggccaaauucuguauuuggaccaaaacgcggggcugagguauucguauucucagucgacaaccuuaaggccaacuccuccgggauccucaaauucggucccgaucuaucgc

SEQ ID NO: 114 - RNA, 25 nt, Barley yellow dwarf virus
agaacacacgcguccgccacuacgu

SEQ ID NO: 115 - RNA, 25 nt, Barley yellow dwarf virus
gucacacgcguccgccacuacggca

SEQ ID NO: 116 - RNA, 150 nt, Barley yellow dwarf virus
auggaggaucuucacguuaucgccguuuguauucuugcuuugacugugcucucugggguaggcgcuguuuugaguugcugccguuggugcugcagcaauccuuuuccucccucccucucuucuguucaagcaaaagacucucgaucugug

SEQ ID NO: 117 - RNA, 25 nt, Barley yellow dwarf virus
gagagacaaucaaaaauaucgaggg

SEQ ID NO: 118 - RNA, 21 nt, Barley yellow dwarf virus
gagggagcuucggcucaguga

SEQ ID NO: 119 - RNA, 25 nt, Barley yellow dwarf virus
guuaggcgcggugaucggcugcuga

SEQ ID NO: 120 - RNA, 150 nt, Cucumber mosaic virus
auggacaaaucugaaucaaccagugccggucguaaucgucgacgucguccgcgucgugguucccgcuccgcucccuccuccgcggaugcuaacuuuagagucuugucgcagcagcuuucgcgacuuaauaagacguuagcagcuggucgu

SEQ ID NO: 121 - RNA, 25 nt, Cucumber mosaic virus
guuccugccuccucggacuuauccg

SEQ ID NO: 122 - RNA, 25 nt, Cucumber mosaic virus
uugccgcuaucucugcuauguuugc

SEQ ID NO: 123 - RNA, 25 nt, Cucumber mosaic virus
guccucguguauucaaaagacgaug

SEQ ID NO: 124 - RNA, 150 nt, Cucumber mosaic virus
auggacaaaucuggaucucccaaugcuaguagaaccucccggcgucgucgcccgcguagagguucucgguccgcuucuggugcggaugcaggguugcgugcuuugacucagcagaugcugagacucaauaaaacccucgccauuggucgu

SEQ ID NO: 125 - RNA, 150 nt, Cucumber mosaic virus
auggaauugaacgaaggcgcaaugacaaacgucgaacuccagcuggcucgcaugauggaggugaggagacaaagacgaaagucucacaagaagaaucgacgggaacgaggucacaaaagucccagcgagagagcgcguucaaaucucagg

SEQ ID NO: 126 - RNA, 25 nt, Cucumber mosaic virus
cugaggcuccucgguuuacguuacc

SEQ ID NO: 127 - RNA, 25 nt, Cucumber mosaic virus
uaaugaaugggcggaaggugcguuu

SEQ ID NO: 128 - RNA, 25 nt, Cucumber mosaic virus
guucggaacugauagagauguacca

SEQ ID NO: 129 - RNA, 22 nt, Cucumber mosaic virus
auuuugacgacacagauugguu

SEQ ID NO: 130 - RNA, 25 nt, Cucumber mosaic virus
ggcuuuccaagguaccaguaggacu

SEQ ID NO: 131 - RNA, 21 nt, Cucumber mosaic virus
aacucaacaguccucagcggc

SEQ ID NO: 132 - RNA, 21 nt, Cucumber mosaic virus
uucgaaagauauuauuuagcc

SEQ ID NO: 133 - RNA, 21 nt, Cucumber mosaic virus
gaagccauuaagaaaauggcu

SEQ ID NO: 134 - RNA, 21 nt, Cucumber mosaic virus
cugagugugaccuaggccggc

SEQ ID NO: 135 - RNA, 21 nt, Cucumber mosaic virus
aucauuggaugcgcgcugaua

SEQ ID NO: 136 - RNA, 21 nt, Cucumber mosaic virus
auuucaguccggccccucguu

SEQ ID NO: 137 - RNA, 21 nt, Cucumber mosaic virus
ugcuuccuucuuuaagucugg

SEQ ID NO: 138 - RNA, 21 nt, Cucumber mosaic virus
gauguuggugaauuaugcuca

SEQ ID NO: 139 - RNA, 150 nt, Cucumber mosaic virus
ucgcaauauuuguuaacgaaugcgaagauuaaucaaaaugcgcgcagugaguccgaggauuuaaauguugagagcccucccgccgcaaucgggaguucguccgcgucccgcuccgaagccuucagaccgcaggugguuaacggucuuuag

SEQ ID NO: 140 - RNA, 150 nt, Cucumber mosaic virus
uacaugagugucccucaaguguuaugugcuguuacucgaacaguuuccauugaugcugaagggucuuugagaauuuacuuagcugaucuaggcgacaaggaguuaucucccauagaugggcaaugcguuucguuacauaaccaugaucuu

SEQ ID NO: 141 - RNA, 25 nt, Cucumber mosaic virus
ccacagcuagcguauguaguaauug

SEQ ID NO: 142 - RNA, 25 nt, Cucumber mosaic virus
cauaucgcagcugggaagacucuag

SEQ ID NO: 143 - RNA, 25 nt, Cucumber mosaic virus
uaagcgguguuuugcugucguuauc

SEQ ID NO: 144 - RNA, 25 nt, Cucumber mosaic virus
gucagcuguugcucgccuguugaag

SEQ ID NO: 145 - RNA, 21 nt, Cucumber mosaic virus
gucuuuccaaccgacguauga

SEQ ID NO: 146 - RNA, 150 nt, Pepino mosaic virus
auggaaaaccaaccuacagcuucuaacccaucaaauguaccaccaacugcugcucaagcuggugcccagagcccagccgacuucucaaauccuaauacagcuccuucccuaagugauuugaagaagaucaaauacgugucaacugucacu

SEQ ID NO: 147 - RNA, 25 nt, Pepino mosaic virus
caauugaugcaccuccugaacuuua

SEQ ID NO: 148 - RNA, 25 nt, Pepino mosaic virus
cacguggucacaugggcggcgccaa

SEQ ID NO: 149 - RNA, 26 nt, Pepino mosaic virus
guaacuacaucaccacccuuggugaa

SEQ ID NO: 150 - RNA, 21 nt, Pepino mosaic virus
cugaugugcaaaguucaaaau

SEQ ID NO: 151 - RNA, 21 nt, Pepino mosaic virus
acugggcaaaauugggcuauc

SEQ ID NO: 152 - RNA, 21 nt, Pepino mosaic virus
agucacaaauccagcuagucu

SEQ ID NO: 153 - RNA, 150 nt, Pepino mosaic virus
ggcaagaucuucacugccauggguuuagcagccaaugagaccggaccugccaugugggaccucgcucgugcuuaugcugaugugcaaaguucaaaaucugcacaacuuauaggugccacaccauccaacccugcuuugucuagacgugca

SEQ ID NO: 154 - RNA, 150 nt, Pepino mosaic virus
auggaaagaucaacucugauuaauuuacuucaauugcaccacuucgagccaaaacucaguguugaaggaaccauaguugugcacggaauugcaggcacugggaaaaccacuuuacuuaggacuuuauuuucugcuuacccuagcuuaguu

SEQ ID NO: 155 - RNA, 150 nt, Pepino mosaic virus
cuagcaggacuugaguuugcagaaacaacuuucuacugcacaacauuggccgcagcuguugcugaaaauccugcuaagacuuucaucucucugacuagacacacccacaaacucaccauuggggaacuaaaugccaggucuaacuccuag

SEQ ID NO: 156 - RNA, 25 nt, Pepino mosaic virus
gguucaccuaggccuugcuauuuag

SEQ ID NO: 157 - RNA, 21 nt, Pepino mosaic virus
ucacaaguuugcuuaucuugc

SEQ ID NO: 158 - RNA, 21 nt, Pepino mosaic virus
aaauuggcuaucuuuggugac

SEQ ID NO: 159 - RNA, 21 nt, Pepino mosaic virus
cuuagagucccacauuacacu

SEQ ID NO: 160 - RNA, 21 nt, Pepino mosaic virus
ugaaguugaccccacugagca

SEQ ID NO: 161 - RNA, 21 nt, Pepino mosaic virus
gaccuuaauauagucucaguu

SEQ ID NO: 162 - RNA, 21 nt, Pepino mosaic virus
cuuuguuucugaccaaguggu

SEQ ID NO: 163 - RNA, 150 nt, Pepino mosaic virus
augccaggucuaacuccuagagcugaccucacugacacauacaaaaucauugccauugcuuucuuguugucagcuugcauuuacuuccaaaauagccacuaccaaccuguugcuggagacaacuugcaccguuugccuuuugguggccaa

SEQ ID NO: 164 - RNA, 21 nt, Pepino mosaic virus
ucaagacggcaccaaaaagau

SEQ ID NO: 165 - RNA, 21 nt, Pepino mosaic virus
auauccuauuuuccacaacag

SEQ ID NO: 166 - RNA, 21 nt, Pepino mosaic virus
aaccauugucaggccaucauu

SEQ ID NO: 167 - RNA, 21 nt, Pepino mosaic virus
gucauacuuucauucuggaaa

SEQ ID NO: 168 - RNA, 21 nt, Pepino mosaic virus
aaauuaaauguccucauacuu

SEQ ID NO: 169 - RNA, 150 nt, Pepino mosaic virus
aauguccucauacuuaucuucauucucacguuggguauuguccucaccaauaaauuuaguuuuagcuuuagucguacuacucaccagcauucuugcuauaacacacauucagcaaccaacaauacacaaccauugucaggccaucauuga

SEQ ID NO: 170 - RNA, 150 nt, Pepino mosaic virus
auguccucauacuuaucuucauucucacguuggguauuguccucaccaauaaauuuaguuuuagcuuuagucguacuacucaccagcauucuugcuauaacacacauucagcaaccaacaauacacaaccauugucaggucaucauugac

SEQ ID NO: 171 - RNA, 21 nt, Pepino mosaic virus
aauagucauaacaaauuguga

SEQ ID NO: 172 - RNA, 21 nt, Pepino mosaic virus
aguuuuccuaaauuugaaaau

SEQ ID NO: 173 - RNA, 21 nt, Pepino mosaic virus
accauguacgcgauagacgca

SEQ ID NO: 174 - RNA, 21 nt, Pepino mosaic virus
ugagaaagaacuagcugcuca

SEQ ID NO: 175 - RNA, 150 nt, Pepino mosaic virus
augccugacacaacaccuguugcugccacuucaagugcaccacccacagccaaagaugcuggugccaaagcuccuucugacuucucaaaucccaauacagcuccuagucucagugauuugaagaaagucaaguaugucuccaccgugacc

SEQ ID NO: 176 - RNA, 21 nt, Pepino mosaic virus
cuaucacgccgagcccuugcu

SEQ ID NO: 177 - RNA, 150 nt, Pepino mosaic virus
uccguggccacaccagcugaaauugaagcccuaggcaaaaucuucaccgcuaugggccuugccgccaaugagacugguccggccaugugggaucuagcucgugcauaugcugaugugcagaguucuaaaucggcacagcugauuggagcu

SEQ ID NO: 178 - RNA, 150 nt, Cotton leaf curl virus
augucgaagcgagcugcagauaucgucauuucuacgcccgcgucgaaaguacgccggcgucugaacuucggcagcccauacaccaaccguguugcugcccccauuguccgcgucacaaaacaacaggcauggacaaacaggccuaugaac

SEQ ID NO: 179 - RNA, 21 nt, Cotton leaf curl virus
aggaagcccagaauguaucgg

SEQ ID NO: 180 - RNA, 21 nt, Cotton leaf curl virus
uguacagaaguccggauguuc

SEQ ID NO: 181 - RNA, 21 nt, Cotton leaf curl virus
aggguugugaaggcccaugua

SEQ ID NO: 182 - RNA, 21 nt, Cotton leaf curl virus
uacagucuuuugaguccagac

SEQ ID NO: 183 - RNA, 25 nt, Cotton leaf curl virus
agaaucacaccgagaaugcauugau

SEQ ID NO: 184 - RNA, 21 nt, Cotton leaf curl virus
cagguguugaggaaauggcau

SEQ ID NO: 185 - RNA, 21 nt, Cotton leaf curl virus
caacccuguuuaugcuacguu

SEQ ID NO: 186 - RNA, 25 nt, Cotton leaf curl virus
guaagguaauguguauuucugaugu

SEQ ID NO: 187 - RNA, 21 nt, Cotton leaf curl virus
gugucgguuugacccaucgua

SEQ ID NO: 188 - RNA, 150 nt, Cotton leaf curl virus
uuaauugaaauuacaccgagauuguucagauauuugaggacuugguuuuugaauacccuuaagaaaagaccagucugaggcuguaaggucguccagauucggaagguuagaaaacacuugugcagucccagagcuuuccgcguguuguag

SEQ ID NO: 189 - RNA, 21 nt, Cotton leaf curl virus
aucggucuaucaagugugaug

SEQ ID NO: 190 - RNA, 21 nt, Cotton leaf curl virus
guacuuugauugguaccugag

SEQ ID NO: 191 - RNA, 21 nt, Cotton leaf curl virus
auugacagauaauaagaacac

SEQ ID NO: 192 - RNA, 21 nt, Cotton leaf curl virus
aagaucuacucuccuccuccu

SEQ ID NO: 193 - RNA, 21 nt, Cotton leaf curl virus
cgccauucccugcuugagcug

SEQ ID NO: 194 - RNA, 21 nt, Cotton leaf curl virus
aucuugaaauaaaggggauuu

SEQ ID NO: 195 - RNA, 21 nt, Cotton leaf curl virus
ccccugugcgugaauccaugg

SEQ ID NO: 196 - RNA, 150 nt, Cotton leaf curl virus
augugggauccacuauuaaacgaauucccugauacgguucacggguuucgguguaugcuuucugugaaauauuugcaacuuuugucgcaggauuauucaccggauacgcuuggguacgaguuaauacgggauuuaauuuguauuuuacgc

SEQ ID NO: 197 - RNA, 21 nt, Cotton leaf curl virus
auguacagaaguccagauguu

SEQ ID NO: 198 - RNA, 21 nt, Cotton leaf curl virus
ugcccccauuguccgcgucac

SEQ ID NO: 199 - RNA, 21 nt, Cotton leaf curl virus
guacgccggcgucugaacuuc

SEQ ID NO: 200 - RNA, 21 nt, Cotton leaf curl virus
caacaggcauggacaaacagg

SEQ ID NO: 201 - RNA, 21 nt, Cotton leaf curl virus
uaugaacaggaagcccaggau

SEQ ID NO: 202 - RNA, 21 nt, Cotton leaf curl virus
auuaugucgaagcgagcugcc

SEQ ID NO: 203 - RNA, 21 nt, Cotton leaf curl virus
ucgucauuucuacgcccgcgu

SEQ ID NO: 204 - RNA, 25 nt, Cotton leaf curl virus
ucggcagcccauacaccagccgugc

SEQ ID NO: 205 - RNA, 150 nt, Tomato yellow leaf curl virus
augucgaagcgacuaggcgauauaaucauuuccacgcccgucucgaagguucgccgaaggcugaacuucgacagcccauacagcagccgugcugcuguccccauuguccaaggcacaaacaagcgacgaucauggacguacaggcccaug

SEQ ID NO: 206 - RNA, 21 nt, Tomato yellow leaf curl virus
uuuuaauauguucgauaauga

SEQ ID NO: 207 - RNA, 21 nt, Tomato yellow leaf curl virus
uaacuuauaaucaucaggagg

SEQ ID NO: 208 - RNA, 21 nt, Tomato yellow leaf curl virus
uuucuaugauucaauaucaaa

SEQ ID NO: 209 - RNA, 21 nt, Tomato yellow leaf curl virus
aaaacgccuuguuauuguaua

SEQ ID NO: 210 - RNA, 21 nt, Tomato yellow leaf curl virus
uuugcgugauagguuucaagu

SEQ ID NO: 211 - RNA, 21 nt, Tomato yellow leaf curl virus
guggaugugaaggcccaugua

SEQ ID NO: 212 - RNA, 21 nt, Tomato yellow leaf curl virus
uaagagguucuguguuaaauc

SEQ ID NO: 213 - RNA, 150 nt, Tomato yellow leaf curl virus
aaguccagucuuaugagcaacgggaugauauuaagcacacugguauuguucguuguguuagugauguuacucguggaucuggaauuacucauagaguggguaagagguucuguguuaaaucgauauauuuuuuagguaaagucuggaugg

SEQ ID NO: 214 - RNA, 150 nt, Tomato yellow leaf curl virus
ugaugagaaaauuucaugcaacaguuauuggugggcccucuggaaugaaggaacaggcauuaguuaagagauuuuuuaaaauuaacagucauguaacuuauaaucaucaggaggcagccaaguacgagaaccauacugaaaacgccuugu

SEQ ID NO: 215 - RNA, 21 nt, Tomato yellow leaf curl virus
acgcaugccucuaauccagug

SEQ ID NO: 216 - RNA, 150 nt, Tomato yellow leaf curl virus
augugggacccacuucuaaaugaauuuccugaaucuguucacggauuucguuguauguuagcuauuaaauauuugcaguccguugaggaaacuuacgagcccaauacauugggccacgauuuaauuagggaucuuauaucuguuguaagg

SEQ ID NO: 217 - RNA, 21 nt, Tomato yellow leaf curl virus
cccgugacuaugucgaagcga

SEQ ID NO: 218 - RNA, 21 nt, Tomato yellow leaf curl virus
caggcgauauaaucauuucca

SEQ ID NO: 219 - RNA, 21 nt, Tomato yellow leaf curl virus
cccgucucgaagguucgccga

SEQ ID NO: 220 - RNA, 21 nt, Tomato yellow leaf curl virus
ugaacuucgacagcccauaca

SEQ ID NO: 221 - RNA, 21 nt, Tomato yellow leaf curl virus
auuguccaaggcacaaacaag

SEQ ID NO: 222 - RNA, 21 nt, Tomato yellow leaf curl virus
aucauggacguacaggcccau

SEQ ID NO: 223 - RNA, 21 nt, Tomato yellow leaf curl virus
uacagaauguaucgaaguccu

SEQ ID NO: 224 - RNA, 150 nt, Tomato yellow leaf curl virus
augcaaccuucgucacccucuacgagccacuguucgcaaguaucaaucaagguccaacacaagauagccaagaagaaaccaauaaggcguaagcguguagaccuagacuguggcugcucauacuaccuccaccucaacugcaacaaucau

SEQ ID NO: 225 - RNA, 21 nt, Tomato yellow leaf curl virus
cgcacaggggaacucaucacu

SEQ ID NO: 226 - RNA, 21 nt, Tomato yellow leaf curl virus
uccucaggcagagaauggcgu

SEQ ID NO: 227 - RNA, 21 nt, Tomato yellow leaf curl virus
ugggagauaaacaauccccuc

SEQ ID NO: 228 - RNA, 21 nt, Tomato yellow leaf curl virus
uucaagauaacagaacacagc

SEQ ID NO: 229 - RNA, 21 nt, Tomato yellow leaf curl virus
uggucauuucuuaagaguauu

SEQ ID NO: 230 - RNA, 21 nt, Tomato yellow leaf curl virus
ucagauaagauucaaccacaa

SEQ ID NO: 231 - RNA, 21 nt, Tomato yellow leaf curl virus
caggaagguaauggggauuca

SEQ ID NO: 232 - RNA, 150 nt, Tomato yellow leaf curl virus
gccauuucuaaugaaccacgacaucauuuccauucagauaagauucaaccacaacaucaggaagguaauggggauucacaaauguuuucucaacuuccgaauuuggacgacauuacagccucagacuggucauuucuuaagaguauuuag

SEQ ID NO: 233 - RNA, 150 nt, Wheat streak mosaic virus
ucgagugaggccgcaaccgacgcugucuuggcggcagcaaaugcaggaacugguagugcaucgaguaguggaagcacucagucaagucagagcgcaaguacugcuagcggaucagggaguucaccaucaggaucagguucuggagcagcg

SEQ ID NO: 234 - RNA, 21 nt, Wheat streak mosaic virus
guuucaaugguguaaacgcuc

SEQ ID NO: 235 - RNA, 21 nt, Wheat streak mosaic virus
uuugugcaagugcagaacagc

SEQ ID NO: 236 - RNA, 21 nt, Wheat streak mosaic virus
ugaucgaggagaguguucgaa

SEQ ID NO: 237 - RNA, 21 nt, Wheat streak mosaic virus
gugcuaagcaucaacaauauu

SEQ ID NO: 238 - RNA, 21 nt, Wheat streak mosaic virus
aggcacgacugagugcgggua

SEQ ID NO: 239 - RNA, 21 nt, Wheat streak mosaic virus
agaugaugugagggagaacac

SEQ ID NO: 240 - RNA, 21 nt, Wheat streak mosaic virus
uugcaguaaacaggcuacgcg

SEQ ID NO: 241 - RNA, 150 nt, Wheat streak mosaic virus
guggaucagguucuggaucagcacaaacacaaucuaauaacguaucugucauggcuggccucgacacgggaggagcuaagacagaucaaggaucaggaucaaaagggacgggugguucauucacaucgaaucccgugcgaacuggaggcc

SEQ ID NO: 242 - RNA, 150 nt, Wheat streak mosaic virus
cacaacugaacaacucaacaccuggauaaaagaggcaucugaagggcuugacgugacagaggauguuuucauaaacaccuuacuuccaggaugggucuaccacugcauaaucaacacaacgagcccagagaacagagcacuaggaacuug

SEQ ID NO: 243 - RNA, 21 nt, Wheat streak mosaic virus
augaggacaacucacgucaug

SEQ ID NO: 244 - RNA, 21 nt, Wheat streak mosaic virus
gcaacggaugugcaagaucag

SEQ ID NO: 245 - RNA, 21 nt, Wheat streak mosaic virus
uuaguguuuccagcaccaaag

SEQ ID NO: 246 - RNA, 21 nt, Wheat streak mosaic virus
ugaaaugauaaauaacaugau

SEQ ID NO: 247 - RNA, 21 nt, Wheat streak mosaic virus
ggacaaugagcagcaacucga

SEQ ID NO: 248 - RNA, 21 nt, Wheat streak mosaic virus
aaaaccuaucauaccaagggg

SEQ ID NO: 249 - RNA, 21 nt, Wheat streak mosaic virus
ugugauuucaucaugcgcggu

SEQ ID NO: 250 - RNA, 25 nt, Wheat streak mosaic virus
agccauuaugcgccacuuuggugag

SEQ ID NO: 251 - RNA, 150 nt, Wheat streak mosaic virus
ggcaaagcagcacgcaacaggagugcaaaacgacaaucagcaagagaccaaaagauggagcgugguaacgaauacacauacuacgaugcuggugacaccuuguauaauggaguucaagagaauaugaaucaugcaccagacuggaccgau

SEQ ID NO: 252 - RNA, 21 nt, Wheat streak mosaic virus
ugaaaaagaagugucguacga

SEQ ID NO: 253 - RNA, 21 nt, Wheat streak mosaic virus
aaagcgaguuuuggccaucuu

SEQ ID NO: 254 - RNA, 21 nt, Wheat streak mosaic virus
cgauccucacaauacuggcac

SEQ ID NO: 255 - RNA, 21 nt, Wheat streak mosaic virus
cagucgaauucaaggacauag

SEQ ID NO: 256 - RNA, 150 nt, Wheat streak mosaic virus
ucaggaaaggagacaagguucgugaaguugcauuggcaccucacaagccaaaccaagucaacaagcgugggcuaccugucggacaugcugaucacagaggagaguggagacaaacacagccuucauuugaaaaagaagugucguacgaga

SEQ ID NO: 257 - RNA, 25 nt, Wheat streak mosaic virus
uuugcagaaagccuucacagaaaug

SEQ ID NO: 258 - RNA, 21 nt, Wheat streak mosaic virus
caaacuucucagugcaccagg

SEQ ID NO: 259 - RNA, 150 nt, Wheat streak mosaic virus
ggugcacguucacuugaucauauccaucagaaucaagucauccucguugaagacaaucagcaguuaaaugggcuaauaguugggaacauacucuuggcgccauaucauuucacacgagguaugaggaacagagaggagaaggagacacgc

SEQ ID NO: 260 - RNA, 21 nt, Wheat streak mosaic virus
caauaagcgacuugguuaguu

SEQ ID NO: 261 - RNA, 21 nt, Wheat streak mosaic virus
accuaaaaguugugggauucc

SEQ ID NO: 262 - RNA, 150 nt, Wheat streak mosaic virus
auaaccuuggagggaaaggugagaauuauuucacaccgauaacuauugaggucauggauuucuuagcugaaaagucugugacaccgcuugugccauggaaguucucagacgagcaaguugacuuaugugguuuaauugcggccaauggag

SEQ ID NO: 263 - RNA, 21 nt, Wheat streak mosaic virus
cacaguuuggaacguacaauc

SEQ ID NO: 264 - RNA, 150 nt, Wheat streak mosaic virus
aagcaugucacaaaauuuacaaugauggaucugguagcauuaaccuugccuccaacauuucaagcaagacggaaacucaaauguuucagaccaccaagggaaggagagcgagcaauguuggugaccaugcaguacgagaaagcaggaugg

SEQ ID NO: 265 - RNA, 21 nt, Wheat streak mosaic virus
uuucaacaggaccaggugacu

SEQ ID NO: 266 - RNA, 25 nt, Wheat streak mosaic virus
aaucagcagaaacaacaaucacacc

SEQ ID NO: 267 - RNA, 21 nt, Wheat streak mosaic virus
acaaauacccauucaccaaaa

SEQ ID NO: 268 - RNA, 21 nt, Wheat streak mosaic virus
ccauaguagcaguagcagacc

SEQ ID NO: 269 - DNA, 657 bp, Cucumber mosaic virus
atggacaaatctgaatcaaccagtgctggtcgtaaccgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtagacctgggtacacgttcacatctattaccctaaagccaccaaaaatagaccgtgggtcttattacggtaaaaggttgttactacctgattcagtcacggaatatgataagaagcttgtttcgcgcattcaaattcgagttaatcctttgccgaaatttgattctaccgtgtgggtgacagtccgtaaagttcctgcctcctcggacttatccgttgccgccatctctgctatgttcgcggacggagcctcaccggtactggtttatcagtatgccgcatctggagtccaagccaacaacaaactgttgtatgatctttcggcgatgcgcgctgatataggtgacatgagaaagtacgccgtcctcgtgtattcaaaagacgatgcgctcgagacggacgagctagtacttcatgttgacatcgagcaccaacgcattcccacatctggagtgctcccagtctga

SEQ ID NO: 270 - DNA, 657 bp, Cucumber mosaic virus
atggacaaatctgaatcaaccagtgctggtcgtaaccgtcgacgtcgtccgcgtcgtggttcccgctccgcttcctcctcctcggatgctaactttagagtcttgtcgcagcagctttcgcgacttaataagacgttagcagctggtcgtccaactattaaccacccaacctttgtagggagtgaacgctgtaaacctggatacacgttcacatctattaccctaaagccaccaaaaatagaccgcgggtcttattacggtaaaaggttgttattacctgattcagtcacggaatatgataagaaacttgtttcgcgcattcaaattcgagttaatcctttgccgaaatttgattctaccgtgtgggtgacagtccgtaaagttcctgcctcctcggacttatccgttgccgccatctctgctatgtttgcggacggagcctcaccggtactggtttatcagtacgctgcatctggagtccaagctaacaacaaattgttgtatgatctttcggcgatgcgcgctgatataggcgacatgagaaagtacgccgtcctcgtgtattcaaaagacgatgcgctcgagacggacgagctggtacttcatgttgacgtcgagcaccaacgcattcccacgtctggggtgctcccagtctaa

SEQ ID NO: 271 - DNA, 657 bp, Cucumber mosaic virus; identical to SEQ ID NO: 269

SEQ ID NO: 272 - DNA, 657 bp, Cucumber mosaic virus
atggacaaatctgaatcaaccagtgctggtcgtaaccatcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtagacctgggtacacgttcacatctattaccctaaagccaccaaaaatagaccgtgagtcttattacggtaaaaggttgttactacctgattcagtcacggaatatgataagaagcttgtttcgcgcattcaaattcgagttaatcctttgccgaaatttgattctaccgtgtgggtgacagtccgtaaagttcctgcctcctcggacttatccgttgccgccatctctgctatgttcgcggacggagcctcaccggtactggtttatcagtatgccgcatctggagtccaagccaacaacaaactgttgtttgatctttcggcgatgcgcgctgatataggtgacatgagaaagtacgccgtcctcgtgtattcaaaagacgatgcgctcgagacggacgagctagtacttcatgttgacatcgagcaccaacgcattcccacatctggagtgctcccagtctga

SEQ ID NO: 273 - DNA, 657 bp, Cucumber mosaic virus
atggacaaatctgaatcaaccagtgctggtcgtaaccgtcgacgtcgtttgcgtcgtggttcccgctccgccccctcctcctcggatgctaactttagagtcttgtcgcagcaactttcgcgacttaataagacgttgtcagctggtcgtccaactattaaccacccaacctttgtaggaactgaacgttgtaaacctggatacacgttcacatctattacctcaaagccaccaaaaatagaccgcgggtcttattatggtaaaaggttgttattacctgattcagtcacagaatatgataagaaacttgtttcgcgcattcaaattcgagttaatcctttgccgaaatttgattctaccgtgtgggtgacagtccgtaaagttcctgcctcctcggacttatccgttcccgccatctctgctatgtttccggacggaccctcaccggtactggtttatcagtatgctgcatctggagtccaagctaacaacaaactgttgtatgatctttcggcgatgcgcgctgatataggcgacatgagaaagtacgccgtcctcgtgtacgcaaaagacgatgcactcgcgacggacgagctggtacttcatgttgacgtcgagcaccaacgcattcccacgtctggggtcctcccagtatga

SEQ ID NO: 274 - DNA, 840 bp, Cucumber mosaic virus
atggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgataaggctatttcagtccggcccctcgttcccgaagtaacccacggtcgtattgcttctttctttaagtctggatatgatgttggtgaattatgctcaaaaggatacatgagtgtccctcaagtgttatgtgctgttactcgaacagtttccactgatgctgaagggtctttgagaatttacttagctgatctaggcgacaaggagttatctcccatagatgggcaatgcgtttcgttacataaccatgatcttcccgctttggtgtctttccaaccgacgtatgattgtcctatggaaacagttgggaatcgtaagcggtgttttgctgtcgttatcgaaagacatggttacattgggtttaccggtaccacagctagcgtgtgtagtaattggcaagcaaggttttcatctaagaataacaactacactcatatcgcagctgggaagactctagtactgcctttcaacagattagctgagcaaacaaaaccgtcagctgtcgctcgcctgttgaagtcgcaattgaacaacattgaatcttcgcaatatttgttaacgaatgcgaagtttaatcaaaatgcgcgcagtgagtccgaggatttaaatgttgagagccctcccgccgcaatcgggagttcttccgcgtcccgctccgaagccttcagaccgcaggtggttaacggtctttag

SEQ ID NO: 275 - DNA, 657 bp, Cucumber mosaic virus; identical to SEQ ID NO: 269

SEQ ID NO: 276 - DNA, 657 bp, Cucumber mosaic virus
atggacaaatctgaatcaaccagtgctggtcgtaaccgtcgacgtcgcttgcgtcgtggttcccgctccgccccctcctccgcggatgccaactttagagtcttgtcgcagcagctttcgcgacttaacaagacgttgtcagccggtcgtccaactattaaccacccaacctttgtagggagtgagcgttgtaaacctgggtacacgttcacatctattaccctaaagccaccaaaaatagaccgtgggtcttattatggtaaaaggttgttattacctgattcagtcacggaatatgataagaaacttgtttcgcgcattcaaattcgagttaatcctttgccgaaatttgattctaccgtgtgggtgacagtccgtaaagttcctgcctcctcggacttatccgttgccgccatctctgctatgtttgcggacggagcctcaccggtactggtttatcagtatgctgcatctggagttcaagctaacaacaaactgttgtatgatctttcggcgatgcgcgctgatataggcgacatgagaaagtacgccgtcctcgtgtactcaaaagacgatacgctcgagacggacgagctggtacttcatgttgacgtcgagcaccaacgcattcccacgtctggggtgctcccagtctga

SEQ ID NO: 277 - DNA, 657 bp, Cucumber mosaic virus
atggacaaatctgaatcaaccagtgctggtcgtaaccgtcgacgtcgtccgcgtcgtggttcccgctccgctccctcctccgcggatgctaactttagagtcttgtcgcagcagctttcgcgacttaataagacgttagcagctggtcgtccaactattaaccacccaacctttgtagggagtgaacgctgtagacctgggtacacgttcacatctattaccctaaagccaccaaaaatagaccgtgggtcttattacggtaaaaggttgttattacctgattcagtcacggaatatgataagaagcttgtttcgcgcattcaaattcgagttaatcctttgccgaaatttgattctaccgtgtgggtgacagtccgtaaagtttctgcctcctcggacttatccgttgccgccatctctgctatgttcgcggacggagcctcaccggtactggtttatcagtatgccgcatctggagtccaagcaaacaacaaattgttgtatgatctttcggcgatgcgcgctgatataggtgacatgagaaagtacgccgtcctcgtgtattcaaaagacgatgcgctcgagacggacgagctagtacttcatgttgacatcgagcaccaacgtattcccacatctggagtgctcccagtctga

SEQ ID NO: 278 - DNA, 657 bp, Cucumber mosaic virus
  unsure (1)..(657): unsure at all n locations
atggacaaatctgaatcaaccagtgccggtcgtagccgtcgacgtcgtccgcgtcgtggttcccgctccgccctctcctcngcggatgctaactttagagtcttgtcgcaacagctttcgcgacttaataagacgttagcagctggtcgtccaactattaaccacccaacctttgtagggagtgaacgctgtaaacctgggtacacgttcacatctatcaccctaaagccaccaaaaatagaccgtgggtcttattatggtaaaaggttgttattacctgattcagtcacggaatatgataagaaacttgtttcgcgcatccaaattcgagttaatcctttgccgaaatttgattcaaccgtgtgggtgacagtccgtaaagttcctgcctcctcggacttatccgttgccgccatctctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatccggagtgctcccagtctga

SEQ ID NO: 279 - DNA, 657 bp, Cucumber mosaic virus
atggacaaatctggatcaaccagtgccggtcgtagccgtcgacgtcgtccgcgtcgtggttcccgctccgccccctcctccgcggatgctaactttagagtcttgtcgcagcatctttcgcgacttaataagacgttagcagctggtcgtccaactattaaccacccaacctttgtagggagtgaacgctgtaaacctgggtacacgttcacatctatcaccctaaagccaccaaaaatagaccgagggtcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcctcggacttatccgttgccgccatctctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatccggagtgctcccgacttag

SEQ ID NO: 280 - DNA, 657 bp, Cucumber mosaic virus
atggacaaatctgaatcaaccagtgccggtcgtagccgtcgacgtcgtccgcgtcgtggttcccgctccgccccctcctccgcggatgctaactttagagtcttgtcgcagcatctttcgcgacttaataagacgttagcagctggtcgtccaactattaaccacccaacctttgtagggagtgaacgctgtaaacctgggtacacgttcacatctatcaccctaaagccaccaaaaatagaccgagggtcttattatggtaaaaggttgttattacctgattcagtcacggaatatgataacaaacttgtttcgcgcattcaaattcgagttaatcctttgccgaaatttgattcaaccgtgtgggtgacagtccgtaaagttcctgcctcctcggacttatccgttgccgccatctctgctatgtttgcggacggagcctcaccggtactggtttatcagtacgctgcatctggagtccaaagctacaacaaactgttgtatgatctttcgccgatgcgcgctgatataagcgacatgagaaagtcccccgtcctcgtgtattcaaaagacgatgcactcgagacagacgagttagtacttcatgttgacgtcgagcaccaacgtattcccacatccggagtgctcccagtctga

SEQ ID NO: 281 - DNA, 657 bp, Cucumber mosaic virus
atggacaaatctgaatcaaccagtgctggtcgtaaccgtcgacgtcgtccgcgtcgtggttcccgctccgccccctcctccgcggatgctaactttagagtcttgtcgcagcaactttcgcgactaaacaagacgttagcagctggtcgtccaaccattaaccacccaacctttgtggggagtgaacgctgtaaacctgggtacacgttctcatctattaccctgaagccaccgaaaatagaccgtgggtcttattatggtaaaaggttgttattacctgattcagtcacggagttcgataagaaacttgtttcgcgcattcaaattcgagttaatcctttgccgaaatttgattctaccgtgtgggttacggtccgtaaagttcctgcctcctcggacctatccgtcgccgccatctctgctatgtttgcggacggagcctcaccggtactggttaatcagtatgctgcatccggcgtccaagccaacaacaagttgttgtatgatctttcagcgatgcgagctgatattggcgacatgagaaagtacgccgtcctcgtgtattcaaaagacgatgcgctcgagacggatgaactggtacttcatgtcgacattgagcaccaacgcattcccacgtctggagtgctcccagtgtga

SEQ ID NO: 282 - DNA, 657 bp, Cucumber mosaic virus
atggacaaatctgaatcaaccagtgctggtcgtaaccgtcggcgtcgtccgcgtcgtggttcccgctccgcttcctcctccgcggatgctaactttagagtcttgtcgcagcaactttcgcgacttaacaagacattagcaactggtcgtccaactattaaccacccaacctttgtgggtagtgagcgttgtaaacctggatacacgttcacctcgattaccctgaagccaccaaagatagacaaaggatcttactatggcaaaaggttgttacttcctgattcagtcacagagttcgataagaagcttgtttcgcgcattcaaattcgagttaatcctttgccgaaatttgattctaccgtgtgggtgacagtccgcaaagttcctgcctcatcggacttatccgttaccgccatctctgctatgttcgcggacggagcctcaccggtactggtttatcagtatgcagcatccggagtccaagccaacaataaattgttgtatgatctttcggcgatgcgcgctgatattggtgacatgagaaagtacgccgtgctcgtgtattcaaaagacgatgcgctcgagacggacgaattggtacttcatgtcgacattgagcaccaacgtattcccacatctggggtgctcccagtttga

SEQ ID NO: 283 - DNA, 840 bp, Cucumber mosaic virus
atggctttccaaggtaccagcaggactttaactcaacagtcctcagcggctacgtctgacgaacttcaaaagatattatttagccctgaagccattaagaaaatggctgctgagtgtgacctaggccggcatcactggatgcgcgctgataatgcaatttcagtccggcccctcgttcccgaagtaacccacggtcgtattgcttccttctttaagtctggatatgatgctggtgaattgtgctctaaaggatgtgtgagtgtccctcaagtgttgtgtgctgttactcgaacagtctctaccgatgctgaagggtctttgagaatttacctacctgatttaggcgataaggagctatctcccatagataagcaatgtgtcacattacataaccatcatcttcccgctttagtgtctttccaaccgacgtatgattgtcctatggagacagttgggaatcgaaagcggtgttttgctgtcgtcatcgaaagacatggttatattgggtataccggcaccacagctagcgtgtgtagtaattggcaagcacggttttcttctaagaataacaactacactcatatcgcagctgggaagactctagtactgccattcaacagattagctgagcaaacgaaaccgtcagccgtcgctcgcctgttgaagtcgcaattgaataacatgggatcttcgcaatacgttttgacggattcgaagattaatcaaaatgcgcgcagtgagtccgaggaattaaatgttgagagccctcccgccgcaatcgggagttctgtcgcgtcccgcttcgaatccttcagaccgcaggtggtcaacggtctttag

SEQ ID NO: 284 - DNA, 657 bp, Cucumber mosaic virus
atggacaaatctgaatcaaccagtgccggtcgtaatcgtcgacgtcgtccgcgtcgtggttcccgctccgctccctcctccgcggatgctaactttagagtcttgtcgcagcagctttcgcgacttaataagacgttagcagctggtcgtccaactattaaccacccaacctttgtgggtagtgaacgctgtaaacctgggtacacgttcacatctattaccctgaagccgccgaaaatagaccgcgggtcttattatggtaaaaggttgttgctacctgattcagtcacggagttcgataagaagcttgtttcgcgcattcaaattcgagttaatcctttgccgaaatttgattctaccgtgtgggtgacagtccgtaaagttcctgcctcctcggacttatccgttgccgctatctctgctatgtttgcggacggagcctcaccggtactggtttatcagtatgctgcatccggcgttcaagccaacaacaaattgttgtacgatctttcggcgatgcgcgctgatattggcgacatgagaaagtacgccgtcctcgtgtattcaaaagacgatgcgctcgagacggacgagttggtacttcatgttgacatcgagcaccaacgcattcccacatctggggtgctcccagtttga

SEQ ID NO: 285 - DNA, 657 bp, Cucumber mosaic virus
atggacaaatctgaatcaaccagtgccggtcgtaaccgtcgacgtcgtccgcgtcgcggttcccgctcagcttcctcctccgctgatgctaattttagagtcctgtcgcaacaactttcgcgacttaataagacgttagcagctggtcgtcctaccattaaccacccaacctttgtagggagtgagcgttgtaaacctgggtacacgttcacatctatcaccctgaaaccaccgaaaatagacaaagggtcttattatggtaaaaggttgttacttcctgattcagtcactgagttcgataagaagcttgtttcgcgcattcaaattcgagttaatcctttgccgaaatttgattctaccgtgtgggtgacagtccgtaaagttcctgcctcctcggatttatccgttaccgccatctctgctatgtttgcggacggagcctcaccggtactggtttatcagtacgccgcatccggagttcaagccaacaataaactattatacgatctctcggcgatgcgcgctgatattggtgatatgagaaagtacgccgtcctcgtgtattcaaaagacgatgcgctcgagactgacgagctagtacttcatgttgacatcgagcaccaacgtattcctacatctggggtgctcccagtttga

SEQ ID NO: 286 - DNA, 657 bp, Cucumber mosaic virus
atggacaaatctgaatcaaccagtgccggtcgtaatcgtcgacgtcgtccgcgtcgtggttcccgctccgctccctcctccgcggatgctaactttagagtcttgtcgcagcagctttcgcgacttaacaagacgttagcagctggtcgtccaactattaaccacccaacctttgtgggtagtgaacgctgtaaacctgggtacacgttcacatctattaccctgaagccgccgaaaatagaccgcgggtcttattatggtaaaaggttgttgctacctgattcagtcacggagttcgataagaagcttgtttcgcgcattcaaattcgagttaatcctttgccgaaatttgattctaccgtgtgggtgacagtccgtaaagtttctgcctcctcggacttatccgttgccgctatctctgctatgtttgcggacggagcctcaccggtactggtttatcagtatgctgcatccggcgttcaagccaacaacaaattgttgtacgatctttcggcgatgcgcgctgatattggcgacatgagaaagtacgccgtcctcgtgtattcaaaagacgatgcgctcgagacggacgagttggtacttcatgttgacatcgagcaccaacgcattcccacatctggggtgctcccagtttga

SEQ ID NO: 287 - DNA, 657 bp, Cucumber mosaic virus
atggacaaatctgaatcaaccagcgctggtcgtaaccgtcggcgtcgtccgcgtcgtggttcccgctcccgttcctcctccgcggatgctaactttagagtcctgttgcaacagctttcgcgactcaataagacgttagcagctggtcgtccaaccattaaccaccgaacctttgcgggtagtgaacgatgtaaatccgggtacgcgttcacatctatcaccctgaaaccaccgaaaatagatcgacggtcttattatggtaaagggttgttacttcctgattcggtcactgagttcgataagaagcttgtttcgcgcattcaaatccgagttaatcctttgccgaaattcgattctaccgtgtgggtgacagttcgtaaagttcctgccttctccaacttatccgttgccgccatctctgctatgttcgcggacggagcctcaccggtactggtctatcagtacgccgcatccggagtccaagccaacaacaagttgttgtacgatctttcggcgatgcgcgctgatattggcgccatgagaaagtacgccgttctcgtgtattcaaaagacgatgctctcgagacggatgagctggtacttcatgtcgacattgagcaccaacgcattcccacatcaggggtgctcccagtttga

SEQ ID NO: 288 - DNA, 657 bp, Cucumber mosaic virus
atggacaaatctgactcagccagcgccggtcgtaaccgtcgacgtcgtccgcgtcgtggttcccgctccgcttcctcctccgcggatgttaactttagagtcctgtcgcagcaactttcgcggcttaataagacgttagcagctggtcgtcctactattaaccacccaacctttgtagggagtgagcgttgtaaacctggatacacgttcacgtctattaccctaaggccaccaaaaatagaccgcgggtcttattatggtaaaaggttgtttttacctgagtcagtcacggaattcgataagaaacttgtttcgcgcattcaaattcgagttaatcctttgccgaaatttgattctaccgtgtgggtgacagtccgtaaagttcctgcctcttcggacttgtccgtcgccgccatttctactatgtttgcggacggagcctcaccggtactggtttatcagtatgctgcatccggagtccaagccaacaataaattgttgtatgatctttcgccgatgcgcgctgatattggtgacatgcgtaagtacgccgtactcgtgtattcaaaagacgatgcacttgagtcggatgagctagtgcttcatgtcgacattgagcaccaacgtattcccacatctggagtgctcccagtttga

SEQ ID NO: 289 - DNA, 657 bp, Cucumber mosaic virus
atggacaaatctgaatcaaccagtgccggtcgtaatcgtcgacgtcgtccgcgtcgtggttcccgctccgcctcctcctccgcggatgctacacttagagtcctgtcgcaacagctttcgcgacttaataagacgttagcagctggtcgtcctactattaaccacccaacctttgtgggtagtgaacgttgtaaacctggatacacgttcacctcgattaccctgaagccaccgaaaatagacaaggggtcttattatggcaaaaggttgttacttcctgattcagtcactgagttcgataagaagcttgtttcgcgcattcaagttcgagttaatcctttgccgaaatttgattctaccgtgtgggtgacggtccgtaaagttcctgcctcctcggacctgtccgtatccgccatctctgctatgtttgcggacggagcctcaccggtactggtttatcagtatgctgcatctggcgttcaagccaataacaaattgttatatgatctttcagtgatgcgcgctgatattggtgatatgagaaagtacgccgtgctcgtgtattcaaaagacgatgcgcttgagacggacgaactagtacttcatgtcgacatcgagcaccaacgcattcccacgtctggggtgctcccagtttga

SEQ ID NO: 290 - DNA, 657 bp, Cucumber mosaic virus
atggacaaatctgaatcaaccagtgccggtcgtaatcgtcgacgtcgtccgcgtcgcggttcccgctccgcctcctcctccgcggatgctacacttagagtcctgtcgcaacagctttcgcgacttaataagacgttagcagctggtcgtcctactattaaccacccaacctttgtgggtagtgagcgttgtaaacctggatacacgttcacctcgattaccctgaagccaccgaaaatagacaaggggtcttattatggcaaaaggtggttacttcctgattcagtcactgagttcgataagaagcttgtttcgcgcattcaagttcgagttaatcctttgccgaaatttgattctaccgtgtgggtgacggtccgtaaagttcctgcctcctcggacctgtccgtatccgccatctctgctatgtttgcggacggagcctcaccggtactggtttatcagtatgctgcatctggcgttcaagccaataacaaattgttatatgatctttcagtgatgcgcgctgatattggtgatatgagaaagtacgccgtactcatgtattcaaaagacgatgcgcttgagacggacgaactagtatttcatgtcgacatcgagcaccaacgcattcccacgtctggggtgctcccagtttga

SEQ ID NO: 291 - DNA, 657 bp, Cucumber mosaic virus
atggacaaatctgaatcaaccagtgccggtcgtaatcgtcgacgtcgtctgcgtcgcggttcccgctccgcctcctcctccgcggatgctacatttagagtcctgtcgcaacagctttcgcgacttaataagacgttagcagctggtcgtcctactattaaccacccaacctttgtgggtagtgagcgttgtaaacctggatacacgttcacctcgattaccctgaagccaccgaaaatagacaaggggtcttattatggcaaaaggttgttacttcctgattcagtcactgagttcgataagaagcttgtttcgcgcattcaaattcgagttaatcctttgccgaaatttgattctaccgtgtgggtgacggtccgtaaagttcctgcctcctcggacctgtctgtatccgccatctctgctatgtttgcggacggagcctcaccggtactggtttatcagtatgctgcatctggcgttcaagccaataacaaattgttatatgatctttcagtgatgcgcgctgatattggtgacatgagaaagtacgccgtgctcgtgtattcaaaagacgatgcgcttgagacggacgaactagtacttcatatcgacatcgagcaccaacgcattcccacgtctggggtgctcccagtttga

SEQ ID NO: 292 - DNA, 657 bp, Cucumber mosaic virus
atggacaaatctggatctcccaatgctagtagaacctcccggcgtcgtcgcccgcgtagaggttctcggtccgcttctggtgcggatgcagggttgcgtgctttgactcagcagatgctgaaactcaataaaaccctcgccattggtcgtcccactcttaaccacccaaccttcgcgggtagtgaaagctgtaaacccggttacactttcacatctattaccctgaaaccgcctgaaattgagaaaggttcatattttggtagaaggttgtctttgccagattcagtcacggactatgataagaagcaagtttcgcgcattcaaatcaggattaatcctttgccgaaatttgattctaccgtgtgggttacagttcggaaagtaccttcatcatccgatctttccgtcgccgccatcactgctatgtttggcgatggtaaatcaccggttttggtttatcagtatgctgcgtccggagttcaggccaacaataagttactctataacctgtccgagatgcgtgctgatatcggcgacatgcgtaagtacgccgtcctggtttactcgaaagacgataaactagagaaggacgagattgtacttcatgtcgacgtcgagcatcaacgaattcctatctcacggatgctcccgacttag

SEQ ID NO: 293 - DNA, 657 bp, Cucumber mosaic virus
atggacaaatctggatctcccaatgctagtagaacctcccggcgtcgtcgcccgcgtagaggttctcggtccgcttctggtgcggatgcagggttgcgtgctttgactcagcagatgctgagactcaataaaaccctcgccattggtcgtcccactcttaaccacccaaccttcgtgggtagtgaaagctgtaaacccggttacactttcacatctattaccctgaaaccgcctgaaattgagaaaggttcatattttggtagaaggttgtctttgccagattcagtcacggactatgataagaagcttgtttcgcgcattcaaatcaggattaatcctttgccgaaatttgattctaccgtgtgggttacagttcggaaagtgccttcatcatccgatctttccgtcgccgccatctctgctatgtttggcgatggtaactcaccggttttggtttatcagtatgctgcgtccggagttcaggccaacaataagttactttatgacctgtccgagatgcgtgctgatatcggcgacatgcgtaagtacgccgtcctggtttactcgaaagacgataaactagagaaggacgagattgtacttcatgtcgacgtcgagcatcaacgaattcctatctcacggatgctcccgacttag

SEQ ID NO: 294 - DNA, 657 bp, Cucumber mosaic virus
atggacaaatctgaatctcccaatgctagtagaacctcccggcgtcgtcgcccgcgtagaggttctcggtccgcttctggtgcggatgcagggttgcgtgctttgactcagcagatgctgaaactcaataaaaccctcgccattggtcgtcccactcttaaccacccaaccttcgtgggtagtgcaagctgtaaacccggttacactttcacatctattaccctgaaaccgcctgaaattgagaaaggttcatatttcggtagaaggttgtctttgccagattcagtcacggactatgataagaagcttgtttcgcgcattcaaatcaggattaatcccttgccgaaatttgattctaccgtgtgggttacagttcggaaagtaccttcatcatccgatctttccgtcgccaccatctctgctatgtttggcgatggtaattcaccggttttggtttatcagtatactgcgtccggagttcaggccaacaataagttactttatgacctgtccgagatgcgtgctgatatcggcgacatgcgtaagtacgccgtcctggtttactcgaaagacgataaactagaggaggacgagattgtacttcatgtcgacgtcgagcatcaacgaattcctatctcacggatgctcccgacttag

SEQ ID NO: 295 - DNA, 657 bp, Cucumber mosaic virus
atggacaaatctggatctcccaatgctagtagaacctcccggcgtcgtcgcccgcgtagaggttctcggtccgcttctggtgcggatgcagggttgcgtgctttgactcagcagatgctgaaactcaatagaaccctcgccattggtcgtcccactcttaaccacccaaccttcgtgggtagtgaaagctgtaaacccggttacactttcacatctattaccctgaaaccgcctgaaattgagaaaggttcatattttggtagaaggttgtctttgccagattcagtcacggactatgataagaagcttgtttcgcgcattcaaatcaggattaatcctttgccgaaatttgattctaccgtgtgggttacagttcggaaagtaccttcatcatccgatctttccgtcgccgccatctctgctatgtttggcgatggtaactcaccggttttggtttatcagtatgctgcgtccggagttcaggccaacaataagttactttatgacctgtccgagatgcgtgctgatatcggcgacatgcgtaagtacgccgtcctggtttactcgaaagacgataaactagagaaggacgagattgtacttcatgtcgacgtcgagcatcaacgaattcctatctcacggatgctcccgacttag

SEQ ID NO: 296 - DNA, 657 bp, Cucumber mosaic virus
atggacaaatctggatctcccaatgctagtagaacctcccggcgtcgtcgcccgcgtagaggttctcggtccgcttctggtgcggatgcagggttgcgtgctttgactcagcagatgctgagactcaataaaaccctcgccattggtcgtcccactcttaaccacccaaccttcgtgggtagtgaaagctgtaaacccggttacactttcacattcattaccctgaaaccgcctgaaattgaaaaaggttcatattttggtagaaggttgtctttgccagattcagtcacggactatgataagaagcttgtttcgcgcattcaaatcaggattaatcctttgccgaaatttgattctaccggttgggttacagttcggaaagtaccttcatcatccgatctttccgtcgccgccatctctgctatgttcggtgatggtaactcaccggttttggtttatcagtatgctgcgtccggagttcaggccaacaataagttactttatgacctgtccgagacgcgtgctgatatcggcgacatgcgtaagtacgccgtcctggtttactcgaaagacgataaactagagaaggacgagattgtacttcatgtcgacgtcgagcatcaacgaattcctatctcacggatgctcccgacttag

SEQ ID NO: 297 - DNA, 657 bp, Cucumber mosaic virus
atggacaaatctggatctcccaatgctagtagaacctcccggcgtcgtcgcccgcgtagaggttctcggtccgcttctggtgcggatgctgggttgcgtgctttgactcagcagatgctgaaactcaataaaaccctcgccattggtcgtcccactcttaaccacccaaccttcgtgggtagtgaaagctgtaaacccggttacactttcacatctattaccctgaaaccgcctgaaattgagaaaggttcatattttggtagaaggttgtctttgccagattcagtcacggactatgataagaagcttgtttcgcgcattcaaatcaggattaatcctttgccgaaatttgattctaccgtgtgggttacagttcggaaagtaccttcatcatccgatctttccgtcgccgccatctctgctatgtttggcgatggtaattcaccggttttggtttatcagtatgctgcgtccggagttcaggccaacaataagttactttatgacctgtccgagatgcgtgctgatatcggcgacatgcgtaagtacgccgtcctggtttactcgaaagacgataaactagagaaggacgagattgtacttcatgtcgacgtcgagcatcaacgaattcctatctcacggatgctcccgacttag

SEQ ID NO: 298 - DNA, 657 bp, Cucumber mosaic virus
atggacaaatctggatctcccaatgctagtagaacctcccggcgtcgtcgcccgcgtagaggttctcggtccgcttctggtgcggatgcagggttgcgtgctttgactcagcagatgctgaaactcaataaaaccctcgccattggtcgtcccactcttaaccacccaaccttcgtgggtagtgaaagctgtaaacccggttacactttcacatctattaccctgaaaccgcctgaaattgagaaaggttcatattttggtagaaggttgtctttgccagattcagtcacggactatgataagaagcttgtttcgcgcattcaaatcaggatcaatcctttgccgaaatttgattctaccgtgtgggttacagttcggaaagtaccttcatcatccgatctttccgtcgccgccatctctgctatgtttggcgatggtaattcaccggttttggtttatcagtatgctgcgtccggagttcaggccaacaataaattactttatgacctgtccgagatgcgtgctgatatcggcgacatgcttaagtacgccgtcctggtttactcgaaagacgataagctggagaaggacgagattgtacttcatgtcgacgtcgagcatcaacgaattcctatctcacggatgctcccgacttag

SEQ ID NO: 299 - DNA, 657 bp, Cucumber mosaic virus
atggacaaatctgaatctcccaatgctagtagaacctcccggcgtcgtcgcccgcgtagaggttctcggtccgcttctggtgcggatgcagggttgcgtgctttgactcagcagatgctgaaactcaataaaaccctcgccattggtcgtcccactcttaaccacccaaccttcgtgggtagtgaaagctgtaaacccggttacactttcacatctattaccctgaaaccgcctgaaattgagaaaggttcatatttcggtagaaggttgtctttgccagattcagtcacagactatgataagaagcttgtttcgcgcattcaaatcaggattaatcccttgccgaaatttgattctaccgtgtgggttacagttcggaaagtaccttcatcatccgatctttccgtcgccaccatctctgctatgtttggcgatggtaattcaccggttttggtttatcagtatgctgcgtccggagttcaggccaacaataagttactttatgacctgtccgagatgcgtgctgatatcggcgacatgcgtaagtacgccgtcctggtttactcgaaagacgataaactagaggaggacgagattgtacttcatgtcgacgtcgagcatcaacgaattcctatctcacggatgctcccgacttag

SEQ ID NO: 300 - DNA, 657 bp, Cucumber mosaic virus
atggacaaatctggatctcccaatgctagtagaacctcccggcgtcgtcgcccgcgtagaggttctcggtccgcttctggtgcggatgcagggttgcgtgctttgactcagcagatgctgaagctcaatagaaccctcgccattggtcgtcccactttaatccacccaagcttcgtgggtagtgaaagtggaaaccccggttacaccttcacatctattaccctgaacccgcctgaatttgagaaaggttcatattttggtagaaggttgtctttgccagattcagtcacggactatgataagaagcttgtttcgcgcattcaaatcaggattaatcctttgccgaaatttgattctaccgtgtgggttacagttcggaaagtaccttcatcatccgatctttccgtcgccgccatctctgctatgtttggcgatggtaactcaccggttttggtttatcagtatgctgcgtccggagttcaggccaacaataagttactttatgacctgtccgagatgcgtgctgatatcggcgacatgcgtaagtacgccgtcctggtttactcgaaagacgataaactagagaaggacgagattgtacttcatgtcgacgtcgagcatcaacgaattcctatctcacggatgctcccgacttag

SEQ ID NO: 301 - DNA, 336 bp, Cucumber mosaic virus
atggaattgaacgaaggcgcagtgacaaacgtcgaactccagctggctcgtatgatggaggtgaagagacagagacgaaggtctcacaagaagaatcgacgggaacgaggtcacaaaagtcccagcgagagggcgcgttcaaatctcagactgttccgttttctaccgttttatcagatagacggttcggagctgatagagatgcaccaccgtgcgcgcgcggtggaattgtccgagtctgaggccccttgttttccattatcagcggaagaagaccatgattttgacgatacggattggttcgctggtaatgaatgggcggaaggtgtgttttga

SEQ ID NO: 302 - DNA, 336 bp, Cucumber mosaic virus
atggaattgaacgaaggcgcgatgacaaacgtcgaactccaactggcccgcatggtggaggcgaagagacagagacgaaggtctcacaagaagaatcgacgggaacgatgtcacaaaagtcccagcgagagggcgcgttcaaatctcagactattccgcttcctaccgttctatcaggtagatggttcggaactgatagagatgtaccgccacgtgaacgtggcggaattgtccgggtctgaggccccttgttttacgttgccagcggaagatgaccatgatttcgacgatacagattggttcgctggtaacgaatgggcggaaggagcgttctga

SEQ ID NO: 303 - DNA, 336 bp, Cucumber mosaic virus
atggaattgaacgaaggcgcaatgacaaacgtcgaactccagctggctcgcatgatggaggtgaggagacaaagacgaaagtctcacaagaagaatcgacgggaacgaggtcacaaaagtcccagcgagagagcgcgttcaaatctcaggctattccgatttttaccgttttatcagatagatggttcggaactgatagagatgtaccaccacgcgagtgtggtggaattgtccgagtctgaggctcctcggtttacgttaccagcggaagaagaccatgattttgacgacacagattggttcgctggtaatgaatgggcggaaggtgcgttttga

SEQ ID NO: 304 - DNA, 333 bp, Cucumber mosaic virus
atggaattgaacgaaggcgcagtgacaaacgtcgaactccaactggctcgtatggtggaggtgaaaagacagagacgaaggtctcacatgaagaatcgacgggaacgtggtcacaaaagtcccagcgagagggcgcgttcaaatctcagattgttccgcttcctaccattttatcaggtagatggttcggaactgatagagctccaccatgtgaacatggtggaattatccgaatctgaggcccctcgtttatcgttactggcggaagaagaccatgattttgacgatacggattggttcgctggtaacgagtgggcggaagggtcgttttga

SEQ ID NO: 305 - DNA, 336 bp, Cucumber mosaic virus
atggaattgaacgcaggcgcaatgacaagcgtcgaactccaactagcccgcatggtggaggcgaagagacagagacgaagatctcacaagaagaatcgacgggaacgatgtcacaaaagtcccagcgagagggcgcgttcaaatctcagactgttccgcttcctaccgttctttcaagtagatggtttggaactgatagagatgtaccgccacgcgagcgtggcggaattgtccgagtctgaggccccttgttttccgttgccagcggaagatgaccatgatttcgacgatacagattggttcgctggtaacgagtgggcggaaggagcattctga

SEQ ID NO: 306 - DNA, 333 bp, Cucumber mosaic virus
atggaattgaacgtaggtgcaatgacaaacgtcgaactccaactggctcgtatggtggaggtgaagaagcagagacgaaggtctcacaaacagaatcgacgggaacgaggtcacaaaagtcccagcgagagagcgcgttcaaatctcagactgttccgcttcctaccgttctatcaagtggacggttcggaactgacagggtcatgccgccatgcgaacgtggcggagttacccgagcctgaggcctctcgtttaaagttatcggcggaagaccatgattttgacgatacagattggttcgccggtaacgaatgggcggaaggtgctttctga

SEQ ID NO: 307 - DNA, 333 bp, Cucumber mosaic virus
atggaattgaacgtaggtgcaatgacaaacgtcgaactccaactggctcgtatggtggaggcgaagaagcagagacgaaggtctcacaaacagaatcgacgggaacgaggtcacaaaagtcccagcgagagagcgcgttcaaatctcagactgttccgcttcctaccgttctatcaagtagatggttcggaattgacagggtcatgccgccatgtgaacgtggcggagttacccgagtctgaggcctctcgtttagagttatcggcggaagaccatgattttgacgatacggattggttcgccggtaacgaatgggcggaaggtgctttctga

SEQ ID NO: 308 - DNA, 333 bp, Cucumber mosaic virus
atggaattgaacgtaggtgcaatgacaaacgtcgaactccaactggctcgtatggtggaggtgaagaaacggagacgaagatctcacaaacagaatcgacgggaacgaggtcacaaaagtcccagcgagagagcgcgttcaaatctcagactgttccgcttcctaccgttctatcaagtagatggttcggaactgacagggtcatgccgccatgcgagcgtggcggagttacccgagtctgaggcctctcgtttagagttatcggcggaggaccatgatttcgacgatacagattggttcgccggtaacgaatgggcggaaggtgctttctga

SEQ ID NO: 309 - DNA, 333 bp, Cucumber mosaic virus
atggaattgaacgtaggtgcaatgacaaacgtcgaactccaactggctcgtatggtggaggcgaagaagcagagacgaaggtctcacaaacagaatcgacgggaacgaggtcacaaaagtcccagcgagagagcgcgttcaaatctcagactattccgcttcctacccttttaccaagtagatggttcggaactgacagggtcataccgccatgtgaacgtggcggagttacccgagtctgaggcctctcgtttagagttatcggcggaagaccatgattttgacgatacagattggttcgccggtaacgaatgggcggaaggtgctttctga

SEQ ID NO: 310 - DNA, 840 bp, Cucumber mosaic virus
atggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgataatgttatttcagtccggcccctcgttcccgaggtaacccacggtcgtattgcttccttctttaagtctggatatgatgttggtgaattatgctcaaaaggatacatgagtgtccctcaagtgttatgtgctgttactcgaacagtttccactgatgctgaagggtctttgagaatttacttagctgatctaggcgacaaggagttatctcccatagatgggcaatgcgtttcgttacataaccatgatcttcccgctttggtgtctttccaaccgacgtatgattgtcctatggaaacagttgggaatcgtaagcggtgttttgctgtcgttatcgaaagacatggttacattgggtataccggtaccacagctagcgtgtgtagtaattggcaagcaaggttttcatccaagaataacaactacactcatatcgcagctgggaagactctagtactgcctttcaacagattagctgagcaaacaaaaccgtcagctgtcgctcgcctgttgaagtcgcaattgaacaacattgaatcttcgcaatatttgttaacgaatgcgaagattaatcaaaatgcgcgcagtgagtccgaggatttaaatgttgagagccctcccgccgcaatcgggagttcttccgcgtcccgctccgaagccttcagaccgcaggtggttaacggtctttag

SEQ ID NO: 311 - DNA, 840 bp, Cucumber mosaic virus
atggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgataatgctatttcagtccggcccctcgttcccgaggtaacccacggtcgtattgcttccttctttaagtctggatatgatgttggtgaattatgctcaaaaggatacatgagtgtccctcaagtgttatgtgctgttactcgaacagtttccactgatgctgaagggtctttgagaatttacttagctgatctaggcgacaaggagttatctcccatagatgggcaatgcgtttcgttacataaccatgatcttcccgctttggtgtctttccaaccgacgtatgattgtcctatggaaacagttgggaatcgtaagcggtgttttgctgtcgttatcgaaagacatggttacattgggtataccggtaccacagctagcgtgtgtagtaattggcaagcaagtttttcatccaagaataacaactacactcatatcgcagctgggaagactctagtactgcctttcaacagattagctgagcaaacaaaaccgtcagctgtcgctcgcctgttgaagtcgcaattgaacaacattgaatcttcgcaatatttgttaacgaatgcgaagattaatcaaaatgcgcgcagtgagtccgaggatttaaatgttgagagccctcccgccgcaatcgggagttcttccgcgtcccgctccgaagccttcagaccgcaggtggttaacggtctttag

SEQ ID NO: 312 - DNA, 140 bp, Cucumber mosaic virus
atggctttccaaggtcccagtaggactttaactcaacagtcctcagcggctacgtctgacgatcttcaaaagatattatttagccctgaagccattaagaaaatggctgctgagtgtgacctaggccggcatcattggat

SEQ ID NO: 313 - DNA, 840 bp, Cucumber mosaic virus
atggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgataatgctatttcagtccggcccctcgttcccgaagtaacccacggtcgtattgcttccttctttaagtctggatatgatgttggtgaattatgctcaaaaggatacatgagtgtccctcaagtgttatgtgctgttactcgaacagtttccactgatgctgaagggtctttgagaatttacttagctgatctaggcgacaaggagttatctcccatagatgggcaatgcgtttcgttacataaccatgatcttcccgctttggtgtctttccaaccgacgtatgattgtcctatggaaacagttgggaatcgtaagcggtgttttgctgtcgttatcgaaagacatggttacattgggtataccggtaccacagctagcgtgtgtagtaattggcaagcaaggttttcatctaagaataacaactacactcatatcgcagctggaaagactctagtactgcctttcaacagattagctgagcaaacaaaaccgtcagctgttgctcgcctgttgaagtcgcaattgaacaacattgaatcttcgcaatatttgttaacgaacgcgaagattaatcaaaatgcgcgcagtgagtccgaggatttaaatgttgagagccctcccgccgcaatcgggagttcttccgcgtcccgctccgaagccttcagaccgcaggtggttaagggtctttag

SEQ ID NO: 314 - DNA, 840 bp, Cucumber mosaic virus
atggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgacaatgctatttcagtccggcccctcgttcccgaagtaacccacggtcgtattgcttccttctttaagtctggatatgatgttggtgaattgtgctcaaaaggatacatgagcgtccctcaagtattatgtgctgttactcgaacagtttccactgatgctgaagggtctttgagaatttacttagctgatctaggcgacaaggagttatctcccatagacgggcaatgcgtttcgttacataaccatgatcttcccgctttggtgtctttccaaccgacgtatgattgtcccatggagacagttgggaatcgtaagcggtgttttgctgttgttatcgaaagacatggttacattgggtataccggtaccacagctagcgtgtgtagtaattggcaagcaaggttttcatccaagaataacaactacactcatatcgcagctgggaagactctagtactgcctttcaacagattagctgagcaaacaaaaccgtcagctgtcgctcgcctgttgaagtcgcaattgaacaacattgaatcttcgcaatatttgttaacgaacgcgaagatcaatcagaatgcgcgtagtgagtccgaggaattaaatgttgagagccctcccgccgcaatcgggagttcttccgcgtcccgctccgaagccttcagaccgcaggtggttaacggtctttag

SEQ ID NO: 315 - DNA, 840 bp, Cucumber mosaic virus
atggctttccaaggtaccagtaggactttaactcaacagtcctcagcggctacgtctgacgatcttcgaaagatattatttagccctgaagccattaagaaaatggctactgagtgtgacctaggccggcatcattggatgcgcgctgataatgctatttcagtccggcccctcgttcccgaagtaacccacggtcgtattgcttccttctttaagtctggatatgatgttggtgaattatgctcaaaaggatacatgagtgtccctcaagtgttatgtgctgttactcgaacagtttccattgatgctgaagggtctttgagaatttacttagctgatctaggcgacaaggagttatctcccatagatgggcaatgcgtttcgttacataaccatgatcttcccgctttggtgtctttccaaccgacgtatgattgtcctatggaaacagttgggaatcgtaagcggtgttttgctgtcgttatcgaaagacatggttacattgggtacaccggtaccacagctagcgtatgtagtaattggcaagcaaggttttcatctaagaataacaactacattcatatcgcagctgggaagactctagtactgcctttcaacagattagctgagcaaacaaaaccgtcagctgttgctcgcctgttgaagtcgcaattgaacaacattgaatcttcgcaatatttgttaacgaatgcgaagattaatcaaaatgcgcgcagtgagtccgaggatttaaatgttgagagccctcccgccgcaatcgggagttcgtccgcgtcccgctccgaagccttcagaccgcaggtggttaacggtctttag

SEQ ID NO: 316 - DNA, 840 bp, Cucumber mosaic virus
atggctttccaaggtaccagtaggactttaactcaacagtcctcagcggctacgtctgacgatcttcaaaagatattatttagccctgaagccattaagaaaatggctactgagtgtgacctaggccggcatcattggatgcgcgctgataatgctatttcagtccggcccctcgttcccgaagtaacccacagtcgtattgcatccttctttaaatctggatatgatgttggtgaattgtgctcaaaaggatacatgagcgtccctcaagtgttatgtgctgttactcggacagtctctactgatgctgaagggtccttgagaatttacttagctgatctaggtgacaaggagttatctcctatagatgggcaatgcgtttcgttacacaaccatgatcttcccgctttggtgtctttccaacctacgtacgactgtcctatggaaacagttgggaatcgtaagcggtgttttgctctcgttatcgaaagacatggttacattgggtataccggtaccacagctagcgtgtgtagtaattggcaagcaaggttttcttctaagaataacaactacactcatatcgcagctgggaagactctagtactgcctttcaacagattagctgagcaaacaaaaccgtcagctgtcgctcgcctgttgaagtcgcaattgaacaacattgaatcttcgcaatatctgttaacgaacgcgaagattaatcaaaatgcgcgcagtgagtccgaggaattaaatgttgagagccctcccgccgcaatcgggagttcttccgcgtcccgctccgaagccttcagagcgcaggtggttaacggtctttag

SEQ ID NO: 317 - DNA, 714 bp, Pepino mosaic virus
atgcctgacacaacacctgttgctgccacttcaagtgcaccacccacagccaaagatgctggtgccaaagctccttctgacttctcaaatcccaatacagctcctagtctcagtgatttgaagaaagtcaagtatgtctccaccgtgacctccgtggccacaccagctgaaattgaagccctaggcaaaatcttcaccgctatgggccttgccgccaatgagactggtccggccatgtgggatctagctcgtgcatatgctgatgtgcagagttctaaatcggcacagctgattggagctaccccttccaaccctgcactatcacgccgagcccttgctgctcagtttgatcgaatcaatataacccccaggcaattttgcatgtactttgccaaagttgtttggaacatacttctcgacagcaacattccaccagcaaattgggccaaacttggttaccaagaagatacaaaatttgctgcatttgacttcttcgatggagtcaccaaccctgccagcctgcagcctgctgatggtcttatcaggcagccaaatgaaaaagaactagctgctcactccgtagctaagtacggcgccttggctaggcaaaagatctccacaggtaattatattaccacacttggagaagtcacacgtggacacatgggaggagctaacaccatgtacgcgatagacgcaccccctgaactttaa

SEQ ID NO: 318 - DNA, 714 bp, Pepino mosaic virus
atggaaaaccaacctacagcttctaacccatcaaatgtaccaccaactgctgctcaagctggtgcccagagcccagccgacttctcaaatcctaatacagctccttccctaagtgatttgaagaagatcaaatacgtgtcaactgtcacttcagttgccacgcctgctgaaattgaggcccttggcaagatcttcactgccatgggtttagcagccaatgagaccggacctgccatgtgggacctcgctcgtgcttatgctgatgtgcaaagttcaaaatctgcacaacttataggtgccacaccatccaaccctgctttgtctagacgtgcacttgctgcacagtttgatcgtatcaatatcacacccagacaattctgcatgtattttgcaaaaattgtttggaacatactgttagacagcaatgtgccacctgccaactgggcaaaattgggctatcaggaagataccaagtttgctgcttttgacttctttgatggagtcacaaatccagctagtctacagcctgcagatggcctaatcaggcagcccaatgaaaaagagcttgctgctcactcggttgctaaatatggtgcccttgcccgccagaaaatatccactggtaactacatcaccacccttggtgaagttacacgtggtcacatgggcggcgccaacactatgtacgcaattgatgcacctcctgaactttaa

SEQ ID NO: 319 - DNA, 420 bp, Pepino mosaic virus
atggaaaaccaacctacagcttctaacccatcagatgtaccaccaactgctgctcaagctggtgcccagagcccagccgacttctcaaatcctaatacagctccttccctaagtgatttgaagaagatcaaatacgtgtcaactgtcacttcagttgccacgcctgctgaaattgaggcccttggcaagatctttattgccatgggtttagcagccaatgagaccggacctgccatgtgggacctcgctcgtgcttatgctgatgtgcaaagttcaaaatctgcacaacttataggtgccacaccatccaaccctgctttgtctagacgtgcacttgctgcacagtttgatcgtatcaatatcacacccagacaattctgcatgtattttgcaaaaattgtttggaacatactgttagac

SEQ ID NO: 320 - DNA, 420 bp, Pepino mosaic virus
atgcctgacacaacacctgttgctgccacttcaagtgcaccacccacagccaaagatgctggtgccaaagctccttctgacttctcaaatcccaatacagctcctagtctcagtgatttgaagaaagtcaagtatgtctccaccgtgacctccgtggccacaccagttgaaattgaagccctaggcaaaatcttcaccgctatgggccttgccgccaatgagactggtccggccatgtgggatctagctcgtgcatatgctgatgtgcagagttctaaatcggcacagctgattggagctaccccttccaaccctgcactatcacgccgagcccttgctgctcagtttgatcgaatcaatataacccccaggcaattttgcatgtactttgccaaagttgtttggaacatacttctcgac

SEQ ID NO: 321 - DNA, 560 bp, Pepino mosaic virus
atgcctgacacaacacctgttgctgccacttcaagtgcaccacctacagccaaagatgctggtgccaaagctccttctgacttctcaaatcccaatacagctcctagtctcagtgatttgaagaaagtcaagtatgtctccacagtgacttccgtggccacaccagctgaaattgaagccctaggcaaaatcttcaccgctatgggccttgccgccaatgagactggtccggcgatgtgggatctagctcgtgcgtatgctgatgtgcagagctctaaatccgcacagctgattggtgctaccccttccaaccctgcattatcacgccgagcccttgctgctcagtttgatcgaatcaatataacacccaggcaattttgcatgtactttgctaaagttgtttggaacatccttctcgacagcaatattccaccagcaaattgggctaaacttggttaccaagaagatacaaaatttgctgcatttgacttcttcgatggagtcaccaaccctgccagcctgcagcctgctgatggtctcatcaggcaaccaaatgagaa

SEQ ID NO: 322 - DNA, 420 bp, Pepino mosaic virus
atggaaaaccaacctacagcttctaacccatcagatgtaccaccaactgctgctcaagctggtgcccagagcccagccgacttctcaaatcctaatacagctccttccctaagtgatttgaagaagatcaaatacgtgtcaactgtcacttcagttgccacgcctgctgaaattgaggcccttggcaagatctttactgccatgggtttagcagccaatgagaccggacctgccatgtgggacctcgctcgtgcttatgctgatgtgcaaagttcaaaatctgcacaacttataggtgccacaccatccaaccctgctttgtctagacgtgcacttgctgcacagtttgatcgtatcaatatcacacccagacaattctgcatgtattttgcaaaaattgtttggaacatactgttagac

SEQ ID NO: 323 - DNA, 705 bp, Pepino mosaic virus
atggaaagatcaactctgattaatttacttcaattgcaccacttcgagccaaaactcagtgttgaaggaaccatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 324 - DNA, 705 bp, Pepino mosaic virus
atggaaagatcaactctgattaatttacttcaattgcaccacttcgagccaaaactcagtgttgaaggaaccatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagactaagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 325 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccactagctcagaggaactagccggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 326 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaacccaccattggggaactaaatgccaggtctaactcctag

SEQ ID NO: 327 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccaccagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 328 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatagttgaattctttaacccttttgaagttgaccccactgagcatatctctgcctctgaagaagaagtcttggactttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 329 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttgggctttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 330 - DNA, 705 bp, Pepino mosaic virus; identical to SEQ ID NO: 329

SEQ ID NO: 331 - DNA, 705 bp, Pepino mosaic virus; identical to SEQ ID NO: 329

SEQ ID NO: 332 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttccagaaccaaaattggctatctttggtgacccctgtcaatgcacatacattgagagacttagagtcccacattacacttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 333 - DNA, 705 bp, Pepino mosaic virus
atggaaagatcaactctgattaatttacttcaattgcaccacttcgagccaaaactcagtgttgaaggaatcatagttgtgcacggaattgcaggcactgggaaaaccactttacttaggactttattttctgcttaccctagcttagttataggttcacctaggccttgctatttagataaacaaaacaaaatttcacaagtttgcttatcttgctttcccaatacccattgtgatattgtcgatgagtatcatttgctagaaagttttctagaaccaaaattggctatctttggtgacccctgtcaatgcacatacattgagagacttagagtcccacattacatttccttcagaactcatagatttggaaagtcaactgctgagattttgaacaaactgtttgaccttaatatagtctcagttaagaaagaagacgacatcgttgaattctttaacccttttgaagttgaccccactgagcatatctctgcctctgaagaagaagtcttggactttgtttctgaccaagtggtgaccactagctcagaggaactagcaggacttgagtttgcagaaacaactttctactgcacaacattggccgcagctgttgctgaaaatcctgctaagactttcatctctctgactagacacacccacaaactcaccattggggaactaaatgccaggtctaactcctag

SEQ ID NO: 334 - DNA, 372 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgccttttggtggccaatatcaagacggcaccaaaaagatatcctattttccacaacagcagtcatactttcattctggaaacaaattaaatgtcctcatacttatcttcattctcacgttgggtattgtcctcaccaataaatttagttttagctttagtcgtactactcaccagcattcttgctataacacacattcagcaaccaacaatacacaaccattgtcaggccatcattga

SEQ ID NO: 335 - DNA, 372 bp, Pepino mosaic virus; identical to SEQ ID NO: 334

SEQ ID NO: 336 - DNA, 280 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttatcttcattctcacgttgggtattgtcctcaccaataaattta

SEQ ID NO: 337 - DNA, 280 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgcctttcggtggccaatatcaagacggcaccaaaaagatatcttattttccacaacagcagtcatactttcattctggaaacaaattaaatgtcctcatacttatcttcattctcacattgggtattgtcctcaccaataaattta

SEQ ID NO: 338 - DNA, 280 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttattttcattctcacattgggtattgtcctcaccaataaattta

SEQ ID NO: 339 - DNA, 372 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgccttttggtggccaatatcaagacggcaccaaaaagatatcttattttccacaacagcaatcatactttcattctggaaacaaattaaatgtcctcatacttatcttcattctcacattgggtattgtcctcaccaataaatttagttttagctttagtcgtactactcaccagcattcttgctataacacacattcagcaaccaacaatacacaaccattgtcaggtcatcattga

SEQ ID NO: 340 - DNA, 372 bp, Pepino mosaic virus
atgccaggtctaactcctagagctgacctcactgacacatacaaaattattgccattgctttcttgttgtcagcttgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttatcttcattctcacattgggtattgtcctcaccaataaatttagttttagctttagtcgtactactcaccagcattcttgctataacacacattcagcaaccaacaatacacaaccattgtcaggtcatcattga

SEQ ID NO: 341 - DNA, 372 bp, Pepino mosaic virus
atgccaggtctaactcctagagctgacctcactgacacatacaaaatcatcgccattgctttcttgttgtcagcttgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttatcttcattctcacattgggtattgtcctcaccaataaatttagttttagctttagtcgtactactcaccagcattcttgctataacacacattcagcaaccaacaatacacaaccattgtcaggtcatcattga

SEQ ID NO: 342 - DNA, 372 bp, Pepino mosaic virus
atgccaggtctaactcctagagctgacctcactgacacatacaaaatcattgccattgctttcttgttgtcagcctgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttatcttcattctcacattgggtattgtcctcaccaataaatttagttttagctttagtcgtactactcaccagcattcttgctataacacacattcagcaaccaacaatacacaaccattgtcaggtcatcattga

SEQ ID NO: 343 - DNA, 372 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttatcttcattctcacattgggtattgtcctcaccaataaatttagttttagctttagtcgtactactcaccagcattcttgctataacacacattcagcaaccaacaatacacaaccattgtcaggtcaccattga

SEQ ID NO: 344 - DNA, 372 bp, Pepino mosaic virus
atgccaggtctaactcctagagctgacctcactgacacatacaaaatcattgccattgctttcttgttgtcagcttgcatttacttccaaaatagccactaccaacctgttgctggagacaacttgcaccgtttgccttttggtggccaatatcaagacggcaccaaaaagatatcttattttccacaacagcagtcatactttcattctggaaacaaattaaatgtcctcatacttatcttcattctcacattgggtattgtcctcaccaataaatttagttttagctttagtcgtactactcaccagcattcttgctataacacacattcatcaaccaacaatacacaaccattgtcaggtcatcattga

SEQ ID NO: 345 - DNA, 255 bp, Pepino mosaic virus
atgtcctcatacttatcttcattctcacgttgggtattgtcctcaccaataaatttagttttagctttagtcgtactactcaccagcattcttgctataacacacattcagcaaccaacaatacacaaccattgtcaggtcatcattgacggtgctgcaatagtcataacaaattgtgagaacacaccagaagtgcttaaagcaatcaacttctccccttggaacgggttaagttttcctaaatttgaaaattaa

SEQ ID NO: 346 - DNA, 255 bp, Pepino mosaic virus
atgtcctcatacttatcttcattctcacgttgggtattgtcctcaccaataaatttagttttagctttagtcgtactactcaccagcattcttgctataacacacattcagcaaccaacaatacacaaccattgtcaggtcaccattgacggggctgcaatagtcataacaaattgtgagaacacaccagaattgcttaaagcaatcaacttctccccttggaacgggttaagttttcctaaatttgaaaattaa

SEQ ID NO: 347 - DNA, 255 bp, Pepino mosaic virus
atgtcctcatacttatcttcattctcacattgggtattgtcctcaccaataaatttagttttagctttagtcgtactactcaccagcattcttgctataacacacattcagcaaccaacaatacacaaccattgtcaggtcatcattgacggtgctgcaatagtcataacaaattgtgagaacacaccagaagtgcttaaagcaatcaacttctccccttggaacgggttaagttttcctaaatttgaaaattaa

SEQ ID NO: 348 - DNA, 255 bp, Pepino mosaic virus
atgtcctcatacttatcttcattctcacattgggtatcgtcctcaccaataaatttagttttagctttagtcgtactactcaccagcattcttgctataacacacattcagcaaccaacaatacacaaccattgtcaggtcatcattgacggtgctgcaatagtcataacaaattgtgagaacacaccagaagtgcttaaagcaatcaacttctccccttggaacgggttaagttttcctaaatttgaaaattaa

SEQ ID NO: 349 - DNA, 420 bp, Pepino mosaic virus
atgcctgacacaacacctgttgctgccacttcaagtgcaccacccacagccaaagatgctggtgccaaagctccttctgacttctcaaatcccaatacagctcctagtctcagtgatttgaagaaagtcaagtatgtctccaccgtgacctccgtggccacaccagctgaaattgaagccctaggcaaaatcttcaccgctatgggccttgccgccaatgagactggtccggccatgtgggatctagctcgtgcatatgctgatgtgcagagttctaaatcggcacagctgattggagctaccccttccaaccctgcactatcacgccgagcccttgctgctcagtttgatcgaatcaatataacccccaggcaattttgcatgtactttgccaaagttgtttggaacatacttctcgac

SEQ ID NO: 350 - DNA, 603 bp, Barley yellow dwarf
atgaattcagtaggtcgtagaggacctagacgcgcaaatcaaaatggcacaagaaggaggcgccgtagaacagttcggccagtggttgtggtccaacccaatcgagcaggacccagacgacgaaatggtcgacgcaagggaagaggaggggcaaattttgtatttagaccaacaggcgggactgaggtattcgtattctcagttgacaaccttaaagccaactcctccggggcaatcaaattcggccccagtctatcgcaatgcccagcgctttcagacggaatactcaagtcctaccatcgttacaagatcacaagtatccgagttgagtttaagtcacacgcgtccgccaatacggcaggcgctatctttattgagctcgacaccgcgtgcaagcaatcagccctgggtagctacattaattccttcaccatcagcaagaccgcctccaagaccttccggtcagaggcaattaatgggaaggaattccaggaatcaacgatagaccaattttggatgctctacaaggccaatggaactaccactgacacggcaggacaatttatcattacgatgagtgtcagtttgatgacggccaaatag

SEQ ID NO: 351 - DNA, 603 bp, Barley yellow dwarf; identical to SEQ ID NO: 350

SEQ ID NO: 352 - DNA, 531 bp, Barley yellow dwarf
ccagtggttgtggtcaatcccaatcgagcaggacccagacgacgaaatggtcgacgcgcaggaagaagagggccagaatctatacctggatcagcaggcaggactgaggtattcatattcttagtcgacaaccttaaagccaactcttccgggacaatcaaattcggccccagtctatcgcaatgcccagcgctttcagacggaatacttaagtcctaccaccgttacaagatcacaagtatccgtgttgagtttaagtcacacgcgtcccccactacgtcgggcgctatctttgttgaactcgacaccgcgtgcaagcaatcagccctgggtagcaaaattaattccttcaccatcagcaaaactgcctccaaatccttcagagccgaggcgattaatgggaaggacttccaagaatcaacgatagaccagttctggctactataccaggcaaatgggacaactactgacactgctggacaatttataatagggataaatgtcagtatgttgactccaatataggtagac

SEQ ID NO: 353 - DNA, 531 bp, Barley yellow dwarf
ccagtggttgtggtccaaccgaatcgaacaggacccagacgaagaaatgtgctacgcgcaggtagaagagggccagagtctatacctggatcagcaggcaggacagaactattcatattctcagtcgacaaccttaaagccaactcttccgggacaatcaaattctgccccagtctatcgcactgcccagcgctttcagacggaatacttaagtcctaccaccgatacaagatcacaagtatccgtgttgagtttgagtcacacgcgtccaccactacgtcgggcgctatctttgttgaactcgacaccgcgtgcgagcaggcgggccggggaagctacactaattccttcaccatcagcaaaactgcctccaaatccttcagattcaagtcgattaatgggaaggacttccaaggatcaacgatcgaccagatctggctactatacaaggcaaatgggacaactactgacactgctgggcaatttaatatcaggatagatgtcactatgctgactcccaaataggtagac

SEQ ID NO: 354 - DNA, 139 bp, Barley yellow dwarf
atgaattcagtaggccgtagaggacctagacgagcaaatcaaaatggcccaagaaggcggcgccgtagagcaattcggccagtggttgtggtccaacccaatcgaacaggacccagacgaagaaatggtcgacgtccag

SEQ ID NO: 355 - DNA, 593 bp, Barley yellow dwarf
atgagtttagtaggccgtagaaataaccgcaggagaaatggcccaaggagagcaaggcgcgttagcgcagttcggagaatggttgtggtccaacccaatcgagccggacccaaacgacgaactcgtcgacgcacaagaggaggaggggcaaatcttatatctggaccagcaggcaggactgaggtattcgtattctcagtcaacgaccttaaggccaactcctcagggacaatcaagttcggtcccgacctttcgcaatgcccagcgctttcaggtggaatactcaagtcctaccaccgttacaagatcacaaacgtcaaggttgagtttaagtcacacgcgtccgccaatacagtcggcgcaatgtttgttgaactcgacacttcgtgctcacaatcaaccttgggtagctacattaactcattcaccatctcaaaatcagcaaccaaaaccttcaccgcccaacagattgacgggaaggaattcagggagagcacggtgaaccaattttacatgctatacaaggcgaacggtactacgtcggacaccgccgggcaattcatcatcacaatacgcgttgccaatatgactcc

SEQ ID NO: 356 - DNA, 600 bp, Barley yellow dwarf
atgaattcagtaggccgtagaggacctagaagagcaaacaatggcacacgaaggcggcgccgtcgagcaattcggccagtggttgtggtccagcccaatcgaacaggacccagacgacgaaatgctcgacgcgcaagaggaagaaggccaggttctgtatttggaccaaaacgcggggctgaggtattcgtattctcagtcgacaaccttaaggccaactcctccgggatcctcaaattcggtcccgatttatcgcaatgcccagcggtttcagacggagtacttaagtcctaccacaattacaagatctcaagtatcaacgttgagtttagaacacacgcgtccgccactacgtcgggcgctatgtttattgaactcgacacctcgtgcaagcaatcagccttatctagctacattaactcactcaccatcagcaaatccgcctcaaagtccttccgcgcaacggagattggagggacccagttccaggcgacatcggtgaatcaattctttttattatacaaggcaaatggcactaccactgacattgcagggcagttcattatcaggattgagcttcacctgatgactgccaaatag

SEQ ID NO: 357 - DNA, 603 bp, Barley yellow dwarf
atgaattcagtaggccgtagaggacctagacgcgcaaatcaaaatggcccaagaaggaggcgccgtagaacagttcggccagtggttgtggtccaacccaatcgagcaggacccagacgacgaaatggtcgacgcaagggaagaggaggggcaaatcctgtatttagaccaacaggcgggactgaggtattcgtattctcagttgacaaccttaaagccaactcttccggggcaatcaaattcggccccagtctatcgcaatgcccagcgctttcagacggaatactcaagtcctaccatcgttacaagatcacaagtatccgagttgagtttaagtcacacgcgtccgccactacggccggcgctatctttattgaactcgacaccgcgtgcaagcaatcagccctgggtagctacattaactcattcaccatcagcaagaccgcctccaaggtcttccggtcagaggcaattaacgggaaggaattccaggaatcaacgatagaccaattttggatgctctacaaggccaatggaaccaccactgacacggcaggacaattcatcattacgatgagtgtcagtttgatgacggccaaatag

SEQ ID NO: 358 - DNA, 600 bp, Barley yellow dwarf
atgaattcagtaggccgtagaggacctagaagagcaaacaatggcacaagaaggcggcgccgtagagcaattcggccagtggttgtggtccaatccaatcgagcaggacccagacgacgaaatgctagacgcgcaagaggaagaaggccaaattctgtatttggaccaaaacgcggggctgaggtattcgtattctcagtcgacaaccttaaagccaattcctccgggatcatcaaattcggtcccgatttatcgcaatgcccagcggtttcagacggagtacttaagtcctaccacaattacaagatctcaagtgtcaacgttgagtttaagtcacacgcgtcctcaactacgtccggcgctatgtttattgaactcgacacctcgtgcaagcaatcagccttggctagctacattaactcattcaccatcagcaaatctgcctcaaagtccttcaaagcgacggagattggagggacccaattccaggcatcctcggtgaatcagttctttctactatataaagccaatggcaccacgagtgacactgcaggacagttcatcatcaaacttgaaatacatctgatgactgccaaatag

SEQ ID NO: 359 - DNA, 600 bp, Barley yellow dwarf
atgaattcagtaggccgtagaggacctagaagagcaaataatggcacacgaaggcggcgccgtagagcaattcggccagtggttgtggtccagcccaatcgaacaggacccagacgacgaaatgctcgacgcgcaagaggaagaaggccaggttctgtatttggaccaaaacgcggggctgaggtattcgtattctcagtcgacaaccttaaggccaactcctccgggatcctcaaattcggtcccgatttatcgcaatgcccagcggtttcagacggagtacttaagtcctaccacaattacaagatctcaagtatcaacgttgagtttagaacacacgcgtccgccactacgtcgggcgctatgtttattgaactcgacacctcgtgcaagcaatcagccttatctagctacattaactcattcaccatcagcaaatccgcctcaaagtccttccgcgcagcggagattggagggacccagttccaggcgacatcggtgaatcaattctttcttttatataaggcaaatggcactactactgatattgcagggcagttcatcatcagaattgagcttcatctgatgactgccaaatag

SEQ ID NO: 360 - DNA, 600 bp, Barley yellow dwarf
atgaattcagtaggccgtagaggacctagaagagcaaacaatggcacacaaaggcggcgccgtcgagcaattcggccagtggttgtggtccagcccaatcgaacaggacccagacgacgaaatgctagacgcgcaagaggaagaaggccaggttctgtatttggaccaaaacgcggggctgaggtattcgtattctcagtcgacaaccttaaggccaactcctccgggatcctcaaattcggtcccgatttatcgcaatgcccagcggtttcagacggagtacttaagtcctaccacaattacaagatctcaagtatcaacgttgagtttagaacacacgcgtccgccactacgtcgggcgctatgtttattgaactcgacacctcgtgcaagcaatcagccttatctagctacattaactcattcaccatcagcaaatccgcctcaaagtccttccgcgcagcggagattggagggacccagttccaggcgacatcggtgaatcaattctttcttttgtataaggcaaatggcactacatctgatattgcagggcagttcatcatcagaattgagcttcatctaatgactgccaaatag

SEQ ID NO: 361 - DNA, 600 bp, Barley yellow dwarf
atgaattcagtaggccgtagaggacctagaagagcaaacaatggcacacgaaggcggcgccgtcgagcaattcggccagtggttgtggtccagcccaatcgaacaggacccagacgacgaaatgctagacgcgcaagaggaagaaggccaggttctgtatttggaccaaaacgcggggctgaggtattcgtattctcagtcgacaaccttaaggccaactcctccgggatcctcaaattcggtcccgatttatcgcaatgcccagcggtttcagacggagtacttaagtcctaccacaattacaagatctcaagtatcaacgttgagtttagaacacacgcgtccgccactacgtcgggcgctatgtttattgaactcgacacctcgtgcaagcaatcagccttatctagctacattaactcattcaccatcagcaaatcagcctcaaagtccttccgcgcagcggagattggagggacccagttccaggcgacatcggtgaatcaattctttcttttgtacaaggcaaatggcactacggctgatattgcagggcagttcatcatcagaattgagcttcatctaatgactgccaaatag

SEQ ID NO: 362 - DNA, 139 bp, Barley yellow dwarf; identical to SEQ ID NO: 354

SEQ ID NO: 363 - DNA, 501 bp, Barley yellow dwarf
gagtcatattggcaacgcgtattgtgatgatgaattgcccggcggtgtccgacgtagtaccgttcgccttgtatagcatgtaaaattggttcaccgtgctctccctgaattccttcccgtcaatctgttgggcggtgaaggttttggttgctgattttgagatggtgaatgagttaatgtagctacccaaggttgattgtgagcacgaagtgtcgagttcaacaaacattgcgccgactgtattggcggacgcgtgtgacttaaactcaaccttgacgtttgtgatcttgtaacggtggtaggacttgagtattccacctgaaagcgctgggcattgcgaaaggtcgggaccgaacttgattgttcctgaggagttggccttaaggtcgttgactgagaatacgaatacctcagtcctgcctgctggtccagatataagatttgcccctcctcctcttgtgcgtcgacgagttcgtcgtttgggtccggctcgattgggtt

SEQ ID NO: 364 - DNA, 502 bp, Barley yellow dwarf
caacccaatcgagcaggacccagacgacgaaatggtcgacgcaagggaagaggaggggcaaatcctgtatttagaccaacaggcgggactgaggtattcgtattctcagttgacaaccttaaagccaactcctccggggcaatcaaattcggccccagtctatcgcaatgcccagcgctttcagacggaatactcaagtcctaccatcgttacaagatcacaagtatccgagttgagtttaagtcacacgcgtccgccactacggcaggcgctatctttattgagctcgacaccgcgtgcaagcaatcagccctgggtagctacattaattccttcaccatcagcaagaccgcctccaaaaccttccggtcagaggcaattaatgggaaggaattccaggaatcaacgatagaccaattctggatgctctacaaggccaatggaaccaccactgacacggcaggacaatttatcattacgatgagtgtcagtttgatgacgg

SEQ ID NO: 365 - DNA, 412 bp, Barley yellow dwarf
ctatcgtcgattcttggaattccttcccattaatcgcctcggctctgaaggacttggaggcagttttgctgatggtgaaggaattaatgtagctacccagggctgattgcttgcacgcggtgtcgagttcaacaaagatagcgcccgacgtagtggcggacgcgtgtgacttaaactcaacacggatacttgtgatcttgtaacggtggtaggacttaagtattccgtctgaaagcgctgggcattgcgatagactgggaccgaatttgattgtcccggaagagttggctttaaggttgtcgactgagaatatgaatacctcagtcctgcctgttgatccaggtatagaatttggccctcttcttcctgtgcgtcgaccatttcgtcgtctgggtcctgctcgattgggttg

SEQ ID NO: 366 - DNA, 412 bp, Barley yellow dwarf
caacccaatcgagccggacccaaacgacgaactcgtcgacgcacaagaggaggaggggcaaatcttatatctggaccagcaggcaggactgaggtattcgtattctcagtcaacgaccttaaggccaactcctcagggacaatcaagttcggtcccgacctttcgcaatgcccagcgctttcaggtggaatactcaagtcctaccaccgttacaagatcacaaacgtcaaggttgagtttaagtcacacgcgtccgccaatacagtcggcgcaatgtttgttgaactcgacacttcgtgctcacaatcaaccttgggtagctacattaactcattcaccatctcaaaatcagcaaccaaaaccttcaccgcccaacagattgacgggaaggaattcagggagagcacggtga

SEQ ID NO: 367 - DNA, 502 bp, Barley yellow dwarf
caacccaatcgagccggacccaaacgacgaactcgtcgacgcacaagaggaggaggggcaaatcttatatctggaccagcaggcaggactgaggtatttgtattctcagtcaacgaccttaaggccaactcctcagggacaatcaagttcggtcccgacctttcgcaatgcccagcgctttcaggtggaatactcaagtcctaccaccgttacaagatcacaaacgtcaaggttgagtttaagtcacacgcgtccgccaatacagtcggcgcaatgtttgttgaactcgacacttcgtgctcacaatcaaccttgggtagctacattaactcattcaccatctcaaaatcagcaaccaaaaccttcaccgcccaacagattgacgggaaggaattcagggagagcacggtgaaccaattttacatgctatacaaggcgaacggtactacgtcggacaccgccgggcaattcatcatcacaatacgcgttgccaatatgactc

SEQ ID NO: 368 - DNA, 490 bp, Barley yellow dwarf
tgacactcatcgtaatgataaattgtcctgccgtgtcagtggtggttccattggccttgtagagcatccaaaattggtctatcgttgattcctggaattccttcccattaattgcctctgaccggaaggtcttggaggcggtcttgctgatggtgaaggaattaatgtagctacccaaggctgattgcttgcacgcggtgtcgagctcaataaagatagcgcctgccgtagtggcggacgcgtgtgacttaaactcaactcggatacttgtgatcttgtaacgatggtaggacttgagtattccgtctgaaagcgctgggcattgcgatagactggggccgaatttgattgccccggaggagttggctttaaggttgtcaactgagaatacgaatacctcagtcccgcctgttggtctaaatacaggatttgcccctcctcttcccttgcgtcgaccatttcgtcgtctgggtcctgctcgattgggttg

SEQ ID NO: 369 - DNA, 502 bp, Barley yellow dwarf
ccgtcatcaaactgacactcatcgtaatgataaattgtcctgccgtgtcagtggtggttccattggccttgtagagcatccaaaattggtctatcgttgattcctggaattccttcccattaattgcctctgaccggaaggtcttggaggcggtcttgctgatggtgaaggaattaatgtagctacccagggctgattgcttgcacgcggtgtcgagctcaataaagatagcgcctgccgtagtggcggacgcgtgtgacttaaactcaactcggatacttgtgatcttgtaacgatggtaggacttgagtattccgtctgaaagcgctgggcattgcgatagactggggccgaatttgattgccccggaggagttggctttaaggttgtcaactgagaatacgaatacctcagtcccgcctgttggtctaaatacaggatttgcccctcctcttcccttgcgtcgaccatttcgtcgtctgggtcctgctcgattgggttg

SEQ ID NO: 370 - DNA, 593 bp, Barley yellow dwarf; identical to SEQ ID NO: 355

SEQ ID NO: 371 - DNA, 593 bp, Barley yellow dwarf
atgaatttagtaggccgtagaaataaccgcaggagaaatggcccaaggagagcaaggcgcgttagcgcagttcggagaatggttgtggtccaacccaatcgagccggacccaaacgacgaactcgtcgacgcacaagaggaggaggggcaaatcttatatctggaccagcaggcaggactgaggtattcgtattctcagtcaacgaccttaaggccaactcctcaggaacaatcaagttcggtcccgacctttcgcaatgcccagcgctttcaggtggaatactcaagtcctaccaccgttacaagatcacaaacgtcaaggttgagtttaagtcacacgcgtccgccaatacagtcggcgcaatgtttgttgaactcgacacttcgtgctcacaatcaaccttgggtagctacattaactcattcaccatctcaaaatcagcaaccaaaaccttcaccgcccaacagattgacgggaaggaattcagggagagcacggtgaaccaattttacatgctatacaaggcgaacggtactacgtcggacaccgccgggcaattcatcatcacaatacgcgttgccaatatgactcc

SEQ ID NO: 372 - DNA, 603 bp, Barley yellow dwarf
atgaattcagtaggccgtagaggacctagacgcgcaaatcaaaatggcacaagaaggaaacgccgtagaacagttcggccagtggttgtggtccaacccaatcgagcaggacccagacgacgaaatggtcgacgcaagggaagaggaggggcaaatcctgtatttagaccaacaggcgggactgaggtattcgtgttctcagtcgataaccttaaagccaactcttccggggcaatcaaattcggccccagtctatcgcaatgcccagcgctttcagacggaatacttaagtcctaccaccgttacaagatcacaagtatccgtgttgagtttaagtcacacgcgtccgcaactacggccggcgctatctttgttgaactcgacaccgcgtgcaaacaatcagccctggctagctacattaattccttcacaatcagcaggaccgcctcaaaggtcttcagagccgaagcgattaacggcaaggaattccaggaatcaacgatagaccagttttggatgctctacaaggccaatggaactaccactgacacggcaggacaattcattatcacgatgagtgtcagtttgatgacggccaaatag

SEQ ID NO: 373 - DNA, 596 bp, Barley yellow dwarf
atgaattcagtaggccgtagaggacctagacgcgcaaatcaaaatggcccaagaaggcggcgccgtagaacagttcggccagtggttgtggtccaacccaatcgagcaggacccagacgacgaaatggtcgacgcaagggaggaggaggggcaaatactgtatttagaccaacaggcgggactgaggtattcgtattctcagttgacaacattaaagccaactcctccggggcaatcaaattcggccccagtctatcgcaatgcccagcgctttcagacggaatactcaagtcctaccatcgttacaagatcacaagtatccgagttgagtttaagtcacacgcgtccgccactacggcaggcgctatctttattgagctcgacaccgcgtgcaagcaatcagccctgggtagctacattaattccttcaccatcagcaagaccgcctccaagaccttccggtcagaggcaattaatgggaaggaattccaggaatcaacgatagaccaattttggatgctctacaaggccaacgaaaccaccaccgacacggcaggacaatttatcattacgatgagagtcagtttgatgacggc

SEQ ID NO: 374 - DNA, 462 bp, Barley yellow dwarf
atggcacaagaaggaggcgccgtagaacagttcggccagtggttgtggtccaacccaatcgagcaggacccagacgacgaaatggtcgacgcaagggaagaggaggggcaaatcctgtatttagaccaacaggcgggactgaggtattcgtattctcagttgacaaccttaaagccaactcctccggggcaatcaaattcggccccagtctatcgcaatgcccagcgctttcagacggaatactcaagtcctaccatcgttacaagatcacaagtatccgagttgagtttaagtcacacgcgtccgccactacggcaggcgctatctttattgagctcgacaccgcgtgcaagcaatcagccctgggtagctacattaattccttcaccatcagcaagaccgcctccaagaccttccggtcagaggcaattaatgggaaggaattccaggaatcaacgatag

SEQ ID NO: 375 - DNA, 462 bp, Barley yellow dwarf
atggcacaagaaggaggcgccgtagaacagttcggccagtggttgtggtccaacccaatcgagcaggacccagacgacgaaatggtcgacgcaagggaagaggaggggcaaatcctgtatttagaccaacaggcggggctgaggtattcgtattctcagtcgacaaccttaaagccaactcttccggggcaatcaaattcggccccagtctatcgcaatgcccagcgctttcagacggaatacttaagtcctaccaccgttacaagatcacaagtatccgtgttgagtttaagtcacacgcgtccgcaacaacggccggcgctatctttattgaactcgacaccgcgtgcaaacaatcagccctggctagctacattaattccttcacaatcagcaagaccgcctcaaaggtcttcagagccgaagcgattaacgggaaggaattccaggaatcaacgatag

SEQ ID NO: 376 - DNA, 462 bp, Barley yellow dwarf; identical to SEQ ID NO: 375

SEQ ID NO: 377 - DNA, 462 bp, Barley yellow dwarf
atggcacaagaaggcggcgccgtagagcagttcggccagtggttgtggtccaacccaatcgaacaggacccagacgacgaaatgctagacgcgcaagaggaagaaggccaaattctgtatttggaccaaaacgcggggctgaggtattcgtattctcagtcgacaaccttaaggccaactcctccgggatcctcaaattcggtcccgatctatcgcaatgcccagcggtttcagacggagtacttaagtcctaccacaattacaagatctcaagtatcaacgttgagtttagaacacacgcgtccgccactacgtcgggcgctatgtttattgagctcgacacctcgtgcaagcaatcagccttatctagctacattaactcattcaccatcagcaaatcagcctcaaagtccttccgcgcagcggagattggagggacccagttccaggcgacatcggtga

SEQ ID NO: 378 - DNA, 400 bp, Barley yellow dwarf
gcaggacccagacgacgaaatggtcgacgcaagggaagaggaggggcaaatcctgtatttagaccaacaggcgggactgaggtattcgtattctcagttgacaaccttaaagccaactcctccggggcaatcaaattcggccccagtctatcgcaatgcccagcgctttcagacggaatactcaagtcctaccatcgttacaagatcacaagtatccgagttgagtttaagtcacacgcgtccgccactacggcaggcgctatctttattgagctcgacaccgcgtgcaagcaatcagccctgggtagctacattaattccttcaccatcagcaagaccgcctccaagaccttccggtcagaggcaattaatgggaaggaattccaggaatcaacgatag

SEQ ID NO: 379 - DNA, 192 bp, Barley yellow dwarf
atggaggatcttcacgttatcgccgtttgtattcttgctttgactgtgctctctggggtaggcgctgttttgagttgctgccgttggtgctgcagcaatccttttcctccctccctctcttctgttcaagcaaaagactctcgatctgtgcgagagacaatcaaaaatatcgagggagcttcggctcagtga

SEQ ID NO: 380 - DNA, 126 bp, Barley yellow dwarf
atggatgacctccatgttatcgccgtttgtatttttgctctaactgtgttgacagggttaggcgcggtgatcggctgttgcgccggttgccttctcccccctccctccttccgctcttctgtttaa

SEQ ID NO: 381 - DNA, 129 bp, Barley yellow dwarf
atggatgacctccacgttatcgccgtttgtatgcttgctctgaccgtgttgacagggttaggcgctgtagtcggctgctgtgtcggctgcatccaatcccccttcccttccccccgcccttccttttaa

SEQ ID NO: 382 - DNA, 129 bp, Barley yellow dwarf
atggaagatcttcacgttatcgccgcttgtatgcttgctttgactgtgctctcaggggtaggcgctatcctgagctgttgcaaatggtgcttcagctcttcctttccctccccccgcgcttctctttag

SEQ ID NO: 383 - DNA, 120 bp, Barley yellow dwarf
atggatgatctccatgtcattgctgtttgtatgcttgccatgactactttcacagcagtgggagttgtgcctggttgctgcattggttgtattgaagccctttgtggcagtaaacgctaa

SEQ ID NO: 384 - DNA, 123 bp, Barley yellow dwarf
atggatgatcttcgcgttatcgccctctgtgctctgtcaccaactatactgtttaccattgtgttggtgagtagttggtgtgcaagctgttgtaagttcatagacgcagcttgttctgtgtaa

SEQ ID NO: 385 - DNA, 87 bp, Barley yellow dwarf
atggatgacctccacgttatcgccgtttgtatttttttttttgctctaactgtgctgacagggttaggcgcggtgatcggctgctga

SEQ ID NO: 386 - DNA, 777 bp, Tomato yellow leaf curl virus
atgtcgaagcgactaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcacactggtattgttcgttgtgttagtgatgttactcgtggatctggaattactcatagagtgggtaagaggttctgtgttaaatcgatatattttttaggtaaagtctggatggatgaaaatatcaagaagcagaatcacactaatcaggtcatgttctttttggtccgtgatagaaggccctatggaagcagcccaatggattttggacaggtttttaatatgttcgataatgagcccagtaccgcaaccgtgaagaatgatttgcgtgataggtttcaagtgatgagaaaatttcatgcaacagttattggtgggccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttctatgattcaatatcaaattaa

SEQ ID NO: 387 - DNA, 777 bp, Tomato yellow leaf curl virus
atgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttagggaaagtctggatggatgaaaatatcaagaagcagaatcacactaatcaggtcatgttcttcttagtccgtgatagaaggccctatggaagcagcccaatggattttggacaggtttttaatatgttcgataatgagcccagtaccgcaaccgtgaagaatgatttgcgggataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttactgtatatggcatgtacgcatgcctcgaatccagtgtatgcaactatgaaaatacgcatctatttctatgattcaatatcaaattaa

SEQ ID NO: 388 - DNA, 777 bp, Tomato yellow leaf curl virus
atgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttagggaaagtctggatggatgaaaatatcaagaagcagaatcacactaatcaggtcatgttcttcttagtccgtgatagaaggccctatggaagcagcccaatggattttggacaggtttttaatatgttcgataatgagcctagtaccgcaaccgtgaagaatgatttgcgggataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttactgtatatggcatgtacgcatgcctcgaatccagtgtatgcaactatgaaaatacgcatctatttctatgattcaatatcaaattaa

SEQ ID NO: 389 - DNA, 777 bp, Tomato yellow leaf curl virus
atgtcgaagcgaccaggcgatataatcatttccacgcccgtctcgaaggttcgccgaaggctgaacttcgacagcccatacagcacccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttagggaaagtctggatggatgaaaatatcaagaagcagaatcacactaatcaggtcatgttcttcttagtccgtgatagaaggccctatggaagcagcccaatggattttggacaggtttttaatatgttcgataatgagcccagtaccgcaaccgtgaagaatgatttgcgggataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttactgtatatggcatgtacgcatgcctcgaatccagtgtatgcaactatgaaaatacgcatctatttctatgattcaatatcaaattaa

SEQ ID NO: 390 - DNA, 777 bp, Tomato yellow leaf curl virus
atgtcgaagcgaccaggcgatataatcatttccacgcccgtctcgaaagttcgccgaaggctgaacttcgacagcccatacagcaaccgtgctgctgtccccattgtccaaggcacaaacaagcgacgatcatggacgtacaggcccatgtaccgaaagcccagaatatacagaatgtatcgaagtcctgatgttccccgtggatgtgaaggcccatgtaaagtccagtcttatgagcagcgggatgatattaagcacactggtgttgttcgttgtgttagtgatgttactcgtggatctggaattactcacagagtcggtaagaggttctgtgttaaatcgatatattttttaggtaaagtctggatggatgaaaatatcaagaagcagaatcacactaatcaggtcatgttctttttggtccgtgatagaaggccctatggaagcagtccaatggattttggacaggtttttaatatgttcgataatgagcccagtaccgcaactgtgaagaatgatttgcgtgataggtttcaagtgatgaggaaatttcatgcaacagttattggtgggccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttctatgattcaatatcaaattaa

SEQ ID NO: 391 - DNA, 777 bp, Tomato yellow leaf curl virus
atgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttagggaaagtctggatggatgaaaatatcaagaagcagaatcacactaatcaggtcatgttcttcttagtccgtgatagaaggccctatggaagcagcccaatggattttggacaggtttttaatatgttcgataatgagcccagtaccgcaaccgtgaagaatgatttgcgggataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttttatgattcaatatcaaattaa

SEQ ID NO: 392 - DNA, 774 bp, Tomato yellow leaf curl virus
atgccgaagcgaacaggcgatatactaatgtcaagcccgctctcgaagtttcgtcgaaaactgaacttcgacagcccgtataccagccgtgctgctgcccccactgtccaaggcatcaagcgtcgatcatggacttacaggcccatgtatcgaaagccgcggatgtacagaatgtacagaagccctgatgtcccgtttggttgtgaaggtccttgtaaagtccagtcgtatgagcagcgtgacgacgtcaagcataccggtgttgttcgttgtgttagtgatgtaactaggggttctggtattacacatagagtaggtaaacggttttgtattaagtcaatctatattttagggaagatttggatggatgaaaatataaaaaaacaaaatcatactaaccaggtcatgttctttttagtacgagaccgaaggccgtatggaactagtcctatggattttggtcaagtttttaacatgtttgataatgaacctagtacggctactgtgaagaacgatttaagggataggtaccaagtaatgaggaagttccatgccacggtggtaggtggtccgtcagggatgaaggagcagtgtctgttgaagaggttttttaaagttaatacccatgtagtttataatcatcaagagcaggcgaagtatgaaaaccatactgagaatgcgttgttgttgtatatggcatgtactcatgcttctaacccagtgtatgctacgttgaaaatacgtatctatttttatgatgctgtaacaaattaa

SEQ ID NO: 393 - DNA, 783 bp, Tomato yellow leaf curl virus
atgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttaggtaaagtctggatggatgaaaatatcaagaagcagaatcacactaatcaggtcatgttcttcttggtccgtgatagaaggccctatggaaacagcccaatggattttggacaggtttttaatatgttcgataatgagcccagtaccgcaaccgtgaagaatgatttgcgtgataggtttcaagtgatgaggaaatttcatgctacagttattggtgggccctctggaatgaaggaacaggcattagttaagagattttttaaaattaacagtcatgtaactttatttatattcattcaggaggcagcaaagtacgagaaccatactgaaaacgccttgttattgtatatggcatgtacgcatgcctctaatccagtgtatgcaactatgaaaatacgcatctatttctatgattcaatatcaaattaa

SEQ ID NO: 394 - DNA, 774 bp, Tomato yellow leaf curl virus
atgccgaagcgaaccggcgatatactaatttcaacgcccgtctcgaaggttcgtcgaagactgaacttcgacagcccgtataccagccgtgctgctgcccccactgtccaaggcatcaagcgtcgatcatggacttacaggcccatgtatcgaaagccgcggatgtacagaatgtacagaagccctgatgtacctccgggttgtgaaggtccctgtaaagtgcagtcgtacgagcagcgtgatgacgtcaagcataccggtgttgtgcgttgtgttagtgatgtaactaggggttctggtattactcatagagttggtaaacgtttttgtatcaagtcaatttatatattaggaaagatttggatggatgaaaacataaaaaaacaaaatcatactaaccaagtgatgtttttccttgttcgagaccgaaggccttatggaactagtcctatggattttggtcaagtttttaacatgtttgataatgaacccagtactgctacggtgaagaacgacttacgggataggtatcaagtaatgaggaagtttcatgctacggttgttggaggtccgtcagggatgaaggagcagtgtttgctgaagagattttttaaaattaatacccatgtagtttataatcaccaagagcaggcgaagtatgaaaatcatactgagaatgccttgttattgtatatggcttgtactcatgcttctaacccagtgtacgctacgttgaaaatacgtatttatttttatgatgctgtaacaaattaa

SEQ ID NO: 395 - DNA, 351 bp, Tomato yellow leaf curl virus
atgtgggacccacttctaaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctga

SEQ ID NO: 396 - DNA, 771 bp, Tomato yellow leaf curl virus
atgtcgaagcgtccagcagatattctcatttccacgcccgtctcgaaagtacgtcgccgtctgaacttcgacagcccatacaacagccgtgctgctgtccccactgtccgcgtcacaaaagggcagatatggaagaaccgacctgcatacagaaagcccaggttctacagaatgtatagaagtcctgatgtccctaagggatgtgagggtccatgtaaagtgcaatctttcgatgcgaagaacgacattggtcatatgggcaaggtaatctgtctgtctgacgttacccgtggtattgggcttactcatcgagttggcaagcgtttctgtgtcaagtcactttattttgtcgggaagatctggatggatgaaaatattaaggttaagaatcacactaataccgttttattttggatagttagggatcggcgtcctactggaacgcctaatgattttcagcaggtctttaatgtatatgataatgaacccagcactgctactgtaaagaacgaccagcgtgatcgtttccaggttataaggaggtttcaggcaacggtgactggtggacaatatgcagctaaggagcaggcgattattagaaagttttatcgtgttaataattatgtagtttacaatcaccaggaagctgggaagtacgagaaccatactgaaaatgctttgttgttgtatatggcatgtactcatgcctctaatcctgtgtatgctactttgaaagtcagaagttatttctatgactcagtgacgaattaa

SEQ ID NO: 397 - DNA, 777 bp, Tomato yellow leaf curl virus
atgtcgaagcgaccaggcgatataatcatttccacgcccgtctcgaaggttcgccgaaggctgaacttcgacagcccatacagcaaccgtgctgctgtccccattgtccaaggcacaaacaagcgacgatcatggacgtacaggcccatgtaccgaaagcccagaatatacagaatgtatcgaagtcctgatattccccgtggatgtgaaggcccatgtaaagtccagtcttatgagcagcgggatgatattaagcacactggtgttgttcgttgtgttagtgatgttactcgtggatctggaattactcacagagtcggtaagaggttctgtgttaaatcgatatattttttaggtaaagtctggatggatgaaaatatcaagaagcagaatcacactaatcaggtcatgttctttttggtccgtgatagaaggccctatggaagcagtccaatggattttggacaggtttttaatatgttcgataatgagcccagtactgcaactgtgaagaatgatttgcgtgataggtttcaagtgatgaggaaatttcatgctacagttattggtgggccctctggaatgaaggaacaggcattagttaagagattttttaaaattaacagtcatgtaacttataatcatcaggaggcagccaagtacgagaaccatactgaaaacgccttgttattgtatatggcatgtacgcatgcctctaatccagtgtatgcaactatgaaaatacgcatctatttctatgattcaatatcaaattaa

SEQ ID NO: 398 - DNA, 351 bp, Tomato yellow leaf curl virus; identical to SEQ ID NO: 395

SEQ ID NO: 399 - DNA, 351 bp, Tomato yellow leaf curl virus; identical to SEQ ID NO: 395

SEQ ID NO: 400 - DNA, 351 bp, Tomato yellow leaf curl virus; identical to SEQ ID NO: 395

SEQ ID NO: 401 - DNA, 351 bp, Tomato yellow leaf curl virus; identical to SEQ ID NO: 395

SEQ ID NO: 402 - DNA, 351 bp, Tomato yellow leaf curl virus; identical to SEQ ID NO: 395

SEQ ID NO: 403 - DNA, 351 bp, Tomato yellow leaf curl virus
atgtgggacccacttctaaatgaatttcctgaatctgttcacggatttcgttgtatgttagctattaaatatttgcagtccgttgaggaaacttacgagcccaatacattgggccacgatttaattagagatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctga

SEQ ID NO: 404 - DNA, 351 bp, Tomato yellow leaf curl virus
atgtgggacccacttctaaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtttcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctga

SEQ ID NO: 405 - DNA, 351 bp, Tomato yellow leaf curl virus
atgtgggatccacttctaaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccggaagcccagaatatacagaatgtatcgaagccctga

SEQ ID NO: 406 - DNA, 351 bp, Tomato yellow leaf curl virus
atgtgggacccacttcttaatgagtttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctga

SEQ ID NO: 407 - DNA, 351 bp, Tomato yellow leaf curl virus
atgtgggacccacttctaaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcaaccgtgctgctgtccccattgtccaaggcacaaacaagcgacgatcatggacgtacaggcccatgtaccgaaagcccagaatatacagaatgtatcgaagtcctga

SEQ ID NO: 408 - DNA, 351 bp, Tomato yellow leaf curl virus; identical to SEQ ID NO: 406

SEQ ID NO: 409 - DNA, 351 bp, Tomato yellow leaf curl virus; identical to SEQ ID NO: 406

SEQ ID NO: 410 - DNA, 351 bp, Tomato yellow leaf curl virus; identical to SEQ ID NO: 405

SEQ ID NO: 411 - DNA, 351 bp, Tomato yellow leaf curl virus; identical to SEQ ID NO: 407

SEQ ID NO: 412 - DNA, 351 bp, Tomato yellow leaf curl virus; identical to SEQ ID NO: 407

SEQ ID NO: 413 - DNA, 408 bp, Tomato yellow leaf curl virus
ctaaatactcttaagaaacgaccagtctgaggctgtaatgtcgtccaaattcggaagttgagaaaacatttgtgaatccccaataccttcctgatgttgtggttgaatcttatctgaatggaaatgatgtcgtggttcattagaaatggccgctggctgtgttctgttatcttgaaatagaggggattgtttatctcccagataaaaacgccattctctgcctgaggagcagtgatgagttcccctgtgcgtgaatccatgattgttgcagttgaggtggaggtagtatgagcagccacagtctaggtctacacgcttacgccttattggtttcttcttggctatcttgtgttggaccttgattgatacttgcgaacagtggttcgtagagggtgacgaaggttgcat

SEQ ID NO: 414 - DNA, 408 bp, Tomato yellow leaf curl virus
ctaaatactcttaagaaacgaccagtctgaggctgtaatgtcgtccaaattcggaagttgagaaaacatttgtgaatccccaataccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattgttgcagttgaggtggaggtagtatgagcagccacagtctaggtctacacgcttacgccttattggtttcttcttggctatcttgtgttggaccttgattgatacttgcgaacagtggctcgtagagggtgacgaaggttgcat

SEQ ID NO: 415 - DNA, 408 bp, Tomato yellow leaf curl virus; identical to SEQ ID NO: 414

SEQ ID NO: 416 - DNA, 408 bp, Tomato yellow leaf curl virus
ctaaatactcttaagaaatgaccagtctgaggatgtaatgtcgtccaaattcggaagttgagaaaacatttgtgaatccccatgaccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcat

SEQ ID NO: 417 - DNA, 408 bp, Tomato yellow leaf curl virus
ctaaatactcttaagaaacgaccagtctgaggctgtaatgtcgtccaaattcggaagttgagaaaacatttgtgaatccccattaccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgagatggaggtagtatgagcagccacagtctaggtctacacgcttacgccttattggtttcttcttggctatcttgtgttggaccttgattgatacttgcgaacagtggctcgtagagggtgacgaaggttgcat

SEQ ID NO: 418 - DNA, 408 bp, Tomato yellow leaf curl virus
ctaaatactcttaagaaacgaccagtctgaggctgtaatgtcgtccaaattcggaagttgagaaaacatttgtgaatccccaataccttcctgatgttgtggttgaatcttatctgaatggaaatgatgtcgtggttcattagaaatggcctctggctgtgttctgttatcttgaaatagaggggattgtttatctcccagataaaaacgccattctctgcttgaggagcagtgatgagttcccctgtgcgtgaatccatgattgttgcagttgatgtggaggtagtatgagcagccacagtctaggtctacacgcttacgccttattggtttcttcttggctatcttgtgttggaccttgattgatacttgcgaacagtggctcgtagagggtgacgaaggttgcat

SEQ ID NO: 419 - DNA, 408 bp, Tomato yellow leaf curl virus; identical to SEQ ID NO: 418

SEQ ID NO: 420 - DNA, 408 bp, Tomato yellow leaf curl virus
ctaaatactcttaagaaacgaccagtctgaggctgtaatgtcgtccaaattcggaagttgagaaaacatttgtgaatccccattaccttcctgatgttgtggttgaatcttatctgaatggaaatgatgtcgtggttcattagaaatggcctctgactgtgttctgttatcttgaaatagaggggattgtttatctcccagataaaaacgccattctctgcctgaggagcagtgatgagttcccctgtgcgtgaatccatgattattgcagttgaggtcgaggtagtatgagcatccacagtctaggtctacacgcttacgccttattggtttcttcttggctatcttgtgttggaccttgattgatacttgcgaacagtggctcgtagagggtgacgaaggttgcat

SEQ ID NO: 421 - DNA, 408 bp, Tomato yellow leaf curl virus
ctaaatactcttaagaaacgaccagtctgaggctgtaatgtcgtccaaattcggaagttgagaaaacatttgtgaatccccattaccttcctgatgttgtggttgaatcttatctgaatggaaatgatgtcgtggttcattagaaatggcctcgggctgtggtctgttatcttgaaatagaggggattgtttatctcccagataaaaacgccattctttgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcat

SEQ ID NO: 422 - DNA, 771 bp, Cotton leaf curl virus
atgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaagcccaggatgtacaggatgtacagaagtccagatgttcctagaggatgtgaaggtccatgtaaggttcagtcgtttgagtccagacatgatattcagcatataggtaaagtaatgtgtgttagtgatgttactcgtggtactgggctgacccatagagttggtaagagattttgtgtcaagtctgtttatgtgttgggtaagatatggatggatgagaacattaagacgaagaatcacacgaatagtgtgatgtttttcttggttagagatcgtagacctgttgataaacctcaagattttggagaggtatttaatatgtttgataatgagcccagtacggcgactgtgaagaatgttcatcgtgataggtatcaagttctgcgcaaatggtatgcaactgtcaccggtggacaatacgcttcaaaggaacaggctttggtcaagaagtttgtcagagttaacaattatgttgtttacaatcaacaggaagcaggaaaatacgagaatcatacggaaaatgcgttaatgctttatatggcttgtactcacgctagcaaccctgtttatgctacgttgaagattaggatatatttttatgactctgtaacgaattga

SEQ ID NO: 423 - DNA, 771 bp, Cotton leaf curl virus
atgtcgaagcgagctgcagatatcgtcatttctacgcccgcgtcgaaagtacgccggcgtctgaacttcggcagcccatacaccaaccgtgttgctgcccccattgtccgcgtcacaaaacaacaggcatggacaaacaggcctatgaacaggaagcccagaatgtatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatattaagaccaagaatcacacgaattcggtgatgttctttttagtccgcgatcgacgtcctgttgacaaacctcaggattttggtgaggtattcaatatgtttgacaacgaacccagtacagcaactgtgaagaatagtcatagggaccgttaccaggtgttgaggaaatggcatgcaaccgttacgggtggtcaatatgctagtaaggaacaggctttggtcaagaagtttgtcagagttaacaattatgttgtttacaatcaacaggaagcagggaaatacgagaatcatacggaaaatgcgttaatgctttatatggcttgtactcacgctagcaaccctgtttatgctacgttggagattaggatatatttttatgactctgtaacgaattga

SEQ ID NO: 424 - DNA, 771 bp, Cotton leaf curl virus
atgtcgaagcgagctgcagatatcgtcatttctacgcccgcgtcgaaagtacgccggcgtctgaacttcggcagcccatacaccaaccgtgttgctgcccccattgtccgcgtcacaaaacaacaggcatggacaaacaggcctatgaacaggaagcccagaatgtatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatattaagaccaacaatcacacgaattcggtgatgttctttttagtccgcgatcgacgtcctgttgacaaacctgaggattttggtgaggtattcaatatgtttgacaacgaacccagtacagcaactgtgaagaatagtcatagggaccgttaccaggtgttgaggaaatggcatgcaaccgttacgggtggtcaatatgctagtaaggaacaggctttggtcaagaagtttgtcagagttaacaattatgttgtttacaatcaacaggaagcaggaaaatacgagaatcacaccgagaatgcattgatgctttacatggcttgtactcacgctagcaaccctgtttatgctacgttgaagattaggatatatttttatgactctgtaacgaattga

SEQ ID NO: 425 - DNA, 771 bp, Cotton leaf curl virus
atgtcgaagcgagctgccgatatcgtcatttctacgcccgcgtcgaaagtacgccggcgtctgaacttcggcagcccatacaccagccgtgttgctgcccccattgtccgcgtcacaaaacaacaggcatggacaaacaggcctatgaacaggaagcccaggatgtacaggatgtacagaagtccagatgttcctagaggatgtgaaggtccatgtaaggttcagtcgtttgagtcgagacatgatgtcgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaagaatcacacgaattcggtgatgttctttttagtccgcgatcgacgtcctgttgacaaacctcaggattttggtgaggtattcaatatgtttgacaacgaacctagtacagcaactgtgaagaatagtcatagggaccgttaccaggtgttgaggaaatggcatgcaaccgttacgggtggtcaatatgctagtaaggaacaagctttggtcaagaagtttgtcagagttaacaattatgttgtttacaatcaacaggaagcagggaaatacgagaatcatacggaaaatgcgttaatgctatatatggcttgtactcacgctagcaaccctgtttatgctacgttgaagattaggatatatttttatgactctataacgaattga

SEQ ID NO: 426 - DNA, 771 bp, Cotton leaf curl virus
atgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaagcccagaatgtatcggatgtacagaagtccggatgttccaaagggttgtgaaggcccatgtaaggtacaatcttttgagtcgagacatgatgtcgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaagaatcacacgaattcggtgatgttctttttagtccgcgatcgacgtcctgttgacaaacctcaggattttggtgaggtattcaatatgtttgacaacgaacctagtacagcaactgtgaagaatagtcatagggaccgttaccaggtgttgaggaaatggcatgcaaccgttacgggtggtcaatatgctagtaaggaacaagctttggtcaagaagtttgtcagagttaacaattatgttgtttacaatcaacaggaagcagggaaatacgagaatcatacggaaaatgcgttaatgctttatatggcttgtactcacgctagcaaccctgtttatgctacgttgaagattaggatatatttttatgactctgtaacgaattga

SEQ ID NO: 427 - DNA, 771 bp, Cotton leaf curl virus
atgtcgaagcgagctgcagatatcgtcatttcaacgcccgcgtcgaaagtacgtcggcgtctgaacttcggcagcccatacaccaaccgtgttgctgtccccattgtccgcgtcacaaaacaacaggcatggacaaacaggcctatgaacaggaagcccagaatatatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaggaatcacacgaattcggtcatgttctttttagttcgcgatcgacgacctgttgacaaacctcaggattttggtgaggtattcaatatgtttgataacgaacccagtacagcaactgtgaagaatagtcatagggaccgttatcaggtgttgaggaaatggcatgcaaccgttacgggtggtcaatatgcgagtaaggaacaggctttggtcaagaagtttgtcagagttaacaattatgttgtttacaatcaacaggaagcaggaaaatacgagaatcatacggaaaatgcgttaatgctttatatggcttgtacccacgctagcaaccctgtttatgctacgttgaagattaggatatatttttatgactctgtaacgaattga

SEQ ID NO: 428 - DNA, 771 bp, Cotton leaf curl virus; identical to SEQ ID NO: 427

SEQ ID NO: 429 - DNA, 771 bp, Cotton leaf curl virus
atgtcgaagcgagctgccgatatcgtcatttctacgcccgcgtcgaaagtacgtcggcgtctgaacttcggcagcccatacaccagccgtgttgctgcccccattgtccgcgtcacaaagcaacaggcatggacaaacagacctatgaacaggaagcccagaatgtatcggatgtacagaagtccggatgttccaaggggttgtgagggtccctgtaaggtacaatcgtttgagtccagacatgatgttgtccatattggtaaggtgatgtgtatttctgatgttacgcgtggagtcggtttgacccatcgtattggtaaacgtttttgtgtcaagtcagtttatgttttaggtaagatatggatggatgaaaatataaagaccaagaatcacacgaattctgtgatgttcttcttagtccgcgaccgtcgtcctgttgacaaacctcaggattttggtgaggtattcaatatgtttgacaacgaacccagtacagcaactgtggagaatagtcatagggatcgttaccaggtgttgaggaaatggcatgcaaccgtcacgggtggtcaatatgcgagtaaggaacaggctttggtcaagaagtttgtcagagttaacaattatgttgtttacaaccagcaggaggcaggaaaatacgagaatcacaccgagaatgcattgatgctttatatggcttgtacccatgctagtaacccagtttatgctacgcttaagattcggatttatttttatgactctgtaacgaattga

SEQ ID NO: 430 - DNA, 771 bp, Cotton leaf curl virus
atgtcgaagcgagctgcagatatcgtcatttcaacgcccgcgtcgaaagtacgtcggcgtctgaacttcggcagcccatacaccaaccgtgttgctgtccccattgtccgcgtcacaaaacaacaggcatggacaaacaggcctatgaacaggaagcccagaatatatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaggaatcacacgaattcggtcatgttctttttagttcgcgatcgacgacctgttgacaaacctgaggattttggtgaggtattcaatatgtttgataacgaacccagtacagcaactgtgaagaatagtcatagggaccgttatcaggtgttgaggaaatggcatgcaaccgttacgggtggtcaatatgcgagtaaggaacaggctttggtcaagaagtttgtcagagttaacaattatgttgtttacaatcaacaggaagcaggaaaatacgagaatcatacggaaaatgcgttaatgctttatatggcttgtacccacgctagcaaccctgtttatgctacgttgaagattaggatatatttttatgactctgtaacgaattga

SEQ ID NO: 431 - DNA, 771 bp, Cotton leaf curl virus
atgtcgaagcgagctgcagatatcgtcatttcaacgcccgcgtcgaaagtacgtcggcgtctgaacttcggcaacccatacaccaaccgtgttgctgtccccattgtccgcgtcacaaaacaacaggcatggacaaacaggcctatgaacaggaagcccagaatatatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatataaagacgaggaatcacacgaattcggtcatgttctttttagttcgcgatcgacgacctgttgacaaacctcaggattttggtgaggtattcaatatgtttgataacgaacccagtacagcaactgtgaagaatagtcatagggaccgttatcaggtgttgaggaaatggcatgcaaccgttacgggtggtcaatatgcgagtaaggaacaggctttggtcaagaagtttgtcagagttaacaattatgttgtttacaatcaacaggaagcaggaaaatacgagaatcatacggaaaatgcgttaatgctttatatggcttgtacccacgctagcaaccctgtttatgctacgttgaagattaggatatatttttatgactctgtaacgaattga

SEQ ID NO: 432 - DNA, 771 bp, Cotton leaf curl virus
atgtcgaagcgagctgcagatatcgtcatttcaacgcccgcgtcgaaagtacgtcggcgtctgaacttcggcagcccatacaccaaccgtgttgctgtccccattgtccgcgtcacaaaacaacaggcatggacaaacaggcctatgaacaggaagcccagaatatatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaggaatcacacgaattcggtcatgttctttttagttcgcgatcgacgacctgttgacaaacctcaggattttggtgaggtattcaatatgtttgataacgaacccagtacagcaactgtgaagaatagtcatagggaccgttatcaggtgttgaggaaatggcatgcaaccgttacgggtggtcaatatgcgagtaaggaagaggctttggtcaagaagtttgtcagagttaacaattatgttgtttacaatcaacaggaagcaggaaaatacgagaatcatacggaaaatgcgttaatgctttatatggcttgtacccacgctagcaaccctgtttatgctacgttgaagattaggatatatttttatgactctgtaacgaattga

SEQ ID NO: 433 - DNA, 771 bp, Cotton leaf curl virus
atgtcgaagcgagctgcagatatcgtcatttctacgcccgcgtcgaaagtacgtcggcgtctgaacttcggcagcccatacaccagccgtgctgctgcccccattgtccgcgtcacaaaacaacaggcatggacaaacaggcctatgaacaggaaacccagaatgtaccggatgtacagaagtccggatgttccaaggggttgtgaaggcccatgtaaggtacaatcgtttgagtccagacatgatgttgtccatattggtaaggtaatgtgtatttctgatgttacgcgtggagtcggtttgacccatcgtattggtaaacgtttttgtgtcaagtcagtttatgttttaggtaagatatggatggatgaaaatataaagaccaagaatcacacgaattctgtgatgttcttctttgtccgcgaccgtcgtcctgttgacaaacctcaggattttggtgaagtattcaatatgtttgacaacgaacccagtacagcaactgtgaagaatagtcatagggatcgttaccaggtgttgaggaaatggcatgcaaccgtcacgggtggtcaatatgcgagtaaggaacaggctttggtcaagaagtttgtcagagttaacaattatgttgtttacaaccagcaggaggcaggaaaatacgaaaatcacaccgagaatgcattgatgctttatatggcttgtacccatgctagtaacccagtttatgctacgcttaagattcggatatatttttatgactctgtaacgaattga

SEQ ID NO: 434 - DNA, 771 bp, Cotton leaf curl virus
atgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaaacccagaatgtaccggatgtacagaagtccggatgttccaaggggatgtgagggtccctgtaaggtacagtcgtttgaatctcgacacgatgtcgttcatattggtaaggtaatgtgtatttcggatgttacgcgtggagtcggtttgacccatcgtataggtaagcgtttttgtgtcaagtcagtttatgttttaggtaagatatggatggacgagaacatcaagaccaagaaccatacgaattcggtgatgtttttccttgttcgtgatcgacgaccggtagataaaccacaagattttggtgaagtatttaatatgtttgataacgagcccagtacggcgaccgtgaagaacatgcatagggatcggtaccaggtgttgaggaaatggcatgcaaccgttactggtggtcaatatgcgagtaaggagcaggcattggtcaagaagtttgttagggttaacaactacgttgtttacaaccagcaggaagcaggaaaatacgagaatcacaccgagaatgcattgatgctttatatggcttgtacccatgctagtaacccagtttatgctacgcttaagattagaatatatttttatgactctgtaacgaactaa

SEQ ID NO: 435 - DNA, 771 bp, Cotton leaf curl virus; identical to SEQ ID NO: 434

SEQ ID NO: 436 - DNA, 771 bp, Cotton leaf curl virus
atgtcgaagcgagctgcagatatcgtcatttcaacgcccgcgtcgaaagtacgtcggcgtctgaacttcggcagcccatacaccaaccgtgttgctgtccccattgtccgcgtcacaaaacaacaggcatggacaaacaggcctatgaacaggaagcccagaatatatcggatgtacagaagtccggatgttccaaagggttgtgaaggcccatgtaaggtacagtcttttgagtccagacatgatgttgttcatattggtacggtaatgtgtatttctgatgttactcgtggtgtcggtttgacccatcgtattggtaaacgtttttgtgtcaagtcagtttatgttttaggtaagatatggatggatgaaaatataaagaccaggaatcacacgaattcagtcatgttctttttagttcgcgatcgacgacctgttgacaaacctcaggattttggtgaggtattcaatatgtttgataacgaacccagtacagcaactgtgaagaatagtcatagggaccgttatcaggtgttgaggaaatggcatgcaaccgttacgggtggtcaatatgcgagtaaggaacaggctttggtcaagaagtttgtcagagttaacaattatgttgtttacaatcaacaggaagcaggaaaatacgagaatcatacggaaaatgcgttaatgctttatatggcttgtacccacgctagcaaccctgtttatgctacgttgaagattaggatatatttttatgactctgtaacgaattga

SEQ ID NO: 437 - DNA, 771 bp, Cotton leaf curl virus
atgtcgaagcgagctgcagatatcgtcatttcaacgcccgcgtcgaaagtacgtcggcgtctgaacttcggcagcccatacaccaaccgtgttgctgtccccattgtccgcgtcacaaaacaacaggcatggacaaacaggcctatgaacaggaagcccagaatatatcggatgtacagaagtccggatgttccaaagggttgtgaaggcccatgtaaggtacagtcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaggaatcacacgaattcggtcatgttctttttagttcgcgatcgacgacctgttgacaaacctcaggattttggtgaggtattcaatatgtttgataacgaacccagtacagcaactgtgaagaatagtcatagggaccgttatcaggtgttgaggaaatggcatgcaaccgttacgggtggtcaatatgcgagtaaggaacaggctttggtcaagaagtttgtcagagttaacaattatgttgtttacaatcaacaggaagcaggaaaatacgagaatcatacggaaaatgcgttaatgctttatatggcttgtacccacgctagcaaccctgtttatgctacgttgaagattaggatatatttttatgactttgtaacgaattga

SEQ ID NO: 438 - DNA, 771 bp, Cotton leaf curl virus
atgtcgaagcgagctgccgatatcgtcatttctacgcccgcgtcgaaagtacgccggcgtctgaacttcggcagcccatacaccagccgtgctgctgcccccattgtccgcgtcacaaaacaacaagcatggacaaacaggcctatgaacaggaagcccagaatgtatcggatgtacagaagtccggatgttccaaagggttgtgaaggcccatgtaaggtacaatcttttgagtcgagacatgatgtcgttcatattggtaaggtaatgtgtatttctgatgttactcgtggtgtcggtttgacccatcgtattggtaaacgtttttgtgtcaagtcagtttatgttttaggtaagatatggatggatgaaaatataaagaccaagaatcacacgaattcggtgatgttctttttagtccgcgatcgacgtcctgttgacaaacctcaggactttggtgaggtatttaatatgtttgacattgaacccagtacagcgactgtgaagaatagtcatagggaccgttaccaggtgttgaggaaatggcatgcaaccgttacgggtggtcaatatgcatcgaaggaacaggctttggtgaagaagtttatcagagttaacaattatgttgtttacaatcaacaggaagcaggaaaatacgagaatcatacagaaaatgcgttaatgctttatatggcttgtactcacgctagcaaccctgtttatgctacgttgaagattaggatatatttttatgactctgtaacgaattga

SEQ ID NO: 439 - DNA, 420 bp, Cotton leaf curl virus
atgtcgaagcgagctgcagatatcgtcatttctacgcccgcgtcgaaagtacgtcggcgtctgaacttcggcagcccatacaccaaccgtgttgctgcccccattgtccgcgtcacaaaacaacaggcatggacaaacaggcctatgaacaggaagcccagaatgtatcggatgtacagaagtccggatgttccaaggggttgtgaaggcccatgtaaggtacaatcttttgagtccagacatgatgttgttcatattggtaaggtaatgtgtatttctgatgttactcgtggtgtcggtttgacccatcgtattggtaaacgtttttgtgtcaagtcagtttatgttttaggtaagatatggatggatgaaaatattaagaccaagaatcatacgaattcggtgatgttctttttagtc

SEQ ID NO: 440 - DNA, 280 bp, Cotton leaf curl virus
atgtgggatccactattaaacgaattccctgatacggttcacgggtttcggtgtatgctttctgtgaaatatttgcaacttttgtcgcaggattattcaccggatacgcttgggtacgagttaatacgggatttaatttgtattttacgctcccgtagttatgtcgaagcgagctgccgatatcgtcatttctacgcccgcgtcgaaagtacgccggcgtctgaacttcggcagcccatacaccagccgtgctgctgcccccattgtccgcgtcacaaaa

SEQ ID NO: 441 - DNA, 280 bp, Cotton leaf curl virus; identical to SEQ ID NO: 440

SEQ ID NO: 442 - DNA, 280 bp, Cotton leaf curl virus
atgtgggatccactattaaacgaattccctgatacggttcacgggtttcggtgtatgctttctgtgaaatatttgcaacttttgtcgcaggattattcaccggatacgcttgggtacgagttaatacgggatttaatttgtattttacgctcccgtaattatgtcgaagcgagctgccgatatcgtcatttctacgcccgcgtcgaaagtacgccggcgtctgaacttcggcagcccatacaccagccgtgctgctgcccccattgtccgcgtcacaaaa

SEQ ID NO: 443 - DNA, 357 bp, Cotton leaf curl virus
atgtgggatccactattaaacgaattccctgatacggttcacgggtttcggtgtatgctttctgtgaaatatttgcaacttttgtcgcaggattattcaccggatacgcttgggtacgagttaatacgggatttaatttgtattttacgctcccgtagttatgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaagcccaggatgtacaggatgtacagaagtccagatgttcctag

SEQ ID NO: 444 - DNA, 357 bp, Cotton leaf curl virus
atgtgggatccactattaaacgaattccctgatacggttcacgggtttcggtgtatgctttctgtgaaatatttgcaacttttgtcgcaggattattcaccggatacgcttgggtacgagttaatacgggatttaatttgtattttacgctcccgtaattatgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaagcccaggatgtacaggatgtacagaagtccagatgttcctag

SEQ ID NO: 445 - DNA, 357 bp, Cotton leaf curl virus
atgtgggatccactattaaacgaattccctgatacggttcacgggtttcggtgtatgctttctgtgaaatatttgcaacttttgtcgcaggattattcaccggatacgcttgggtacgagttaatacgggatttaatttgtattttgcgctcccgtaattatgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaagcccaggatgtacaggatgtacagaagtccagatgttcctag

SEQ ID NO: 446 - DNA, 366 bp, Cotton leaf curl virus
atgtgggatccactattaaacgaattccctgatacggttcacgggtttcggtgtatgctttctgtgaaatatttgcaacttttgtcgcaggattattcaccggatacgcttgggtacgagttaatacgggatttaatttgtattttacgctcccgtaattatgtcgaagcgagctgccgatatcgtcatttctacgcccgcgtcgaaagtacgccggcgtctgaacttcggcagcccatacaccagccgtgctgctgcccccattgtccgcgtcacaaaacaacaggcatggacaaacaggcctatgaacaggaagcccagaatgtatcggatgtacagaagtccggatgttccaaagggttgtga

SEQ ID NO: 447 - DNA, 280 bp, Cotton leaf curl virus
ttaattgaaattacaccgagattgttcagatatttgaggacttggtttttgaatacccttaagaaaagaccagtctgaggctgtaaggtcgtccagattcggaaggttagaaaacacttgtgcagtcccagagctttccgcgtgttgtagttgaactggatcctgatcgtgagtatgtccatattcgtcgtgaatggacggttgacgtggctgatgatcttgaaataaaggggatttggaacctcccagatatatgcgccattccctgcttgagctgcag

SEQ ID NO: 448 - RNA, 100 nt, Tomato spotted wilt virus
gucuaagguuaagcucacuaaggaaagcauuguugcuuuguugacacaaggcaaagaccuugaguuugaggaagaucagaaucugguagcauucaacuuc

SEQ ID NO: 449 - RNA, 100 nt, Tomato spotted wilt virus
gguagcauucaacuucaagacuuuuugucuggaaaaccuugaccagaucaaaaagaugagcauuauuucaugucugacauuccugaagaaucgucagagc

SEQ ID NO: 450 - RNA, 100 nt, Tomato spotted wilt virus
gaagaaucgucagagcauaaugaagguuauuaagcaaagugauuuuacuuuugguagaauuaccauaaagaaaacuucagacagaauuggagccacugac

SEQ ID NO: 451 - RNA, 102 nt, Tomato spotted wilt virus
gacagaauuggagccacugacaugaccuucagaaggcuugauagcuugaucagggucaggcuuguugaggaaacugggaauucugagaaucucaauacuauc

SEQ ID NO: 452 - RNA, 101 nt, Tomato spotted wilt virus
gaaucucaauacuaucaaaucuaagauugcuucccacccuuugauucaagccuauggauuaccucuugaugaugcaaagucugugaggcuugccauaaugc

SEQ ID NO: 453 - RNA, 102 nt, Tomato spotted wilt virus
gccauaaugcuaggagguagcuuaccucuuauugcuucaguugauagcuuugagaugaucaguguugucuuggcuauauaucaggaugcaaaauacaaggac

SEQ ID NO: 454 - RNA, 109 nt, Tomato spotted wilt virus
gcaaaauacaaggaccucgggaucgacccaaagaaguaugacaccagggaagccuuaggaaaaguuugcacugugcugaaaagcaaagcauuugaaaugaaugaagauc

SEQ ID NO: 455 - RNA, 102 nt, Tomato spotted wilt virus
gaaaugaaugaagaucaggugaagaaggggaaagaguaugcugcuauacuuagcuccagcaauccuaaugcuaaaggaaguauugcuauggaacauuacagc

SEQ ID NO: 456 - RNA, 98 nt, Tomato spotted wilt virus
gcuaaaggaaguauugcuauggaacauuacagcgaaacucuuaacaaguucuaugaaauguuugggguuaaaaaacaggcaaaacucacagaacuugc

SEQ ID NO: 457 - RNA, 104 nt, Tomato spotted wilt virus
guugacucuuuucgguaauaaggggucuucuaagucugccagaaaggaugaagguccuuuaguuucacuugcuaaacauaaugguaauguugaagucucuaagc

SEQ ID NO: 458 - RNA, 101 nt, Tomato spotted wilt virus
gaagucucuaagccauggucuucuucugaugaaaagcuugcuuugacuaaagcuauggauacauccaaaggaaagauacuguugaacacagagggaacauc

SEQ ID NO: 459 - RNA, 100 nt, Tomato spotted wilt virus
gaacacagagggaacaucuuccuuuggaaccuaugaaucugauucuaucacagaaucagaggguuaugaucuuucugcaagaaugauaguagauacaaac

SEQ ID NO: 460 - RNA, 102 nt, Tomato spotted wilt virus
guagauacaaaccaucauaucucaaacuggaaaaaugaucuuuuuguuggcaacgggaagcaaaacgcaaauaaggucaucaagaucuguccaacuugggac

SEQ ID NO: 461 - RNA, 100 nt, Tomato spotted wilt virus
guccaacuugggacagcagaaaacaauauaugaugauuuccaggauugugauaugggucugccccacuauaccaaacccuacagggaaacuugugguugc

SEQ ID NO: 462 - RNA, 97 nt, Tomato spotted wilt virus
gaaacuugugguugcccuggucgaucccaacaugccaucugaaaagcaagucauucugaagggucaggggacaauagcugauccuauauguuuuguc

SEQ ID NO: 463 - RNA, 102 nt, Tomato spotted wilt virus
gauccuauauguuuugucuuuuaucugaacuggucuauuccgaaaaugaauaacacuccagaaaacugcugucagcugcauuugaugugcagccaagaauac

SEQ ID NO: 464 - RNA, 98 nt, Tomato spotted wilt virus
gccaagaauacaagaaggggguuucuuuugguagugucauguauucuuggacaaaggaguuuugugauucacccagagcugauaaagauaaaaguugc

SEQ ID NO: 465 - RNA, 99 nt, Tomato spotted wilt virus
gauaaaaguugcauggucauaccucuaaacagagcuauuagagcuagaucucaagcauucauugaggcuugcaagcugauaauuccuaaaggaaacagc

SEQ ID NO: 466 - RNA, 110 nt, Tomato spotted wilt virus
ggaaacagcgagaagcagauuaaaaaacagcuuaaagaauugagcucaaaucuugagagaucaguugaagaagaagaggaagggauuucugauaguguugcucaguuauc

SEQ ID NO: 467 - RNA, 101 nt, Tomato spotted wilt virus
gucuucaaguguuuaugagucaaucauucagacaagagcuucagucuggggaucaacugcaucugguaaagcuguuguagauucuuacuggauucaugaac

SEQ ID NO: 468 - RNA, 101 nt, Tomato spotted wilt virus
gauucaugaacuugguacugguucucaacuaguucagacccagcuguauucugauucaagaagcaaaagcagcuuuagcuauacugcaaaaguaggagauc

SEQ ID NO: 469 - RNA, 98 nt, Tomato spotted wilt virus
guaggagaucuuccuugugaagaagaagagauucucucucagcauguguauauccccauuuuugaugauauugauuuuagcaucaauauugaugacuc

SEQ ID NO: 470 - RNA, 100 nt, Tomato spotted wilt virus
gaugacucuguucuggcacuaucuguuugcucaaauacagucaaugcuaauggagugaaacaucaaggucauuugaagguuuugucuccugcucagcucc

SEQ ID NO: 471 - RNA, 96 nt, Tomato spotted wilt virus
gcucagcuccacucuauuggaucuaucaugaacagaucugacauuacagaccgauuccagcuccaagaaaaagacauaauucccaaugacagauac

SEQ ID NO: 472 - RNA, 101 nt, Tomato spotted wilt virus
gacauaauucccaaugacagauacauugaagcugcaaacaaaggcucuuugucuugugucaaagagcauaccuauaagaucgagaugugcuauaaucaagc

SEQ ID NO: 473 - RNA, 103 nt, Tomato spotted wilt virus
gugcuauaaucaagcuuuaggcaaagugaauguucuaucuccuaacagaaauguccaugaauggcuguacaguuucaagccaaauuucaaucaaguugaaagc

SEQ ID NO: 474 - RNA, 104 nt, Tomato spotted wilt virus
guugaaagcaacaacagaacugugaauucucuugcagugaaaucucuacucaugucagcagaaaacaacaucaugccuaacucucaagcuuuugucaaagcuuc

SEQ ID NO: 475 - RNA, 98 nt, Tomato spotted wilt virus
gucaaagcuuccacugauucucauuucaagcugagccucuggcuaaggguuccaaaaguuuugaagcaaauuuccauucagaaauuguucaagguugc

SEQ ID NO: 476 - RNA, 97 nt, Tomato spotted wilt virus
gaaauuguucaagguugcaggagaugaaacaaauaaaacauuuuauuuaucuauugccugcauuccaaaccauaacaguguugagacagcuuuaaac

SEQ ID NO: 477 - RNA, 103 nt, Tomato spotted wilt virus
gagacagcuuuaaacauuacuguuauuugcaagcaucagcucccaauucguaaauguggagcuccuuuugaauuaucaaugauguuuucugauuuaaaggagc

SEQ ID NO: 478 - RNA, 102 nt, Tomato spotted wilt virus
gauuuaaaggagccuuacaacauuguucaugauccuucauauccccaaaggauuguccaugcucugcuugaaacucacacaucuuuugcacaaguucuuugc

SEQ ID NO: 479 - RNA, 99 nt, Tomato spotted wilt virus
gcacaaguucuuugcaacaacuugcaagaagaugugaucauuuacacuuugaacaaccaugagcuaacuccuagaaaguuagauuuaggugaaacaacc

SEQ ID NO: 480 - RNA, 104 nt, Tomato spotted wilt virus
gauuuaggugaaacaaccuugaauuacggugaagaugccuacaaaaggaaauauuuccuuucaaaaacacuugaaugucuuccaucuaacacacaaacuauuuc

SEQ ID NO: 481 - RNA, 100 nt, Tomato spotted wilt virus
gaaugucuuccaucuaacacacaaacuauuucuuauuuagacagcauccaaauccccuccuggaagauagacuuugccaggggagaaauuaaaauuucuc

SEQ ID NO: 482 - RNA, 109 nt, Tomato spotted wilt virus
gagaaauuaaaauuucuccgcaaucuauuucaguugcaaaaucuuuguuaaagcuugauuuaagcaggaucaaaaagaaagaaucuaagauuaaggaagcauaugcuuc

SEQ ID NO: 483 - RNA, 102 nt, Cucumber Mosaic Virus
gaaacuuguuucgcgcauucaaauucgaguuaaucccuugccgaaauuugauucuaccgugugggugacaguccguaaaguuccugccuccucggacuuauc